SEQUENCE LISTING (37 sequences)

SEQ ID NO: 1 - protein, 5 aa, synthetic construct
GGGGS

SEQ ID NO: 2 - protein, 10 aa, synthetic construct
GGGGSGGGGS

SEQ ID NO: 3 - protein, 15 aa, synthetic construct
GGGGSGGGGSGGGGS

SEQ ID NO: 4 - protein, 20 aa, synthetic construct
GGGGSGGGGSGGGGSGGGGS

SEQ ID NO: 5 - protein, 8 aa, synthetic construct
GGGGGGGG

SEQ ID NO: 6 - protein, 6 aa, synthetic construct
GGGGGG

SEQ ID NO: 7 - protein, 15 aa, synthetic construct
EAAAKEAAAKEAAAK

SEQ ID NO: 8 - protein, 4 aa, synthetic construct
EAAK

SEQ ID NO: 9 - protein, 8 aa, synthetic construct
EAAKEAAK

SEQ ID NO: 10 - protein, 12 aa, synthetic construct
EAAKEAAKEAAK

SEQ ID NO: 11 - protein, 46 aa, synthetic construct
AEAAAKEAAAKEAAAKEAAAKALEAEAAAKEAAAKEAAAKEAAAKA

SEQ ID NO: 12 - protein, 12 aa, synthetic construct
AEAAAKEAAAKA

SEQ ID NO: 13 - protein, 6 aa, synthetic construct
GGGGSG

SEQ ID NO: 14 - protein, 594 aa, synthetic construct
MASSTPSSSATSSNAGADPNTTNLRPTTYDTWCGVAHGCTRKLGLKICGFLQRTNSLEEKSRLVSAFKERQSSKNLLSCENSDRDARFRRTETDFSNLFARDLLPAKNGEEQTVQFLLEVVDILLNYVRKTFDRSTKVLDFHHPHQLLEGMEGFNLELSDHPESLEQILVDCRDTLKYGVRTGHPRFFNQLSTGLDIIGLAGEWLTSTANTNMFTYEIAPVFVLMEQITLKKMREIVGWSSKDGDGIFSPGGAISNMYSIMAARYKYFPEVKTKGMAAVPKLVLFTSEQSHYSIKKAGAALGFGTDNVILIKCNERGKIIPADFEAKILEAKQKGYVPFYVNATAGTTVYGAFDPIQEIADICEKYNLWLHVDAAWGGGLLMSRKHRHKLNGIERANSVTWNPHKMMGVLLQCSAILVKEKGILQGCNQMCAGYLFQPDKQYDVSYDTGDKAIQCGRHVDIFKFWLMWKAKGTVGFENQINKCLELAEYLYAKIKNREEFEMVFNGEPEHTNVCFWYIPQSLRGVPDSPQRREKLHKVAPKIKALMMESGTTMVGYQPQGDKANFFRMVISNPAATQSDIDFLIEEIERLGQDL

SEQ ID NO: 15 - protein, 585 aa, synthetic construct
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 16 - protein, 110 aa, synthetic construct
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 17 - protein, 355 aa, synthetic construct
MDFLHRNGVLIIQHLQKDYRAYYTFLNFMSNVGDPRNIFFIYFPLCFQFNQTVGTKMIWVAVIGDWLNLIFKWILFGHRPYWWVQETQIYPNHSSPCLEQFPTTCETGPGSPSGHAMGASCVWYVMVTAALSHTVCGMDKFSITLHRLTWSFLWSVFWLIQISVCISRVFIATHFPHQVILGVIGGMLVAEAFEHTPGIQTASLGTYLKTNLFLFLFAVGFYLLLRVLNIDLLWSVPIAKKWCANPDWIHIDTTPFAGLVRNLGVLFGLGFAINSEMFLLSCRGGNNYTLSFRLLCALTSLTILQLYHFLQIPTHEEHLFYVLSFCKSASIPLTVVAFIPYSVHMLMKQSGKKSQ

SEQ ID NO: 18 - protein, 369 aa, synthetic construct
MEFLERTYLVNDKAAKMYAFTLESVELQQKPVNKDQCPRERPEELESGGMYHCHSGSKPTEKGANEYAYAKWKLCSASAICFIFMIAEVVGGHIAGSLAVVTDAAHLLIDLTSFLLSLFSLWLSSKPPSKRLTFGWHRAEILGALLSILCIWVVTGVLVYLACERLLYPDYQIQATVMIIVSSCAVAANIVLTVVLHQRCLGHNHKEVQANASVRAAFVHALGDLFQSISVLISALIIYFKPEYKIADPICTFIFSILVLASTITILKDFSILLMEGVPKSLNYSGVKELILAVDGVLSVHSLHIWSLTMNQVILSAHVATAASRDSQVVRREIAKALSKSFTMHSLTIQMESPVDQDPDCLFCEDPCD

SEQ ID NO: 19 - protein, 979 aa, synthetic construct
MRRPRRPGGLGGSGGLRLLLCLLLLSSRPGGCSAVSAHGCLFDRRLCSHLEVCIQDGLFGQCQVGVGQARPLLQVTSPVLQRLQGVLRQLMSQGLSWHDDLTQYVISQEMERIPRLRPPEPRPRDRSGLAPKRPGPAGELLLQDIPTGSAPAAQHRLPQPPVGKGGAGASSSLSPLQAELLPPLLEHLLLPPQPPHPSLSYEPALLQPYLFHQFGSRDGSRVSEGSPGMVSVGPLPKAEAPALFSRTASKGIFGDHPGHSYGDLPGPSPAQLFQDSGLLYLAQELPAPSRARVPRLPEQGSSSRAEDSPEGYEKEGLGDRGEKPASPAVQPDAALQRLAAVLAGYGVELRQLTPEQLSTLLTLLQLLPKGAGRNPGGVVNVGADIKKTMEGPVEGRDTAELPARTSPMPGHPTASPTSSEVQQVPSPVSSEPPKAARPPVTPVLLEKKSPLGQSQPTVAGQPSARPAAEEYGYIVTDQKPLSLAAGVKLLEILAEHVHMSSGSFINISVVGPALTFRIRHNEQNLSLADVTQQAGLVKSELEAQTGLQILQTGVGQREEAAAVLPQTAHSTSPMRSVLLTLVALAGVAGLLVALAVALCVRQHARQQDKERLAALGPEGAHGDTTFEYQDLCRQHMATKSLFNRAEGPPEPSRVSSVSSQFSDAAQASPSSHSSTPSWCEEPAQANMDISTGHMILAYMEDHLRNRDRLAKEWQALCAYQAEPNTCATAQGEGNIKKNRHPDFLPYDHARIKLKVESSPSRSDYINASPIIEHDPRMPAYIATQGPLSHTIADFWQMVWESGCTVIVMLTPLVEDGVKQCDRYWPDEGASLYHVYEVNLVSEHIWCEDFLVRSFYLKNVQTQETRTLTQFHFLSWPAEGTPASTRPLLDFRRKVNKCYRGRSCPIIVHCSDGAGRTGTYILIDMVLNRMAKGVKEIDIAATLEHVRDQRPGLVRSKDQFEFALTAVAEEVNAILKALPQ

SEQ ID NO: 20 - protein, 285 aa, synthetic construct
MMEAIKKKMQMLKLDKENALDRAEQAEAEQKQAEERSKQLEDELAAMQKKLKGTEDELDKYSEALKDAQEKLELAEKKAADAEAEVASLNRRIQLVEEELDRAQERLATALQKLEEAEKAADESERGMKVIENRALKDEEKMELQEIQLKEAKHIAEEADRKYEEVARKLVIIEGDLERTEERAELAESKCSELEEELKNVTNNLKSLEAQAEKYSQKEDKYEEEIKILTDKLKEAETRAEFAERSVAKLEKTIDDLEDELYAQKLKYKAISEELDHALNDMTSI

SEQ ID NO: 21 - protein, 144 aa, synthetic construct
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 22 - protein, 1390 aa, synthetic construct
MVKEQFRETDVAKKISHICFGMKSPEEMRQQAHIQVVSKNLYSQDNQHAPLLYGVLDHRMGTSEKDRPCETCGKNLADCLGHYGYIDLELPCFHVGYFRAVIGILQMICKTCCHIMLSQEEKKQFLDYLKRPGLTYLQKRGLKKKISDKCRKKNICHHCGAFNGTVKKCGLLKIIHEKYKTNKKVVDPIVSNFLQSFETAIEHNKEVEPLLGRAQENLNPLVVLNLFKRIPAEDVPLLLMNPEAGKPSDLILTRLLVPPLCIRPSVVSDLKSGTNEDDLTMKLTEIIFLNDVIKKHRISGAKTQMIMEDWDFLQLQCALYINSELSGIPLNMAPKKWTRGFVQRLKGKQGRFRGNLSGKRVDFSGRTVISPDPNLRIDEVAVPVHVAKILTFPEKVNKANINFLRKLVQNGPEVHPGANFIQQRHTQMKRFLKYGNREKMAQELKYGDIVERHLIDGDVVLFNRQPSLHKLSIMAHLARVKPHRTFRFNECVCTPYNADFDGDEMNLHLPQTEEAKAEALVLMGTKANLVTPRNGEPLIAAIQDFLTGAYLLTLKDTFFDRAKACQIIASILVGKDEKIKVRLPPPTILKPVTLWTGKQIFSVILRPSDDNPVRANLRTKGKQYCGKGEDLCANDSYVTIQNSELMSGSMDKGTLGSGSKNNIFYILLRDWGQNEAADAMSRLARLAPVYLSNRGFSIGIGDVTPGQGLLKAKYELLNAGYKKCDEYIEALNTGKLQQQPGCTAEETLEALILKELSVIRDHAGSACLRELDKSNSPLTMALCGSKGSFINISQMIACVGQQAISGSRVPDGFENRSLPHFEKHSKLPAAKGFVANSFYSGLTPTEFFFHTMAGREGLVDTAVKTAETGYMQRRLVKSLEDLCSQYDLTVRSSTGDIIQFIYGGDGLDPAAMEGKDEPLEFKRVLDNIKAVFPCPSEPALSKNELILTTESIMKKSEFLCCQDSFLQEIKKFIKGVSEKIKKTRDKYGINDNGTTEPRVLYQLDRITPTQVEKFLETCRDKYMRAQMEPGSAVGALCAQSIGEPGTQMTLKTFHFAGVASMNITLGVPRIKEIINASKAISTPIITAQLDKDDDADYARLVKGRIEKTLLGEISEYIEEVFLPDDCFILVKLSLERIRLLRLEVNAETVRYSICTSKLRVKPGDVAVHGEAVVCVTPRENSKSSMYYVLQFLKEDLPKVVVQGIPEVSRAVIHIDEQSGKEKYKLLVEGDNLRAVMATHGVKGTRTTSNNTYEVEKTLGIEAARTTIINEIQYTMVNHGMSIDRRHVMLLSDLMTYKGEVLGITRFGLAKMKESVLMLASFEKTADHLFDAAYFGQKDSVCGVSECIIMGIPMNIGTGLFKLLHKADRDPNPPKRPLIFDTNEFHIPLVT

SEQ ID NO: 23 - protein, 1133 aa, synthetic construct
MDVLAEEFGNLTPEQLAAPIPTVEEKWRLLPAFLKVKGLVKQHIDSFNYFINVEIKKIMKANEKVTSDADPMWYLKYLNIYVGLPDVEESFNVTRPVSPHECRLRDMTYSAPITVDIEYTRGSQRIIRNALPIGRMPIMLRSSNCVLTGKTPAEFAKLNECPLDPGGYFIVKGVEKVILIQEQLSKNRIIVEADRKGAVGASVTSSTHEKKSRTNMAVKQGRFYLRHNTLSEDIPIVIIFKAMGVESDQEIVQMIGTEEHVMAAFGPSLEECQKAQIFTQMQALKYIGNKVRRQRMWGGGPKKTKIEEARELLASTILTHVPVKEFNFRAKCIYTAVMVRRVILAQGDNKVDDRDYYGNKRLELAGQLLSLLFEDLFKKFNSEMKKIADQVIPKQRAAQFDVVKHMRQDQITNGMVNAISTGNWSLKRFKMDRQGVTQVLSRLSYISALGMMTRISSQFEKTRKVSGPRSLQPSQWGMLCPSDTPEGEACGLVKNLALMTHITTDMEDGPIVKLASNLGVEDVNLLCGEELSYPNVFLVFLNGNILGVIRDHKKLVNTFRLMRRAGYINEFVSISTNLTDRCVYISSDGGRLCRPYIIVKKQKPAVTNKHMEELAQGYRNFEDFLHESLVEYLDVNEENDCNIALYEHTINKDTTHLEIEPFTLLGVCAGLIPYPHHNQSPRNTYQCAMGKQAMGTIGYNQRNRIDTLMYLLAYPQKPMVKTKTIELIEFEKLPAGQNATVAVMSYSGYDIEDALVLNKASLDRGFGRCLVYKNAKCTLKRYTNQTFDKVMGPMLDAATRKPIWRHEILDADGICSPGEKVENKQVLVNKSMPTVTQIPLEGSNVPQQPQYKDVPITYKGATDSYIEKVMISSNAEDAFLIKMLLRQTRRPEIGDKFSSRHGQKGVCGLIVPQEDMPFCDSGICPDIIMNPHGFPSRMTVGKLIELLAGKAGVLDGRFHYGTAFGGSKVKDVCEDLVRHGYNYLGKDYVTSGITGEPLEAYIYFGPVYYQKLKHMVLDKMHARARGPRAVLTRQPTEGRSRDGGLRLGEMERDCLIGYGASMLLLERLMISSDAFEVDVCGQCGLLGYSGWCHYCKSSCHVSSLRIPYACKLLFQELQSMNIIPRLKLSKYNE

SEQ ID NO: 24 - protein, 534 aa, synthetic construct
MTQAEIKLCSLLLQEHFGEIVEKIGVHLIRTGSQPLRVIAHDTGTSLDQVKKALCVLVQHNLVSYQVHKRGVVEYEAQCSRVLRMLRYPRYIYTTKTLYSDTGELIVEELLLNGKLTMSAVVKKVADRLTETMEDGKTMDYAEVSNTFVRLADTHFVQRCPSVPTTENSDPGPPPPAPTLVINEKDMYLVPKLSLIGKGKRRRSSDEDAAGEPKAKRPKYTTDNKEPIPDDGIYWQANLDRFHQHFRDQAIVSAVANRMDQTSSEIVRTMLRMSEITTSSSAPFTQPLSSNEIFRSLPVGYNISKQVLDQYLTLLADDPLEFVGKSGDSGGGMYVINLHKALASLATATLESVVQERFGSRCARIFRLVLQKKHIEQKQVEDFAMIPAKEAKDMLYKMLSENFMSLQEIPKTPDHAPSRTFYLYTVNILSAARMLLHRCYKSIANLIERRQFETKENKRLLEKSQRVEAIIASMQATGAEEAQLQEIEEMITAPERQQLETLKRNVNKLDASEIQVDETIFLLESYIECTMKRQ

SEQ ID NO: 25 - protein, 398 aa, synthetic construct
MSEGNAAGEPSTPGGPRPLLTGARGLIGRRPAPPLTPGRLPSIRSRDLTLGGVKKKTFTPNIISRKIKEEPKEEVTVKKEKRERDRDRQREGHGRGRGRPEVIQSHSIFEQGPAEMMKKKGNWDKTVDVSDMGPSHIINIKKEKRETDEETKQILRMLEKDDFLDDPGLRNDTRNMPVQLPLAHSGWLFKEENDEPDVKPWLAGPKEEDMEVDIPAVKVKEEPRDEEEEAKMKAPPKAARKTPGLPKDVSVAELLRELSLTKEEELLFLQLPDTLPGQPPTQDIKPIKTEVQGEDGQVVLIKQEKDREAKLAENACTLADLTEGQVGKLLIRKSGRVQLLLGKVTLDVTMGTACSFLQELVSVGLGDSRTGEMTVLGHVKHKLVCSPDFESLLDHKHR

SEQ ID NO: 26 - protein, 708 aa, synthetic construct
MANEEDDPVVQEIDVYLAKSLAEKLYLFQYPVRPASMTYDDIPHLSAKIKPKQQKVELEMAIDTLNPNYCRSKGEQIALNVDGACADETSTYSSKLMDKQTFCSSQTTSNTSRYAAALYRQGELHLTPLHGILQLRPSFSYLDKADAKHREREAANEAGDSSQDEAEDDVKQITVRFSRPESEQARQRRVQSYEFLQKKHAEEPWVHLHYYGLRDSRSEHERQYLLCPGSSGVENTELVKSPSEYLMMLMPPSQEEEKDKPVAPSNVLSMAQLRTLPLADQIKILMKNVKVMPFANLMSLLGPSIDSVAVLRGIQKVAMLVQGNWVVKSDILYPKDSSSPHSGVPAEVLCRGRDFVMWKFTQSRWVVRKEVATVTKLCAEDVKDFLEHMAVVRINKGWEFILPYDGEFIKKHPDVVQRQHMLWTGIQAKLEKVYNLVKETMPKKPDAQSGPAGLVCGDQRIQVAKTKAQQNHALLERELQRRKEQLRVPAVPPGVRIKEEPVSEEGEEDEEQEAEEEPMDTSPSGLHSKLANGLPLGRAAGTDSFNGHPPQGCASTPVARELKAFVEATFQRQFVLTLSELKRLFNLHLASLPPGHTLFSGISDRMLQDTVLAAGCKQILVPFPPQTAASPDEQKVFALWESGDMSDQHRQVLLEIFSKNYRVRRNMIQSRLTQECGEDLSKQEVDKVLKDCCVSYGGMWYLKGTVQS

SEQ ID NO: 27 - protein, 316 aa, synthetic construct
MAEVKVKVQPPDADPVEIENRIIELCHQFPHGITDQVIQNEMPHIEAQQRAVAINRLLSMGQLDLLRSNTGLLYRIKDSQNAGKMKGSDNQEKLVYQIIEDAGNKGIWSRDIRYKSNLPLTEINKILKNLESKKLIKAVKSVAASKKKVYMLYNLQPDRSVTGGAWYSDQDFESEFVEVLNQQCFKFLQSKAETARESKQNPMIQRNSSFASSHEVWKYICELGISKVELSMEDIETILNTLIYDGKVEMTIIAAKEGTVGSVDGHMKLYRAVNPIIPPTGLVRAPCGLCPVFDDCHEGGEISPSNCIYMTEWLEF

SEQ ID NO: 28 - protein, 223 aa, synthetic construct
MAGNKGRGRAAYTFNIEAVGFSRGEKLPDVVLKPPPLFPDTDYKPVPLKTGEDEDYMLALKQELRETVKRLPYFIEPPEEKQDDIERYSKRYMKVYKEEWVPDWRRLPREMMPRKKCKKGDPKSKPSKAAAKATSLINSADVLKTIEELEKRGEGERSDEENEEKEGSKEKDKDDEEDGEEDAEQEDYDEEEQEEENDYINSYFDNGDDFGVDSDDNMDEATY

SEQ ID NO: 29 - protein, 204 aa, synthetic construct
MFVLVEMVDTVRIPPWQFERKLNDSIAEELNKKLANKVVYNVGLCICLFDITKLEDAYVFPGDGASHTKVHFRCVVFHPFLDEILIGKIKGCSPEGVHVSLGFFDDILIPPESLQQPAKFDEAEQVWVWEYETEEGAHDLYMDTGEEIRFRVVDESFVDTSPTGPSSADATTSSEELPKKEAPYTLVGSISEPGLGLLSWWTSN

SEQ ID NO: 30 - protein, 108 aa, synthetic construct
MLLFCPGCGNGLIVEEGQRCHRFACNTCPYVHNITRKVTNRKYPKLKEVDDVLGGAAAWENVDSTAESCPKCEHPRAYFMQLQTRSADEPMTTFYKCCNAQCGHRWRD

SEQ ID NO: 31 - protein, 765 aa, synthetic construct
MSGDHLHNDSQIEADFRLNDSHKHKDKHKDREHRHKEHKKEKDREKSKHSNSEHKDSEKKHKEKEKTKHKDGSSEKHKDKHKDRDKEKRKEEKVRASGDAKIKKEKENGFSSPPQIKDEPEDDGYFVPPKEDIKPLKRPRDEDDADYKPKKIKTEDTKKEKKRKLEEEEDGKLKKPKNKDKDKKVPEPDNKKKKPKKEEEQKWKWWEEERYPEGIKWKFLEHKGPVFAPPYEPLPENVKFYYDGKVMKLSPKAEEVATFFAKMLDHEYTTKEIFRKNFFKDWRKEMTNEEKNIITNLSKCDFTQMSQYFKAQTEARKQMSKEEKLKIKEENEKLLKEYGFCIMDNHKERIANFKIEPPGLFRGRGNHPKMGMLKRRIMPEDIIINCSKDAKVPSPPPGHKWKEVRHDNKVTWLVSWTENIQGSIKYIMLNPSSRIKGEKDWQKYETARRLKKCVDKIRNQYREDWKSKEMKVRQRAVALYFIDKLALRAGNEKEEGETADTVGCCSLRVEHINLHPELDGQEYVVEFDFLGKDSIRYYNKVPVEKRVFKNLQLFMENKQPEDDLFDRLNTGILNKHLQDLMEGLTAKVFRTYNASITLQQQLKELTAPDENIPAKILSYNRANRAVAILCNHQRAPPKTFEKSMMNLQTKIDAKKEQLADARRDLKSAKADAKVMKDAKTKKVVESKKKAVQRLEEQLMKLEVQATDREENKQIALGTSKLNYLDPRITVAWCKKWGVPIEKIYNKTQREKFAWAIDMADEDYEF

SEQ ID NO: 32 - protein, 359 aa, synthetic construct [sequence omitted]

SEQ ID NO: 33 - protein, 427 aa, synthetic construct
METLCLRASFWLALVGCVISDNPERYSTNLSNHVDDFTTFRGTELSFLVTTHQPTNLVLPSNGSMHNYCPQQTKITSAFKYINTVISCTIFIVGMVGNATLLRIIYQNKCMRNGPNALIASLALGDLIYVVIDLPINVFKLLAGRWPFDHNDFGVFLCKLFPFLQKSSVGITVLNLCALSVDRYRAVASWSRVQGIGIPLVTAIEIVSIWILSFILAIPEAIGFVMVPFEYRGEQHKTCMLNATSKFMEFYQDVKDWWLFGFYFCMPLVCTAIFYTLMTCEMLNRRNGSLRIALSEHLKQRREVAKTVFCLVVIFALCWFPLHLSRILKKTVYNEMDKNRCELLSFLLLMDYIGINLATMNSCINPIALYFVSKKFKNCFQSCLCCCCYQSKSLMTSVPMNGTSIQWKNHDQNNHNTDRSSHKDSMN

SEQ ID NO: 34 - protein, 140 aa, synthetic construct
MGPRRRSRKPEAPRRRSPSPTPTPGPSRRGPSLGASSHQHSRRRQGWLKEIRKLQKSTHLLIRKLPFSRLAREICVKFTRGVDFNWQAQALLALQEAAEAFLVHLFEDAYLLTLHAGRVTLFPKDVQLARRIRGLEEGLG

SEQ ID NO: 35 - protein, 599 aa, synthetic construct
MGPKRRQLTFREKSRIIQEVEENPDLRKGEIARRFNIPPSTLSTILKNKRAILASERKYGVASTCRKTNKLSPYDKLEGLLIAWFQQIRAAGLPVKGIILKEKALRIAEELGMDDFTASNGWLDRFRRRHGVVSCSGVARARARNAAPRTPAAPASPAAVPSEGSGGSTTGWRAREEQPPSVAEGYASQDVFSATETSLWYDFLPDQAAGLCGGDGRPRQATQRLSVLLCANADGSEKLPPLVAGKSAKPRAGQAGLPCDYTANSKGGVTTQALAKYLKALDTRMAAESRRVLLLAGRLAAQSLDTSGLRHVQLAFFPPGTVHPLERGVVQQVKGHYRQAMLLKAMAALEGQDPSGLQLGLTEALHFVAAAWQAVEPSDIAACFREAGFGGGPNATITTSLKSEGEEEEEEEEEEEEEEGEGEEEEEEGEEEEEEGGEGEELGEEEEVEEEGDVDSDEEEEEDEESSSEGLEAEDWAQGVVEAGGSFGAYGAQEEAQCPTLHFLEGGEDSDSDSEEEDDEEEDDEDEDDDDDEEDGDEVPVPSFGEAMAYFAMVKRYLTSFPIDDRVQSHILHLEHDLVHVTRKNHARQAGVRGLGHQS

SEQ ID NO: 36 - protein, 943 aa, synthetic construct
MAASGLDHLKNGYRRRFCRPSRARDINTEQGQNVLEILQDCFEEKSLANDFSTNSTKSVPNSTRKIKDTCIQSPSKECQKSHPKSVPVSSKKKEASLQFVVEPSEATNRSVQAHEVHQKILATDVSSKNTPDSKKISSRNINDHHSEADEEFYLSVGSPSVLLDAKTSVSQNVIPSSAQKRETYTFENSVNMLPSSTEVSVKTKKRLNFDDKVMLKKIEIDNKVSDEEDKTSEGQERKPSGSSQNRIRDSEYEIQRQAKKSFSTLFLETVKRKSESSPIVRHAATAPPHSCPPDDTKLIEDEFIIDESDQSFASRSWITIPRKAGSLKQRTISPAESTALLQGRKSREKHHNILPKTLANDKHSHKPHPVETSQPSDKTVLDTSYALIGETVNNYRSTKYEMYSKNAEKPSRSKRTIKQKQRRKFMAKPAEEQLDVGQSKDENIHTSHITQDEFQRNSDRNMEEHEEMGNDCVSKKQMPPVGSKKSSTRKDKEESKKKRFSSESKNKLVPEEVTSTVTKSRRISRRPSDWWVVKSEESPVYSNSSVRNELPMHHNSSRKSTKKTNQSSKNIRKKTIPLKRQKTATKGNQRVQKFLNAEGSGGIVGHDEISRCSLSEPLESDEADLAKKKNLDCSRSTRSSKNEDNIMTAQNVPLKPQTSGYTCNIPTESNLDSGEHKTSVLEESGPSRLNNNYLMSGKNDVDDEEVHGSSDDSKQSKVIPKNRIHHKLVLPSNTPNVRRTKRTRLKPLEYWRGERIDYQGRPSGGFVISGVLSPDTISSKRKAKENIGKVNKKSNKKRICLDNDERKTNLMVNLGIPLGDPLQPTRVKDPETREIILMDLVRPQDTYQFFVKHGELKVYKTLDTPFFSTGKLILGPQEEKGKQHVGQDILVFYVNFGDLLCTLHETPYILSTGDSFYVPSGNYYNIKNLRNEESVLLFTQIKR

SEQ ID NO: 37 - protein, 249 aa, synthetic construct
MASRRMETKPVITCLKTLLIIYSFVFWITGVILLAVGVWGKLTLGTYISLIAENSTNAPYVLIGTGTTIVVFGLFGCFATCRGSPWMLKLYAMFLSLVFLAELVAGISGFVFRHEIKDTFLRTYTDAMQTYNGNDERSRAVDHVQRSLSCCGVQNYTNWSTSPYFLEHGIPPSCCMNETDCNPQDLHNLTVAATKVNQKGCYDLVTSFMETNMGIIAGVAFGIAFSQLIGMLLACCLSRFITANQYEMV

(19) United States
(12) Patent Application Publication
(10) Pub. No.: US 2024/0342260 A1
(43) Pub. Date: Oct. 17, 2024

(54) TISSUE-DIRECTED ANTIBODIES AND METHODS OF USING THE SAME

(71) Applicant: ZAG Bio, Inc., Cambridge, MA (US)

(72) Inventors: Alan Crane, Cambridge, MA (US); John Kulman, Cambridge, MA (US); Joanne L. Viney, Cambridge, MA (US)

(21) Appl. No.: 18/638,402

(22) Filed: Apr. 17, 2024

Related U.S. Application Data
(60) Provisional application No. 63/496,535, filed on Apr. 17, 2023.

(51) Int. Cl. A61K 39/00; A61P 37/06; C07K 14/47; C07K 14/705; C07K 16/18; C07K 16/26; C07K 16/28; C07K 16/30

(52) U.S. Cl. CPC A61K 39/0008; A61P 37/06; C07K 14/4713; C07K 14/70503; C07K 16/18; C07K 16/26; C07K 16/28; C07K 16/2803; C07K 16/283; C07K 16/2833; C07K 16/2851; C07K 16/2896; C07K 16/30; A61K 2039/505; A61K 2039/577; A61K 2039/6056; C07K 2317/52; C07K 2317/71; C07K 2319/33; C07K 2319/40

(57) ABSTRACT

Embodiments provided herein, provide for polypeptides, pharmaceutical compositions, and methods that can be used to target at least two types of cells to modulate the activity of the same to treat disorders, such as autoimmune disorders.

### CROSS-REFERENCE TO RELATED APPLICATIONS

This application claims priority to U.S. Provisional Application No. 63/496,535, filed Apr. 17, 2023, which is hereby incorporated by reference in its entirety.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on Apr. 17, 2024, is named “ZAG-001WO_SEQ.xml” and is 44,802 bytes in size.

### FIELD

The embodiments provided herein relate to compositions that target different cells to regulate an immune response.

### BACKGROUND

The thymus is the primary lymphoid organ responsible for T cell development and education. The development of T cells that bear the αβ form of the T cell receptor occurs exclusively within the thymus. Intrathymic T cell maturation proceeds from fetal liver- or bone marrow-derived haematopoietic stem cells and occurs via a differentiation program readily characterized by changes to cell surface phenotype, proliferation status and functionality. Key events in T cell development include lineage commitment, selection events, and thymic emigration. Thymic epithelial cells (TECs) are a key component of the thymic stroma. TECs in the thymic cortex (cTECs) are specialized for T cell positive selection and ensure that mature T cells are self-MHC restricted. Medullary TECs (mTECs) or dendritic cells are involved in T cell negative selection wherein apoptosis is induced in T cells showing a high degree of self-avidity (Klein, L., Hinterberger, M., Wirnsberger, G., & Kyewski, B. (2009). Antigen presentation in the thymus for positive selection and central tolerance induction. Nature Reviews Immunology, 9(12), 833-844. https://doi.org/10.1038/nri2669; and Hinterberger, M., Aichinger, M., Prazeres da Costa, O., Voehringer, D., Hoffmann, R., & Klein, L. (2010). Autonomous role of medullary thymic epithelial cells in central CD4+ T cell tolerance. Nature Immunology, 11(6), 512-519. https://doi.org/10.1038/ni.1874; which are hereby incorporated by reference in their entirety). mTECs play a vital role in shaping the T cell repertoire by promoting the conversion of antigen-specific thymocytes into antigen-specific regulatory T cells (Tregs), which in turn suppress the immune response to self-antigens in peripheral tissues. mTECs achieve this by expressing and presenting tissue-specific antigens (TSAs) to developing thymocytes, leading to the selection of T cells that recognize these antigens as self. Thymocytes that recognize self-antigens with high affinity are either eliminated through negative selection or converted into Tregs through a process called agonist selection. The resulting antigen-specific Tregs migrate to the peripheral tissues, where they exert a bystander effect by suppressing immune responses to self-antigens, thereby maintaining self-tolerance and preventing autoimmune diseases. Thus, TEC-mediated selection promotes a self-tolerant and highly diverse T cell repertoire that can recognize foreign antigens presented by self-MHC molecules. Together, positive and negative selection provide a balance to create a broadly reactive T cell receptor repertoire with a low—but not absent—potential for self-reactivity. Regulation of self-reactive T cells is an especially attractive approach for the treatment of autoimmune diseases. The embodiments provided for herein fulfill these needs as well as others.

### SUMMARY

In some embodiments, molecules for inducing antigen-specific thymic Tregs against an antigen of interest are provided, which can, for example be administered to a subjectsystemically. In some embodiments, the molecules comprise a thymic cell targeting domain linked to an antigen of interest or a domain that binds to the antigen of interest.

In some embodiments, molecules comprising a cell targeting domain linked to an antigen of interest or a domain that binds to the antigen of interest are provided. In some embodiments, the cell targeting domain binds to a thymic cell. In some embodiments, the cell targeting domain binds specifically to a thymic cell.

In some embodiments, provided herein is a molecule comprising a cell targeting domain linked to an antigen of interest or a domain that binds to the antigen of interest, wherein the cell targeting domain binds to a thymic cell, a splenic cell, a dendritic cell, or a B cell.

In some embodiments, provided herein is a molecule for inducing tolerance against an antigen of interest, wherein the molecule comprises a cell targeting domain linked to the antigen of interest or a domain that binds to the antigen of interest, wherein the targeting domain binds to a thymic cell, a splenic cell, a dendritic cell, or a B cell.

In some embodiments, provided herein is a molecule for inducing antigen-specific Tregs against an antigen of interest, wherein the molecule comprises a cell targeting domain linked to the antigen of interest or a domain that binds to the antigen of interest, wherein the targeting domain binds to a thymic cell, a splenic cell, a dendritic cell, or a B cell.

In some embodiments, provided herein is a molecule for inducing antigen-specific Tregs against an antigen of interest, wherein the molecule comprises a cell targeting domain linked to the antigen of interest or a domain that binds to the antigen of interest, wherein the targeting domain binds to a thymic cell, a splenic cell, a dendritic cell, or a B cell, wherein the molecule does not significantly induce antigen-specific T_{eff} cells.

In some embodiments, provided herein is a molecule comprising: a) a cell targeting domain that binds to cadherin-1 (CDH1), Trop2, CD74, EpCAM, syndecan 1 (SDC1), PlgR, CD23 antigen (FCER2), macrophage receptor MARCO, C-type lectin domain family 7 (CLEC7A), CD24, CD70, sialic acid-binding Ig-like lectin 10 (SIGLEC10), milk fat globule EGF and factor V/VIII domain containing (MFGE8), EGF like repeats and discoidin domains 3 (EDIL3), growth arrest-specific gene 6 (GAS6), T-cell membrane protein 4 (TIM4); b) an autoimmune, alloimmune, allergic, inflammatory, or anti-drug immune response associated antigen of interest; and c) optionally an Fc region, wherein the cell targeting domain binds to a thymic cell, a splenic cell, a dendritic cell, or a B cell.

In some embodiments, provided herein are pharmaceutical compositions comprising the same. In some embodiments, provided herein are methods of using the same.

### BRIEF DESCRIPTION OF THE DRAWINGS

FIG. 1 illustrates non-limiting embodiments as provided for herein.

FIG. 2A shows changes to population (shown as percentage CD4+ FoxP3+ T cells of CD4+ T cells) of CD4+ FoxP3+ T cells following treatment.

FIG. 2B shows changes to population (shown as percentage of live CD4+ T cells) of CD4+ T cells following treatment.

FIG. 3 shows changes to populations of CD4+ T cells and CD4+ FoxP3+ T cells following treatment.

FIG. 4A shows changes to population of CD4+ FoxP3+ T cells following treatment.

FIG. 4B shows changes to population of CD4+ T cells following treatment.

FIG. 5A shows changes to population of CD4+ FoxP3+ T cells following treatment.

FIG. 5B shows changes to population of CD4+ T cells following treatment.

FIG. 6A shows changes to population of CD4+ T cells following treatment.

FIG. 6B shows changes to population of CD4+ FoxP3+ T cells following treatment.

FIG. 7A shows changes to population of CD4+ T cells following treatment.

FIG. 7B shows changes to population of CD4+ FoxP3+ T cells following treatment.

FIG. 8A shows changes to population of CD4+ T cells at days 3 and 10 following treatment.

FIG. 8B shows changes to population of CD4+ FoxP3+ T cells at days 3 and 10 following treatment.

FIG. 9A shows changes to EAE score following treatment.

FIG. 9B shows changes to EAE score following treatment.

FIG. 10A shows changes to population of CD4+ T cells following treatment.

FIG. 10B shows changes to population of CD4+ FoxP3+ T cells following treatment.

FIG. 10C shows changes to absolute numbers (shown as total number of CD4+ T cells) of CD4+ T cells following treatment.

FIG. 10D shows changes to absolute numbers of CD4+ FoxP3+ T cells following treatment.

FIG. 11A shows changes to population of CD4+ FoxP3+ T cells following treatment.

FIG. 11B shows changes to population of CD4+ FoxP3+CD73− T cells following treatment.

FIG. 11C shows changes to absolute numbers of CD4+ FoxP3+ T cells following treatment.

FIG. 11D shows changes to absolute numbers of CD4+ FoxP3+CD73− T cells following treatment.

FIG. 12A shows changes to population of CD4+ T cells following treatment.

FIG. 12B shows changes to population of CD4+ FoxP3+ T cells following treatment.

FIG. 12C shows changes to absolute numbers of CD4+ T cells following treatment.

FIG. 12D shows changes to absolute numbers of CD4+ FoxP3+ T cells following treatment.

FIG. 13A shows changes to population of CD4+ T cells following treatment.

FIG. 13B shows changes to absolute numbers of CD4+ T cells following treatment.

FIG. 13C shows changes to population of CD4+ FoxP3+ T cells following treatment.

FIG. 13D shows changes to absolute numbers of CD4+ FoxP3+ T cells following treatment.

FIG. 13E shows changes to population of CD4+ FoxP3+CD73− T cells following treatment.

FIG. 13F shows changes to absolute numbers of CD4+ FoxP3+CD73− T cells following treatment.

FIG. 14A shows changes to population of CD4+ T cells following treatment.

FIG. 14B shows changes to population of CD4+ FoxP3+ T cells following treatment.

FIG. 14C shows changes to absolute numbers of CD4+ T cells following treatment.

FIG. 14D shows changes to absolute numbers of CD4+ FoxP3+ T cells following treatment.

FIG. 15A shows changes to population of CD4+ T cells following treatment.

FIG. 15B shows changes to absolute numbers of CD4+ T cells following treatment.

FIG. 15C shows changes to population of CD4+ FoxP3+ T cells following treatment.

FIG. 15D shows changes to absolute numbers of CD4+ FoxP3+ T cells following treatment.

FIG. 15E shows changes to population of CD4+ FoxP3+CD73− T cells following treatment.

FIG. 15F shows changes to absolute numbers of CD4+ FoxP3+CD73− T cells following treatment.

FIG. 16A shows changes to population of CD4+ T cells following treatment.

FIG. 16B shows changes to population of CD4+ FoxP3+ T cells following treatment.

FIG. 16C shows changes to absolute numbers of CD4+ T cells following treatment.

FIG. 16D shows changes to absolute numbers of CD4+ FoxP3+ T cells following treatment.

FIG. 17A shows changes to population of CD4+ T cells following treatment.

FIG. 17B shows changes to absolute numbers of CD4+ T cells following treatment.

FIG. 17C shows changes to population of CD4+ FoxP3+ T cells following treatment.

FIG. 17D shows changes to absolute numbers of CD4+ FoxP3+ T cells following treatment.

FIG. 17E shows changes to population of CD4+ FoxP3+CD73− T cells following treatment.

FIG. 17F shows changes to absolute numbers of CD4+ FoxP3+CD73− T cells following treatment.

FIG. 18A shows changes to EAE score following treatment.

FIG. 18B shows changes to EAE score following treatment.

FIG. 19A shows changes to EAE score following treatment.

FIG. 19B shows changes to EAE score following treatment.

FIG. 19C shows changes to EAE score following treatment.

### DETAILED DESCRIPTION

As used herein and unless otherwise indicated, the term “about” means that the numerical value is approximate and small variations would not significantly affect the practice of the disclosed embodiment. Where a numerical limitation is used, unless indicated otherwise by the context, “about” means the numerical value can vary by ±10% and remain within the scope of the disclosed embodiments.

As used herein and in the appended claims, the singular forms “a”, “an” and “the” include plural reference unless the context clearly dictates otherwise.

As used herein, the term “animal” includes, but is not limited to, humans and non-human vertebrates such as wild, domestic, and farm animals. Accordingly, as used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “antigen” refers to any molecule that the immune system can recognize and respond to. In some embodiments, antigen refers to an antigen of interest, such as a polypeptide, a molecule, a substance, an antibody, a ligand, a payload, or similar.

As used herein, the term “contacting” means bringing together of two elements in an in vitro system or an in vivo system. For example, “contacting” a therapeutic compound with an individual or patient or cell includes the administration of the compound or composition to an individual or patient, such as a human, as well as, for example, introducing a compound into a sample containing a cellular or purified preparation containing target.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any composition or method that recites the term “comprising” should also be understood to also describe such compositions as consisting, consisting of, or consisting essentially of the recited components or elements.

As used herein, the term “individual,” “subject,” or “patient,” which can be used interchangeably, means any animal, including mammals, such as mice, rats, other rodents, rabbits, dogs, cats, swine, cattle, sheep, horses, or primates, such as humans.

As used herein, the term “inhibit” refers to a result, symptom, or activity being reduced as compared to the activity or result in the absence of the compound that is inhibiting the result, symptom, or activity. In some embodiments, the result, symptom, or activity, is inhibited by about, or, at least, 10%, 20%, 30%, 40%, 50%, 60%, 70%, 80%, 90%, 95%, or 99%. A result, symptom, or activity can also be inhibited if it is completely eliminated or extinguished.

As used herein, the phrase “in need thereof” means that the subject has been identified as having a need for the particular method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, or condition is prevalent.

As used herein, the phrase “integer from X to Y” means any integer that includes the endpoints. For example, the phrase “integer from 1 to 5” means 1, 2, 3, 4, or 5.

As used herein, the phrase “self-antigen” refers to any polypeptide, protein or molecule that is produced by an subject's own body and is typically recognized as “self” by the immune system. A “self-antigen”, can also refer to as a tissue antigen, and means a polypeptide, protein or molecule that is produced by an individual's own body and is present within or on the surface of the cells of a specific tissue or organ. These tissue antigens are recognized as “self” by the immune system and are typically tolerated without provoking an immune response.

“Specific binding” or “specifically binds to” or is “specific for” a particular antigen, target, or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen, target, or an epitope can be exhibited, for example, by an antibody, or the antigen-binding fragment thereof, having a K_{D} for an antigen or epitope of at least about 10⁻⁴M, at least about 10⁻⁵M, at least about 10⁻⁶ M, at least about 10⁻⁷M, at least about 10⁻⁸M, at least about 10⁻⁹M, alternatively at least about 10⁻¹⁰ M, at least about 10⁻¹¹M, at least about 10⁻¹²M, or lesser, where K_{D} refers to the equilibrium dissociation constant of a particular antibody, or the antigen-binding fragment thereof, -target interaction. Typically, an antibody, or the antigen-binding fragment thereof, that specifically binds an antigen or target will have a K_{D} that is, or at least, 2-, 4-, 5-, 10-, 20-, 50-, 100-, 500-, 1000-, 5,000-, 10,000-, or more times greater for a control molecule relative to the antigen or epitope.

In some embodiments, specific binding for a particular antigen, target, or an epitope can be exhibited, for example, by an antibody, or the antigen-binding fragment thereof, having a K_{A}for a target, antigen, or epitope of at least 2-, 4-, 5-, 20-, 50-, 100-, 500-, 1000-, 5,000-, 10,000-or more times greater for the target, antigen, or epitope relative to a control, where K_{A} refers to the equilibrium association constant of a particular antibody, or the antigen-binding fragment thereof, -antigen interaction.

As provided herein, the compounds and compositions provided for herein can be used in methods of treatment as provided herein. As used herein, the terms “treat,” “treated,” or “treating” mean both therapeutic treatment and prophylactic measures wherein the object is to slow down (lessen) an undesired physiological condition, disorder or disease, or obtain beneficial or desired clinical results. For purposes of these embodiments, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival, as applicable for a specific disease, as compared to expected survival if not receiving treatment. Thus, “treatment of an autoimmune condition” or “treating autoimmunity” means an activity that alleviates or ameliorates any of the primary phenomena or secondary symptoms associated with the autoimmune condition other condition described herein when the terms “treat,” “treated,” or “treating” are used in conjunction with such condition.

As used herein, the term “thymic cells” refers to cells that ordinarily form part of, or are located in, the thymus, including, but not limited to, those listed in Table 1. Within the general category of thymic cells are both thymic stromal cells and cells of hematopoietic origin (such as T cell progenitors, immature T cells, and mature T cells) that reside within the thymus for at least some period of time—for example as part of the T cell maturation and T cell education processes that occur within the thymus.

TABLE 1

| Thymic Cells | Cell Marker Profile | Cell Type |
|---|---|---|
| Hematopoietic | CD45+ | T cell progenitors and T cells (mature |
| Lineage Cells | | and immature) |
| | CD45+ CD3+ | T cells (mature) |
| | CD45+ CD3+ CD4+ | CD4+ T cells (mature) |
| | CD45+ CD3+ CD8+ | CD8+ T cells (mature) |
| | CD45+ CD3+ CD4+ CD8+ | Double-positive T cells (DP) |
| | | (mature) |
| | CD45+ CD4− CD8− | Double-negative T cells (DN) |
| | | (immature) |
| | CD45+ CD4− CD8− CD44+ CD25− | Double-negative type 1 cells (DN1) |
| | | (immature) |
| | CD45+ CD4− CD8− CD44+ CD25+ | Double-negative type 2 cells (DN2) |
| | | (immature) |
| | CD45+ CD4− CD8− CD44− CD25+ | Double-negative type 3 cells (DN3) |
| | | (immature) |
| | CD45+ CD4− CD8− CD44− CD25− | Double-negative type cells (DN4) |
| | | (immature) |
| Epithelial | CD45− EpCAM+ Sca1+ (α6+ or α6−) | Thymic epithelial progenitor cells |
| Cells | | (TEPC) |
| | CD45− EpCAM+ Sca1+ (α6+ or α6−) | Proliferating thymic epithelial |
| | Ki67+ | progenitor cells (TEPC) |
| | CD45− EpCAM+ | Thymic epithelial cells (TEC) |
| | CD45− EpCAM+ Ki67+ | Proliferating thymic epithelial cells |
| | | (TEC) |
| | CD45− EpCam+ K5+ K8+ | Thymic epithelial progenitor cells |
| | | (TEPC) |
| | CD45− EpCAM+ | Cortical thymic epithelial cells |
| | MHCII⁺Ly51⁺UEA1^{lo} | (cTEC) |
| | CD45− EpCAM+ Ki67+ | Proliferating cortical thymic |
| | MHCII⁺Ly51⁺UEA1^{lo} | epithelial cells (cTEC) |
| | CD45− | Medullary thymic epithelial cells |
| | EpCAM+ MHCII⁺Ly51^{lo}UEA1⁺ | (mTEC) |
| | CD45− EpCAM+ Ki67+ | Proliferating medullary thymic |
| | MHCII⁺Ly51^{lo}UEA1⁺ | epithelial cells (mTEC) |

In some embodiments, the thymic cell is a medullary thymic epithelial cell (mTEC), a mimetic mTEC, an AIRE+ mTEC, an AIRE-mTEC, a mTEC progenitor cell, a mimetic mTEC progenitor cell, junction thymic epithelial cell (jTEC), thymic progenitor epithelial cell (TPEC), proliferating TPEC, thymic epithelial cell (TEC), proliferating TEC, cortical thymic epithelial cell (cTEC), proliferating cTEC, or proliferating mTEC.

Non-limiting examples of mimetic mTEC include, but are not limited to, basal (skin/lung) mTEC, enterocyte/hepatocyte mTEC, ciliated mTEC, ionocyte mTEC, keratinocyte mTEC, microfold mTEC, muscle mTEC, neuroendocrine mTEC, parathyroid mTEC, secretory mTEC, thyroid mTEC, and tuft mTEC. In some embodiments, the mimetic mTEC is a basal (skin/lung) mTEC. In some embodiments, the mimetic mTEC is an enterocyte/hepatocyte mTEC. In some embodiments, the mimetic mTEC is a ciliated mTEC. In some embodiments, the mimetic mTEC is an ionocyte mTEC. In some embodiments, the mimetic mTEC is a keratinocyte mTEc. In some embodiments, the mimetic mTEC is a microfold mTEC. In some embodiments, the mimetic mTEC is a muscle mTEC. In some embodiments, the mimetic mTEC is a neuroendocrine mTEC. In some embodiments, the mimetic mTEC is a parathyroid mTEC. In some embodiments, the mimetic mTEC is a secretory mTEC. In some embodiments, the mimetic mTEC is a thyroid mTEC. In some embodiments, the mimetic mTEC is a tuft mTEC.

In some embodiments, the basal (skin/lung) mTEC is a mimetic cell resembling basal cells of the skin and lung that putatively give rise, as in the periphery, to more terminally differentiated mimetic cells, such as keratinocyte mTECs and secretory/ionocyte mTECs.

In some embodiments, the enterocyte/hepatocyte mTEC is a Hnf4a+ Hnf4g+ mimetic cell expressing transcripts associated with gut enterocytes and liver hepatocytes, such as Vil1, Aldob, and Apoa4. These cells putatively give rise to microfold mTECs (Michelson D A, Mathis D, Thymic mimetic cells: tolerogenic masqueraders, Trends in Immunology, October 2022, Vol. 43, No. 10).

In some embodiments, the ciliated mTEC is a Foxj1+Rfx2+ Trp73+ mimetic cell exhibiting polarized cilia, line respiratory cysts in the thymus, and express transcripts associated with ciliated cells, such as Dynlrb2, Pifo, and Tubb4b. Without wishing to be bound to a particular theory, expression of a model antigen in ciliated mTECs is sufficient to induce cognate T cell tolerance (Michelson D A, Mathis D, Thymic mimetic cells: tolerogenic masqueraders, Trends in Immunology, October 2022, Vol. 43, No. 10).

In some embodiments, the ionocyte mTEC is a Foxi1+Foxi2+ mimetic cell expressing transcripts (Cftr, Slc12a2, Atp6v1b1) associated with ionocytes, and ion channel rich cells found in the kidney and lung epithelium (Michelson D A, Mathis D, Thymic mimetic cells: tolerogenic masqueraders, Trends in Immunology, October 2022, Vol. 43, No. 10).

In some embodiments, the keratinocyte mTEC is a Grhl1+ mimetic cell morphologically resembling skin keratinocytes, producing microscopically detectable Krt10+ cornified bodies that in humans are known as Hassall's corpuscles (Michelson D A, Mathis D, Thymic mimetic cells: tolerogenic masqueraders, Trends in Immunology, October 2022, Vol. 43, No. 10).

In some embodiments, the microfold mTEC is a Spib+ Sox8+ mimetic cell morphologically resembling Peyer's patch microfold cells, with dendritic processes and an associated ‘lymphocyte pocket’ (Michelson D A, Mathis D, Thymic mimetic cells: tolerogenic masqueraders, Trends in Immunology, October 2022, Vol. 43, No. 10).

In some embodiments, the muscle mTEC is a Myog+ mimetic cell morphologically resembling and expressing transcripts (Ckm, Des, Myl1) associated with skeletal muscle. Without wishing to be bound to a particular theory, expression of a model antigen in muscle mTECs is sufficient to induce cognate T cell tolerance (Michelson D A, Mathis D, Thymic mimetic cells: tolerogenic masqueraders, Trends in Immunology, October 2022, Vol. 43, No. 10).

In some embodiments, the neuroendocrine mTEC is a Foxa2+ Foxa3+ Insm1+ Ascl1+ mimetic cell expressing transcripts encoding neuroendocrine markers (Scg5, Snap25, Chga, Stxbp51) and possessing abundant secretory granules. Under the umbrella of neuroendocrine mTECs, further heterogeneity exists including Ptf1a+ pancreatic-like, Cdx2+ enteroendocrine-like, Pax6+, and Sox11+ subsets (Michelson D A, Mathis D, Thymic mimetic cells: tolerogenic masqueraders, Trends in Immunology, October 2022, Vol. 43, No. 10).

In some embodiments, the parathyroid mTEC is a Gcm2+ mimetic cell expressing microscopically detectable parathyroid hormone (PTH) (Michelson D A, Mathis D, Thymic mimetic cells: tolerogenic masqueraders, Trends in Immunology, October 2022, Vol. 43, No. 10).

In some embodiments, the secretory mTEC is a Foxa1+ Spdef+ mimetic cell expressing transcripts (Gabrp, Aqp4, Scgb3a2, Sftpd, Muc5ac, Muc5b) associated with mixed secretory cell types, including goblet cells, club cells, and alveolar epithelial cells (Michelson D A, Mathis D, Thymic mimetic cells: tolerogenic masqueraders, Trends in Immunology, October 2022, Vol. 43, No. 10).

In some embodiments, the thyroid mTEC is a mimetic cell identified as thyroglobulin- and calcitonin-expressing cell (Michelson D A, Mathis D, Thymic mimetic cells: tolerogenic masqueraders, Trends in Immunology, October 2022, Vol. 43, No. 10).

In some embodiments, the tuft mTEC is a Pou2f3+ mimetic cell expressing markers of tuft cells (IL-25, ChAT, Dclk1); mediate tolerance to tufT cell-restricted antigens and control the accumulation of type 2 ILCs and NKT cells in the thymus (Michelson D A, Mathis D, Thymic mimetic cells: tolerogenic masqueraders, Trends in Immunology, October 2022, Vol. 43, No. 10).

Medullary thymic epithelial cells (mTECs) ectopically express a plethora of peripheral-tissue antigens (PTAs), which drive deletion or phenotypic diversion of self-reactive immature T cells during thymic differentiation (Michelson D A, et al., Thymic epithelial cells co-opt lineage-defining transcription factors to eliminate autoreactive T cells, 2022, Cell 185, 1-17). Failure of PTA expression causes multiorgan autoimmunity. T cells undergo positive selection to ensure capability to bind to MHC complexes. In this process, cells in the thymus display short peptides, on their own MHC class I and class II molecules, allowing immature T cells to bind. If T cell receptors (TCRs) are incapable of binding to the antigen, the T cell undergoes apoptosis. If the TCR successfully binds to the MHC complexes on the thymic cells, the T cell receives survival signals and is thus positively selected. Further, this positive selection process also determines if a T cell will become a CD8+ T cell or a CD4+ T cell. Without wishing to be bound by a particular theory, if a TCR complex binds strongly to MHC class II, the complex will send intracellular signals to induce the expression of a protein called ThPOK. This protein reduces the expression of another key protein, called Runx3, responsible for driving CD8 expression. Because low Runx3 causes low CD8, these ThPOK+, Runx3− cells become CD4+. If, however, a developing T cell does not bind strongly to MHC class II, ThPOK levels will be low and thus Runx3 levels will be high, pushing the T cell to differentiate into a CD8+ cell.

Without wishing to be bound by a particular theory, there are two basic classes of MHC molecules in mammals, MHC class I and MHC class II. Both classes are large protein complexes formed by the association of two separate proteins. Each class includes transmembrane domains that anchor the complex into the cell membrane. MHC class 1 molecules are formed from two non-covalently associated proteins, the α chain and β2-microglobulin. The α chain comprises three distinct domains, α1, α2 and α3. The three-dimensional structure of the α1 and α2 domains forms the groove into which antigen fits for presentation to T cells. The α3 domain is an Ig-fold like domain that contains a transmembrane sequence that anchors the α chain into the cell membrane of the antigen presenting cell (APC). MHC class I complexes, when associated with antigen (and in the presence of appropriate co-stimulatory signals) stimulate CD8 cytotoxic T cells, which function to kill any cell which they specifically recognize. The two proteins which associate non-covalently to form MHC class II molecules are termed the α and β chains. The α chain comprises α1 and α2 domains, and the β chain comprises β1 and β2 domains. The cleft into which the antigen fits is formed by the interaction of the α1 and β1 domains. The α2 and β2 domains are transmembrane Ig-fold like domains that anchor the α and β chains into the cell membrane of the APC. MHC class II complexes, when associated with antigen (and in the presence of appropriate co-stimulatory signals) stimulate CD4 T cells. The primary functions of CD4 T cells are to initiate the inflammatory response, to regulate other cells in the immune system, and to provide help to B cells for antibody, or the antigen-binding fragment thereof, synthesis.

In humans, MHC molecules (with the exception of class I β2-microglobulin) are encoded by the HLA region, which is located on chromosome 6 and constitutes over 100 genes. There are 3 class I MHC α chain protein loci, termed HLA-A, -B and -C. There are also 3 pairs of class II MHC α and β chain loci, termed HLA-DR (A and B), HLA-DP (A and B), and HLA-DQ (A and B). In rats, the class I a gene is termed RT1.A, while the class II genes are termed RT1.B α and RT1.B β. The key role that MHC complexes play in triggering immune recognition has led to the development of methods by which these complexes are used to modulate the immune response.

For example, activated T cells which recognize “self” antigens (autoantigens) are known to play a key role in autoimmune diseases (such as rheumatoid arthritis) and neurodegenerative diseases of autoimmune etiology (such as multiple sclerosis). Building on the observation that isolated MHC class II molecules (loaded with the appropriate antigen) can substitute for APCs carrying the MHC class II complex and can bind to antigen-specific T cells, a number of researchers have proposed that isolated MHC/antigen complexes may be used to treat autoimmune disorders. Thus U.S. Pat. Nos. 5,194,425, and 5,284,935, each of which is hereby incorporated by reference in its entirety, disclose the use of isolated MHC class II complexes loaded with a specified autoantigen and conjugated to a toxin to eliminate T cells that are specifically immunoreactive with autoantigens.

MHC Class II antigens (or “MHC II antigens”) are major histocompatibility antigens, which are expressed on the surface of antigen-presenting cells, including dendritic cells, macrophages, B lymphocytes and certain epithelial cell types (e.g. cortical and medullary thymic epithelial cells). MHC molecules are essential for the presentation of antigenic peptides to CD4 T lymphocytes. Their expression is up-regulated on activated antigen-presenting cells. In certain contexts (e.g. an inflammatory context), MHC Class II is expressed by other cell types (e.g. endothelial cells, islet beta cells) that do not usually express MHC Class II. MHC Class II is expressed on the cell surface as a heterodimer consisting of an alpha chain and a beta chain. The tertiary structure of the molecule is such that a peptide-binding ‘groove’ or ‘cleft’ is formed between the α1 and β1 domains of the alpha and beta chains respectively. The region of the groove is highly polymorphic, resulting in a high degree of allelic variation between MHC Class II molecules within outbred populations. Conventionally, peptides presented by MHC Class II are derived from extracellular proteins, which have been endocytosed by the cell, and are usually at least around 15 amino acids in length. It is within the endocytic compartment of a cell that the peptides are loaded into the groove, forming an MHC-peptide complex, which is subsequently transported to the cell surface. The MHC-peptide complex is recognized by a T cell receptor on the surface of a T lymphocyte. In humans, MHC Class II antigens are also called Human Leukocyte Antigens (HLA), examples of which are HLA-DM, HLA-DO, HLA-DP, HLA-DQ and HLA-DR. Examples of MHC Class II antigens in inbred laboratory mouse strains are IA and IE. Developing T cells in the thymus are presented with peptides bound to MHC molecules, to which they may be able to bind. While a moderate degree of binding leads to survival and positive selection, TCRs that bind too strongly to these MHC complexes are destined for the opposite fate. When TCRs bind with high enough affinity to the MHC complexes in the thymus, the intracellular signaling is so strong that it leads to cell death, thereby eradicating immature T cells that have a high likelihood of being self-reactive and attacking our own cells. mTECs are uniquely important effectors of negative selection because they ectopically express thousands of peripheral tissue antigens (PTAs) in a mosaic fashion, allowing immature T cells to broadly sample self antigens prior to their release into the periphery, promoting deletion of autoreactive T cell clones or their conversion into regulatory T cells (Tregs), and thereby preventing autoimmunity.

In some embodiments, the molecule binds to one or more cells. In some embodiments, the molecule binds to one or more thymic cells. In some embodiments, the molecule binds to at least one thymic cells. In some embodiments, the one or more, or at least one thymic cell is a medullary thymic epithelial cell (mTEC), a mimetic mTEC, an AIRE+ mTEC, an AIRE− mTEC, a mTEC progenitor cell, a mimetic mTEC progenitor cell, junction thymic epithelial cell (jTEC), thymic progenitor epithelial cell (TPEC), proliferating TPEC, thymic epithelial cell (TEC), proliferating TEC, cortical thymic epithelial cell (cTEC), proliferating cTEC, or proliferating mTEC. In some embodiments, the molecule binds to a thymic cell and at least one more cell. In some embodiments, the molecule binds to a thymic cell and a dendritic cell.

In some embodiments, the molecule binds to a first thymic cells, and a second thymic cell. In some embodiments, the first thymic cell is selected from a medullary thymic epithelial cell (mTEC), a mimetic mTEC, an AIRE+ mTEC, an AIRE− mTEC, a mTEC progenitor cell, a mimetic mTEC progenitor cell, junction thymic epithelial cell (jTEC), thymic progenitor epithelial cell (TPEC), proliferating TPEC, thymic epithelial cell (TEC), proliferating TEC, cortical thymic epithelial cell (cTEC), proliferating cTEC, or proliferating mTEC; and the second thymic cell is selected from a medullary thymic epithelial cell (mTEC), a mimetic mTEC, an AIRE+ mTEC, an AIRE− mTEC, a mTEC progenitor cell, a mimetic mTEC progenitor cell, junction thymic epithelial cell (jTEC), thymic progenitor epithelial cell (TPEC), proliferating TPEC, thymic epithelial cell (TEC), proliferating TEC, cortical thymic epithelial cell (cTEC), proliferating cTEC, or proliferating mTEC. In some embodiments, the molecule binds to a thymic cell and at least one more cell. In some embodiments, the molecule binds to a splenic cell. In some embodiments, the molecule binds to a dendritic cell. In some embodiments, the molecule binds to a B cell. In some embodiments, the molecule binds to a thymic cell and a dendritic cell. In some embodiments, the molecule binds to a thymic cell, splenic cell, dendritic cell, and/or B cell. In some embodiments, the molecule binds to a thymic cell, a dendritic cell, and/or a B cell.

Provided herein are polypeptides, and molecules, e.g., that can be used as therapeutics that include 1 or 2 cell targeting domains that bind to at least 1 receptor on the surface of a thymic cell, such as an epithelial thymic cell; and 1 or 2 antigens of interest. In some embodiments, the compound comprises 1 or 2 antigens of interest, such as an antibody, or the antigen-binding fragment thereof, an antigen, a payload, or a fragment thereof. In some embodiments, the compound binds to at least 1 cell surface receptor molecule. In some embodiments, the compound binds to 2 different cell surface receptor molecules. In some embodiments, the compound binds to 2 cell surface receptor molecules, wherein said cell surface receptor molecules are of the same type. In some embodiments, the compound can comprise at least 1 antigen of interest. In some embodiments, the compound can comprise 2 antigens of interest. In some embodiments, the antigen of interest is an antibody, or the antigen-binding fragment thereof, an antigen, a payload, or a fragment thereof. In some embodiments, the antibody, or the antigen-binding fragment thereof, is bound to an antigen.

As used herein, the term “cell targeting domain” refers to a polypeptide, such as an antibody, or the antigen-binding fragment thereof, that binds to a receptor present on the surface of a thymic cell. In some embodiments, the cell targeting domain binds to a receptor on the surface of the thymic cell. In some embodiments, non-limiting examples of thymic cells include, but are not limited to, medullary thymic epithelial cell (mTEC), a mimetic mTEC, an AIRE+ mTEC, an AIRE− mTEC, a mTEC progenitor cell, a mimetic mTEC progenitor cell, junction thymic epithelial cell (jTEC), thymic progenitor epithelial cell (TPEC), proliferating TPEC, thymic epithelial cell (TEC), proliferating TEC, cortical thymic epithelial cell (cTEC), proliferating cTEC, or proliferating mTEC. In some embodiments, the thymic cell is a mTEC. In some embodiments, the thymic cell is a mimetic mTEC. In some embodiments, the thymic cell is an AIRE+ mTEC. In some embodiments, the thymic cell is an AIRE− mTEC. In some embodiments, the thymic cell is a mTEC progenitor cell. In some embodiments, the thymic cell is a mimetic mTEC progenitor cell. In some embodiments, the thymic cell is a basal (skin/lung) mTEC. In some embodiments, the thymic cell is an enterocyte/hepatocyte mTEC. In some embodiments, the thymic cell is a ciliated mTEC. In some embodiments, the thymic cell is an ionocyte mTEC. In some embodiments, the thymic cell is a keratinocyte mTEC. In some embodiments, the thymic cell is a microfold mTEC. In some embodiments, the thymic cell is a muscle mTEC. In some embodiments, the thymic cell is a neuroendocrine mTEC. In some embodiments, the thymic cell is a parathyroid mTEC. In some embodiments, the thymic cell is a secretory mTEC. In some embodiments, the thymic cell is a thyroid mTEC. In some embodiments, the thymic cell is a tuft mTEC.

In some embodiments, the molecule comprises one or more cell targeting domains. In some embodiments, the molecule comprises 1, or 2 cell targeting domains. In some embodiments, the cell targeting domains bind to the same cell receptor. In some embodiments, the cell targeting domains bind to different cell receptors. For example, if the molecule comprises two cell targeting domains that bind to different cell receptors, the first cell targeting domain can bind to a first cell receptor and the second cell targeting domain can bind to a second cell receptor that is different from the first. In some embodiments, the cell targeting domain is an antibody, or the antigen-binding fragment thereof. In some embodiments, the antibody, or the antigen-binding fragment thereof, is a Fab format antibody, or the antigen-binding fragment thereof. In some embodiments, the antibody, or the antigen-binding fragment thereof, is a scFv antibody, or the antigen-binding fragment thereof. In some embodiments, the antibody, or the antigen-binding fragment thereof, is an antibody, or the antigen-binding fragment thereof, as provided for herein.

In some embodiments, provided herein is a molecule comprising a cell targeting domain linked to an antigen of interest or a domain that binds to the antigen of interest, wherein the cell targeting domain binds to a thymic cell.

In some embodiments, provided herein is a molecule comprising a cell targeting domain linked to an antigen of interest or a domain that binds to the antigen of interest, wherein the cell targeting domain binds to a thymic cell, a splenic cell, a dendritic cell, or a B cell.

In some embodiments, provided herein is a molecule for inducing tolerance against an antigen of interest, wherein the molecule comprises a cell targeting domain linked to the antigen of interest or a domain that binds to the antigen of interest, wherein the targeting domain binds to a thymic cell, a splenic cell, a dendritic cell, or a B cell.

In some embodiments, provided herein is a molecule for inducing antigen-specific Tregs against an antigen of interest, wherein the molecule comprises a cell targeting domain linked to the antigen of interest or a domain that binds to the antigen of interest, wherein the targeting domain binds to a thymic cell, a splenic cell, a dendritic cell, or a B cell.

In some embodiments, provided herein is a molecule for inducing antigen-specific Tregs against an antigen of interest, wherein the molecule comprises a cell targeting domain linked to the antigen of interest or a domain that binds to the antigen of interest, wherein the targeting domain binds to a thymic cell, a splenic cell, a dendritic cell, or a B cell, wherein the molecule does not significantly induce antigen-specific T_{eff} cells.

In some embodiments, provided herein is a molecule for inducing antigen-specific thymic Treg cells by systemic administration, wherein the molecule comprises a thymic cell targeting domain linked to an antigen of interest or a domain that binds to the antigen of interest.

Without wishing to be bound to any particular theory, targeted delivery of an antigen to thymic epithelial cells (TECs) is a strategy aimed at facilitating antigen uptake, processing, and presentation on the cell surface, ultimately promoting antigen-specific T cell selection. Once the targeted antigen reaches the TECs, it is internalized through various endocytic mechanisms, including, but not limited to, receptor-mediated endocytosis, phagocytosis, or macropinocytosis. After internalization, the antigen is transported to endosomal or lysosomal compartments within the TECs, where it undergoes proteolytic processing. This processing generates antigenic peptides, or fragment thereof, that can bind to major histocompatibility complex (MHC) molecules. The generated antigenic peptides, or fragment thereof, are loaded onto MHC molecules within specialized compartments, such as the endoplasmic reticulum for MHC class I or endosomal compartments for MHC class II. Once the peptide-MHC complex is formed, it is transported to the TEC surface and presented to developing thymocytes. TECs expressing MHC class I molecules present the peptides to CD8+ T cells, while those expressing MHC class II molecules present the peptides to CD4+ T cells. The interaction between the peptide-MHC complex on TECs and the T cell receptor (TCR) on developing thymocytes drives the selection of T cells with appropriate antigen specificity. Thymocytes with low or intermediate affinity for self-antigens undergo positive selection, ensuring the survival of T cells capable of recognizing antigens in the context of self-MHC. Thymocytes with high affinity for self-antigens undergo negative selection, either being eliminated through apoptosis or diverted into regulatory T cell (Treg) differentiation to maintain self-tolerance (Klein, L., Hinterberger, M., Wirnsberger, G., & Kyewski, B. (2009). Antigen presentation in the thymus for positive selection and central tolerance induction. Nature Reviews Immunology, 9(12), 833-844.
https://doi.org/10.1038/nri2669; Mathis, D., & Benoist, C. (2009). Aire. Annual Review of Immunology, 27, 287-312.
https://doi“dot”org/10“dot”1146/annurev“dot”immunol“dot”25“dot”022106“dot”141532; Takaba, H., & Takayanagi, H. (2017). The Mechanisms of T Cell Selection in the Thymus. Trends in Immunology, 38(11), 805-816.
https://doi“dot”org/10“dot”1016/j“dot”it“dot”2017“dot”08“dot”012; Starr, T. K., Jameson, S. C., & Hogquist, K. A. (2003). Positive and negative selection of T cells. Annual Review of Immunology, 21, 139-176.
https://doi“dot”org/10“dot”1146/annurev“dot”immunol“dot”21“dot”120601“dot”141107; Roche, P. A., & Furuta, K. (2015). The ins and outs of MHC class II-mediated antigen processing and presentation. Nature Reviews Immunology, 15(4), 203-216.
https://doi“dot”org/10“dot”1038/nri3818; and Neefjes, J., Jongsma, M. L., Paul, P., & Bakke, O. (2011). Towards a systems understanding of MHC class I and MHC class II antigen presentation. Nature Reviews Immunology, 11(12), 823-836. https://doi“dot”org/10“dot”1038/nri3084; each of which are hereby incorporated by reference in its entirety).

In some embodiments, the molecule binds to a cell receptor. In some embodiments, the molecule binds to a thymic cell receptor. In some embodiments, the cell receptor is expressed on the surface of the cell. In some embodiments, the cell receptor is expressed on the surface of the thymic cell. In some embodiments, the cell receptor is expressed on the surface of an exosome. In some embodiments, the receptor is expressed on the surface of a thymic-derived exosome. Without wishing to be bound to any particular theory, thymic-derived exosomes are small, membrane-bound vesicles released by thymic cells, carrying various biological molecules like proteins, lipids, and nucleic acids. These exosomes can interact with B cells via surface receptors, such as those provided herein (Théry, C., Ostrowski, M., & Segura, E. (2009). Membrane vesicles as conveyors of immune responses. Nature Reviews Immunology, 9(8), 581-593. https://doi“dot”org/10“dot”1038/nri2567; which is hereby incorporated by reference in its entirety). B cells can uptake thymic-derived exosomes through endocytosis. Upon interaction, B cells internalize the exosomes by forming an invagination on their plasma membrane, which engulfs the exosome and forms a vesicle called an endosome within the B cell. Inside the B cell, the exosomal contents are released and can influence B cell function, differentiation, and interaction with other immune cells, ultimately contributing to immune regulation and response. B cell-induced tolerance is an essential mechanism by which the immune system prevents autoimmunity and maintains self-tolerance. B cells, as part of the adaptive immune response, produce antibodies that recognize and neutralize foreign antigens. However, they must also avoid reacting to self-antigens to prevent autoimmune reactions. Through a series of tightly regulated selection processes, including central and peripheral tolerance mechanisms, self-reactive B cells are either removed, rendered unresponsive, or undergo receptor editing to change their antigen specificity. By controlling these processes, the immune system ensures that B cells contribute to an effective defense against pathogens while minimizing the risk of autoimmunity and maintaining self-tolerance.

In some embodiments, the molecule that binds to the cell receptor comprises a native ligand. In some embodiments, the cell targeting domain that binds to the cell receptor comprises a native ligand. In some embodiments, the ligand is a native soluble ligand, or a fragment thereof. In some embodiments, the ligand is a fragment of a native membrane-tethered ligand, or a fragment thereof. In some embodiments, the membrane-tethered ligand may be anchored to the cell membrane through a transmembrane polypeptide or a glycosylphosphatidylinositol (GPI) anchor. In some embodiments, the fragment of the native membrane-tethered ligand is an ectodomain fragment.

In some embodiments, the cell receptor is selected from: CD166 antigen (CD166; encoded for by ALCAM gene, UniProt: Q13740), OX-2 membrane glycoprotein (encoded for by CD200 gene, UniProt: P41217), T-lymphocyte activation antigen CD86 (encoded for by CD86 gene, UniProt: P42081), epithelial cell adhesion molecule (EpCAM, encoded for by EPCAM gene, UniProt: P16422), receptor activator nuclear factor-κB (RANK, encoded for by TNFRSF11A gene, GeneBank: AAB86809.1), C-C motif chemokine (CCL19, encoded for by CCL19 gene, UniProt: Q99731), C-C motif chemokine 21 (CCL21, encoded for by CCL21 gene, UniProt: P84444), lymphotoxin beta receptor (LTβR, encoded for by LTBR gene, GeneBank: AKL90505.1), MHC class II (MHCII, encoded for by MHCII gene, UniProt: P01903), cluster of differentiation 40 (CD40, encoded for by CD40 gene, UniProt: P29965), cluster of differentiation 80 (CD80, encoded for by CD80 gene, UniProt: P33681), cluster of differentiation 86 (CD86, encoded for by CD86 gene, UniProt: P42081), lymphocyte antigen 6D (encoded for by LY6D gene, UniProt: Q14210), polymeric immunoglobulin receptor (PlgR, encoded for by PIGR gene, UniProt: P01833), L1 cell adhesion molecule (L1CAM, encoded for by LICAM gene, UniProt: P32004), leukemia inhibitory factor receptor (LIFR, encoded for by LIFR gene, UniProt: P42702), integrin alpha-6 (encoded for by ITGA6 gene, UniProt: P23229), integrin beta (encoded for by ITGB4 gene, UniProt: P16144), podoplanin (encoded for by PDPN gene, UniProt: Q86YL7), chemokine (C-C motif) receptor 7 (CCR7, encoded for by CCR7 gene, UniProt: P32248), signal transducer (CD24, encoded for by CD24 gene, UniProt: P25063), CD63 molecule (CD63, encoded for by CD63 gene, UniProt: P08962), CD70 molecule (CD70, encoded for by CD70 gene, UniProt: P32970), H-2 class II histocompatibility antigen gamma chain (CD74, encoded for by CD74 gene, UniProt: P04233), T-lymphocyte activation antigen CD80 (CD80, encoded for by CD80 gene, UniProt: P33681), tetraspanin (CD81, encoded for by CD81 gene, UniProt: P60033), CD9 antigen (CD9, encoded for by CD9 gene, UniProt: P21926), cadherin-1 (encoded for by CDH1 gene, UniProt: P12830), cadherin-2 (encoded for by CDH2 gene, UniProt: P19022), C-type lectin domain family 7 member A (CLEC7A; dectin-1, encoded for by CLEC7A gene, UniProt: Q9BXN2), receptor protein-tyrosine kinase (HER3, encoded for by ERBB3 gene, UniProt: P21860), CD23 antigen (FCER2; encoded for by FCER2 gene, UniProt: P06734), interleukin 20 receptor subunit beta (IL-20RB, encoded for by IL20RB gene, UniProt: Q6UXLO), lymphocyte antigen 6D (encoded for by LY6D gene, UniProt: Q14210), lymphocyte antigen 75 (encoded for by LY75 gene, UniProt: 060449), LY6/PLAUR domain containing 3 (encoded for by LYPD3 geme, UniProt: 095274), macrophage receptor MARCO (“MARCO”; encoded for by MARCO gene, UniProt: Q9UEW3), oncostatin-M-specific receptor subunit beta (encoded for by OSMR gene, UniProt: Q99650), syndecan 1 (SCD1; encoded for by SDC1 gene, UniProt: P18827), syndecan-4 (encoded for by SDC4 gene, UniProt: P31431), kunitz-type protease inhibitor 2 (HAI-2, encoded for by SPINT2 gene, UniProt: 043291), tumor-associated calcium signal transducer 2 (Trop2, encoded for by TACSTD2 gene, UniProt: P09758), extracellular calcium-sensing receptor (CaSR, encoded for by CASR gene, UniProt: P41180), acetylcholine receptor subunit alpha (nAChRalpha1, encoded for by CHRNA1 gene, UniProt: P02708), acetylcholine receptor subunit delta (encoded for by CHRND gene, UniProt: Q07001), claudin-1 (encoded for by CLDN1 gene, UniProt: 095832), claudin-4 (encoded for by CLDN4 gene, UniProt: 014493), claudin-6 (encoded for by CLDN6 gene, UniProt: P56747), immunoglobulin superfamily member 1 (encoded for by IGSF1 gene, UniProt: Q8N6C5), large neutral amino acids transporter small subunit 2 (encoded for by SLC7A8 gene, UniProt: Q9UHI5), somatostatin receptor type 2 (encoded for by SSTR2 gene, UniProt: P30874), trimeric intracellular cation channel type A (encoded for by TMEM38A gene, UniProt: Q9H6F2), sialic acid-binding Ig-like lectin 10 (SIGLEC10, encoded for by SIGLEC10 gene, UniProt: Q96LC7), growth arrest-specific gene 6 (GAS6; encoded for by gene GAS6, UniProt: Q14393), T-cell membrane protein 4 (TIM4; encoded for by gene TIMD4, UniProt: Q96H15), or any receptor on the surface of the thymic epithelial cell, and any combination thereof. The accession numbers provided for here, such as the UniProt numbers in this paragraph and throughout the present application, are incorporated by reference in its entirety, including the amino acid sequence provided therein.

In some embodiments, the cell receptor is selected from cadherin-1 (CDH1), Trop2, CD74, EpCAM, syndecan 1 (SDC1), PlgR, CD23 antigen (FCER2), macrophage receptor MARCO, CD166 antigen, OX-2 membrane glycoprotein, T-lymphocyte activation antigen CD86, RANK, CCL19, CCL21, LTβR, MHCII, CD40, CD80, CD86, lymphocyte antigen 6D, L1CAM, LIFR, integrin alpha-6, integrin beta-4, podoplanin, CCR7, CD24, CaSR, CD63, nAChRalpha1, CD70, acetylcholine receptor subunit delta, claudin-1, CD80, claudin-4, CD81, claudin-6, CD9, immunoglobulin superfamily member 1, large neutral amino acids transporter small subunit 2, cadherin-2, somatostatin receptor type 2, C-type lectin domain family 7 (CLEC7A), TRIC-A, HER3, IL-20RB, lymphocyte antigen 6D, DEC-205, LY6/PLAUR domain containing 3, oncostatin-M-specific receptor subunit beta, syndecan 1, syndecan-4, HAI-2, sialic acid-binding Ig-like lectin 10 (SIGLEC10), milk fat globule EGF and factor V/VIII domain containing (MFGE8), growth arrest-specific gene 6 (GAS6), or T-cell membrane protein 4 (TIM4), and any combination thereof.

In some embodiments, the cell receptor is selected from CaSR, nAChRalpha1, acetylcholine receptor subunit delta, claudin-1, claudin-4, claudin-6, immunoglobulin superfamily member 1, large neutral amino acids transporter small subunit 2, somatostatin receptor type 2, TRIC-A, and any combination thereof.

In some embodiments, the cell targeting domain binds to cadherin-1 (CDH1), Trop2, CD74, EpCAM, syndecan 1 (SDC1), PlgR, CD23 antigen (FCER2), macrophage receptor MARCO, CD166 antigen, OX-2 membrane glycoprotein, T-lymphocyte activation antigen CD86, RANK, CCL19, CCL21, LTβR, MHCII, CD40, CD80, CD86, lymphocyte antigen 6D, L1CAM, LIFR, integrin alpha-6, integrin beta-4, podoplanin, CCR7, CD24, CaSR, CD63, nAChRalpha1, CD70, acetylcholine receptor subunit delta, claudin-1, CD80, claudin-4, CD81, claudin-6, CD9, immunoglobulin superfamily member 1, large neutral amino acids transporter small subunit 2, cadherin-2, somatostatin receptor type 2, C-type lectin domain family 7 (CLEC7A), TRIC-A, HER3, IL-20RB, lymphocyte antigen 6D, DEC-205, LY6/PLAUR domain containing 3, oncostatin-M-specific receptor subunit beta, syndecan 1, syndecan-4, HAI-2, sialic acid-binding Ig-like lectin 10 (SIGLEC10), milk fat globule EGF and factor V/VIII domain containing (MFGE8), growth arrest-specific gene 6 (GAS6), or T-cell membrane protein 4 (TIM4), or any combination thereof.

In some embodiments, the cell targeting domain binds to CD166 antigen. In some embodiments, the cell targeting domain binds to OX-2 membrane glycoprotein. In some embodiments, the cell targeting domain binds to T-lymphocyte activation antigen CD86. In some embodiments, the cell targeting domain binds to EpCAM. In some embodiments, the cell targeting domain binds to RANK. In some embodiments, the cell targeting domain binds to CCL19. In some embodiments, the cell targeting domain binds to CCL21. In some embodiments, the cell targeting domain binds to LTβR. In some embodiments, the cell targeting domain binds to MHCII. In some embodiments, the cell targeting domain binds to CD40. In some embodiments, the cell targeting domain binds to CD80. In some embodiments, the cell targeting domain binds to CD86. In some embodiments, the cell targeting domain binds to lymphocyte antigen 6D. In some embodiments, the cell targeting domain binds to PlgR. In some embodiments, the cell targeting domain binds to LICAM. In some embodiments, the cell targeting domain binds to LIFR. In some embodiments, the cell targeting domain binds to integrin alpha-6. In some embodiments, the cell targeting domain binds to integrin beta-4. In some embodiments, the cell targeting domain binds to podoplanin. In some embodiments, the cell targeting domain binds to CCR7. In some embodiments, the cell targeting domain binds to CD24. In some embodiments, the cell targeting domain binds to CD63 on the surface of the thymic-derived exosome. In some embodiments, the cell targeting domain binds to CD70. In some embodiments, the cell targeting domain binds to CD74. In some embodiments, the cell targeting domain binds to CD80. In some embodiments, the cell targeting domain binds to CD81 on the surface of the thymic-derived exosome. In some embodiments, the cell targeting domain binds to CD9 on the surface of the thymic-derived exosome. In some embodiments, the cell targeting domain binds to cadherin-1. In some embodiments, the cell targeting domain binds to cadherin-2. In some embodiments, the cell targeting domain binds to dectin-1. In some embodiments, the cell targeting domain binds to EPCAM. In some embodiments, the cell targeting domain binds to HER3. In some embodiments, the cell targeting domain binds to CD23 antigen. In some embodiments, the cell targeting domain binds to IL-20RB. In some embodiments, the cell targeting domain binds to lymphocyte antigen 6D. In some embodiments, the cell targeting domain binds to DEC-205. In some embodiments, the cell targeting domain binds to LY6/PLAUR domain containing 3. In some embodiments, the cell targeting domain binds to macrophage receptor MARCO. In some embodiments, the cell targeting domain binds to oncostatin-M-specific receptor subunit beta. In some embodiments, the cell targeting domain binds to syndecan 1. In some embodiments, the cell targeting domain binds to syndecan-4. In some embodiments, the cell targeting domain binds to HAI-2. In some embodiments, the cell targeting domain binds to Trop2. In some embodiments, the cell targeting domain binds to CaSR. In some embodiments, the cell targeting domain binds to nAChRalpha1. In some embodiments, the cell targeting domain binds to acetylcholine receptor subunit delta. In some embodiments, the cell targeting domain binds to claudin-1. In some embodiments, the cell targeting domain binds to claudin-4. In some embodiments, the cell targeting domain binds to claudin-6. In some embodiments, the cell targeting domain binds to immunoglobulin superfamily member 1. In some embodiments, the cell targeting domain binds to large neutral amino acids transporter small subunit 2. In some embodiments, the cell targeting domain binds to somatostatin receptor type 2. In some embodiments, the cell targeting domain binds to TRIC-A. In some embodiments, the cell targeting domain binds to sialic acid-binding Ig-like lectin 10 (SIGLEC10). In some embodiments, the cell targeting domain binds to milk fat globule EGF and factor V/VIII domain containing (MFGE8). In some embodiments, the cell targeting domain binds to growth arrest-specific gene 6 (GAS6), or T-cell membrane protein 4 (TIM4).

In some embodiments, the cell targeting domain binds to, or specifically binds to, TROP2, CDH1, EpCAM, MARCO, or CD74.

In some embodiments, the cell targeting domain binds to cadherin-1 (CDH1), Trop2, CD74, EpCAM, syndecan 1 (SDC1), PlgR, CD23 antigen (FCER2), macrophage receptor MARCO, CD166 antigen, OX-2 membrane glycoprotein, T-lymphocyte activation antigen CD86, RANK, CCL19, CCL21, LTβR, MHCII, CD40, CD80, CD86, lymphocyte antigen 6D, L1CAM, LIFR, integrin alpha-6, integrin beta-4, podoplanin, CCR7, CD24, CaSR, CD63, nAChRalpha1, CD70, acetylcholine receptor subunit delta, claudin-1, CD80, claudin-4, CD81, claudin-6, CD9, immunoglobulin superfamily member 1, large neutral amino acids transporter small subunit 2, cadherin-2, somatostatin receptor type 2, C-type lectin domain family 7 (CLEC7A), TRIC-A, HER3, IL-20RB, lymphocyte antigen 6D, DEC-205, LY6/PLAUR domain containing 3, oncostatin-M-specific receptor subunit beta, syndecan 1, syndecan-4, HAI-2, sialic acid-binding Ig-like lectin 10 (SIGLEC10), milk fat globule EGF and factor V/VIII domain containing (MFGE8), growth arrest-specific gene 6 (GAS6), or T-cell membrane protein 4 (TIM4), or any combination thereof, on the surface of a cell. In some embodiments, the cell is a thymic cell, a splenic cell, a dendritic cell, or a B cell.

In some embodiments, the cell targeting domain binds to a receptor on the surface of a thymic cell. In some embodiments, the cell targeting domain binds to a receptor on the surface of a thymic epithelial cell. In some embodiments, the cell targeting domain binds to CD166 antigen on the surface of the thymic cell. In some embodiments, the cell targeting domain binds to OX-2 membrane glycoprotein on the surface of the thymic cell. In some embodiments, the cell targeting domain binds to OX-2 membrane glycoprotein on the surface of the thymic cell. In some embodiments, the cell targeting domain binds to T-lymphocyte activation antigen CD86 on the surface of the thymic cell. In some embodiments, the cell targeting domain binds to EpCAM on the surface of the thymic cell. In some embodiments, the cell targeting domain binds to RANK on the surface of the thymic cell. In some embodiments, the cell targeting domain binds to CCL19 on the surface of the thymic cell. In some embodiments, the cell targeting domain binds to CCL21 on the surface of the thymic cell. In some embodiments, the cell targeting domain binds to LTβR on the surface of the thymic cell. In some embodiments, the cell targeting domain binds to MHCII on the surface of the thymic cell. In some embodiments, the cell targeting domain binds to CD40 on the surface of the thymic cell. In some embodiments, the cell targeting domain binds to CD80 on the surface of the thymic cell. In some embodiments, the cell targeting domain binds to CD86 on the surface of the thymic cell. In some embodiments, the cell targeting domain binds to lymphocyte antigen 6D on the surface of the thymic cell. In some embodiments, the cell targeting domain binds to PlgR on the surface of the thymic cell. In some embodiments, the cell targeting domain binds to L1CAM on the surface of the thymic cell. In some embodiments, the cell targeting domain binds to LIFR on the surface of the thymic cell. In some embodiments, the cell targeting domain binds to integrin alpha-6 on the surface of the thymic cell. In some embodiments, the cell targeting domain binds to integrin beta-4 on the surface of the thymic cell. In some embodiments, the cell targeting domain binds to podoplanin on the surface of the thymic cell. In some embodiments, the cell targeting domain binds to CCR7 on the surface of the thymic cell. In some embodiments, the cell targeting domain binds to CD24 on the surface of the thymic cell. In some embodiments, the cell targeting domain binds to CD63 on the surface of the thymic-derived exosome. In some embodiments, the cell targeting domain binds to CD70 on the surface of the thymic cell. In some embodiments, the cell targeting domain binds to CD74 on the surface of the thymic cell. In some embodiments, the cell targeting domain binds to CD80 on the surface of the thymic cell. In some embodiments, the cell targeting domain binds to CD81 on the surface of the thymic-derived exosome. In some embodiments, the cell targeting domain binds to CD9 on the surface of the thymic-derived exosome. In some embodiments, the cell targeting domain binds to cadherin-1 on the surface of the thymic cell. In some embodiments, the cell targeting domain binds to cadherin-2 on the surface of the thymic cell. In some embodiments, the cell targeting domain binds to dectin-1 on the surface of the thymic cell. In some embodiments, the cell targeting domain binds to EPCAM on the surface of the thymic cell. In some embodiments, the cell targeting domain binds to HER3 on the surface of the thymic cell. In some embodiments, the cell targeting domain binds to CD23 antigen on the surface of the thymic cell. In some embodiments, the cell targeting domain binds to IL-20RB on the surface of the thymic cell. In some embodiments, the cell targeting domain binds to lymphocyte antigen 6D on the surface of the thymic cell. In some embodiments, the cell targeting domain binds to DEC-205 on the surface of the thymic cell. In some embodiments, the cell targeting domain binds to LY6/PLAUR domain containing 3 on the surface of the thymic cell. In some embodiments, the cell targeting domain binds to macrophage receptor MARCO on the surface of the thymic cell. In some embodiments, the cell targeting domain binds to oncostatin-M-specific receptor subunit beta on the surface of the thymic cell. In some embodiments, the cell targeting domain binds to syndecan 1 on the surface of the thymic cell. In some embodiments, the cell targeting domain binds to syndecan-4 on the surface of the thymic cell. In some embodiments, the cell targeting domain binds to HAI-2 on the surface of the thymic cell. In some embodiments, the cell targeting domain binds to Trop2 on the surface of the thymic cell. In some embodiments, the cell targeting domain binds to CaSR on the surface of the thymic cell. In some embodiments, the cell targeting domain binds to nAChRalpha1 on the surface of the thymic cell. In some embodiments, the cell targeting domain binds to acetylcholine receptor subunit delta on the surface of the thymic cell. In some embodiments, the cell targeting domain binds to claudin-1 on the surface of the thymic cell. In some embodiments, the cell targeting domain binds to claudin-4 on the surface of the thymic cell. In some embodiments, the cell targeting domain binds to claudin-6 on the surface of the thymic cell. In some embodiments, the cell targeting domain binds to immunoglobulin superfamily member 1 on the surface of the thymic cell. In some embodiments, the cell targeting domain binds to large neutral amino acids transporter small subunit 2 on the surface of the thymic cell. In some embodiments, the cell targeting domain binds to somatostatin receptor type 2 on the surface of the thymic cell. In some embodiments, the cell targeting domain binds to TRIC-A on the surface of the thymic cell. In some embodiments, the cell targeting domain binds to sialic acid-binding Ig-like lectin 10 (SIGLEC10) on the surface of the thymic cell. In some embodiments, the cell targeting domain binds to milk fat globule EGF and factor V/VIII domain containing (MFGE8) on the surface of the thymic cell. In some embodiments, the cell targeting domain binds to growth arrest-specific gene 6 (GAS6), or T-cell membrane protein 4 (TIM4) on the surface of the thymic cell.

In some embodiments, the cell targeting domain is an antibody, or the antigen-binding fragment thereof, that binds to a receptor on the surface of a cell. In some embodiments, the cell targeting domain is an antibody, or the antigen-binding fragment thereof, that binds to a receptor on the surface of a thymic cell. In some embodiments, the cell targeting domain is an antibody, or the antigen-binding fragment thereof, that binds to cadherin-1 (CDH1), Trop2, CD74, EpCAM, syndecan 1 (SDC1), PlgR, CD23 antigen (FCER2), macrophage receptor MARCO, CD166 antigen, OX-2 membrane glycoprotein, T-lymphocyte activation antigen CD86, RANK, CCL19, CCL21, LTβR, MHCII, CD40, CD80, CD86, lymphocyte antigen 6D, L1CAM, LIFR, integrin alpha-6, integrin beta-4, podoplanin, CCR7, CD24, CaSR, CD63, nAChRalpha1, CD70, acetylcholine receptor subunit delta, claudin-1, CD80, claudin-4, CD81, claudin-6, CD9, immunoglobulin superfamily member 1, large neutral amino acids transporter small subunit 2, cadherin-2, somatostatin receptor type 2, C-type lectin domain family 7 (CLEC7A), TRIC-A, HER3, IL-20RB, lymphocyte antigen 6D, DEC-205, LY6/PLAUR domain containing 3, oncostatin-M-specific receptor subunit beta, syndecan 1, syndecan-4, HAI-2, sialic acid-binding Ig-like lectin 10 (SIGLEC10), milk fat globule EGF and factor V/VIII domain containing (MFGE8), growth arrest-specific gene 6 (GAS6), or T-cell membrane protein 4 (TIM4). In some embodiments, the cell targeting domain is an antibody, or the antigen-binding fragment thereof, selected from an anti-CD166 antigen antibody, or the antigen-binding fragment thereof, an anti-OX-2 membrane glycoprotein antibody, or the antigen-binding fragment thereof, and anti-T-lymphocyte activation antigen CD86 antibody, or the antigen-binding fragment thereof, an anti-EpCAM antibody, or the antigen-binding fragment thereof, an anti-RANK antibody, or the antigen-binding fragment thereof, an anti-CCL19 antibody, or the antigen-binding fragment thereof, an anti-CCL21 antibody, or the antigen-binding fragment thereof, an anti-LTβR antibody, or the antigen-binding fragment thereof, an anti-MHCII antibody, or the antigen-binding fragment thereof, an anti-CD40 antibody, or the antigen-binding fragment thereof, an anti-CD80 antibody, or the antigen-binding fragment thereof, an anti-CD86 antibody, or the antigen-binding fragment thereof, an anti-lymphocyte antigen 6D antibody, or the antigen-binding fragment thereof, an anti-PlgR antibody, or the antigen-binding fragment thereof, an anti-L1CAM antibody, or the antigen-binding fragment thereof, an anti-LIFR antibody, or the antigen-binding fragment thereof, an anti-integrin alpha-6 antibody, or the antigen-binding fragment thereof, an anti-integrin beta-4 antibody, or the antigen-binding fragment thereof, an anti-podoplanin antibody, or the antigen-binding fragment thereof, an anti-CCR7 antibody, or the antigen-binding fragment thereof, an anti-CD24 antibody, or the antigen-binding fragment thereof, an anti-CaSR antibody, or the antigen-binding fragment thereof, an anti-CD63 antibody, or the antigen-binding fragment thereof, an anti-nAChRalpha1 antibody, or the antigen-binding fragment thereof, an anti-CD70 antibody, or the antigen-binding fragment thereof, an anti-acetylcholine receptor subunit delta antibody, or the antigen-binding fragment thereof, an anti-CD74 antibody, or the antigen-binding fragment thereof, an anti-claudin-1 antibody, or the antigen-binding fragment thereof, an anti-CD80 antibody, or the antigen-binding fragment thereof, an anti-claudin-4 antibody, or the antigen-binding fragment thereof, an anti-CD81 antibody, or the antigen-binding fragment thereof, an anti-claudin-6 antibody, or the antigen-binding fragment thereof, an anti-CD9 antibody, or the antigen-binding fragment thereof, an anti-immunoglobulin superfamily member 1 antibody, or the antigen-binding fragment thereof, an anti-cadherin-1 antibody, or the antigen-binding fragment thereof, an anti-large neutral amino acids transporter small subunit 2 antibody, or the antigen-binding fragment thereof, an anti-cadherin-2 antibody, or the antigen-binding fragment thereof, an anti-somatostatin receptor type 2 antibody, or the antigen-binding fragment thereof, an anti-dectin-1 antibody, or the antigen-binding fragment thereof, an anti-TRIC-A antibody, or the antigen-binding fragment thereof, an anti-EPCAM antibody, or the antigen-binding fragment thereof, an anti-HER3 antibody, or the antigen-binding fragment thereof, an anti-CD23 antigen antibody, or the antigen-binding fragment thereof, an anti-IL-20RB antibody, or the antigen-binding fragment thereof, an anti-lymphocyte antigen 6D antibody, or the antigen-binding fragment thereof, an anti-DEC-205 antibody, or the antigen-binding fragment thereof, an anti-LY6/PLAUR domain containing 3 antibody, or the antigen-binding fragment thereof, an anti-macrophage receptor MARCO antibody, or the antigen-binding fragment thereof, an anti-oncostatin-M-specific receptor subunit beta antibody, or the antigen-binding fragment thereof, an anti-syndecan 1 antibody, or the antigen-binding fragment thereof, an anti-syndecan-4 antibody, or the antigen-binding fragment thereof, an anti-HAI-2 antibody, or the antigen-binding fragment thereof, an anti-Trop2 antibody, or the antigen-binding fragment thereof, or any combination thereof.

In some embodiments, the cell targeting domain is an antibody, or the antigen-binding fragment thereof, that binds to a thymic epithelial cell receptor. In some embodiments, the cell targeting domain is an anti-CD166 antigen antibody, or the antigen-binding fragment thereof. In some embodiments, the cell targeting domain is an anti-OX-2 membrane glycoprotein antibody, or the antigen-binding fragment thereof. In some embodiments, the cell targeting domain is an anti-T-lymphocyte activation antigen CD86 antibody, or the antigen-binding fragment thereof. In some embodiments, the cell targeting domain is an anti-EpCAM antibody, or the antigen-binding fragment thereof. In some embodiments, the cell targeting domain is an anti-RANK antibody, or the antigen-binding fragment thereof. In some embodiments, the cell targeting domain is an anti-CCL19/21 antibody, or the antigen-binding fragment thereof. In some embodiments, the cell targeting domain is an anti-LTβR antibody, or the antigen-binding fragment thereof. In some embodiments, the cell targeting domain is an anti-MHCII antibody, or the antigen-binding fragment thereof. In some embodiments, the cell targeting domain is an anti-CD40 antibody, or the antigen-binding fragment thereof. In some embodiments, the cell targeting domain is an anti-CD80 antibody, or the antigen-binding fragment thereof, an anti-CD86 antibody, or the antigen-binding fragment thereof. In some embodiments, the cell targeting domain is an anti-lymphocyte antigen 6D antibody, or the antigen-binding fragment thereof. In some embodiments, the cell targeting domain is an anti-PlgR antibody, or the antigen-binding fragment thereof. In some embodiments, the cell targeting domain is an anti-L1CAM antibody, or the antigen-binding fragment thereof. In some embodiments, the cell targeting domain is an anti-LIFR antibody, or the antigen-binding fragment thereof. In some embodiments, the cell targeting domain is an anti-integrin alpha-6 antibody, or the antigen-binding fragment thereof. In some embodiments, the cell targeting domain is an anti-integrin beta-4 antibody, or the antigen-binding fragment thereof. In some embodiments, the cell targeting domain is an anti-podoplanin antibody, or the antigen-binding fragment thereof. In some embodiments, the cell targeting domain is an anti-CCR7 antibody, or the antigen-binding fragment thereof. In some embodiments, the cell targeting domain is an anti-CD24 antibody, or the antigen-binding fragment thereof. In some embodiments, the cell targeting domain is an anti-CaSR antibody, or the antigen-binding fragment thereof. In some embodiments, the cell targeting domain is an anti-CD63 antibody, or the antigen-binding fragment thereof. In some embodiments, the cell targeting domain is an anti-nAChRalpha1 antibody, or the antigen-binding fragment thereof. In some embodiments, the cell targeting domain is an anti-CD70 antibody, or the antigen-binding fragment thereof. In some embodiments, the cell targeting domain is an anti-acetylcholine receptor subunit delta antibody, or the antigen-binding fragment thereof. In some embodiments, the cell targeting domain is an anti-CD74 antibody, or the antigen-binding fragment thereof. In some embodiments, the cell targeting domain is an anti-claudin-1 antibody, or the antigen-binding fragment thereof. In some embodiments, the cell targeting domain is an anti-CD80 antibody, or the antigen-binding fragment thereof. In some embodiments, the cell targeting domain is an anti-claudin-4 antibody, or the antigen-binding fragment thereof. In some embodiments, the cell targeting domain is an anti-CD81 antibody, or the antigen-binding fragment thereof. In some embodiments, the cell targeting domain is an anti-claudin-6 antibody, or the antigen-binding fragment thereof. In some embodiments, the cell targeting domain is an anti-CD9 antibody, or the antigen-binding fragment thereof. In some embodiments, the cell targeting domain is an anti-immunoglobulin superfamily member 1 antibody, or the antigen-binding fragment thereof. In some embodiments, the cell targeting domain is an anti-cadherin-1 antibody, or the antigen-binding fragment thereof. In some embodiments, the cell targeting domain is an anti-large neutral amino acids transporter small subunit 2 antibody, or the antigen-binding fragment thereof. In some embodiments, the cell targeting domain is an anti-cadherin-2 antibody, or the antigen-binding fragment thereof. In some embodiments, the cell targeting domain is an anti-somatostatin receptor type 2 antibody, or the antigen-binding fragment thereof. In some embodiments, the cell targeting domain is an anti-dectin-1 antibody, or the antigen-binding fragment thereof. In some embodiments, the cell targeting domain is an anti-TRIC-A antibody, or the antigen-binding fragment thereof. In some embodiments, the cell targeting domain is an anti-HER3 antibody, or the antigen-binding fragment thereof. In some embodiments, the cell targeting domain is an anti-CD23 antigen antibody, or the antigen-binding fragment thereof. In some embodiments, the cell targeting domain is an anti-IL-20RB antibody, or the antigen-binding fragment thereof. In some embodiments, the cell targeting domain is an anti-lymphocyte antigen 6D antibody, or the antigen-binding fragment thereof. In some embodiments, the cell targeting domain is an anti-DEC-205 antibody, or the antigen-binding fragment thereof. In some embodiments, the cell targeting domain is an anti-LY6/PLAUR domain containing 3 antibody, or the antigen-binding fragment thereof. In some embodiments, the cell targeting domain is an anti-macrophage receptor MARCO antibody, or the antigen-binding fragment thereof. In some embodiments, the cell targeting domain is an anti-oncostatin-M-specific receptor subunit beta antibody, or the antigen-binding fragment thereof. In some embodiments, the cell targeting domain is an anti-syndecan 1 antibody, or the antigen-binding fragment thereof. In some embodiments, the cell targeting domain is an anti-syndecan-4 antibody, or the antigen-binding fragment thereof. In some embodiments, the cell targeting domain is an anti-HAI-2 antibody, or the antigen-binding fragment thereof. In some embodiments, the cell targeting domain is an anti-Trop2 antibody, or the antigen-binding fragment thereof. In some embodiments, the cell targeting domain is an anti-SIGLEC10 antibody, or the antigen-binding fragment thereof. In some embodiments, the cell targeting domain is an anti-MFGE8 antibody, or the antigen-binding fragment thereof. In some embodiments, the cell targeting domain is an anti-GAS6 antibody, or the antigen-binding fragment thereof. In some embodiments, the cell targeting domain is an anti-TIM4 antibody, or the antigen-binding fragment thereof.

In some embodiments, the anti-Trop2 antibody, or an antigen-binding fragment thereof, is Sp293 antibody clone available from Abcam (cat. no. ab310311). In some embodiments, the anti-E-cadherin (CDH1) antibody, or an antigen-binding fragment thereof, is DECMA-1 antibody clone available from ThermoFisher (cat. no. 14-3249-82). In some embodiments, the anti-MARCO antibody, or an antigen-binding fragment thereof, is the anti-MARCO antibody, or the antigen-binding fragment thereof, as disclosed in U.S. Publication No. 20220153832, which is hereby incorporated by reference in its entirety. In some embodiments, the anti-DEC205 (LY75) antibody, or an antigen-binding fragment thereof, is HD83 antibody clone available from BioLegend (cat. no. 359202). In some embodiments, the anti-CD74 antibody, or an antigen-binding fragment thereof, is ln1/CD74 antibody clone available from BioLegend (cat. no. 151002). In some embodiments, the anti-EpCAM antibody, or an antigen-binding fragment thereof, is G8.8 antibody clone available from BioLegend (cat. no. 118211). In some embodiments, the anti-FcGRII (FCER2) antibody, or an antigen-binding fragment thereof, is B3B4 antibody clone available from ThermoFisher (cat. no. 14-0232-81). In some embodiments, the anti-PlGR antibody, or an antigen-binding fragment thereof, is the anti-PlGR antibody, or the antigen-binding fragment thereof, as disclosed in U.S. Publication No. 20220112276, which is hereby incorporated by reference in its entirety.

In some embodiments, the molecule comprises a cell targeting domain that binds to any one of CD166 antigen, OX-2 membrane glycoprotein, T-lymphocyte activation antigen CD86, EpCAM, RANK, CCL19, CCL21, LTβR, MHCII, CD40, CD80, CD86, lymphocyte antigen 6D, PlgR, L1CAM, LIFR, integrin alpha-6, integrin beta-4, podoplanin, CCR7, CD24, CaSR, CD63, nAChRalpha1, CD70, acetylcholine receptor subunit delta, CD74, claudin-1, CD80, claudin-4, CD81, claudin-6, CD9, immunoglobulin superfamily member 1, cadherin-1, large neutral amino acids transporter small subunit 2, cadherin-2, somatostatin receptor type 2, dectin-1, TRIC-A, HER3, CD23 antigen, IL-20RB, lymphocyte antigen 6D, DEC-205, LY6/PLAUR domain containing 3, macrophage receptor MARCO, oncostatin-M-specific receptor subunit beta, syndecan 1, syndecan-4, HAI-2, or Trop2; and a second cell targeting domain that binds to any one of CD166 antigen, OX-2 membrane glycoprotein, T-lymphocyte activation antigen CD86, EpCAM, RANK, CCL19, CCL21, LTβR, MHCII, CD40, CD80, CD86, lymphocyte antigen 6D, PlgR, L1CAM, LIFR, integrin alpha-6, integrin beta-4, podoplanin, CCR7, CD24, CaSR, CD63, nAChRalpha1, CD70, acetylcholine receptor subunit delta, CD74, claudin-1, CD80, claudin-4, CD81, claudin-6, CD9, immunoglobulin superfamily member 1, cadherin-1, large neutral amino acids transporter small subunit 2, cadherin-2, somatostatin receptor type 2, dectin-1, TRIC-A, HER3, CD23 antigen, IL-20RB, lymphocyte antigen 6D, DEC-205, LY6/PLAUR domain containing 3, macrophage receptor MARCO, oncostatin-M-specific receptor subunit beta, syndecan 1, syndecan-4, HAI-2, or Trop2.

In some embodiments, the molecule comprises a first cell targeting domain that binds to any one of cadherin-1 (CDH1), Trop2, CD74, EpCAM, syndecan 1 (SDC1), PlgR, CD23 antigen (FCER2), macrophage receptor MARCO, CD166 antigen, OX-2 membrane glycoprotein, T-lymphocyte activation antigen CD86, RANK, CCL19, CCL21, LTβR, MHCII, CD40, CD80, CD86, lymphocyte antigen 6D, L1CAM, LIFR, integrin alpha-6, integrin beta-4, podoplanin, CCR7, CD24, CaSR, CD63, nAChRalpha1, CD70, acetylcholine receptor subunit delta, claudin-1, CD80, claudin-4, CD81, claudin-6, CD9, immunoglobulin superfamily member 1, large neutral amino acids transporter small subunit 2, cadherin-2, somatostatin receptor type 2, C-type lectin domain family 7 (CLEC7A), TRIC-A, HER3, IL-20RB, lymphocyte antigen 6D, DEC-205, LY6/PLAUR domain containing 3, oncostatin-M-specific receptor subunit beta, syndecan 1, syndecan-4, HAI-2, sialic acid-binding Ig-like lectin 10 (SIGLEC10), milk fat globule EGF and factor V/VIII domain containing (MFGE8), growth arrest-specific gene 6 (GAS6), or T-cell membrane protein 4 (TIM4); and a second cell targeting domain that binds to any one of cadherin-1 (CDH1), Trop2, CD74, EpCAM, syndecan 1 (SDC1), PlgR, CD23 antigen (FCER2), macrophage receptor MARCO, CD166 antigen, OX-2 membrane glycoprotein, T-lymphocyte activation antigen CD86, RANK, CCL19, CCL21, LTβR, MHCII, CD40, CD80, CD86, lymphocyte antigen 6D, L1CAM, LIFR, integrin alpha-6, integrin beta-4, podoplanin, CCR7, CD24, CaSR, CD63, nAChRalpha1, CD70, acetylcholine receptor subunit delta, claudin-1, CD80, claudin-4, CD81, claudin-6, CD9, immunoglobulin superfamily member 1, large neutral amino acids transporter small subunit 2, cadherin-2, somatostatin receptor type 2, C-type lectin domain family 7 (CLEC7A), TRIC-A, HER3, IL-20RB, lymphocyte antigen 6D, DEC-205, LY6/PLAUR domain containing 3, oncostatin-M-specific receptor subunit beta, syndecan 1, syndecan-4, HAI-2, sialic acid-binding Ig-like lectin 10 (SIGLEC10), milk fat globule EGF and factor V/VIII domain containing (MFGE8), growth arrest-specific gene 6 (GAS6), or T-cell membrane protein 4 (TIM4).

In some embodiments, the molecule comprises a first cell targeting domain that binds to any one of Trop2, macrophage receptor MARCO, cadherin-1 (CDH1), Trop2, CD74, EpCAM, syndecan 1 (SDC1), PlgR, CD23 antigen (FCER2), CD166 antigen, OX-2 membrane glycoprotein, T-lymphocyte activation antigen CD86, RANK, CCL19, CCL21, LTβR, MHCII, CD40, CD80, CD86, lymphocyte antigen 6D, L1CAM, LIFR, integrin alpha-6, integrin beta-4, podoplanin, CCR7, CD24, CaSR, CD63, nAChRalpha1, CD70, acetylcholine receptor subunit delta, claudin-1, CD80, claudin-4, CD81, claudin-6, CD9, immunoglobulin superfamily member 1, large neutral amino acids transporter small subunit 2, cadherin-2, somatostatin receptor type 2, C-type lectin domain family 7 (CLEC7A), TRIC-A, HER3, IL-20RB, lymphocyte antigen 6D, DEC-205, LY6/PLAUR domain containing 3, oncostatin-M-specific receptor subunit beta, syndecan 1, syndecan-4, HAI-2, sialic acid-binding Ig-like lectin 10 (SIGLEC10), milk fat globule EGF and factor V/VIII domain containing (MFGE8), growth arrest-specific gene 6 (GAS6), or T-cell membrane protein 4 (TIM4).

In some embodiments, the molecule comprises a first cell targeting domain that binds to any one of CaSR, nAChRalpha1, acetylcholine receptor subunit delta, claudin-1, claudin-4, claudin-6, immunoglobulin superfamily member 1, large neutral amino acids transporter small subunit 2, somatostatin receptor type 2, or TRIC-A; and a second cell targeting domain that binds to any one of CaSR, nAChRalpha1, acetylcholine receptor subunit delta, claudin-1, claudin-4, claudin-6, immunoglobulin superfamily member 1, large neutral amino acids transporter small subunit 2, somatostatin receptor type 2, or TRIC-A.

In some embodiments, the molecule comprises a ligand amino acid sequence, or a fragment thereof. As used herein, the term “ligand” refers to a receptor or cell surface protein's natural binding partner. In some embodiments, a ligand, or a fragment thereof, can be the full amino acid sequence, the ectodomain amino acid sequence, or the extracellular amino acid sequence. In some embodiments, the extracellular amino acid sequence does not comprise the GPI anchor. In some embodiments, the ligand, or a fragment thereof, binds to a thymic cell receptor. In some embodiments, the ligand, or a fragment thereof, binds to a thymic cell surface receptor. In some embodiments, the ligand, or a fragment thereof, binds to EpCAM, RANK, CCL19, CCL21, LTβR, MHCII, CD40, CD80, CD86, lymphocyte antigen 6D, PlgR, L1CAM, LIFR, integrin alpha-6, integrin beta-4, podoplanin, CCR7, CD24, CaSR, CD63, nAChRalpha1, CD70, acetylcholine receptor subunit delta, CD74, claudin-1, CD80, claudin-4, CD81, claudin-6, CD9, immunoglobulin superfamily member 1, cadherin-1, large neutral amino acids transporter small subunit 2, cadherin-2, somatostatin receptor type 2, dectin-1, TRIC-A, HER3, CD23 antigen, IL-20RB, lymphocyte antigen 6D, DEC-205, LY6/PLAUR domain containing 3, macrophage receptor MARCO, oncostatin-M-specific receptor subunit beta, syndecan 1, syndecan-4, HAI-2, or Trop2; and a second cell targeting domain that binds to any one of EpCAM, RANK, CCL19, CCL21, LTβR, MHCII, CD40, CD80, CD86, lymphocyte antigen 6D, PlgR, L1CAM, LIFR, integrin alpha-6, integrin beta-4, podoplanin, CCR7, CD24, CaSR, CD63, nAChRalpha1, CD70, acetylcholine receptor subunit delta, CD74, claudin-1, CD80, claudin-4, CD81, claudin-6, CD9, immunoglobulin superfamily member 1, cadherin-1, large neutral amino acids transporter small subunit 2, cadherin-2, somatostatin receptor type 2, dectin-1, TRIC-A, HER3, CD23 antigen, IL-20RB, lymphocyte antigen 6D, DEC-205, LY6/PLAUR domain containing 3, macrophage receptor MARCO, oncostatin-M-specific receptor subunit beta, syndecan 1, syndecan-4, HAI-2, Trop2, sialic acid-binding Ig-like lectin 10 (SIGLEC10), milk fat globule EGF and factor V/VIII domain containing (MFGE8), growth arrest-specific gene 6 (GAS6), or T-cell membrane protein 4 (TIM4).

In some embodiments, the ligand, or a fragment thereof, binds to EpCAM. In some embodiments, the ligand, or a fragment thereof, binds to RANK. In some embodiments, the ligand, or a fragment thereof, binds to CCL19. In some embodiments, the ligand, or a fragment thereof, binds to CCL21. In some embodiments, the ligand, or a fragment thereof, binds to LTBR. In some embodiments, the ligand, or a fragment thereof, binds to MHCII. In some embodiments, the ligand, or a fragment thereof, binds to CD40. In some embodiments, the ligand, or a fragment thereof, binds to CD80. In some embodiments, the ligand, or a fragment thereof, binds to CD86. In some embodiments, the ligand, or a fragment thereof, binds to lymphocyte antigen 6D. In some embodiments, the ligand, or a fragment thereof, binds to PlgR.

In some embodiments, the ligand, or a fragment thereof, binds to L1CAM. In some embodiments, the ligand, or a fragment thereof, binds to LIFR. In some embodiments, the ligand, or a fragment thereof, binds to integrin alpha-6. In some embodiments, the ligand, or a fragment thereof, binds to integrin beta-4. In some embodiments, the ligand, or a fragment thereof, binds to podoplanin. In some embodiments, the ligand, or a fragment thereof, binds to CCR7. In some embodiments, the ligand, or a fragment thereof, binds to CD24. In some embodiments, the ligand, or a fragment thereof, binds to CaSR. In some embodiments, the ligand, or a fragment thereof, binds to CD63. In some embodiments, the ligand, or a fragment thereof, binds to nAChRalpha1. In some embodiments, the ligand, or a fragment thereof, binds to CD70. In some embodiments, the ligand, or a fragment thereof, binds to acetylcholine receptor subunit delta. In some embodiments, the ligand, or a fragment thereof, binds to CD74. In some embodiments, the ligand, or a fragment thereof, binds to claudin-1. In some embodiments, the ligand, or a fragment thereof, binds to CD80. In some embodiments, the ligand, or a fragment thereof, binds to claudin-4. In some embodiments, the ligand, or a fragment thereof, binds to CD81. In some embodiments, the ligand, or a fragment thereof, binds to claudin-6. In some embodiments, the ligand, or a fragment thereof, binds to CD9. In some embodiments, the ligand, or a fragment thereof, binds to immunoglobulin superfamily member 1. In some embodiments, the ligand, or a fragment thereof, binds to cadherin-1. In some embodiments, the ligand, or a fragment thereof, binds to large neutral amino acids transporter small subunit 2. In some embodiments, the ligand, or a fragment thereof, binds to cadherin-2. In some embodiments, the ligand, or a fragment thereof, binds to somatostatin receptor type 2. In some embodiments, the ligand, or a fragment thereof, binds to dectin-1. In some embodiments, the ligand, or a fragment thereof, binds to TRIC-A. In some embodiments, the ligand, or a fragment thereof, binds to HER3. In some embodiments, the ligand, or a fragment thereof, binds to CD23 antigen. In some embodiments, the ligand, or a fragment thereof, binds to IL-20RB. In some embodiments, the ligand, or a fragment thereof, binds to lymphocyte antigen 6D. In some embodiments, the ligand, or a fragment thereof, binds to DEC-2. In some embodiments, the ligand, or a fragment thereof, binds to LY6/PLAUR domain containing 3. In some embodiments, the ligand, or a fragment thereof, binds to macrophage receptor MARCO. In some embodiments, the ligand, or a fragment thereof, binds to oncostatin-M-specific receptor subunit beta. In some embodiments, the ligand, or a fragment thereof, binds to syndecan 1. In some embodiments, the ligand, or a fragment thereof, binds to syndecan-4. In some embodiments, the ligand, or a fragment thereof, binds to HAI-2. In some embodiments, the ligand, or a fragment thereof, binds to Trop2. In some embodiments, the ligand, or a fragment thereof, binds to sialic acid-binding Ig-like lectin 10 (SIGLEC10). In some embodiments, the ligand, or a fragment thereof, binds to milk fat globule EGF and factor V/VIII domain containing (MFGE8). In some embodiments, the ligand, or a fragment thereof, binds to growth arrest-specific gene 6 (GAS6). In some embodiments, the ligand, or a fragment thereof, binds to T-cell membrane protein 4 (TIM4).

In some embodiments, the polypeptide comprises a first ligand amino acid sequence, or a fragment thereof, that binds to any one of CD166 antigen, OX-2 membrane glycoprotein, T-lymphocyte activation antigen CD86, EpCAM, RANK, CCL19, CCL21, LTβR, MHCII, CD40, CD80, CD86, lymphocyte antigen 6D, PlgR, L1CAM, LIFR, integrin alpha-6, integrin beta-4, podoplanin, CCR7, CD24, CaSR, CD63, nAChRalpha1, CD70, acetylcholine receptor subunit delta, CD74, claudin-1, CD80, claudin-4, CD81, claudin-6, CD9, immunoglobulin superfamily member 1, cadherin-1, large neutral amino acids transporter small subunit 2, cadherin-2, somatostatin receptor type 2, dectin-1, TRIC-A, HER3, CD23 antigen, IL-20RB, lymphocyte antigen 6D, DEC-205, LY6/PLAUR domain containing 3, macrophage receptor MARCO, oncostatin-M-specific receptor subunit beta, syndecan 1, syndecan-4, HAI-2, or Trop2; and a second ligand amino acid sequence, or a fragment thereof, that binds to any one of CD166 antigen, OX-2 membrane glycoprotein, T-lymphocyte activation antigen CD86, EpCAM, RANK, CCL19, CCL21, LTβR, MHCII, CD40, CD80, CD86, lymphocyte antigen 6D, PlgR, L1CAM, LIFR, integrin alpha-6, integrin beta-4, podoplanin, CCR7, CD24, CaSR, CD63, nAChRalpha1, CD70, acetylcholine receptor subunit delta, CD74, claudin-1, CD80, claudin-4, CD81, claudin-6, CD9, immunoglobulin superfamily member 1, cadherin-1, large neutral amino acids transporter small subunit 2, cadherin-2, somatostatin receptor type 2, dectin-1, TRIC-A, HER3, CD23 antigen, IL-20RB, lymphocyte antigen 6D, DEC-205, LY6/PLAUR domain containing 3, macrophage receptor MARCO, oncostatin-M-specific receptor subunit beta, syndecan 1, syndecan-4, HAI-2, Trop2, sialic acid-binding Ig-like lectin 10 (SIGLEC10), milk fat globule EGF and factor V/VIII domain containing (MFGE8), growth arrest-specific gene 6 (GAS6), or T-cell membrane protein 4 (TIM4).

In some embodiments, the polypeptide comprises a first ligand amino acid sequence, or a fragment thereof, that binds to any one of CD166 antigen, OX-2 membrane glycoprotein, T-lymphocyte activation antigen CD86, EpCAM, CD24, CD63, CD70, CD74, CD80, CD81, CD9, cadherin-1, cadherin-2, dectin-1, HER3, CD23 antigen, IL-20RB, lymphocyte antigen 6D, DEC-205, LY6/PLAUR domain containing 3, macrophage receptor MARCO, oncostatin-M-specific receptor subunit beta, syndecan 1, syndecan-4, HAI-2, or Trop2; and a second ligand amino acid sequence, or a fragment thereof, that binds to any one of CD166 antigen, OX-2 membrane glycoprotein, T-lymphocyte activation antigen CD86, EpCAM, CD24, CD63, CD70, CD74, CD80, CD81, CD9, cadherin-1, cadherin-2, dectin-1, HER3, CD23 antigen, IL-20RB, lymphocyte antigen 6D, DEC-205, LY6/PLAUR domain containing 3, macrophage receptor MARCO, oncostatin-M-specific receptor subunit beta, syndecan 1, syndecan-4, HAI-2, Trop2, sialic acid-binding Ig-like lectin 10 (SIGLEC10), milk fat globule EGF and factor V/VIII domain containing (MFGE8), growth arrest-specific gene 6 (GAS6), or T-cell membrane protein 4 (TIM4).

In some embodiments, the ligand, or a fragment thereof, is selected from milk fat globule EGF and factor V/VIII domain containing (MFGE8), T-cell membrane protein 4 (TIM4), growth arrest-specific gene 6 (GAS6), CD6, cell surface glycoprotein CD200 receptor 1, P-selectin, sialic acid-binding Ig-like lectin 10 (SIGLEC10), metalloproteinase inhibitor 1, lysosome-associated membrane glycoprotein 1, lysosome-associated membrane glycoprotein 2, integrin beta-1, CD27 antigen, macrophage migration inhibitory factor, CD44 antigen, cytotoxic T-lymphocyte protein 4, programmed cell death 1 ligand 1, T-cell-specific surface glycoprotein CD28, T-lymphocyte activation antigen CD86, complement receptor type 2, prostaglandin F2 receptor negative regulator, immunoglobulin superfamily member 8, integrin beta-1, B-lymphocyte antigen CD19, CD44 antigen, T-lymphocyte activation antigen CD80, T-cell-specific surface glycoprotein CD28, cytotoxic T-lymphocyte protein 4, membrane cofactor protein, proheparin-binding EGF-like growth factor, immunoglobulin superfamily member 8, prostaglandin F2 receptor negative regulator, integrin alpha-3, integrin alpha-6, integrin beta-1, epidermal growth factor receptor, integrin alpha-E, killer cell lectin-like receptor subfamily G member 1, hepatocyte growth factor receptor, cadherin-2, fibroblast growth factor receptor 1, low-density lipoprotein receptor-related protein 5, integrin beta-4, epithelial cell adhesion molecule, cadherin-1, pro-neuregulin-1, pro-neuregulin-2, complement receptor type 2, integrin alpha-X, integrin beta-2, neurogenic locus notch homolog protein 2, interleukin-19, interleukin-20, interleukin-24, receptor-type tyrosine-protein phosphatase C, anterior gradient protein 2 homolog, secretoglobin family 3A member 2, cardiotrophin-1, leukemia inhibitory factor, oncostatin-M, kappa-casein, fibroblast growth factor 2, extracellular glycoprotein lacritin, fibroblast growth factor 2, fibroblast growth factor 2, hepatocyte growth factor, hepatocyte growth factor activator, trypsin-3, prostasin, suppressor of tumorigenicity 14 protein, and transmembrane protease serine 13.

In some embodiments, the ligand, or a fragment thereof, is CD6. In some embodiments, the ligand, or a fragment thereof, is cell surface glycoprotein CD200 receptor 1. In some embodiments, the ligand, or a fragment thereof, is P-selectin. In some embodiments, the ligand, or a fragment thereof, is sialic acid-binding Ig-like lectin 10. In some embodiments, the ligand, or a fragment thereof, is metalloproteinase inhibitor 1. In some embodiments, the ligand, or a fragment thereof, is lysosome-associated membrane glycoprotein 1. In some embodiments, the ligand, or a fragment thereof, is lysosome-associated membrane glycoprotein 2. In some embodiments, the ligand, or a fragment thereof, is integrin beta-1. In some embodiments, the ligand, or a fragment thereof, is CD27 antigen. In some embodiments, the ligand, or a fragment thereof, is macrophage migration inhibitory factor. In some embodiments, the ligand, or a fragment thereof, is CD44 antigen. In some embodiments, the ligand, or a fragment thereof, is cytotoxic T-lymphocyte protein 4. In some embodiments, the ligand, or a fragment thereof, is programmed cell death 1 ligand 1. In some embodiments, the ligand, or a fragment thereof, is T-cell-specific surface glycoprotein CD28. In some embodiments, the ligand, or a fragment thereof, is T-lymphocyte activation antigen CD86. In some embodiments, the ligand, or a fragment thereof, is complement receptor type 2. In some embodiments, the ligand, or a fragment thereof, is prostaglandin F2 receptor negative regulator. In some embodiments, the ligand, or a fragment thereof, is immunoglobulin superfamily member 8. In some embodiments, the ligand, or a fragment thereof, is integrin beta-1. In some embodiments, the ligand, or a fragment thereof, is B-lymphocyte antigen CD19. In some embodiments, the ligand, or a fragment thereof, is CD44 antigen. In some embodiments, the ligand, or a fragment thereof, is T-lymphocyte activation antigen CD80. In some embodiments, the ligand, or a fragment thereof, is T-cell-specific surface glycoprotein CD28. In some embodiments, the ligand, or a fragment thereof, is cytotoxic T-lymphocyte protein 4. In some embodiments, the ligand, or a fragment thereof, is membrane cofactor protein. In some embodiments, the ligand, or a fragment thereof, is proheparin-binding EGF-like growth factor. In some embodiments, the ligand, or a fragment thereof, is immunoglobulin superfamily member 8. In some embodiments, the ligand, or a fragment thereof, is prostaglandin F2 receptor negative regulator. In some embodiments, the ligand, or a fragment thereof, is integrin alpha-3. In some embodiments, the ligand, or a fragment thereof, is integrin alpha-6. In some embodiments, the ligand, or a fragment thereof, is integrin beta-1. In some embodiments, the ligand, or a fragment thereof, is epidermal growth factor receptor. In some embodiments, the ligand, or a fragment thereof, is integrin alpha-E. In some embodiments, the ligand, or a fragment thereof, is killer cell lectin-like receptor subfamily G member 1. In some embodiments, the ligand, or a fragment thereof, is hepatocyte growth factor receptor. In some embodiments, the ligand, or a fragment thereof, is cadherin-2. In some embodiments, the ligand, or a fragment thereof, is fibroblast growth factor receptor 1. In some embodiments, the ligand, or a fragment thereof, is low-density lipoprotein receptor-related protein 5. In some embodiments, the ligand, or a fragment thereof, is integrin beta-4. In some embodiments, the ligand, or a fragment thereof, is epithelial cell adhesion molecule. In some embodiments, the ligand, or a fragment thereof, is cadherin-1. In some embodiments, the ligand, or a fragment thereof, is pro-neuregulin-1. In some embodiments, the ligand, or a fragment thereof, is pro-neuregulin-2. In some embodiments, the ligand, or a fragment thereof, is complement receptor type 2. In some embodiments, the ligand, or a fragment thereof, is integrin alpha-X. In some embodiments, the ligand, or a fragment thereof, is integrin beta-2. In some embodiments, the ligand, or a fragment thereof, is neurogenic locus notch homolog protein 2. In some embodiments, the ligand, or a fragment thereof, is interleukin-19. In some embodiments, the ligand, or a fragment thereof, is interleukin-20. In some embodiments, the ligand, or a fragment thereof, is interleukin-24. In some embodiments, the ligand, or a fragment thereof, is receptor-type tyrosine-protein phosphatase C. In some embodiments, the ligand, or a fragment thereof, is anterior gradient protein 2 homolog. In some embodiments, the ligand, or a fragment thereof, is secretoglobin family 3A member 2. In some embodiments, the ligand, or a fragment thereof, is cardiotrophin-1. In some embodiments, the ligand, or a fragment thereof, is leukemia inhibitory factor. In some embodiments, the ligand, or a fragment thereof, is oncostatin-M. In some embodiments, the ligand, or a fragment thereof, is kappa-casein. In some embodiments, the ligand, or a fragment thereof, is fibroblast growth factor 2. In some embodiments, the ligand, or a fragment thereof, is extracellular glycoprotein lacritin. In some embodiments, the ligand, or a fragment thereof, is fibroblast growth factor 2. In some embodiments, the ligand, or a fragment thereof, is fibroblast growth factor 2. In some embodiments, the ligand, or a fragment thereof, is hepatocyte growth factor. In some embodiments, the ligand, or a fragment thereof, is hepatocyte growth factor activator. In some embodiments, the ligand, or a fragment thereof, is trypsin-3. In some embodiments, the ligand, or a fragment thereof, is prostasin. In some embodiments, the ligand, or a fragment thereof, is suppressor of tumorigenicity 14 protein. In some embodiments, the ligand, or a fragment thereof, is transmembrane protease serine 13.

In some embodiments, the polypeptide comprises a first ligand amino acid sequence, or a fragment thereof, selected from any one of CD6, cell surface glycoprotein CD200 receptor 1, P-selectin, sialic acid-binding Ig-like lectin 10, metalloproteinase inhibitor 1, lysosome-associated membrane glycoprotein 1, lysosome-associated membrane glycoprotein 2, integrin beta-1, CD27 antigen, macrophage migration inhibitory factor, CD44 antigen, cytotoxic T-lymphocyte protein 4, programmed cell death 1 ligand 1, T-cell-specific surface glycoprotein CD28, T-lymphocyte activation antigen CD86, complement receptor type 2, prostaglandin F2 receptor negative regulator, immunoglobulin superfamily member 8, integrin beta-1, B-lymphocyte antigen CD19, CD44 antigen, T-lymphocyte activation antigen CD80, T-cell-specific surface glycoprotein CD28, cytotoxic T-lymphocyte protein 4, membrane cofactor protein, proheparin-binding EGF-like growth factor, immunoglobulin superfamily member 8, prostaglandin F2 receptor negative regulator, integrin alpha-3, integrin alpha-6, integrin beta-1, epidermal growth factor receptor, integrin alpha-E, killer cell lectin-like receptor subfamily G member 1, hepatocyte growth factor receptor, cadherin-2, fibroblast growth factor receptor 1, low-density lipoprotein receptor-related protein 5, integrin beta-4, epithelial cell adhesion molecule, cadherin-1, pro-neuregulin-1, pro-neuregulin-2, complement receptor type 2, integrin alpha-X, integrin beta-2, neurogenic locus notch homolog protein 2, interleukin-19, interleukin-20, interleukin-24, receptor-type tyrosine-protein phosphatase C, anterior gradient protein 2 homolog, secretoglobin family 3A member 2, cardiotrophin-1, leukemia inhibitory factor, oncostatin-M, kappa-casein, fibroblast growth factor 2, extracellular glycoprotein lacritin, fibroblast growth factor 2, fibroblast growth factor 2, hepatocyte growth factor, hepatocyte growth factor activator, trypsin-3, prostasin, suppressor of tumorigenicity 14 protein, and transmembrane protease serine 13; and a second ligand amino acid sequence, or a fragment thereof, selected from any one of CD6, cell surface glycoprotein CD200 receptor 1, P-selectin, sialic acid-binding Ig-like lectin 10, metalloproteinase inhibitor 1, lysosome-associated membrane glycoprotein 1, lysosome-associated membrane glycoprotein 2, integrin beta-1, CD27 antigen, macrophage migration inhibitory factor, CD44 antigen, cytotoxic T-lymphocyte protein 4, programmed cell death 1 ligand 1, T-cell-specific surface glycoprotein CD28, T-lymphocyte activation antigen CD86, complement receptor type 2, prostaglandin F2 receptor negative regulator, immunoglobulin superfamily member 8, integrin beta-1, B-lymphocyte antigen CD19, CD44 antigen, T-lymphocyte activation antigen CD80, T-cell-specific surface glycoprotein CD28, cytotoxic T-lymphocyte protein 4, membrane cofactor protein, proheparin-binding EGF-like growth factor, immunoglobulin superfamily member 8, prostaglandin F2 receptor negative regulator, integrin alpha-3, integrin alpha-6, integrin beta-1, epidermal growth factor receptor, integrin alpha-E, killer cell lectin-like receptor subfamily G member 1, hepatocyte growth factor receptor, cadherin-2, fibroblast growth factor receptor 1, low-density lipoprotein receptor-related protein 5, integrin beta-4, epithelial cell adhesion molecule, cadherin-1, pro-neuregulin-1, pro-neuregulin-2, complement receptor type 2, integrin alpha-X, integrin beta-2, neurogenic locus notch homolog protein 2, interleukin-19, interleukin-20, interleukin-24, receptor-type tyrosine-protein phosphatase C, anterior gradient protein 2 homolog, secretoglobin family 3A member 2, cardiotrophin-1, leukemia inhibitory factor, oncostatin-M, kappa-casein, fibroblast growth factor 2, extracellular glycoprotein lacritin, fibroblast growth factor 2, fibroblast growth factor 2, hepatocyte growth factor, hepatocyte growth factor activator, trypsin-3, prostasin, suppressor of tumorigenicity 14 protein, and transmembrane protease serine 13.

In some embodiments, the antigen of interest is an auto-antigen, a self-antigen, a tolerance inducing antigen, or a fragment thereof. In some embodiments, the antigen is an auto-antigen, or a fragment thereof. In some embodiments, the antigen is a self-antigen, or a fragment thereof. In some embodiments, the antigen is a tolerance inducing antigen, or a fragment thereof.

In some embodiments, the auto-antigen, self-antigen, tolerance inducing antigen, or a fragment thereof, is encoded for by a gene selected from GAD1, GAD2, INS, G6PC2, SLC30A8, PTPRN, TPM3, POLR3A, POLR3B, POLR3C, POLR3D, POLR3E, POLR3F, POLR3G, POLR3H, POLR3K, TOP1, AGTR1, EDNRA, CENPA, CENPB, CENPC, TSPAN7, GRIN1, FGF23, GRIA1, THSR, CSF2, IFNG, GRIA2, COL4A3, THSD7A, CNTNAP2, LGL1, PLA2R, MUSK, LRP4, AQP4, TRIM21, TROVE2, SNRPA1, SNRBP70, SNRPD3, HARS, TARS, ARS, IFIH1, EXOSC9, EXOSC107, PTRN, SCL30A8, PPIL2, MSH1, H4, H2AFY, H2AFJ, HIST1H2BJ, HIST1H2BA, HIST1H1C, H1F0, NCL, RCC1, CBX1, PURA, SF3B3, RMB8A, PRPF19, PRPF40A, HNRNPR, HNRNPL, HNRNPUL2, SYNCRIP, SNRNP70, EFTUD2, USP39, DHX15, SFRS1, SFRS7, SFRS2, TRA2B, TRA2A, ELAVL1, MRPL2, RPL18, MRPS27, EEF1A2, EIF3D, SSB, LRP2, MYH9, MYH10, ACTA1, ACTN4, ACTN1, LMNB2, LMNA, LMNB1, CSPG4, SPNA2, SPNB2, CLTC, TPM4, TPM1, TUBB2B, TUBA1A, MSN, PLEC1, VIM, AGRN, EPB41, TAGLN2, GM5414, ARG9A, HSPA5, HSPA11, HSP90AB1, PRDX1, YWHAE, YWHAG, YWHAH, YWHAB, SFN, ANXA2, AIFM1, DLST, DLAT, DBT, BCKDHA, AGMAT, ATP1A1, ATP1B1, ATP1A3, CPT2, PDIA4, P4HB, PDIA6, CALR, CDH16, CANX, ALDH1L1, GANAB, PSMA8, PSMA3, PSMA4, PSMA6, PSMA7, PSMA2, PSMA1, PSMA5, PSMB4, PSMB1, PSMB5, PSMB6, PSMB7, PSMB2, VCP, SERPINB5, PPIB, POR, MT-CO2, ILF2, APEH, COQ9, PDZK1, ACOX3, PCCA, ANPEP, CPS1, S100A11, GGT1, TGM2, GBA, GAA, GLA, NAGA, IDUA, HEXA, SMPD1, ASAH1, GALC, GALNS, ARSB, IDS, HGSNAT, GUSB, HYAL1, SIAL1, GNPTAB, GNPTG, G6PC, SLC37A4, ATP7B, PAH, ABCA4, MYOC, F8, F9, ADAMTS13, VWF, GPIBA, GPIBB, GP5, GP6, GP9, ITGA2B, ITGB3, PF4, HLA-A, HLA-B, HLA-C, HLA-E, HLA-L, HLA-J, HLA-K, HLA-H, HLA-G, HLA-DM, HLA-DO, HLA-DP, HLA-DQ, and HLA-DR. In some embodiments, the auto-antigen, self-antigen, tolerance inducing antigen, or a fragment thereof, is encoded for by the GRIN1 gene. In some embodiments, the auto-antigen, self-antigen, tolerance inducing antigen, or a fragment thereof, is encoded for by the GAD1 gene. In some embodiments, the auto-antigen, self-antigen, tolerance inducing antigen, or a fragment thereof, is encoded for by the GAD2 gene. In some embodiments, the auto-antigen, self-antigen, tolerance inducing antigen, or a fragment thereof, is encoded for by the G6PC2 gene. In some embodiments, the auto-antigen, self-antigen, tolerance inducing antigen, or a fragment thereof, is encoded for by the SLC30A8 gene. In some embodiments, the auto-antigen, self-antigen, tolerance inducing antigen, or a fragment thereof, is encoded for by the PTPRN gene. In some embodiments, the auto-antigen, self-antigen, tolerance inducing antigen, or a fragment thereof, is encoded for by the POLR3B gene. In some embodiments, the auto-antigen, self-antigen, tolerance inducing antigen, or a fragment thereof, is encoded for by the POLR3C gene. In some embodiments, the auto-antigen, self-antigen, tolerance inducing antigen, or a fragment thereof, is encoded for by the POLR3D gene. In some embodiments, the auto-antigen, self-antigen, tolerance inducing antigen, or a fragment thereof, is encoded for by the POLR3E gene. In some embodiments, the auto-antigen, self-antigen, tolerance inducing antigen, or a fragment thereof, is encoded for by the POLR3F gene. In some embodiments, the auto-antigen, self-antigen, tolerance inducing antigen, or a fragment thereof, is encoded for by the POLR3G gene. In some embodiments, the auto-antigen, self-antigen, tolerance inducing antigen, or a fragment thereof, is encoded for by the POLR3H gene. In some embodiments, the auto-antigen, self-antigen, tolerance inducing antigen, or a fragment thereof, is encoded for by the AGTR1 gene. In some embodiments, the auto-antigen, self-antigen, tolerance inducing antigen, or a fragment thereof, is encoded for by the EDNRA gene. In some embodiments, the auto-antigen, self-antigen, tolerance inducing antigen, or a fragment thereof, is encoded for by the CENPC gene. In some embodiments, the auto-antigen, self-antigen, tolerance inducing antigen, or a fragment thereof, is encoded for by the FGF23 gene. In some embodiments, the auto-antigen, self-antigen, tolerance inducing antigen, or a fragment thereof, is encoded for by the GRIA1 gene. In some embodiments, the auto-antigen, self-antigen, tolerance inducing antigen, or a fragment thereof, is encoded for by the THSR gene. In some embodiments, the auto-antigen, self-antigen, tolerance inducing antigen, or a fragment thereof, is encoded for by the CSF2 gene. In some embodiments, the auto-antigen, self-antigen, tolerance inducing antigen, or a fragment thereof, is encoded for by the IFNG gene. In some embodiments, the auto-antigen, self-antigen, tolerance inducing antigen, or a fragment thereof, is encoded for by the GRIA2 gene. In some embodiments, the auto-antigen, self-antigen, tolerance inducing antigen, or a fragment thereof, is encoded for by the COL4A3 gene. In some embodiments, the auto-antigen, self-antigen, tolerance inducing antigen, or a fragment thereof, is encoded for by the THSD7A gene. In some embodiments, the auto-antigen, self-antigen, tolerance inducing antigen, or a fragment thereof, is encoded for by the CNTNAP2 gene. In some embodiments, the auto-antigen, self-antigen, tolerance inducing antigen, or a fragment thereof, is encoded for by the LGL1 gene. In some embodiments, the auto-antigen, self-antigen, tolerance inducing antigen, or a fragment thereof, is encoded for by the PLA2R gene. In some embodiments, the auto-antigen, self-antigen, tolerance inducing antigen, or a fragment thereof, is encoded for by the MUSK gene. In some embodiments, the auto-antigen, self-antigen, tolerance inducing antigen, or a fragment thereof, is encoded for by the LRP4 gene. In some embodiments, the auto-antigen, self-antigen, tolerance inducing antigen, or a fragment thereof, is encoded for by the AQP4 gene. In some embodiments, the auto-antigen, self-antigen, tolerance inducing antigen, or a fragment thereof, is encoded for by the TRIM21 gene. In some embodiments, the auto-antigen, self-antigen, tolerance inducing antigen, or a fragment thereof, is encoded for by the TROVE2 gene. In some embodiments, the auto-antigen, self-antigen, tolerance inducing antigen, or a fragment thereof, is encoded for by the SNRPA1 gene. In some embodiments, the auto-antigen, self-antigen, tolerance inducing antigen, or a fragment thereof, is encoded for by the SNRBP70 gene. In some embodiments, the auto-antigen, self-antigen, tolerance inducing antigen, or a fragment thereof, is encoded for by the SNRPD3 gene. In some embodiments, the auto-antigen, self-antigen, tolerance inducing antigen, or a fragment thereof, is encoded for by the HARS gene. In some embodiments, the auto-antigen, self-antigen, tolerance inducing antigen, or a fragment thereof, is encoded for by the TARS gene. In some embodiments, the auto-antigen, self-antigen, tolerance inducing antigen, or a fragment thereof, is encoded for by the ARS gene. In some embodiments, the auto-antigen, self-antigen, tolerance inducing antigen, or a fragment thereof, is encoded for by the IFIH1 gene. In some embodiments, the auto-antigen, self-antigen, tolerance inducing antigen, or a fragment thereof, is encoded for by the CENPA gene. In some embodiments, the auto-antigen, self-antigen, tolerance inducing antigen, or a fragment thereof, is encoded for by the CENPB gene. In some embodiments, the auto-antigen, self-antigen, tolerance inducing antigen, or a fragment thereof, is encoded for by the EXOSC9 gene. In some embodiments, the auto-antigen, self-antigen, tolerance inducing antigen, or a fragment thereof, is encoded for by the EXOSC107 gene. In some embodiments, the auto-antigen, self-antigen, tolerance inducing antigen, or a fragment thereof, is encoded for by the POLR3A gene. In some embodiments, the auto-antigen, self-antigen, tolerance inducing antigen, or a fragment thereof, is encoded for by the POLR3K gene. In some embodiments, the auto-antigen, self-antigen, tolerance inducing antigen, or a fragment thereof, is encoded for by the PTRN gene. In some embodiments, the auto-antigen, self-antigen, tolerance inducing antigen, or a fragment thereof, is encoded for by the INS gene. In some embodiments, the auto-antigen, self-antigen, tolerance inducing antigen, or a fragment thereof, is encoded for by the GAD2 gene. In some embodiments, the auto-antigen, self-antigen, tolerance inducing antigen, or a fragment thereof, is encoded for by the SCL30A8 gene. In some embodiments, the auto-antigen, self-antigen, tolerance inducing antigen, or a fragment thereof, is encoded for by the TSPAN7 gene. In some embodiments, the auto-antigen, self-antigen, tolerance inducing antigen, or a fragment thereof, is encoded for by the PPIL2 gene. In some embodiments, the auto-antigen, self-antigen, tolerance inducing antigen, or a fragment thereof, is encoded for by the MSH1 gene. In some embodiments, the auto-antigen, self-antigen, tolerance inducing antigen, or a fragment thereof, is encoded for by the H4 gene. In some embodiments, the auto-antigen, self-antigen, tolerance inducing antigen, or a fragment thereof, is encoded for by the H2AFY gene. In some embodiments, the auto-antigen, self-antigen, tolerance inducing antigen, or a fragment thereof, is encoded for by the H2AFJ gene. In some embodiments, the auto-antigen, self-antigen, tolerance inducing antigen, or a fragment thereof, is encoded for by the HIST1H2BJ gene. In some embodiments, the auto-antigen, self-antigen, tolerance inducing antigen, or a fragment thereof, is encoded for by the HIST1H2BA gene. In some embodiments, the auto-antigen, self-antigen, tolerance inducing antigen, or a fragment thereof, is encoded for by the HISTIHIC gene. In some embodiments, the auto-antigen, self-antigen, tolerance inducing antigen, or a fragment thereof, is encoded for by the HIFM gene. In some embodiments, the auto-antigen, self-antigen, tolerance inducing antigen, or a fragment thereof, is encoded for by the NCL gene. In some embodiments, the auto-antigen, self-antigen, tolerance inducing antigen, or a fragment thereof, is encoded for by the TOP1 gene. In some embodiments, the auto-antigen, self-antigen, tolerance inducing antigen, or a fragment thereof, is encoded for by the RCC1 gene. In some embodiments, the auto-antigen, self-antigen, tolerance inducing antigen, or a fragment thereof, is encoded for by the CBX1 gene. In some embodiments, the auto-antigen, self-antigen, tolerance inducing antigen, or a fragment thereof, is encoded for by the PURA gene. In some embodiments, the auto-antigen, self-antigen, tolerance inducing antigen, or a fragment thereof, is encoded for by the SF3B3 gene. In some embodiments, the auto-antigen, self-antigen, tolerance inducing antigen, or a fragment thereof, is encoded for by the RMB8A gene. In some embodiments, the auto-antigen, self-antigen, tolerance inducing antigen, or a fragment thereof, is encoded for by the PRPF19 gene. In some embodiments, the auto-antigen, self-antigen, tolerance inducing antigen, or a fragment thereof, is encoded for by the PRPF40A gene. In some embodiments, the auto-antigen, self-antigen, tolerance inducing antigen, or a fragment thereof, is encoded for by the HNRNPR gene. In some embodiments, the auto-antigen, self-antigen, tolerance inducing antigen, or a fragment thereof, is encoded for by the HNRNPL gene. In some embodiments, the auto-antigen, self-antigen, tolerance inducing antigen, or a fragment thereof, is encoded for by the HNRNPUL2 gene. In some embodiments, the auto-antigen, self-antigen, tolerance inducing antigen, or a fragment thereof, is encoded for by the SYNCRIP gene. In some embodiments, the auto-antigen, self-antigen, tolerance inducing antigen, or a fragment thereof, is encoded for by the SNRNP70 gene. In some embodiments, the auto-antigen, self-antigen, tolerance inducing antigen, or a fragment thereof, is encoded for by the EFTUD2 gene. In some embodiments, the auto-antigen, self-antigen, tolerance inducing antigen, or a fragment thereof, is encoded for by the USP39 gene. In some embodiments, the auto-antigen, self-antigen, tolerance inducing antigen, or a fragment thereof, is encoded for by the DHX15 gene. In some embodiments, the auto-antigen, self-antigen, tolerance inducing antigen, or a fragment thereof, is encoded for by the SFRS1 gene. In some embodiments, the auto-antigen, self-antigen, tolerance inducing antigen, or a fragment thereof, is encoded for by the SFRS7 gene. In some embodiments, the auto-antigen, self-antigen, tolerance inducing antigen, or a fragment thereof, is encoded for by the SFRS2 gene. In some embodiments, the auto-antigen, self-antigen, tolerance inducing antigen, or a fragment thereof, is encoded for by the TRA2B gene. In some embodiments, the auto-antigen, self-antigen, tolerance inducing antigen, or a fragment thereof, is encoded for by the TRA2A gene. In some embodiments, the auto-antigen, self-antigen, tolerance inducing antigen, or a fragment thereof, is encoded for by the ELAVL1 gene. In some embodiments, the auto-antigen, self-antigen, tolerance inducing antigen, or a fragment thereof, is encoded for by the MRPL2 gene. In some embodiments, the auto-antigen, self-antigen, tolerance inducing antigen, or a fragment thereof, is encoded for by the RPL18 gene. In some embodiments, the auto-antigen, self-antigen, tolerance inducing antigen, or a fragment thereof, is encoded for by the MRPS27 gene. In some embodiments, the auto-antigen, self-antigen, tolerance inducing antigen, or a fragment thereof, is encoded for by the EEF1A2 gene. In some embodiments, the auto-antigen, self-antigen, tolerance inducing antigen, or a fragment thereof, is encoded for by the EIF3D gene. In some embodiments, the auto-antigen, self-antigen, tolerance inducing antigen, or a fragment thereof, is encoded for by the SSB gene. In some embodiments, the auto-antigen, self-antigen, tolerance inducing antigen, or a fragment thereof, is encoded for by the LRP2 gene. In some embodiments, the auto-antigen, self-antigen, tolerance inducing antigen, or a fragment thereof, is encoded for by the MYH9 gene. In some embodiments, the auto-antigen, self-antigen, tolerance inducing antigen, or a fragment thereof, is encoded for by the MYH10 gene. In some embodiments, the auto-antigen, self-antigen, tolerance inducing antigen, or a fragment thereof, is encoded for by the ACTA1 gene. In some embodiments, the auto-antigen, self-antigen, tolerance inducing antigen, or a fragment thereof, is encoded for by the ACTN4 gene. In some embodiments, the auto-antigen, self-antigen, tolerance inducing antigen, or a fragment thereof, is encoded for by the ACTN1 gene. In some embodiments, the auto-antigen, self-antigen, tolerance inducing antigen, or a fragment thereof, is encoded for by the LMNB2 gene. In some embodiments, the auto-antigen, self-antigen, tolerance inducing antigen, or a fragment thereof, is encoded for by the LMNA gene. In some embodiments, the auto-antigen, self-antigen, tolerance inducing antigen, or a fragment thereof, is encoded for by the LMNB1 gene. In some embodiments, the auto-antigen, self-antigen, tolerance inducing antigen, or a fragment thereof, is encoded for by the CSPG4 gene. In some embodiments, the auto-antigen, self-antigen, tolerance inducing antigen, or a fragment thereof, is encoded for by the SPNA2 gene. In some embodiments, the auto-antigen, self-antigen, tolerance inducing antigen, or a fragment thereof, is encoded for by the SPNB2 gene. In some embodiments, the auto-antigen, self-antigen, tolerance inducing antigen, or a fragment thereof, is encoded for by the CLTC gene. In some embodiments, the auto-antigen, self-antigen, tolerance inducing antigen, or a fragment thereof, is encoded for by the TPM4 gene. In some embodiments, the auto-antigen, self-antigen, tolerance inducing antigen, or a fragment thereof, is encoded for by the TPM1 gene. In some embodiments, the auto-antigen, self-antigen, tolerance inducing antigen, or a fragment thereof, is encoded for by the TPM3 gene. In some embodiments, the auto-antigen, self-antigen, tolerance inducing antigen, or a fragment thereof, is encoded for by the TUBB2B gene. In some embodiments, the auto-antigen, self-antigen, tolerance inducing antigen, or a fragment thereof, is encoded for by the TUBA1A gene. In some embodiments, the auto-antigen, self-antigen, tolerance inducing antigen, or a fragment thereof, is encoded for by the MSN gene. In some embodiments, the auto-antigen, self-antigen, tolerance inducing antigen, or a fragment thereof, is encoded for by the PLEC1 gene. In some embodiments, the auto-antigen, self-antigen, tolerance inducing antigen, or a fragment thereof, is encoded for by the VIM gene. In some embodiments, the auto-antigen, self-antigen, tolerance inducing antigen, or a fragment thereof, is encoded for by the AGRN gene. In some embodiments, the auto-antigen, self-antigen, tolerance inducing antigen, or a fragment thereof, is encoded for by the EPB41 gene. In some embodiments, the auto-antigen, self-antigen, tolerance inducing antigen, or a fragment thereof, is encoded for by the TAGLN2 gene. In some embodiments, the auto-antigen, self-antigen, tolerance inducing antigen, or a fragment thereof, is encoded for by the GM5414 gene. In some embodiments, the auto-antigen, self-antigen, tolerance inducing antigen, or a fragment thereof, is encoded for by the ARG9A gene. In some embodiments, the auto-antigen, self-antigen, tolerance inducing antigen, or a fragment thereof, is encoded for by the HSPA5 gene. In some embodiments, the auto-antigen, self-antigen, tolerance inducing antigen, or a fragment thereof, is encoded for by the HSPA11 gene. In some embodiments, the auto-antigen, self-antigen, tolerance inducing antigen, or a fragment thereof, is encoded for by the HSP90AB1 gene. In some embodiments, the auto-antigen, self-antigen, tolerance inducing antigen, or a fragment thereof, is encoded for by the PRDX1 gene. In some embodiments, the auto-antigen, self-antigen, tolerance inducing antigen, or a fragment thereof, is encoded for by the YWHAE gene. In some embodiments, the auto-antigen, self-antigen, tolerance inducing antigen, or a fragment thereof, is encoded for by the YWHAG gene. In some embodiments, the auto-antigen, self-antigen, tolerance inducing antigen, or a fragment thereof, is encoded for by the YWHAH gene. In some embodiments, the auto-antigen, self-antigen, tolerance inducing antigen, or a fragment thereof, is encoded for by the YWHAB gene. In some embodiments, the auto-antigen, self-antigen, tolerance inducing antigen, or a fragment thereof, is encoded for by the SFN gene. In some embodiments, the auto-antigen, self-antigen, tolerance inducing antigen, or a fragment thereof, is encoded for by the ANXA2 gene. In some embodiments, the auto-antigen, self-antigen, tolerance inducing antigen, or a fragment thereof, is encoded for by the AIFM1 gene. In some embodiments, the auto-antigen, self-antigen, tolerance inducing antigen, or a fragment thereof, is encoded for by the DLST gene. In some embodiments, the auto-antigen, self-antigen, tolerance inducing antigen, or a fragment thereof, is encoded for by the DLAT gene. In some embodiments, the auto-antigen, self-antigen, tolerance inducing antigen, or a fragment thereof, is encoded for by the DBT gene. In some embodiments, the auto-antigen, self-antigen, tolerance inducing antigen, or a fragment thereof, is encoded for by the BCKDHA gene. In some embodiments, the auto-antigen, self-antigen, tolerance inducing antigen, or a fragment thereof, is encoded for by the AGMAT gene. In some embodiments, the auto-antigen, self-antigen, tolerance inducing antigen, or a fragment thereof, is encoded for by the ATP1A1 gene. In some embodiments, the auto-antigen, self-antigen, tolerance inducing antigen, or a fragment thereof, is encoded for by the ATP1B1 gene. In some embodiments, the auto-antigen, self-antigen, tolerance inducing antigen, or a fragment thereof, is encoded for by the ATP1A3 gene. In some embodiments, the auto-antigen, self-antigen, tolerance inducing antigen, or a fragment thereof, is encoded for by the CPT2 gene. In some embodiments, the auto-antigen, self-antigen, tolerance inducing antigen, or a fragment thereof, is encoded for by the PDIA4 gene. In some embodiments, the auto-antigen, self-antigen, tolerance inducing antigen, or a fragment thereof, is encoded for by the P4HB gene. In some embodiments, the auto-antigen, self-antigen, tolerance inducing antigen, or a fragment thereof, is encoded for by the PDIA6 gene. In some embodiments, the auto-antigen, self-antigen, tolerance inducing antigen, or a fragment thereof, is encoded for by the CALR gene. In some embodiments, the auto-antigen, self-antigen, tolerance inducing antigen, or a fragment thereof, is encoded for by the CDH16 gene. In some embodiments, the auto-antigen, self-antigen, tolerance inducing antigen, or a fragment thereof, is encoded for by the CANX gene. In some embodiments, the auto-antigen, self-antigen, tolerance inducing antigen, or a fragment thereof, is encoded for by the ALDH1L1 gene. In some embodiments, the auto-antigen, self-antigen, tolerance inducing antigen, or a fragment thereof, is encoded for by the GANAB gene. In some embodiments, the auto-antigen, self-antigen, tolerance inducing antigen, or a fragment thereof, is encoded for by the PSMA8 gene. In some embodiments, the auto-antigen, self-antigen, tolerance inducing antigen, or a fragment thereof, is encoded for by the PSMA3 gene. In some embodiments, the auto-antigen, self-antigen, tolerance inducing antigen, or a fragment thereof, is encoded for by the PSMA4 gene. In some embodiments, the auto-antigen, self-antigen, tolerance inducing antigen, or a fragment thereof, is encoded for by the PSMA6 gene. In some embodiments, the auto-antigen, self-antigen, tolerance inducing antigen, or a fragment thereof, is encoded for by the PSMA7 gene. In some embodiments, the auto-antigen, self-antigen, tolerance inducing antigen, or a fragment thereof, is encoded for by the PSMA2 gene. In some embodiments, the auto-antigen, self-antigen, tolerance inducing antigen, or a fragment thereof, is encoded for by the PSMA1 gene. In some embodiments, the auto-antigen, self-antigen, tolerance inducing antigen, or a fragment thereof, is encoded for by the PSMA5 gene. In some embodiments, the auto-antigen, self-antigen, tolerance inducing antigen, or a fragment thereof, is encoded for by the PSMB4 gene. In some embodiments, the auto-antigen, self-antigen, tolerance inducing antigen, or a fragment thereof, is encoded for by the PSMB1 gene. In some embodiments, the auto-antigen, self-antigen, tolerance inducing antigen, or a fragment thereof, is encoded for by the PSMB5 gene. In some embodiments, the auto-antigen, self-antigen, tolerance inducing antigen, or a fragment thereof, is encoded for by the PSMB6 gene. In some embodiments, the auto-antigen, self-antigen, tolerance inducing antigen, or a fragment thereof, is encoded for by the PSMB7 gene. In some embodiments, the auto-antigen, self-antigen, tolerance inducing antigen, or a fragment thereof, is encoded for by the PSMB2 gene. In some embodiments, the auto-antigen, self-antigen, tolerance inducing antigen, or a fragment thereof, is encoded for by the VCP gene. In some embodiments, the auto-antigen, self-antigen, tolerance inducing antigen, or a fragment thereof, is encoded for by the SERPINB5 gene. In some embodiments, the auto-antigen, self-antigen, tolerance inducing antigen, or a fragment thereof, is encoded for by the PPIB gene. In some embodiments, the auto-antigen, self-antigen, tolerance inducing antigen, or a fragment thereof, is encoded for by the POR gene. In some embodiments, the auto-antigen, self-antigen, tolerance inducing antigen, or a fragment thereof, is encoded for by the MT-CO2 gene. In some embodiments, the auto-antigen, self-antigen, tolerance inducing antigen, or a fragment thereof, is encoded for by the ILF2 gene. In some embodiments, the auto-antigen, self-antigen, tolerance inducing antigen, or a fragment thereof, is encoded for by the APEH gene. In some embodiments, the auto-antigen, self-antigen, tolerance inducing antigen, or a fragment thereof, is encoded for by the COQ9 gene. In some embodiments, the auto-antigen, self-antigen, tolerance inducing antigen, or a fragment thereof, is encoded for by the PDZK1 gene. In some embodiments, the auto-antigen, self-antigen, tolerance inducing antigen, or a fragment thereof, is encoded for by the ACOX3 gene. In some embodiments, the auto-antigen, self-antigen, tolerance inducing antigen, or a fragment thereof, is encoded for by the PCCA gene. In some embodiments, the auto-antigen, self-antigen, tolerance inducing antigen, or a fragment thereof, is encoded for by the ANPEP gene. In some embodiments, the auto-antigen, self-antigen, tolerance inducing antigen, or a fragment thereof, is encoded for by the CPS1 gene. In some embodiments, the auto-antigen, self-antigen, tolerance inducing antigen, or a fragment thereof, is encoded for by the S100A11 gene. In some embodiments, the auto-antigen, self-antigen, tolerance inducing antigen, or a fragment thereof, is encoded for by the GGT1 gene. In some embodiments, the auto-antigen, self-antigen, tolerance inducing antigen, or a fragment thereof, is encoded for by the TGM2 gene. In some embodiments, the auto-antigen, self-antigen, tolerance inducing antigen, or a fragment thereof, is encoded for by the GBA gene. In some embodiments, the auto-antigen, self-antigen, tolerance inducing antigen, or a fragment thereof, is encoded for by the GAA gene. In some embodiments, the auto-antigen, self-antigen, tolerance inducing antigen, or a fragment thereof, is encoded for by the GLA gene. In some embodiments, the auto-antigen, self-antigen, tolerance inducing antigen, or a fragment thereof, is encoded for by the NAGA gene. In some embodiments, the auto-antigen, self-antigen, tolerance inducing antigen, or a fragment thereof, is encoded for by the IDUA gene. In some embodiments, the auto-antigen, self-antigen, tolerance inducing antigen, or a fragment thereof, is encoded for by the HEXA gene. In some embodiments, the auto-antigen, self-antigen, tolerance inducing antigen, or a fragment thereof, is encoded for by the SMPD1 gene. In some embodiments, the auto-antigen, self-antigen, tolerance inducing antigen, or a fragment thereof, is encoded for by the ASAH1 gene. In some embodiments, the auto-antigen, self-antigen, tolerance inducing antigen, or a fragment thereof, is encoded for by the GALC gene. In some embodiments, the auto-antigen, self-antigen, tolerance inducing antigen, or a fragment thereof, is encoded for by the GALNS gene. In some embodiments, the auto-antigen, self-antigen, tolerance inducing antigen, or a fragment thereof, is encoded for by the ARSB gene. In some embodiments, the auto-antigen, self-antigen, tolerance inducing antigen, or a fragment thereof, is encoded for by the IDS gene. In some embodiments, the auto-antigen, self-antigen, tolerance inducing antigen, or a fragment thereof, is encoded for by the HGSNAT gene. In some embodiments, the auto-antigen, self-antigen, tolerance inducing antigen, or a fragment thereof, is encoded for by the GUSB gene. In some embodiments, the auto-antigen, self-antigen, tolerance inducing antigen, or a fragment thereof, is encoded for by the HYAL1 gene. In some embodiments, the auto-antigen, self-antigen, tolerance inducing antigen, or a fragment thereof, is encoded for by the SIAL1 gene. In some embodiments, the auto-antigen, self-antigen, tolerance inducing antigen, or a fragment thereof, is encoded for by the GNPTAB gene. In some embodiments, the auto-antigen, self-antigen, tolerance inducing antigen, or a fragment thereof, is encoded for by the GNPTG gene. In some embodiments, the auto-antigen, self-antigen, tolerance inducing antigen, or a fragment thereof, is encoded for by the G6PC gene. In some embodiments, the auto-antigen, self-antigen, tolerance inducing antigen, or a fragment thereof, is encoded for by the SLC37A4 gene. In some embodiments, the auto-antigen, self-antigen, tolerance inducing antigen, or a fragment thereof, is encoded for by the ATP7B gene. In some embodiments, the auto-antigen, self-antigen, tolerance inducing antigen, or a fragment thereof, is encoded for by the PAH gene. In some embodiments, the auto-antigen, self-antigen, tolerance inducing antigen, or a fragment thereof, is encoded for by the ABCA4 gene. In some embodiments, the auto-antigen, self-antigen, tolerance inducing antigen, or a fragment thereof, is encoded for by the MYOC gene. In some embodiments, the auto-antigen, self-antigen, tolerance inducing antigen, or a fragment thereof, is encoded for by the F8 gene. In some embodiments, the auto-antigen, self-antigen, tolerance inducing antigen, or a fragment thereof, is encoded for by the F9 gene. In some embodiments, the auto-antigen, self-antigen, tolerance inducing antigen, or a fragment thereof, is encoded for by the ADAMTS13 gene. In some embodiments, the auto-antigen, self-antigen, tolerance inducing antigen, or a fragment thereof, is encoded for by the VWF gene. In some embodiments, the auto-antigen, self-antigen, tolerance inducing antigen, or a fragment thereof, is encoded for by the GPIBA gene. In some embodiments, the auto-antigen, self-antigen, tolerance inducing antigen, or a fragment thereof, is encoded for by the GPIBB gene. In some embodiments, the auto-antigen, self-antigen, tolerance inducing antigen, or a fragment thereof, is encoded for by the GP5 gene. In some embodiments, the auto-antigen, self-antigen, tolerance inducing antigen, or a fragment thereof, is encoded for by the GP6 gene. In some embodiments, the auto-antigen, self-antigen, tolerance inducing antigen, or a fragment thereof, is encoded for by the GP9 gene. In some embodiments, the auto-antigen, self-antigen, tolerance inducing antigen, or a fragment thereof, is encoded for by the ITGA2B gene. In some embodiments, the auto-antigen, self-antigen, tolerance inducing antigen, or a fragment thereof, is encoded for by the ITGB3 gene. In some embodiments, the auto-antigen, self-antigen, tolerance inducing antigen, or a fragment thereof, is encoded for by the PF4 gene. In some embodiments, the auto-antigen, self-antigen, tolerance inducing antigen, or a fragment thereof, is encoded for by the HLA-A gene. In some embodiments, the auto-antigen, self-antigen, tolerance inducing antigen, or a fragment thereof, is encoded for by the HLA-B gene. In some embodiments, the auto-antigen, self-antigen, tolerance inducing antigen, or a fragment thereof, is encoded for by the HLA-C gene. In some embodiments, the auto-antigen, self-antigen, tolerance inducing antigen, or a fragment thereof, is encoded for by the HLA-E gene. In some embodiments, the auto-antigen, self-antigen, tolerance inducing antigen, or a fragment thereof, is encoded for by the HLA-L gene. In some embodiments, the auto-antigen, self-antigen, tolerance inducing antigen, or a fragment thereof, is encoded for by the HLA-J gene. In some embodiments, the auto-antigen, self-antigen, tolerance inducing antigen, or a fragment thereof, is encoded for by the HLA-K gene. In some embodiments, the auto-antigen, self-antigen, tolerance inducing antigen, or a fragment thereof, is encoded for by the HLA-H gene. In some embodiments, the auto-antigen, self-antigen, tolerance inducing antigen, or a fragment thereof, is encoded for by the HLA-G gene. In some embodiments, the auto-antigen, self-antigen, tolerance inducing antigen, or a fragment thereof, is encoded for by the HLA-DM gene. In some embodiments, the auto-antigen, self-antigen, tolerance inducing antigen, or a fragment thereof, is encoded for by the HLA-DO gene. In some embodiments, the auto-antigen, self-antigen, tolerance inducing antigen, or a fragment thereof, is encoded for by the HLA-DP gene. In some embodiments, the auto-antigen, self-antigen, tolerance inducing antigen, or a fragment thereof, is encoded for by the HLA-DQ gene. In some embodiments, the auto-antigen, self-antigen, tolerance inducing antigen, or a fragment thereof, is encoded for by the HLA-DR gene.

In some embodiments, the auto-antigen, self-antigen, tolerance inducing antigen, or a fragment thereof, is selected from glutamate decarboxylase 1 (GAD), glutamate decarboxylase 2 (GAD65), proinsulin, islet-specific glucose-6-phosphatase catalytic subunit-related protein (IGRP), zinc transporter 8 (Znt8), receptor-type tyrosine-protein phosphatase-like N (IA2), tropomyosin alpha-3 chain (TPM3), granulocyte-macrophage colony-stimulating factor (GMCSF/CSF2), RNA polymerase III complex subunit A (RNAP3), RNA polymerase III complex subunit B (RNAP3), RNA polymerase III complex subunit C (RNAP3), RNA polymerase III complex subunit D (RNAP3), RNA polymerase III complex subunit E (RNAP3), RNA polymerase III complex subunit F (RNAP3), RNA polymerase III complex subunit G (RNAP3), RNA polymerase III complex subunit H (RNAP3), RNA polymerase III complex subunit K (RNAP3), DNA topoisomerase 1 (TOPO1), type-1 angiotensin II receptor (ATIR), endothelin-1 receptor (ETAR), major centromere protein A (CENP), major centromere protein B (CENP), major centromere protein C (CENP), tetraspanin-7 (TSPAN7), 21-hydroxylase (21OH), 17alpha-hydroxylase (17OH), Th/To, ANA, dsDNA, HMGCR, myosin, SSA/Ro, SSB/La, U1RNP, U3RNP, PM-Sci, myelin basic protein (MBP), IL17/IL22, ASMA, actin, amylase α2, GM-CSF, cyclic citrullinated proteins (CCP), snRNP, Ro52, Ro60, La, Jo-1, SRP, IFIH1, CENPA, insulin, RNA-P, U1-70K, Sm-D3, MDA-5, PL7, PL-12, centromere proteins, PM/SCL, RNA polymerase 3 complex, tetraspanin-7, peptidylprolyl isomerase like 2, Mlh1, proteinase-3, immunoglobulin G, immunoglobulin M, myeloperoxidase, acetylcholine receptor (AchR), MUSK, LRP4, HSP90. HSPA5, kallikrein 13, ADAMTS13, IFN-7, IFN-α/ω, IL6, IL12/IL23, desmogleins (e.g., desmoglein-3, desmoglein-1), aquaporin-4, myelin oligodendrocyte glycoprotein (MOG), GRIN1, GRIA1, GRIA2, leucine rich glioma inactivated 1 protein (LGI1), neurexin family protein contactin associated protein 2 (CNTNAP2), collagen (e.g., collagen IV-alpha 3 chain (COL4A3), COL17), BP180, phospholipase A2 receptor (PLA2R), thrombospondin type-1 domain-containing 7A (THSD7A), thyroid hormone stimulating receptor (THSR), NMDAR, FGF23, acid beta-glycosidase, acid alpha-glycosidase, alpha-galactosidase A, alpha-N-acetylgalactosaminidase, alpha-L-iduronohydrolase, beta-hexosaminidase A, sphingomyelin phosphodiesterase 1, N-acetylsphingosine amidohydrolase 1, galactocerebrosidase, N-acetylgalactosamine-sulfate sulfatase, arylsulfatase B, iduronate 2-sulfatase, heparan-alpha-glucosaminide-N-acetyltransferase, beta-glucuronidase, hyaluronidase, sialidase 1, N-acetylglucosamine-1-phosphotransferase alpha/beta, N-acetylglucosamine-1-phosphotransferase gamma, glucose-6-phosphatase catalytic subunit, glucose-6-phosphate transporter, ATP7B, Phenylalanine hydroxylase, ATP-binding cassette subfamily A member 4, myocilin, coagulation factor VIII, coagulation factor IX, von Willebrand factor, glycoprotein Ib alpha, glycoprotein Ib beta, glycoprotein V, glycoprotein VI, glycoprotein IX, glycoprotein IIb, glycoprotein IIIa, platelet factor 4, AAV capsid protein (such as AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.eB, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9, AAV9 K449R (or K449R AAV9), AAV9.16, AAV9.24, AAV9.45, AAbiodisV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74 (also referred to as AAVrh74), AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, 7m8, Spark100, AAVMYO and variants thereof), SpCas9, SaCas9, FnCas12a, LbCas12a, AcCas12a, Cas13b, APOBEC1, E. coli TadA, Moloney Murine Leukemia Virus Reverse Transcriptase (M-MLV RT), HLA-A, HLA-B, HLA-C, HLA-E, HLA-L, HLA-J, HLA-K, HLA-H, HLA-G, HLA-DM, HLA-DO, HLA-DP, HLA-DQ, and HLA-DR.

In some embodiments, the auto-antigen, self-antigen, tolerance inducing antigen, or a fragment thereof, is glutamate decarboxylase 1 (GAD). In some embodiments, the auto-antigen, self-antigen, tolerance inducing antigen, or a fragment thereof, is glutamate decarboxylase 2 (GAD65). In some embodiments, the auto-antigen, self-antigen, tolerance inducing antigen, or a fragment thereof, is zinc transporter 8 (Znt8). In some embodiments, the auto-antigen, self-antigen, tolerance inducing antigen, or a fragment thereof, is receptor-type tyrosine-protein phosphatase-like N (IA2). In some embodiments, the auto-antigen, self-antigen, tolerance inducing antigen, or a fragment thereof, is tropomyosin alpha-3 chain (TPM3). In some embodiments, the auto-antigen, self-antigen, tolerance inducing antigen, or a fragment thereof, is granulocyte-macrophage colony-stimulating factor (GMCSF/CSF2). In some embodiments, the auto-antigen, self-antigen, tolerance inducing antigen, or a fragment thereof, is RNA polymerase III complex subunit A (RNAP3). In some embodiments, the auto-antigen, self-antigen, tolerance inducing antigen, or a fragment thereof, is RNA polymerase III complex subunit B (RNAP3). In some embodiments, the auto-antigen, self-antigen, tolerance inducing antigen, or a fragment thereof, is RNA polymerase III complex subunit C (RNAP3). In some embodiments, the auto-antigen, self-antigen, tolerance inducing antigen, or a fragment thereof, is RNA polymerase III complex subunit D (RNAP3). In some embodiments, the auto-antigen, self-antigen, tolerance inducing antigen, or a fragment thereof, is RNA polymerase III complex subunit E (RNAP3). In some embodiments, the auto-antigen, self-antigen, tolerance inducing antigen, or a fragment thereof, is RNA polymerase III complex subunit F (RNAP3). In some embodiments, the auto-antigen, self-antigen, tolerance inducing antigen, or a fragment thereof, is RNA polymerase III complex subunit G (RNAP3). In some embodiments, the auto-antigen, self-antigen, tolerance inducing antigen, or a fragment thereof, is RNA polymerase III complex subunit H (RNAP3). In some embodiments, the auto-antigen, self-antigen, tolerance inducing antigen, or a fragment thereof, is RNA polymerase III complex subunit K (RNAP3). In some embodiments, the auto-antigen, self-antigen, tolerance inducing antigen, or a fragment thereof, is DNA topoisomerase 1 (TOPO1). In some embodiments, the auto-antigen, self-antigen, tolerance inducing antigen, or a fragment thereof, is type-1 angiotensin II receptor (AT1R). In some embodiments, the auto-antigen, self-antigen, tolerance inducing antigen, or a fragment thereof, is endothelin-1 receptor (ETAR). In some embodiments, the auto-antigen, self-antigen, tolerance inducing antigen, or a fragment thereof, is major centromere protein A (CENP). In some embodiments, the auto-antigen, self-antigen, tolerance inducing antigen, or a fragment thereof, is major centromere protein B (CENP). In some embodiments, the auto-antigen, self-antigen, tolerance inducing antigen, or a fragment thereof, is major centromere protein C (CENP). In some embodiments, the auto-antigen, self-antigen, tolerance inducing antigen, or a fragment thereof, is tetraspanin-7 (TSPAN7). In some embodiments, the auto-antigen, self-antigen, tolerance inducing antigen, or a fragment thereof, is proinsulin. In some embodiments, the auto-antigen, self-antigen, tolerance inducing antigen, or a fragment thereof, is islet-specific glucose-6-phosphatase catalytic subunit-related protein (IGRP). In some embodiments, the auto-antigen, self-antigen, tolerance inducing antigen, or a fragment thereof, is 21-hydroxylase (21OH). In some embodiments, the auto-antigen, self-antigen, tolerance inducing antigen, or a fragment thereof, is 17alpha-hydroxylase (17OH). In some embodiments, the auto-antigen, self-antigen, tolerance inducing antigen, or a fragment thereof, is Th/To. In some embodiments, the auto-antigen, self-antigen, tolerance inducing antigen, or a fragment thereof, is ANA. In some embodiments, the auto-antigen, self-antigen, tolerance inducing antigen, or a fragment thereof, is dsDNA. In some embodiments, the auto-antigen, self-antigen, tolerance inducing antigen, or a fragment thereof, is HMGCR. In some embodiments, the auto-antigen, self-antigen, tolerance inducing antigen, or a fragment thereof, is myosin. In some embodiments, the auto-antigen, self-antigen, tolerance inducing antigen, or a fragment thereof, is SSA/Ro. In some embodiments, the auto-antigen, self-antigen, tolerance inducing antigen, or a fragment thereof, is SSB/La. In some embodiments, the auto-antigen, self-antigen, tolerance inducing antigen, or a fragment thereof, is U1RNP. In some embodiments, the auto-antigen, self-antigen, tolerance inducing antigen, or a fragment thereof, is U3RNP. In some embodiments, the auto-antigen, self-antigen, tolerance inducing antigen, or a fragment thereof, is PM-Sci. In some embodiments, the auto-antigen, self-antigen, tolerance inducing antigen, or a fragment thereof, is myelin basic protein (MBP). In some embodiments, the auto-antigen, self-antigen, tolerance inducing antigen, or a fragment thereof, is IL17/IL22. In some embodiments, the auto-antigen, self-antigen, tolerance inducing antigen, or a fragment thereof, is ASMA. In some embodiments, the auto-antigen, self-antigen, tolerance inducing antigen, or a fragment thereof, is actin. In some embodiments, the auto-antigen, self-antigen, tolerance inducing antigen, or a fragment thereof, is amylase α2. In some embodiments, the auto-antigen, self-antigen, tolerance inducing antigen, or a fragment thereof, is GM-CSF. In some embodiments, the auto-antigen, self-antigen, tolerance inducing antigen, or a fragment thereof, is cyclic citrullinated proteins (CCP). In some embodiments, the auto-antigen, self-antigen, tolerance inducing antigen, or a fragment thereof, is snRNP. In some embodiments, the auto-antigen, self-antigen, tolerance inducing antigen, or a fragment thereof, is Ro52. In some embodiments, the auto-antigen, self-antigen, tolerance inducing antigen, or a fragment thereof, is Ro60. In some embodiments, the auto-antigen, self-antigen, tolerance inducing antigen, or a fragment thereof, is La. In some embodiments, the auto-antigen, self-antigen, tolerance inducing antigen, or a fragment thereof, is Jo-1. In some embodiments, the auto-antigen, self-antigen, tolerance inducing antigen, or a fragment thereof, is SRP. In some embodiments, the auto-antigen, self-antigen, tolerance inducing antigen, or a fragment thereof, is IFIH1. In some embodiments, the auto-antigen, self-antigen, tolerance inducing antigen, or a fragment thereof, is CENPA. In some embodiments, the auto-antigen, self-antigen, tolerance inducing antigen, or a fragment thereof, is RNAP3. In some embodiments, the auto-antigen, self-antigen, tolerance inducing antigen, or a fragment thereof, is insulin. In some embodiments, the auto-antigen, self-antigen, tolerance inducing antigen, or a fragment thereof, is Znt8. In some embodiments, the auto-antigen, self-antigen, tolerance inducing antigen, or a fragment thereof, is RNA-P. In some embodiments, the auto-antigen, self-antigen, tolerance inducing antigen, or a fragment thereof, is U1-70K. In some embodiments, the auto-antigen, self-antigen, tolerance inducing antigen, or a fragment thereof, is Sm-D3. In some embodiments, the auto-antigen, self-antigen, tolerance inducing antigen, or a fragment thereof, is MDA-5. In some embodiments, the auto-antigen, self-antigen, tolerance inducing antigen, or a fragment thereof, is PL7. In some embodiments, the auto-antigen, self-antigen, tolerance inducing antigen, or a fragment thereof, is PL-12. In some embodiments, the auto-antigen, self-antigen, tolerance inducing antigen, or a fragment thereof, is a centromere protein. In some embodiments, the auto-antigen, self-antigen, tolerance inducing antigen, or a fragment thereof, is PM/SCL. In some embodiments, the auto-antigen, self-antigen, tolerance inducing antigen, or a fragment thereof, is RNA polymerase 3 complex. In some embodiments, the auto-antigen, self-antigen, tolerance inducing antigen, or a fragment thereof, is tetraspanin-7. In some embodiments, the auto-antigen, self-antigen, tolerance inducing antigen, or a fragment thereof, is peptidylprolyl isomerase like 2. In some embodiments, the auto-antigen, self-antigen, tolerance inducing antigen, or a fragment thereof, is Mlh1. In some embodiments, the auto-antigen, self-antigen, tolerance inducing antigen, or a fragment thereof, is proteinase-3. In some embodiments, the auto-antigen, self-antigen, tolerance inducing antigen, or a fragment thereof, is immunoglobulin G. In some embodiments, the auto-antigen, self-antigen, tolerance inducing antigen, or a fragment thereof, is immunoglobulin M. In some embodiments, the auto-antigen, self-antigen, tolerance inducing antigen, or a fragment thereof, is myeloperoxidase. In some embodiments, the auto-antigen, self-antigen, tolerance inducing antigen, or a fragment thereof, is an acetylcholine receptor (AchR). In some embodiments, the auto-antigen, self-antigen, tolerance inducing antigen, or a fragment thereof, is MUSK. In some embodiments, the auto-antigen, self-antigen, tolerance inducing antigen, or a fragment thereof, is LRP4. In some embodiments, the auto-antigen, self-antigen, tolerance inducing antigen, or a fragment thereof, is HSP90. In some embodiments, the auto-antigen, self-antigen, tolerance inducing antigen, or a fragment thereof, is HSPA5. In some embodiments, the auto-antigen, self-antigen, tolerance inducing antigen, or a fragment thereof, is kallikrein 13. In some embodiments, the auto-antigen, self-antigen, tolerance inducing antigen, or a fragment thereof, is AT1R. In some embodiments, the auto-antigen, self-antigen, tolerance inducing antigen, or a fragment thereof, is ETAR. In some embodiments, the auto-antigen, self-antigen, tolerance inducing antigen, or a fragment thereof, is ADAMTS13. In some embodiments, the auto-antigen, self-antigen, tolerance inducing antigen, or a fragment thereof, is IFN-γ. In some embodiments, the auto-antigen, self-antigen, tolerance inducing antigen, or a fragment thereof, is IFN-α/o. In some embodiments, the auto-antigen, self-antigen, tolerance inducing antigen, or a fragment thereof, is IL6. In some embodiments, the auto-antigen, self-antigen, tolerance inducing antigen, or a fragment thereof, is IL12/IL23. In some embodiments, the auto-antigen, self-antigen, tolerance inducing antigen, or a fragment thereof, is a desmoglein. In some embodiments, the auto-antigen, self-antigen, tolerance inducing antigen, or a fragment thereof, is desmoglein-3. In some embodiments, the auto-antigen, self-antigen, tolerance inducing antigen, or a fragment thereof, is desmoglein-1. In some embodiments, the auto-antigen, self-antigen, tolerance inducing antigen, or a fragment thereof, is aquaporin-4. In some embodiments, the auto-antigen, self-antigen, tolerance inducing antigen, or a fragment thereof, is myelin oligodendrocyte glycoprotein (MOG). In some embodiments, the auto-antigen, self-antigen, tolerance inducing antigen, or a fragment thereof, is GRIN1. In some embodiments, the auto-antigen, self-antigen, tolerance inducing antigen, or a fragment thereof, is GRIAL. In some embodiments, the auto-antigen, self-antigen, tolerance inducing antigen, or a fragment thereof, is GRIA2. In some embodiments, the auto-antigen, self-antigen, tolerance inducing antigen, or a fragment thereof, is leucine rich glioma inactivated 1 protein (LGI1). In some embodiments, the auto-antigen, self-antigen, tolerance inducing antigen, or a fragment thereof, is neurexin family protein contactin associated protein 2 (CNTNAP2). In some embodiments, the auto-antigen, self-antigen, tolerance inducing antigen, or a fragment thereof, is a collagen. In some embodiments, the auto-antigen, self-antigen, tolerance inducing antigen, or a fragment thereof, is collagen IV-alpha 3 chain (COL4A3). In some embodiments, the auto-antigen, self-antigen, tolerance inducing antigen, or a fragment thereof, is COL17. In some embodiments, the auto-antigen, self-antigen, tolerance inducing antigen, or a fragment thereof, is BP180. In some embodiments, the auto-antigen, self-antigen, tolerance inducing antigen, or a fragment thereof, is phospholipase A2 receptor (PLA2R). In some embodiments, the auto-antigen, self-antigen, tolerance inducing antigen, or a fragment thereof, is thrombospondin type-1 domain-containing 7A (THSD7A). In some embodiments, the auto-antigen, self-antigen, tolerance inducing antigen, or a fragment thereof, is thyroid hormone stimulating receptor (THSR). In some embodiments, the auto-antigen, self-antigen, tolerance inducing antigen, or a fragment thereof, is GMCSF (CSF2). In some embodiments, the auto-antigen, self-antigen, tolerance inducing antigen, or a fragment thereof, is NMDAR. In some embodiments, the auto-antigen, self-antigen, tolerance inducing antigen, or a fragment thereof, is FGF23. In some embodiments, the auto-antigen, self-antigen, tolerance inducing antigen, or a fragment thereof, is acid beta-glycosidase. In some embodiments, the auto-antigen, self-antigen, tolerance inducing antigen, or a fragment thereof, is acid alpha-glycosidase. In some embodiments, the auto-antigen, self-antigen, tolerance inducing antigen, or a fragment thereof, is alpha-galactosidase A. In some embodiments, the auto-antigen, self-antigen, tolerance inducing antigen, or a fragment thereof, is alpha-N-acetylgalactosaminidase. In some embodiments, the auto-antigen, self-antigen, tolerance inducing antigen, or a fragment thereof, is alpha-L-iduronohydrolase. In some embodiments, the auto-antigen, self-antigen, tolerance inducing antigen, or a fragment thereof, is beta-hexosaminidase A. In some embodiments, the auto-antigen, self-antigen, tolerance inducing antigen, or a fragment thereof, is sphingomyelin phosphodiesterase 1. In some embodiments, the auto-antigen, self-antigen, tolerance inducing antigen, or a fragment thereof, is N-acetylsphingosine amidohydrolase 1. In some embodiments, the auto-antigen, self-antigen, tolerance inducing antigen, or a fragment thereof, is galactocerebrosidase. In some embodiments, the auto-antigen, self-antigen, tolerance inducing antigen, or a fragment thereof, is N-acetylgalactosamine-sulfate sulfatase. In some embodiments, the auto-antigen, self-antigen, tolerance inducing antigen, or a fragment thereof, is arylsulfatase B. In some embodiments, the auto-antigen, self-antigen, tolerance inducing antigen, or a fragment thereof, is iduronate 2-sulfatase. In some embodiments, the auto-antigen, self-antigen, tolerance inducing antigen, or a fragment thereof, is heparan-alpha-glucosaminide-N-acetyltransferase. In some embodiments, the auto-antigen, self-antigen, tolerance inducing antigen, or a fragment thereof, is beta-glucuronidase, hyaluronidase. In some embodiments, the auto-antigen, self-antigen, tolerance inducing antigen, or a fragment thereof, is sialidase 1. In some embodiments, the auto-antigen, self-antigen, tolerance inducing antigen, or a fragment thereof, is N-acetylglucosamine-1-phosphotransferase alpha/beta. In some embodiments, the auto-antigen, self-antigen, tolerance inducing antigen, or a fragment thereof, is N-acetylglucosamine-1-phosphotransferase gamma. In some embodiments, the auto-antigen, self-antigen, tolerance inducing antigen, or a fragment thereof, is glucose-6-phosphatase catalytic subunit. In some embodiments, the auto-antigen, self-antigen, tolerance inducing antigen, or a fragment thereof, is glucose-6-phosphate transporter. In some embodiments, the auto-antigen, self-antigen, tolerance inducing antigen, or a fragment thereof, is ATP7B. In some embodiments, the auto-antigen, self-antigen, tolerance inducing antigen, or a fragment thereof, is phenylalanine hydroxylase. In some embodiments, the auto-antigen, self-antigen, tolerance inducing antigen, or a fragment thereof, is ATP-binding cassette subfamily A member 4. In some embodiments, the auto-antigen, self-antigen, tolerance inducing antigen, or a fragment thereof, is myocilin. In some embodiments, the auto-antigen, self-antigen, tolerance inducing antigen, or a fragment thereof, is coagulation factor VIII. In some embodiments, the auto-antigen, self-antigen, tolerance inducing antigen, or a fragment thereof, is coagulation factor IX. In some embodiments, the auto-antigen, self-antigen, tolerance inducing antigen, or a fragment thereof, is a von Willebrand factor. In some embodiments, the auto-antigen, self-antigen, tolerance inducing antigen, or a fragment thereof, is glycoprotein Ib alpha. In some embodiments, the auto-antigen, self-antigen, tolerance inducing antigen, or a fragment thereof, is glycoprotein Ib beta. In some embodiments, the auto-antigen, self-antigen, tolerance inducing antigen, or a fragment thereof, is glycoprotein V. In some embodiments, the auto-antigen, self-antigen, tolerance inducing antigen, or a fragment thereof, is glycoprotein VI. In some embodiments, the auto-antigen, self-antigen, tolerance inducing antigen, or a fragment thereof, is glycoprotein IX. In some embodiments, the auto-antigen, self-antigen, tolerance inducing antigen, or a fragment thereof, is glycoprotein IIb. In some embodiments, the auto-antigen, self-antigen, tolerance inducing antigen, or a fragment thereof, is glycoprotein IIIa. In some embodiments, the auto-antigen, self-antigen, tolerance inducing antigen, or a fragment thereof, is platelet factor 4. In some embodiments, the auto-antigen, self-antigen, tolerance inducing antigen, or a fragment thereof, is an AAV capsid protein (such as AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.eB, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9, AAV9 K449R (or K449R AAV9), AAV9.16, AAV9.24, AAV9.45, AAbiodisV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74 (also referred to as AAVrh74), AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, 7m8, Spark100, AAVMYO and variants thereof). In some embodiments, the auto-antigen, self-antigen, tolerance inducing antigen, or a fragment thereof, is SpCas9. In some embodiments, the auto-antigen, self-antigen, tolerance inducing antigen, or a fragment thereof, is, SaCas9. In some embodiments, the auto-antigen, self-antigen, tolerance inducing antigen, or a fragment thereof, is FnCas12a. In some embodiments, the auto-antigen, self-antigen, tolerance inducing antigen, or a fragment thereof, is LbCas12a. In some embodiments, the auto-antigen, self-antigen, tolerance inducing antigen, or a fragment thereof, is AcCas12a. In some embodiments, the auto-antigen, self-antigen, tolerance inducing antigen, or a fragment thereof, is Cas13b. In some embodiments, the auto-antigen, self-antigen, tolerance inducing antigen, or a fragment thereof, is APOBEC1 (wherein APOBEC1 is from any vertebrate species). In some embodiments, the auto-antigen, self-antigen, tolerance inducing antigen, or a fragment thereof, is E. coli TadA. In some embodiments, the auto-antigen, self-antigen, tolerance inducing antigen, or a fragment thereof, is M-MLV RT. In some embodiments, the auto-antigen, self-antigen, tolerance inducing antigen, or a fragment thereof, is HLA-A. In some embodiments, the auto-antigen, self-antigen, tolerance inducing antigen, or a fragment thereof, is HLA-B. In some embodiments, the auto-antigen, self-antigen, tolerance inducing antigen, or a fragment thereof, is HLA-C. In some embodiments, the auto-antigen, self-antigen, tolerance inducing antigen, or a fragment thereof, is HLA-E. In some embodiments, the auto-antigen, self-antigen, tolerance inducing antigen, or a fragment thereof, is HLA-L. In some embodiments, the auto-antigen, self-antigen, tolerance inducing antigen, or a fragment thereof, is HLA-J. In some embodiments, the auto-antigen, self-antigen, tolerance inducing antigen, or a fragment thereof, is HLA-K. In some embodiments, the auto-antigen, self-antigen, tolerance inducing antigen, or a fragment thereof, is HLA-H. In some embodiments, the auto-antigen, self-antigen, tolerance inducing antigen, or a fragment thereof, is HLA-G. In some embodiments, the auto-antigen, self-antigen, tolerance inducing antigen, or a fragment thereof, is HLA-DM. In some embodiments, the auto-antigen, self-antigen, tolerance inducing antigen, or a fragment thereof, is HLA-DO. In some embodiments, the auto-antigen, self-antigen, tolerance inducing antigen, or a fragment thereof, is HLA-DP. In some embodiments, the auto-antigen, self-antigen, tolerance inducing antigen, or a fragment thereof, is HLA-DQ. In some embodiments, the auto-antigen, self-antigen, tolerance inducing antigen, or a fragment thereof, is HLA-DR.

In some embodiments, the antigen of interest is an antibody, or the antigen-binding fragment thereof, or a fragment thereof.

In some embodiments, the antibody, or the antigen-binding fragment thereof, or a fragment thereof is selected from an anti-glutamate decarboxylase 1 (GAD) antibody, or the antigen-binding fragment thereof, an anti-proinsulin antibody, or the antigen-binding fragment thereof, an anti-islet-specific glucose-6-phosphatase catalytic subunit-related protein (IGRP) antibody, or the antigen-binding fragment thereof, an anti-zinc transporter 8 (Znt8) antibody, or the antigen-binding fragment thereof, an anti-receptor-type tyrosine-protein phosphatase-like N (IA2) antibody, or the antigen-binding fragment thereof, an anti-tropomyosin alpha-3 chain (TPM3) antibody, or the antigen-binding fragment thereof, an anti-granulocyte-macrophage colony-stimulating factor (GMCSF/CSF2) antibody, or the antigen-binding fragment thereof, an anti-RNA polymerase III complex subunit A (RNAP3) antibody, or the antigen-binding fragment thereof, an anti-RNA polymerase III complex subunit B (RNAP3) antibody, or the antigen-binding fragment thereof, an anti-RNA polymerase III complex subunit C (RNAP3) antibody, or the antigen-binding fragment thereof, an anti-RNA polymerase III complex subunit D (RNAP3) antibody, or the antigen-binding fragment thereof, an anti-RNA polymerase III complex subunit E (RNAP3) antibody, or the antigen-binding fragment thereof, an anti-RNA polymerase III complex subunit F (RNAP3) antibody, or the antigen-binding fragment thereof, an anti-RNA polymerase III complex subunit G (RNAP3) antibody, or the antigen-binding fragment thereof, an anti-RNA polymerase III complex subunit H (RNAP3) antibody, or the antigen-binding fragment thereof, an anti-RNA polymerase III complex subunit K (RNAP3) antibody, or the antigen-binding fragment thereof, an anti-DNA topoisomerase 1 (TOPO1) antibody, or the antigen-binding fragment thereof, an anti-type-1 angiotensin II receptor (ATlR) antibody, or the antigen-binding fragment thereof, an anti-endothelin-1 receptor (ETAR), an anti-major centromere protein A (CENP) antibody, or the antigen-binding fragment thereof, an anti-major centromere protein B (CENP) antibody, or the antigen-binding fragment thereof, an anti-major centromere protein C (CENP) antibody, or the antigen-binding fragment thereof, an anti-tetraspanin-7 (TSPAN7) antibody, or the antigen-binding fragment thereof, an anti-21OH antibody, or the antigen-binding fragment thereof, anti-17OH antibody, or the antigen-binding fragment thereof, anti-CCP antibody, or the antigen-binding fragment thereof, anti-Th/To antibody, or the antigen-binding fragment thereof, anti-ANA antibody, or the antigen-binding fragment thereof, anti-dsDNA antibody, or the antigen-binding fragment thereof, anti-HMGCR antibody, or the antigen-binding fragment thereof, anti-myosin antibody, or the antigen-binding fragment thereof, anti-SSA/Ro antibody, or the antigen-binding fragment thereof, anti-SSB/La antibody, or the antigen-binding fragment thereof, anti-U1RNP antibody, or the antigen-binding fragment thereof, anti-CENP antibody, or the antigen-binding fragment thereof, anti-U3RNP antibody, or the antigen-binding fragment thereof, anti-PM-Sci antibody, or the antigen-binding fragment thereof, anti-MBP antibody, or the antigen-binding fragment thereof, anti-IL17 antibody, anti-IL22 antibody, or the antigen-binding fragment thereof, anti-ASMA antibody, or the antigen-binding fragment thereof, anti-actin antibody, or the antigen-binding fragment thereof, anti-amylase α2 antibody, or the antigen-binding fragment thereof, anti-GM-CSF antibody, or the antigen-binding fragment thereof, anti-snRNP antibody, or the antigen-binding fragment thereof, anti-Ro52 antibody, or the antigen-binding fragment thereof, anti-Ro60 antibody, or the antigen-binding fragment thereof, anti-La antibody, or the antigen-binding fragment thereof, anti-Jo-1 antibody, or the antigen-binding fragment thereof, anti-SRP antibody, or the antigen-binding fragment thereof, anti-IFIH1 antibody, or the antigen-binding fragment thereof, anti-CENPA antibody, or the antigen-binding fragment thereof, anti-RNAP3 antibody, or the antigen-binding fragment thereof, anti-TOPO1 antibody, or the antigen-binding fragment thereof, anti-insulin antibody, or the antigen-binding fragment thereof, anti-RNA-P antibody, or the antigen-binding fragment thereof, anti-U1-70K antibody, or the antigen-binding fragment thereof, anti-Sm-D3 antibody, or the antigen-binding fragment thereof, anti-MDA-5 antibody, or the antigen-binding fragment thereof, anti-PL7 antibody, or the antigen-binding fragment thereof, anti-PL-12 antibody, or the antigen-binding fragment thereof, anti-centromere protein antibody, or the antigen-binding fragment thereof, anti-PM/SCL antibody, or the antigen-binding fragment thereof, anti-RNA polymerase 3 complex antibody, or the antigen-binding fragment thereof, anti-desmoglein-1 antibody, or the antigen-binding fragment thereof, anti-tetraspanin-7 antibody, or the antigen-binding fragment thereof, anti-peptidylprolyl isomerase like 2 antibody, or the antigen-binding fragment thereof, anti-Mlh1 antibody, or the antigen-binding fragment thereof, anti-proteinase-3 antibody, or the antigen-binding fragment thereof, anti-immunoglobulin G antibody, or the antigen-binding fragment thereof, anti-immunoglobulin M antibody, or the antigen-binding fragment thereof, anti-myeloperoxidase antibody, or the antigen-binding fragment thereof, anti-AchR antibody, or the antigen-binding fragment thereof, anti-MUSK antibody, or the antigen-binding fragment thereof, anti-LRP4 antibody, or the antigen-binding fragment thereof, anti-HSP90 antibody, or the antigen-binding fragment thereof, anti-HSPA5 antibody, or the antigen-binding fragment thereof, anti-kallikrein 13 antibody, or the antigen-binding fragment thereof, anti-AT1R antibody, or the antigen-binding fragment thereof, anti-ETAR antibody, or the antigen-binding fragment thereof, anti-ADAMTS13 antibody, or the antigen-binding fragment thereof, anti-IFN-7 antibody, or the antigen-binding fragment thereof, anti-IFN-α/ω antibody, or the antigen-binding fragment thereof, anti-IL6 antibody, or the antigen-binding fragment thereof, anti-IL12/IL23 antibody, or the antigen-binding fragment thereof, anti-desmoglein-3 antibody, or the antigen-binding fragment thereof, anti-aquaporin-4 antibody, or the antigen-binding fragment thereof, anti-MOG antibody, or the antigen-binding fragment thereof, anti-GRIN1 antibody, or the antigen-binding fragment thereof, anti-GRIA1 antibody, or the antigen-binding fragment thereof, anti-GRIA2 antibody, or the antigen-binding fragment thereof, anti-LGI1 antibody, or the antigen-binding fragment thereof, anti-CNTNAP2 antibody, or the antigen-binding fragment thereof, anti-COL4A3 antibody, or the antigen-binding fragment thereof, anti-COL17 antibody, or the antigen-binding fragment thereof, anti-BP180 antibody, or the antigen-binding fragment thereof, anti-PLA2R antibody, or the antigen-binding fragment thereof, anti-THSD7A antibody, or the antigen-binding fragment thereof, anti-THSR antibody, or the antigen-binding fragment thereof, anti-GMCSF (CSF2) antibody, or the antigen-binding fragment thereof, anti-NMDAR antibody, or the antigen-binding fragment thereof, anti-FGF23 antibody, or the antigen-binding fragment thereof, anti-acid beta-glycosidase antibody, or the antigen-binding fragment thereof, anti-acid alpha-glycosidase antibody, or the antigen-binding fragment thereof, anti-alpha-galactosidase A antibody, or the antigen-binding fragment thereof, anti-alpha-N-acetylgalactosaminidase antibody, or the antigen-binding fragment thereof, anti-alpha-L-iduronohydrolase antibody, or the antigen-binding fragment thereof, anti-beta-hexosaminidase A antibody, or the antigen-binding fragment thereof, anti-sphingomyelin phosphodiesterase 1 antibody, or the antigen-binding fragment thereof, anti-N-acetylsphingosine amidohydrolase 1 antibody, or the antigen-binding fragment thereof, anti-galactocerebrosidase antibody, or the antigen-binding fragment thereof, anti-N-acetylgalactosamine-sulfate sulfatase antibody, or the antigen-binding fragment thereof, anti-arylsulfatase B antibody, or the antigen-binding fragment thereof, anti-iduronate 2-sulfatase antibody, or the antigen-binding fragment thereof, anti-heparan-alpha-glucosaminide-N-acetyltransferase antibody, or the antigen-binding fragment thereof, anti-beta-glucuronidase antibody, or the antigen-binding fragment thereof, anti-hyaluronidase antibody, or the antigen-binding fragment thereof, anti-sialidase 1 antibody, or the antigen-binding fragment thereof, anti-N-acetylglucosamine-1-phosphotransferase alpha/beta antibody, or the antigen-binding fragment thereof, anti-N-acetylglucosamine-1-phosphotransferase gamma antibody, or the antigen-binding fragment thereof, anti-glucose-6-phosphatase catalytic subunit antibody, or the antigen-binding fragment thereof, anti-glucose-6-phosphate transporter antibody, or the antigen-binding fragment thereof, anti-ATP7B antibody, or the antigen-binding fragment thereof, anti-phenylalanine hydroxylase antibody, or the antigen-binding fragment thereof, anti-ATP-binding cassette subfamily A member 4 antibody, or the antigen-binding fragment thereof, anti-myocilin antibody, or the antigen-binding fragment thereof, anti-coagulation factor VIII antibody, or the antigen-binding fragment thereof, anti-coagulation factor IX antibody, or the antigen-binding fragment thereof, anti-von Willebrand factor antibody, or the antigen-binding fragment thereof, anti-glycoprotein Ib alpha antibody, or the antigen-binding fragment thereof, anti-glycoprotein Ib beta antibody, or the antigen-binding fragment thereof, anti-glycoprotein V antibody, or the antigen-binding fragment thereof, anti-glycoprotein VI antibody, or the antigen-binding fragment thereof, anti-glycoprotein IX antibody, or the antigen-binding fragment thereof, anti-glycoprotein IIb antibody, or the antigen-binding fragment thereof, anti-glycoprotein IIIa antibody, or the antigen-binding fragment thereof, anti-platelet factor 4 antibody, or the antigen-binding fragment thereof, anti-AAV capsid protein antibody, or the antigen-binding fragment thereof, (such as AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.eB, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9, AAV9 K449R (or K449R AAV9), AAV9.16, AAV9.24, AAV9.45, AAbiodisV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74 (also referred to as AAVrh74), AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, 7m8, Spark100, AAVMYO and variants thereof), anti-SpCas9 antibody, or the antigen-binding fragment thereof, anti-SaCas9 antibody, or the antigen-binding fragment thereof, anti-FnCas12a antibody, or the antigen-binding fragment thereof, anti-LbCas12a antibody, or the antigen-binding fragment thereof, anti-AcCas12a antibody, or the antigen-binding fragment thereof, anti-Cas13b antibody, or the antigen-binding fragment thereof, anti-APOBEC1 antibody, or the antigen-binding fragment thereof, anti-E. coli TadA antibody, or the antigen-binding fragment thereof, anti-M-MLV RT antibody, or the antigen-binding fragment thereof, anti-HLA-A antibody, or the antigen-binding fragment thereof, anti-HLA-B antibody, or the antigen-binding fragment thereof, anti-HLA-C antibody, or the antigen-binding fragment thereof, anti-HLA-E antibody, or the antigen-binding fragment thereof, anti-HLA-L antibody, or the antigen-binding fragment thereof, anti-HLA-J antibody, or the antigen-binding fragment thereof, anti-HLA-K antibody, or the antigen-binding fragment thereof, anti-HLA-H antibody, or the antigen-binding fragment thereof, anti-HLA-G antibody, or the antigen-binding fragment thereof, anti-HLA-DM antibody, or the antigen-binding fragment thereof, anti-HLA-DO antibody, or the antigen-binding fragment thereof, anti-HLA-DP antibody, or the antigen-binding fragment thereof, anti-HLA-DQ antibody, or the antigen-binding fragment thereof, anti-HLA-DR antibody, or the antigen-binding fragment thereof.

In some embodiments, the antibody, or the antigen-binding fragment thereof, is the anti-glutamate decarboxylase 1 (GAD) antibody. In some embodiments, the antibody, or the antigen-binding fragment thereof, is the anti-proinsulin antibody. In some embodiments, the antibody, or the antigen-binding fragment thereof, is the anti-islet-specific glucose-6-phosphatase catalytic subunit-related protein (IGRP) antibody. In some embodiments, the antibody, or the antigen-binding fragment thereof, is the anti-zinc transporter 8 (Znt8) antibody. In some embodiments, the antibody, or the antigen-binding fragment thereof, is the anti-receptor-type tyrosine-protein phosphatase-like N (IA2) antibody. In some embodiments, the antibody, or the antigen-binding fragment thereof, is the anti-tropomyosin alpha-3 chain (TPM3) antibody. In some embodiments, the antibody, or the antigen-binding fragment thereof, is the anti-granulocyte-macrophage colony-stimulating factor (GMCSF/CSF2) antibody. In some embodiments, the antibody, or the antigen-binding fragment thereof, is the anti-RNA polymerase III complex subunit A (RNAP3) antibody. In some embodiments, the antibody, or the antigen-binding fragment thereof, is the anti-RNA polymerase III complex subunit B (RNAP3) antibody. In some embodiments, the antibody, or the antigen-binding fragment thereof, is the anti-RNA polymerase III complex subunit C (RNAP3) antibody. In some embodiments, the antibody, or the antigen-binding fragment thereof, is the anti-RNA polymerase III complex subunit D (RNAP3) antibody. In some embodiments, the antibody, or the antigen-binding fragment thereof, is the anti-RNA polymerase III complex subunit E (RNAP3) antibody. In some embodiments, the antibody, or the antigen-binding fragment thereof, is the anti-RNA polymerase III complex subunit F (RNAP3) antibody. In some embodiments, the antibody, or the antigen-binding fragment thereof, is the anti-RNA polymerase III complex subunit G (RNAP3) antibody. In some embodiments, the antibody, or the antigen-binding fragment thereof, is the anti-RNA polymerase III complex subunit H (RNAP3) antibody. In some embodiments, the antibody, or the antigen-binding fragment thereof, is the anti-RNA polymerase III complex subunit K (RNAP3) antibody. In some embodiments, the antibody, or the antigen-binding fragment thereof, is the anti-DNA topoisomerase 1 (TOPO1) antibody. In some embodiments, the antibody, or the antigen-binding fragment thereof, is the anti-type-1 angiotensin II receptor (AT1R) antibody. In some embodiments, the antibody, or the antigen-binding fragment thereof, is the anti-endothelin-1 receptor (ETAR). In some embodiments, the antibody, or the antigen-binding fragment thereof, is the anti-major centromere protein A (CENP) antibody. In some embodiments, the antibody, or the antigen-binding fragment thereof, is the anti-major centromere protein B (CENP) antibody. In some embodiments, the antibody, or the antigen-binding fragment thereof, is the anti-major centromere protein C (CENP) antibody. In some embodiments, the antibody, or the antigen-binding fragment thereof, is the anti-tetraspanin-7 (TSPAN7) antibody. In some embodiments, the antibody, or the antigen-binding fragment thereof, is the anti-21OH antibody, or the antigen-binding fragment thereof. In some embodiments, the antibody, or the antigen-binding fragment thereof, is the anti-17OH antibody, or the antigen-binding fragment thereof. In some embodiments, the antibody, or the antigen-binding fragment thereof, is the anti-CCP antibody, or the antigen-binding fragment thereof. In some embodiments, the antibody, or the antigen-binding fragment thereof, is the anti-Th/To antibody, or the antigen-binding fragment thereof. In some embodiments, the antibody, or the antigen-binding fragment thereof, is the anti-ANA antibody, or the antigen-binding fragment thereof, anti-dsDNA antibody, or the antigen-binding fragment thereof. In some embodiments, the antibody, or the antigen-binding fragment thereof, is the anti-HMGCR antibody, or the antigen-binding fragment thereof. In some embodiments, the antibody, or the antigen-binding fragment thereof, is the anti-myosin antibody, or the antigen-binding fragment thereof. In some embodiments, the antibody, or the antigen-binding fragment thereof, is the anti-SSA/Ro antibody, or the antigen-binding fragment thereof, anti-SSB/La antibody, or the antigen-binding fragment thereof. In some embodiments, the antibody, or the antigen-binding fragment thereof, is the anti-U1RNP antibody, or the antigen-binding fragment thereof. In some embodiments, the antibody, or the antigen-binding fragment thereof, is the anti-CENP antibody, or the antigen-binding fragment thereof. In some embodiments, the antibody, or the antigen-binding fragment thereof, is the anti-U3RNP antibody, or the antigen-binding fragment thereof. In some embodiments, the antibody, or the antigen-binding fragment thereof, is the anti-PM-Sci antibody, or the antigen-binding fragment thereof. In some embodiments, the antibody, or the antigen-binding fragment thereof, is the anti-MBP antibody, or the antigen-binding fragment thereof. In some embodiments, the antibody, or the antigen-binding fragment thereof, is the anti-IL17 antibody, or the antigen-binding fragment thereof. In some embodiments, the antibody, or the antigen-binding fragment thereof, is the anti-IL22 antibody, or the antigen-binding fragment thereof. In some embodiments, the antibody, or the antigen-binding fragment thereof, is the anti-ASMA antibody, or the antigen-binding fragment thereof. In some embodiments, the antibody, or the antigen-binding fragment thereof, is the anti-actin antibody, or the antigen-binding fragment thereof. In some embodiments, the antibody, or the antigen-binding fragment thereof, is the anti-amylase α2 antibody, or the antigen-binding fragment thereof. In some embodiments, the antibody, or the antigen-binding fragment thereof, is the anti-GM-CSF antibody, or the antigen-binding fragment thereof. In some embodiments, the antibody, or the antigen-binding fragment thereof, is the anti-snRNP antibody, or the antigen-binding fragment thereof. In some embodiments, the antibody, or the antigen-binding fragment thereof, is the anti-Ro52 antibody, or the antigen-binding fragment thereof. In some embodiments, the antibody, or the antigen-binding fragment thereof, is the anti-Ro60 antibody, or the antigen-binding fragment thereof. In some embodiments, the antibody, or the antigen-binding fragment thereof, is the anti-La antibody, or the antigen-binding fragment thereof. In some embodiments, the antibody, or the antigen-binding fragment thereof, is the anti-Jo-1 antibody, or the antigen-binding fragment thereof. In some embodiments, the antibody, or the antigen-binding fragment thereof, is the anti-SRP antibody, or the antigen-binding fragment thereof. In some embodiments, the antibody, or the antigen-binding fragment thereof, is the anti-IFIH1 antibody, or the antigen-binding fragment thereof. In some embodiments, the antibody, or the antigen-binding fragment thereof, is the anti-CENPA antibody, or the antigen-binding fragment thereof. In some embodiments, the antibody, or the antigen-binding fragment thereof, is the anti-RNAP3 antibody, or the antigen-binding fragment thereof. In some embodiments, the antibody, or the antigen-binding fragment thereof, is the anti-TOPO1 antibody, or the antigen-binding fragment thereof. In some embodiments, the antibody, or the antigen-binding fragment thereof, is the anti-insulin antibody, or the antigen-binding fragment thereof. In some embodiments, the antibody, or the antigen-binding fragment thereof, is the anti-GAD65 antibody, or the antigen-binding fragment thereof. In some embodiments, the antibody, or the antigen-binding fragment thereof, is the anti-IA2 antibody, or the antigen-binding fragment thereof. In some embodiments, the antibody, or the antigen-binding fragment thereof, is the anti-Znt8 antibody, or the antigen-binding fragment thereof. In some embodiments, the antibody, or the antigen-binding fragment thereof, is the anti-RNA-P antibody, or the antigen-binding fragment thereof. In some embodiments, the antibody, or the antigen-binding fragment thereof, is the anti-U1-70K antibody, or the antigen-binding fragment thereof. In some embodiments, the antibody, or the antigen-binding fragment thereof, is the anti-Sm-D3 antibody, or the antigen-binding fragment thereof. In some embodiments, the antibody, or the antigen-binding fragment thereof, is the anti-MDA-5 antibody, or the antigen-binding fragment thereof. In some embodiments, the antibody, or the antigen-binding fragment thereof, is the anti-PL7 antibody, or the antigen-binding fragment thereof. In some embodiments, the antibody, or the antigen-binding fragment thereof, is the anti-PL-12 antibody, or the antigen-binding fragment thereof. In some embodiments, the antibody, or the antigen-binding fragment thereof, is the anti-centromere protein antibody, or the antigen-binding fragment thereof. In some embodiments, the antibody, or the antigen-binding fragment thereof, is the anti-PM/SCL antibody, or the antigen-binding fragment thereof. In some embodiments, the antibody, or the antigen-binding fragment thereof, is the anti-RNA polymerase 3 complex antibody, or the antigen-binding fragment thereof. In some embodiments, the antibody, or the antigen-binding fragment thereof, is the anti-desmoglein-1 antibody, or the antigen-binding fragment thereof. In some embodiments, the antibody, or the antigen-binding fragment thereof, is the anti-tetraspanin-7 antibody, or the antigen-binding fragment thereof. In some embodiments, the antibody, or the antigen-binding fragment thereof, is the anti-peptidylprolyl isomerase like 2 antibody, or the antigen-binding fragment thereof. In some embodiments, the antibody, or the antigen-binding fragment thereof, is the anti-Mlh1 antibody, or the antigen-binding fragment thereof. In some embodiments, the antibody, or the antigen-binding fragment thereof, is the anti-proteinase-3 antibody, or the antigen-binding fragment thereof. In some embodiments, the antibody, or the antigen-binding fragment thereof, is the anti-immunoglobulin G antibody, or the antigen-binding fragment thereof. In some embodiments, the antibody, or the antigen-binding fragment thereof, is the anti-immunoglobulin M antibody, or the antigen-binding fragment thereof. In some embodiments, the antibody, or the antigen-binding fragment thereof, is the anti-myeloperoxidase antibody, or the antigen-binding fragment thereof. In some embodiments, the antibody, or the antigen-binding fragment thereof, is the anti-AchR antibody, or the antigen-binding fragment thereof. In some embodiments, the antibody, or the antigen-binding fragment thereof, is the anti-MUSK antibody, or the antigen-binding fragment thereof. In some embodiments, the antibody, or the antigen-binding fragment thereof, is the anti-LRP4 antibody, or the antigen-binding fragment thereof. In some embodiments, the antibody, or the antigen-binding fragment thereof, is the anti-HSP90 antibody, or the antigen-binding fragment thereof. In some embodiments, the antibody, or the antigen-binding fragment thereof, is the anti-HSPA5 antibody, or the antigen-binding fragment thereof. In some embodiments, the antibody, or the antigen-binding fragment thereof, is the anti-kallikrein 13 antibody, or the antigen-binding fragment thereof. In some embodiments, the antibody, or the antigen-binding fragment thereof, is the anti-ATIR antibody, or the antigen-binding fragment thereof. In some embodiments, the antibody, or the antigen-binding fragment thereof, is the anti-ETAR antibody, or the antigen-binding fragment thereof. In some embodiments, the antibody, or the antigen-binding fragment thereof, is the anti-ADAMTS13 antibody, or the antigen-binding fragment thereof. In some embodiments, the antibody, or the antigen-binding fragment thereof, is the anti-IFN-7 antibody, or the antigen-binding fragment thereof. In some embodiments, the antibody, or the antigen-binding fragment thereof, is the anti-IFN-α/ω antibody, or the antigen-binding fragment thereof. In some embodiments, the antibody, or the antigen-binding fragment thereof, is the anti-IL6 antibody, or the antigen-binding fragment thereof. In some embodiments, the antibody, or the antigen-binding fragment thereof, is the anti-IL12/IL23 antibody, or the antigen-binding fragment thereof. In some embodiments, the antibody, or the antigen-binding fragment thereof, is the anti-desmoglein-3 antibody, or the antigen-binding fragment thereof. In some embodiments, the antibody, or the antigen-binding fragment thereof, is the anti-aquaporin-4 antibody, or the antigen-binding fragment thereof. In some embodiments, the antibody, or the antigen-binding fragment thereof, is the anti-MOG antibody, or the antigen-binding fragment thereof. In some embodiments, the antibody, or the antigen-binding fragment thereof, is the anti-GRIN1 antibody, or the antigen-binding fragment thereof. In some embodiments, the antibody, or the antigen-binding fragment thereof, is the anti-GRIA1 antibody, or the antigen-binding fragment thereof. In some embodiments, the antibody, or the antigen-binding fragment thereof, is the anti-GRIA2 antibody, or the antigen-binding fragment thereof. In some embodiments, the antibody, or the antigen-binding fragment thereof, is the anti-LGI1 antibody, or the antigen-binding fragment thereof. In some embodiments, the antibody, or the antigen-binding fragment thereof, is the anti-CNTNAP2 antibody, or the antigen-binding fragment thereof. In some embodiments, the antibody, or the antigen-binding fragment thereof, is the anti-COL4A3 antibody, or the antigen-binding fragment thereof. In some embodiments, the antibody, or the antigen-binding fragment thereof, is the anti-COL17 antibody, or the antigen-binding fragment thereof. In some embodiments, the antibody, or the antigen-binding fragment thereof, is the anti-BP180 antibody, or the antigen-binding fragment thereof. In some embodiments, the antibody, or the antigen-binding fragment thereof, is the anti-PLA2R antibody, or the antigen-binding fragment thereof. In some embodiments, the antibody, or the antigen-binding fragment thereof, is the anti-THSD7A antibody, or the antigen-binding fragment thereof. In some embodiments, the antibody, or the antigen-binding fragment thereof, is the anti-THSR antibody, or the antigen-binding fragment thereof. In some embodiments, the antibody, or the antigen-binding fragment thereof, is the anti-GMCSF (CSF2) antibody, or the antigen-binding fragment thereof. In some embodiments, the antibody, or the antigen-binding fragment thereof, is the anti-NMDAR antibody, or the antigen-binding fragment thereof. In some embodiments, the antibody, or the antigen-binding fragment thereof, is the anti-FGF23 antibody, or the antigen-binding fragment thereof. In some embodiments, the antibody, or the antigen-binding fragment thereof, is the anti-acid beta-glycosidase antibody, or the antigen-binding fragment thereof. In some embodiments, the antibody, or the antigen-binding fragment thereof, is the anti-acid alpha-glycosidase antibody, or the antigen-binding fragment thereof. In some embodiments, the antibody, or the antigen-binding fragment thereof, is the anti-alpha-galactosidase A antibody, or the antigen-binding fragment thereof. In some embodiments, the antibody, or the antigen-binding fragment thereof, is the anti-alpha-N-acetylgalactosaminidase antibody, or the antigen-binding fragment thereof. In some embodiments, the antibody, or the antigen-binding fragment thereof, is the anti-alpha-L-iduronohydrolase antibody, or the antigen-binding fragment thereof. In some embodiments, the antibody, or the antigen-binding fragment thereof, is the anti-beta-hexosaminidase A antibody, or the antigen-binding fragment thereof. In some embodiments, the antibody, or the antigen-binding fragment thereof, is the anti-sphingomyelin phosphodiesterase 1 antibody, or the antigen-binding fragment thereof. In some embodiments, the antibody, or the antigen-binding fragment thereof, is the anti-N-acetylsphingosine amidohydrolase 1 antibody, or the antigen-binding fragment thereof. In some embodiments, the antibody, or the antigen-binding fragment thereof, is the anti-galactocerebrosidase antibody, or the antigen-binding fragment thereof. In some embodiments, the antibody, or the antigen-binding fragment thereof, is the anti-N-acetylgalactosamine-sulfate sulfatase antibody, or the antigen-binding fragment thereof. In some embodiments, the antibody, or the antigen-binding fragment thereof, is the anti-arylsulfatase B antibody, or the antigen-binding fragment thereof, anti-iduronate 2-sulfatase antibody, or the antigen-binding fragment thereof. In some embodiments, the antibody, or the antigen-binding fragment thereof, is the anti-heparan-alpha-glucosaminide-N-acetyltransferase antibody, or the antigen-binding fragment thereof. In some embodiments, the antibody, or the antigen-binding fragment thereof, is the anti-beta-glucuronidase antibody, or the antigen-binding fragment thereof. In some embodiments, the antibody, or the antigen-binding fragment thereof, is the anti-hyaluronidase antibody, or the antigen-binding fragment thereof. In some embodiments, the antibody, or the antigen-binding fragment thereof, is the anti-sialidase 1 antibody, or the antigen-binding fragment thereof. In some embodiments, the antibody, or the antigen-binding fragment thereof, is the anti-N-acetylglucosamine-1-anti-phosphotransferase alpha/beta antibody, or the antigen-binding fragment thereof. In some embodiments, the antibody, or the antigen-binding fragment thereof, is the anti-N-acetylglucosamine-1-phosphotransferase gamma antibody, or the antigen-binding fragment thereof. In some embodiments, the antibody, or the antigen-binding fragment thereof, is the anti-glucose-6-phosphatase catalytic subunit antibody, or the antigen-binding fragment thereof. In some embodiments, the antibody, or the antigen-binding fragment thereof, is the anti-glucose-6-phosphate transporter antibody, or the antigen-binding fragment thereof. In some embodiments, the antibody, or the antigen-binding fragment thereof, is the anti-ATP7B antibody, or the antigen-binding fragment thereof. In some embodiments, the antibody, or the antigen-binding fragment thereof, is the anti-Phenylalanine hydroxylase antibody, or the antigen-binding fragment thereof. In some embodiments, the antibody, or the antigen-binding fragment thereof, is the anti-ATP-binding cassette subfamily A member 4 antibody, or the antigen-binding fragment thereof. In some embodiments, the antibody, or the antigen-binding fragment thereof, is the anti-myocilin antibody, or the antigen-binding fragment thereof. In some embodiments, the antibody, or the antigen-binding fragment thereof, is the anti-coagulation factor VIII antibody, or the antigen-binding fragment thereof. In some embodiments, the antibody, or the antigen-binding fragment thereof, is the anti-coagulation factor IX antibody, or the antigen-binding fragment thereof. In some embodiments, the antibody, or the antigen-binding fragment thereof, is the anti-von Willebrand factor antibody, or the antigen-binding fragment thereof. In some embodiments, the antibody, or the antigen-binding fragment thereof, is the anti-glycoprotein Ib alpha antibody, or the antigen-binding fragment thereof. In some embodiments, the antibody, or the antigen-binding fragment thereof, is the anti-glycoprotein Ib beta antibody, or the antigen-binding fragment thereof. In some embodiments, the antibody, or the antigen-binding fragment thereof, is the anti-glycoprotein V antibody, or the antigen-binding fragment thereof. In some embodiments, the antibody, or the antigen-binding fragment thereof, is the anti-glycoprotein VI antibody, or the antigen-binding fragment thereof. In some embodiments, the antibody, or the antigen-binding fragment thereof, is the anti-glycoprotein IX antibody, or the antigen-binding fragment thereof. In some embodiments, the antibody, or the antigen-binding fragment thereof, is the anti-glycoprotein IIb antibody, or the antigen-binding fragment thereof. In some embodiments, the antibody, or the antigen-binding fragment thereof, is the anti-glycoprotein IIIa antibody, or the antigen-binding fragment thereof. In some embodiments, the antibody, or the antigen-binding fragment thereof, is the anti-platelet factor 4 antibody, or the antigen-binding fragment thereof. In some embodiments, the antibody, or the antigen-binding fragment thereof, is the anti-AAV capsid protein (such as AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.eB, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9, AAV9 K449R (or K449R AAV9), AAV9.16, AAV9.24, AAV9.45, AAbiodisV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74 (also referred to as AAVrh74), AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, 7m8, Spark100, AAVMYO and variants thereof) antibody, or the antigen-binding fragment thereof. In some embodiments, the antibody, or the antigen-binding fragment thereof, is the anti-SpCas9 antibody, or the antigen-binding fragment thereof. In some embodiments, the antibody, or the antigen-binding fragment thereof, is the anti-SaCas9 antibody, or the antigen-binding fragment thereof. In some embodiments, the antibody, or the antigen-binding fragment thereof, is the anti-FnCas12a antibody, or the antigen-binding fragment thereof. In some embodiments, the antibody, or the antigen-binding fragment thereof, is the anti-LbCas12a antibody, or the antigen-binding fragment thereof. In some embodiments, the antibody, or the antigen-binding fragment thereof, is the anti-AcCas12a antibody, or the antigen-binding fragment thereof. In some embodiments, the antibody, or the antigen-binding fragment thereof, is the anti-Cas13b antibody, or the antigen-binding fragment thereof. In some embodiments, the antibody, or the antigen-binding fragment thereof, is the anti-APOBEC1 antibody, or the antigen-binding fragment thereof. In some embodiments, the antibody, or the antigen-binding fragment thereof, is the anti-E. coli TadA antibody, or the antigen-binding fragment thereof. In some embodiments, the antibody, or the antigen-binding fragment thereof, is the anti-M-MLV RT antibody, or the antigen-binding fragment thereof. In some embodiments, the antibody, or the antigen-binding fragment thereof, is the anti-HLA-A antibody, or the antigen-binding fragment thereof. In some embodiments, the antibody, or the antigen-binding fragment thereof, is the anti-HLA-B antibody, or the antigen-binding fragment thereof. In some embodiments, the antibody, or the antigen-binding fragment thereof, is the anti-HLA-C antibody, or the antigen-binding fragment thereof. In some embodiments, the antibody, or the antigen-binding fragment thereof, is the anti-HLA-E antibody, or the antigen-binding fragment thereof. In some embodiments, the antibody, or the antigen-binding fragment thereof, is the anti-HLA-L antibody, or the antigen-binding fragment thereof. In some embodiments, the antibody, or the antigen-binding fragment thereof, is the anti-HLA-J antibody, or the antigen-binding fragment thereof. In some embodiments, the antibody, or the antigen-binding fragment thereof, is the anti-HLA-K antibody, or the antigen-binding fragment thereof. In some embodiments, the antibody, or the antigen-binding fragment thereof, is the anti-HLA-H antibody, or the antigen-binding fragment thereof. In some embodiments, the antibody, or the antigen-binding fragment thereof, is the anti-HLA-G antibody, or the antigen-binding fragment thereof. In some embodiments, the antibody, or the antigen-binding fragment thereof, is the anti-HLA-DM antibody, or the antigen-binding fragment thereof. In some embodiments, the antibody, or the antigen-binding fragment thereof, is the anti-HLA-DO antibody, or the antigen-binding fragment thereof. In some embodiments, the antibody, or the antigen-binding fragment thereof, is the anti-HLA-DP antibody, or the antigen-binding fragment thereof. In some embodiments, the antibody, or the antigen-binding fragment thereof, is the anti-HLA-DQ antibody, or the antigen-binding fragment thereof. In some embodiments, the antibody, or the antigen-binding fragment thereof, is the anti-HLA-DR antibody, or the antigen-binding fragment thereof.

In some embodiments, the antigen of interest is an effector peptide or an effector molecule. In some embodiments, the effector peptide or the effector molecule is a peptide or a molecule capable of eliciting an autoimmune response. In some embodiments, the effector peptide or the effector molecule is an antigen, an allergen, or a pollen. In some embodiments, the terms “allergen” and “pollen” may be used interchangeably. In some embodiments, the allergen, or pollen, elicits an allergic reaction. In some embodiments, the allergic reaction is a reaction to abalone, absinthe wormwood, acacia pollen, African whitewood, Alaska Pollock, alder, almond, American cockroach, American house dust mite, American lobster, American oyster, annual mercury grass, apple, apricot, Arizona cypress, arthropod, Asian green mussel, Asian hornet, Asian needle ant, Asian seabass, Asian tiger mosquito, asinus, Atlantic cod, Atlantic herring, Atlantic mackerel, Australian jumper ant, avocado, bacteria, Bahia grass, Baltic cod, banana, banana prawn, barley, beet, bell pepper, Benguela hake, bent grass, Bermuda grass, bigfin reef squid, birch, biting midges, black fire ant, black gram, black mold, black mulberry, black polar, black tiger shrimp, black walnut, blue hake, blue lupin, blue swimmer crab, booklouse, bovine, brazil nut, Brazilian fire ant, bread mold, bread wheat, brook trout, brown garden snail, brown shrimp, buckwheat, bumblebee, California sagebrush, canary grass, capillary wormwood, caridean shrimp, carp, carrot, cashew, cassava, castor bean, cat, cedar, celery, cherry, chicken, chickling vetch, chickpea, Chinese mitten crab, Chinese razor clam, Chinese white shrimp, Chinese date, chironji, chub mackerel, chum salmon, clam, climber, coconut palm, coffee, common amaranth, common buckwheat, common cypress, common mold, common mugwort, common olive, common ragweed, common roundworm, common sagebrush, common timothy, corn, crab, crayfish, crimson seabream, crustacean, cuttlefish, dandruff-associated fungus, danube crayfish, date palm, deep-water cape hake, dessert banana, disk abalone, dog, domestic guinea pig, dust mite, eastern oyster, eggplant, English walnut, European ash, European beech, European chestnut, European hake, European hazelnut, European hop hornbeam, European hornet, European house dust mite, European paper wasp, European pigeon tick, European rabbit, European white birch, fennel, feverfew, field mustard, fish worm, nematode, fishtail palm, flametree, royal Poinciana, flat fish, fleshy prawn, Formosan subterranean termite, frog, fungus, german cockroach, giant bull ant, giant honeybee, giant Madagascar hissing cockroach, giant mealworm beetle, giant mud crab, giant ragweed, giant river prawn, gold kiwi fruit, golden hamster, gould's razor shell, grape, grass carp, greasyback shrimp, greater wax moth, green kiwifruit, green mud crab, hardy kiwi, hardy pecan, hemp, herring worm, hinoki cypress, false cypress, holly oak, honeybee, hornbeam, hornet, horse, horse fly, horsehair crab, house cricket, house dust mite, Indian date, Indian honeybee, Indian mackerel, Indian meal moth, Italian cypress, itch mite, Japanese apricot, Japanese cedar, Japanese cypress, Japanese flying squid, Japanese hop, Japanese horse mackerel, Japanese rice, Japanese turban shell, Japanese white birch, Johnson grass, jumper ant, juniper, Kamchatka crab, Kentucky bluegrass, kidney bean, kiwi, kiwifruit, krill, kuruma shrimp, lemon, lentil, lesser banned hornet, lettuce, lilac, lobster, London plane tree, long-grained rice, lupine, lychee nut, Madagascar periwinkle, maize, mango, mantis shrimp, Mediterranean cypress, mesquite, mesquite pollen, methylotrophic yeast, midge, mite, mollusk, Mongolian oak, mosquito, moth, mould mite, mountain cedar, mouse, Mozambique tilapia, mugwort, mulberry, mung bean, mushroom, muskmelon, mustard, narrowleaf lupine, narrow-leaved blue lupine, narrow-leaved plantain, navel orange, neotropical wasp, netted muskmelon, noble scallop, north pacific hake, northern pink shrimp, oak, ocean perch, octopus, olive tree, orchard grass, oriental plane, papaya, paper wasp, para grass, para rubber tree, parasitic fish worm, parasitic nematode, parasitic roundworm, Patagonian grenadier, pea, peach, peanut, pear, pecan, pellitory of the wall, perennial ryegrass, pig, pigweed, pine, pine moth, pine processionary moth, pineapple, pistachio, pit-viper, plant hybrid, plum, pomegranate, Portuguese oyster, potato, privet, rainbow trout, rapeseed, raspberry, rat, red cedar, red fire ant, red squid, red swamp crayfish, rice, ringworm fungus, rivet wheat, rough pigweed, Russian thistle, rye, saffron crocus, sagebrush, salmon, sapodilla plum, scabies mite, schistosoma, sesame, shaggy mane, shallow-water cape hake, shan yao, sheep's briar, short ragweed, short-tailed mamushi, shrimp, Siberian hamster, silk worm, silver hake, silver wormwood, silverfish, silvery wormwood, skin fungus, smokybrown cockroach, snake venom, snow crab, south American pilchard, southern hake, southern house mosquito, southern pacific hake, soybean, spotless smooth-hound, stone pine, storage mite, strawberry, striped hairy-footed hamster, summer cypress, sunflower, sweet orange, sweet vernal grass, sweet wormwood, swordfish, tall fescue, tapeworm, thistle, tomato, tobacco, tropical fire ant, trumpet lily, tsetse fly, turnip, veined rapa whelk, velvet grass, walleye Pollock, wasp, watermelon, weed, western conenose, western king prawn, western ragweed, wheat, white legged freshwater shrimp, white lupine, white mulberry, white mustard, white oak, white shrimp, whiteface hornet, whiteleg shrimp, wild boar, wild cabbage, winter squash, wolf herring, wolfberry, wormwood, wrinkled Neptune, yak, yeast, yellow fever mosquito, yellow jacket, yellow lupine, yellow mealworm, and yellowfin tuna.

In some embodiments, the allergen, or pollen, is selected from Table 2 of U.S. Provisional Application No. 63/496,535, filed Apr. 17, 2023, which is hereby incorporated by reference in its entirety.

In some embodiments, the allergen, or pollen, has an amino acid sequence as provided in respective Accession number in the Comprehensive Protein Allergen Resource (COMPARE) Database (as accessed at the date of Apr. 17, 2023), which is hereby incorporated by reference in its entirety.

In some embodiments, the antigen of interest comprises a payload. In some embodiments, the payload is an antigen, an allergen, or a pollen. In some embodiments, the payload comprises an antigen is selected from glutamate decarboxylase 1 (GAD), glutamate decarboxylase 2 (GAD65), proinsulin, islet-specific glucose-6-phosphatase catalytic subunit-related protein (IGRP), zinc transporter 8 (Znt8), receptor-type tyrosine-protein phosphatase-like N (IA2), tropomyosin alpha-3 chain (TPM3), granulocyte-macrophage colony-stimulating factor (GMCSF/CSF2), RNA polymerase III complex subunit A (RNAP3), RNA polymerase III complex subunit B (RNAP3), RNA polymerase III complex subunit C (RNAP3), RNA polymerase III complex subunit D (RNAP3), RNA polymerase III complex subunit E (RNAP3), RNA polymerase III complex subunit F (RNAP3), RNA polymerase III complex subunit G (RNAP3), RNA polymerase III complex subunit H (RNAP3), RNA polymerase III complex subunit K (RNAP3), DNA topoisomerase 1 (TOPO1), type-1 angiotensin II receptor (ATIR), endothelin-1 receptor (ETAR), major centromere protein A (CENP), major centromere protein B (CENP), major centromere protein C (CENP), tetraspanin-7 (TSPAN7), 21-hydroxylase (21OH), 17alpha-hydroxylase (17OH), Th/To, ANA, dsDNA, HMGCR, myosin, SSA/Ro, SSB/La, U1RNP, U3RNP, PM-Sci, myelin basic protein (MBP), IL17/IL22, ASMA, actin, amylase α2, GM-CSF, cyclic citrullinated proteins (CCP), snRNP, Ro52, Ro60, La, Jo-1, SRP, IFIH1, CENPA, insulin, RNA-P, U1-70K, Sm-D3, MDA-5, PL7, PL-12, centromere proteins, PM/SCL, RNA polymerase 3 complex, tetraspanin-7, peptidylprolyl isomerase like 2, Mlh1, proteinase-3, immunoglobulin G, immunoglobulin M, myeloperoxidase, acetylcholine receptor (AchR), MUSK, LRP4, HSP90. HSPA5, kallikrein 13, ADAMTS13, IFN-7, IFN-α/ω, IL6, IL12/IL23, desmogleins (e.g., desmoglein-3, desmoglein-1), aquaporin-4, myelin oligodendrocyte glycoprotein (MOG), GRIN1, GRIA1, GRIA2, leucine rich glioma inactivated 1 protein (LGI1), neurexin family protein contactin associated protein 2 (CNTNAP2), collagen (e.g., collagen IV-alpha 3 chain (COL4A3), COL17), BP180, phospholipase A2 receptor (PLA2R), thrombospondin type-1 domain-containing 7A (THSD7A), thyroid hormone stimulating receptor (THSR), NMDAR, FGF23, acid beta-glycosidase, acid alpha-glycosidase, alpha-galactosidase A, alpha-N-acetylgalactosaminidase, alpha-L-iduronohydrolase, beta-hexosaminidase A, sphingomyelin phosphodiesterase 1, N-acetylsphingosine amidohydrolase 1, galactocerebrosidase, N-acetylgalactosamine-sulfate sulfatase, arylsulfatase B, iduronate 2-sulfatase, heparan-alpha-glucosaminide-N-acetyltransferase, beta-glucuronidase, hyaluronidase, sialidase 1, N-acetylglucosamine-1-phosphotransferase alpha/beta, N-acetylglucosamine-1-phosphotransferase gamma, glucose-6-phosphatase catalytic subunit, glucose-6-phosphate transporter, ATP7B, Phenylalanine hydroxylase, ATP-binding cassette subfamily A member 4, myocilin, coagulation factor VIII, coagulation factor IX, von Willebrand factor, glycoprotein Ib alpha, glycoprotein Ib beta, glycoprotein V, glycoprotein VI, glycoprotein IX, glycoprotein IIb, glycoprotein IIIa, platelet factor 4, AAV capsid protein (such as AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.eB, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9, AAV9 K449R (or K449R AAV9), AAV9.16, AAV9.24, AAV9.45, AAbiodisV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74 (also referred to as AAVrh74), AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, 7m8, Spark100, AAVMYO and variants thereof), SpCas9, SaCas9, FnCas12a, LbCas12a, AcCas12a, Cas13b, APOBEC1, E. coli TadA, Moloney Murine Leukemia Virus Reverse Transcriptase (M-MLV RT), HLA-A, HLA-B, HLA-C, HLA-E, HLA-L, HLA-J, HLA-K, HLA-H, HLA-G, HLA-DM, HLA-DO, HLA-DP, HLA-DQ, HLA-DR, any allergen provided herein, and any combination thereof.

In some embodiments, the antigen of interest can be referred to as an antigen binding domain. This can refer to a polypeptide, or an antibody, or the antigen-binding fragment thereof, that can bind to the antigen. For example, the polypeptide, or the antibody, or the antigen-binding fragment thereof, can be a polypeptide, or an antibody, or the antigen-binding fragment thereof, that binds to glutamate decarboxylase 1 (GAD), glutamate decarboxylase 2 (GAD65), proinsulin, islet-specific glucose-6-phosphatase catalytic subunit-related protein (IGRP), zinc transporter 8 (Znt8), receptor-type tyrosine-protein phosphatase-like N (IA2), tropomyosin alpha-3 chain (TPM3), granulocyte-macrophage colony-stimulating factor (GMCSF/CSF2), RNA polymerase III complex subunit A (RNAP3), RNA polymerase III complex subunit B (RNAP3), RNA polymerase III complex subunit C (RNAP3), RNA polymerase III complex subunit D (RNAP3), RNA polymerase III complex subunit E (RNAP3), RNA polymerase III complex subunit F (RNAP3), RNA polymerase III complex subunit G (RNAP3), RNA polymerase III complex subunit H (RNAP3), RNA polymerase III complex subunit K (RNAP3), DNA topoisomerase 1 (TOPO1), type-1 angiotensin II receptor (ATIR), endothelin-1 receptor (ETAR), major centromere protein A (CENP), major centromere protein B (CENP), major centromere protein C (CENP), tetraspanin-7 (TSPAN7), 21-hydroxylase (21OH), 17alpha-hydroxylase (17OH), Th/To, ANA, dsDNA, HMGCR, myosin, SSA/Ro, SSB/La, U1RNP, U3RNP, PM-Sci, myelin basic protein (MBP), IL17/IL22, ASMA, actin, amylase α2, GM-CSF, cyclic citrullinated proteins (CCP), snRNP, Ro52, Ro60, La, Jo-1, SRP, IFIH1, CENPA, insulin, RNA-P, U1-70K, Sm-D3, MDA-5, PL7, PL-12, centromere proteins, PM/SCL, RNA polymerase 3 complex, tetraspanin-7, peptidylprolyl isomerase like 2, Mlh1, proteinase-3, immunoglobulin G, immunoglobulin M, myeloperoxidase, acetylcholine receptor (AchR), MUSK, LRP4, HSP90. HSPA5, kallikrein 13, ADAMTS13, IFN-7, IFN-α/ω, IL6, IL12/IL23, desmogleins (e.g., desmoglein-3, desmoglein-1), aquaporin-4, myelin oligodendrocyte glycoprotein (MOG), GRIN1, GRIA1, GRIA2, leucine rich glioma inactivated 1 protein (LGI1), neurexin family protein contactin associated protein 2 (CNTNAP2), collagen (e.g., collagen IV-alpha 3 chain (COL4A3), COL17), BP180, phospholipase A2 receptor (PLA2R), thrombospondin type-1 domain-containing 7A (THSD7A), thyroid hormone stimulating receptor (THSR), NMDAR, FGF23, acid beta-glycosidase, acid alpha-glycosidase, alpha-galactosidase A, alpha-N-acetylgalactosaminidase, alpha-L-iduronohydrolase, beta-hexosaminidase A, sphingomyelin phosphodiesterase 1, N-acetylsphingosine amidohydrolase 1, galactocerebrosidase, N-acetylgalactosamine-sulfate sulfatase, arylsulfatase B, iduronate 2-sulfatase, heparan-alpha-glucosaminide-N-acetyltransferase, beta-glucuronidase, hyaluronidase, sialidase 1, N-acetylglucosamine-1-phosphotransferase alpha/beta, N-acetylglucosamine-1-phosphotransferase gamma, glucose-6-phosphatase catalytic subunit, glucose-6-phosphate transporter, ATP7B, Phenylalanine hydroxylase, ATP-binding cassette subfamily A member 4, myocilin, coagulation factor VIII, coagulation factor IX, von Willebrand factor, glycoprotein Ib alpha, glycoprotein Ib beta, glycoprotein V, glycoprotein VI, glycoprotein IX, glycoprotein IIb, glycoprotein IIIa, platelet factor 4, AAV capsid protein (such as AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.eB, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9, AAV9 K449R (or K449R AAV9), AAV9.16, AAV9.24, AAV9.45, AAbiodisV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74 (also referred to as AAVrh74), AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, 7m8, Spark100, AAVMYO and variants thereof), SpCas9, SaCas9, FnCas12a, LbCas12a, AcCas12a, Cas13b, APOBEC1, E. coli TadA, Moloney Murine Leukemia Virus Reverse Transcriptase (M-MLV RT), HLA-A, HLA-B, HLA-C, HLA-E, HLA-L, HLA-J, HLA-K, HLA-H, HLA-G, HLA-DM, HLA-DO, HLA-DP, HLA-DQ, and HLA-DR, any allergen provided herein, and any combination thereof. In some embodiments, the antibody, or the antigen-binding fragment thereof, bound to the antigen or pollen, is further internalized by the thymic cell.

In some embodiments, the antigen of interest is an antibody, or antigen-binding fragment thereof; an antigen, or a fragment thereof; an auto-antigen, or a fragment thereof; a self-antigen, or a fragment thereof; a tolerance inducing antigen, or a fragment thereof; or a ligand, or a fragment thereof. In some embodiments, the antigen of interest is an antigen that induces an autoimmune, alloimmune, allergic, inflammatory, or anti-drug immune response. In some embodiments, the autoimmune, alloimmune, allergic, inflammatory, or anti-drug immune response is a disorder or disease selected from the group consisting of diabetes mellitus type 1, ulcerative colitis, Crohn's disease, celiac disease, systemic sclerosis, graft versus host disease (GVHD), rheumatoid arthritis, anti-AAV immune response, asthma, severe asthma. neutrophilic asthma, paucigranulocytic asthma, eosinophilic asthma, osteoarthritis, solid organ transplant, atopic dermatitis, eosinophilic esophagitis, dermatitis herpitoformis, chronic rhinosinusitis, chronic spontaneous urticaria, food allergy, anti-biologic drug immune response, anti-viral immune response, anti-adenoviral immune response, anti-retroviral immune response, anti-lentiviral immune response, and -VLP immune response, Alzheimer's disease, amyotrophic lateral sclerosis (ALS), Parkinson's disease, dementia with Lewy bodies, Duchenne muscular dystrophy, cutaneous lupus myocarditis, postmyocardial infarction syndrome, postpericardiotomy syndrome, subacute bacterial endocarditis, anti-glomerular basement membrane nephritis, interstitial cystitis, lupus nephritis, membranous glomerulonephropathy, chronic kidney disease (“CKD”), autoimmune hepatitis, primary biliary cirrhosis, primary sclerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease (LAD), morphea, pemphigus, pemphigus vulgaris, hidradenitis suppuritiva, pityriasis lichenoides et varioliformis acuta, Mucha-Habermann disease, psoriasis, guttate psoriasis, erythrodermic psoriasis, plaque psoriasis, pustular psoriasis, skin manifestation associated with systemic sclerosis, vitiligo, Addison's disease, autoimmune polyendocrine syndrome (APS) type 1, autoimmune polyendocrine syndrome (APS) type 2, autoimmune polyendocrine syndrome (APS) type 3, autoimmune pancreatitis (AIP), autoimmune thyroiditis, Ord's thyroiditis, Graves' disease, non radiographic axial spondyloarthropathy, autoimmune oophoritis, endometriosis, autoimmune orchitis, Sjogren's syndrome, dermatitis herpatiformis, autoimmune enteropathy, coeliac disease, immune related adverse events (irAEs) secondary to immune checkpoint inhibitors, microscopic colitis, autoimmune thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, sarcoidosis, Schnitzler syndrome, systemic lupus erythematosus (SLE), undifferentiated connective tissue disease (UCTD), dermatomyositis, fibromyalgia, inclusion body myositis, myositis, myasthenia gravis, neuromyotonia, paraneoplastic cerebellar degeneration, polymyositis, acute disseminated encephalomyelitis (ADEM), acute motor axonal neuropathy, anti-N-methyl-D-aspartate (anti-NMDA) receptor encephalitis, balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, oshtoran syndrome, pediatric autoimmune neuropsychiatric disorder associated with streptococcus (PANDAS), progressive inflammatory neuropathy, restless leg syndrome, stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Mdniere's disease, Behcet's disease, eosinophilic granulomatosis with polyangiitis (EGPA), giant cell arteritis, granulmatosis with polyangiitis (GPA), IgA vasculitis (IgAV), Kawasaki's disease, leukocytoclastic vasculitis, lupus vasculitis, rheumatoid vasculitis, microscopic polyangiitis (MPA), polyarteritis nodosa (PAN), polymyalgia rheumaticia, vasculitis, primary immune deficiency, Gaucher disease, Pompe disease, Fabry disease, Schindler disease, Hurler syndrome (MPS1), Tay-Sachs disease, Nieman-Pick disease, Farber lipogranulomatosis, Krabbe disease, Morquio syndrome (MPS4), mucopolysaccharidosis type VI (MPS6), Hunter syndrome (MPS2), Sanfilippo syndrome (MPS3), Sly sundrome (MPS7), hyaluronidase deficiency (MPS9), mucolipidosis type 1 (ML1), I-cell disease, mucolipidosis II (ML2), mucolipidosis III gamma (ML3C), glycogen storage disease Ia, glycogen storage disease Ib, Wilson disease, phenylketonuria, Stargardt disease type I, myocilin-associated glaucoma, hemophilia A, hemophilia B, hereditary or acquired thrombotic thrombocytopathic purpura (TTP), von Willebrand disease, thrombocytopenia platelet autoantigen, heparin-induced thrombocytopenia, tolerance induction for AAV gene therapy, tolerance induction for ex vivo gene editing, tolerance induction for in vivo gene editing, acquired hemophilia, antiphospholipid syndrome, aplastic anemia, autoimmune lymphoproliferative syndrome, autoimmune neutropenia, autoimmune thrombocytopenic purpura, cold agglutinin disease, essential mixed cryoglobulinemia, Evans syndrome, IgG4-related diseases, pure red cell aplasia, warm autoimmune hemolytic anemia, autoimmune gastritis, pernicious anemia, anti-sperm antibodies, autoimmune polyendocrine syndrome type 1, autoimmune polyendocrine syndrome type 2, autoimmune polyendocrine syndrome type 3, autoimmune myocarditis, cryptogenic organizing pneumonia, idiopathic pulmonary fibrosis, IgA nephropathy, post-myocardial infarction syndrome, primary biliary cholangitis, primary idiopathic dilated cardiomyopathy, anti-N-methyl-D-aspartate receptor encephalitis, autoimmune encephalitis, encephalopathy associated with autoimmune thyroid disease, episcleritis, Graves' ophthalmopathy, myelin oligodendrocyte glycoprotein disease, narcolepsy with cataplexy, cutaneous lupus erythematosus, adult-onset Still's disease, ankylosing spondylitis, juvenile arthritis, juvenile dermatomyositis, anti-neutrophil cytoplasmic antibody-associated vasculitis, polymyalgia rheumatic, purpura rheumatic, Takayasu arteritis, transplant, and xenotransplantation.

In some embodiments, the antigen of interest is a diabetes mellitus type 1, ulcerative colitis, Crohn's disease, celiac disease, systemic sclerosis, or graft versus host disease (GVHD) associated antigen. In some embodiments, the antigen of interest is a diabetes mellitus type 1 antigen. In some embodiments, the antigen of interest is an ulcerative colitis antigen. In some embodiments, the antigen of interest is a Crohn's disease antigen. In some embodiments, the antigen of interest is a celiac disease antigen. In some embodiments, the antigen of interest is a systemic sclerosis antigen. In some embodiments, the antigen of interest is a graft versus host disease (GVHD) antigen.

In some embodiments, the diabetes mellitus type 1 antigen is selected from the group consisting of: glutamate decarboxylase 1 (GAD), glutamate decarboxylase 2 (GAD65), proinsulin, islet-specific glucose-6-phosphatase catalytic subunit-related protein (IGRP), zinc transporter 8 (ZNT8), and receptor-type tyrosine-protein phosphatase-like N (IA2).

In some embodiments, the diabetes mellitus type 1 antigen is glutamate decarboxylase 1 (GAD). In some embodiments, the glutamate decarboxylase 1 (GAD) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% at least 99% sequence identity to:

(SEQ ID NO: 14)
MASSTPSSSATSSNAGADPNTTNLRPTTYDTWCGVAHGCTRKLGLKICGF
LORINSLEEKSRLVSAFKERQSSKNLLSCENSDRDARFRRTETDFSNLFA
RDLLPAKNGEEQTVQFLLEVVDILLNYVRKTFDRSTKVLDFHHPHQLLEG
MEGFNLELSDHPESLEQILVDCRDTLKYGVRTGHPRFFNQLSTGLDIIGL
AGEWLISTANTNMFTYEIAPVFVLMEQITLKKMREIVGWSSKDGDGIFSP
GGAISNMYSIMAARYKYFPEVKTKGMAAVPKLVLFTSEQSHYSIKKAGAA
LGFGTDNVILIKCNERGKIIPADFEAKILEAKQKGYVPFYVNATAGITVY
GAFDPIQEIADICEKYNLWLHVDAAWGGGLLMSRKHRHKLNGIERANSVT
WNPHKMMGVLLQCSAILVKEKGILQGCNQMCAGYLFQPDKQYDVSYDTGD
KAIQCGRHVDIFKFWLMWKAKGTVGFENQINKCLELAEYLYAKIKNREEF
EMVENGEPEHTNVCFWYIPQSLRGVPDSPQRREKLHKVAPKIKALMMESG
TTMVGYQPQGDKANFFRMVISNPAATQSDIDFLIEEIERLGQDL.

In some embodiments, the glutamate decarboxylase 1 (GAD) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 14 and does not comprise the N-terminal methionine (M) residue. In some embodiments, the glutamate decarboxylase 1 (GAD) comprises the amino acid sequence of SEQ ID NO: 14.

In some embodiments, the diabetes mellitus type 1 antigen is glutamate decarboxylase 2 (GAD65). In some embodiments, the glutamate decarboxylase 2 (GAD65) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 15)
MASPGSGFWSFGSEDGSGDSENPGTARAWCQVAQKFTGGIGNKLCALLYG
DAEKPAESGGSQPPRAAARKAACACDQKPCSCSKVDVNYAFLHATDLLPA
CDGERPTLAFLQDVMNILLQYVVKSFDRSTKVIDFHYPNELLQEYNWELA
DQPONLEEILMHCQTTLKYAIKTGHPRYFNQLSTGLDMVGLAADWLISTA
NTNMFTYEIAPVFVLLEYVTLKKMREIIGWPGGSGDGIFSPGGAISNMYA
MMIARFKMFPEVKEKGMAALPRLIAFTSEHSHFSLKKGAAALGIGTDSVI
LIKCDERGKMIPSDLERRILEAKQKGFVPFLVSATAGITVYGAFDPLLAV
ADICKKYKIWMHVDAAWGGGLLMSRKHKWKLSGVERANSVTWNPHKMMGV
PLQCSALLVREEGLMQNCNQMHASYLFQQDKHYDLSYDTGDKALQCGRHV
DVFKLWLMWRAKGTTGFEAHVDKCLELAEYLYNIIKNREGYEMVFDGKPQ
HTNVCFWYIPPSLRTLEDNEERMSRLSKVAPVIKARMMEYGTTMVSYQPL
GDKVNFFRMVISNPAATHQDIDFLIEEIERLGQDL.

In some embodiments, the glutamate decarboxylase 2 (GAD65) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 15 and does not comprise the N-terminal methionine (M) residue. In some embodiments, the glutamate decarboxylase 2 (GAD65) comprises the amino acid sequence of SEQ ID NO: 15.

In some embodiments, the diabetes mellitus type 1 antigen is proinsulin. In some embodiments, the proinsulin comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 16)
MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN.

In some embodiments, the proinsulin comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 16 and does not comprise the N-terminal methionine (M) residue. In some embodiments, the proinsulin comprises the amino acid sequence of SEQ ID NO: 16.

In some embodiments, the diabetes mellitus type 1 antigen is islet-specific glucose-6-phosphatase catalytic subunit-related protein (IGRP). In some embodiments, the islet-specific glucose-6-phosphatase catalytic subunit-related protein (IGRP) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 17)
MDFLHRNGVLIIQHLQKDYRAYYTFLNFMSNVGDPRNIFFIYFPLCFQFN
QTVGTKMIWVAVIGDWLNLIFKWILFGHRPYWWVOETQIYPNHSSPCLEQ
FPTTCETGPGSPSGHAMGASCVWYVMVTAALSHTVCGMDKFSITLHRLTW
SFLWSVFWLIQISVCISRVFIATHFPHQVILGVIGGMLVAEAFEHTPGIQ
TASLGTYLKINLFLFLFAVGFYLLLRVLNIDLLWSVPIAKKWCANPDWIH
IDTTPFAGLVRNLGVLFGLGFAINSEMFLLSCRGGNNYTLSFRLLCALTS
LTILQLYHFLQIPTHEEHLFYVLSFCKSASIPLTVVAFIPYSVHMLMKQS
GKKSQ.

In some embodiments, the is islet-specific glucose-6-phosphatase catalytic subunit-related protein (IGRP) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 17 and does not comprise the N-terminal methionine (M) residue. In some embodiments, the islet-specific glucose-6-phosphatase catalytic subunit-related protein (IGRP) comprises the amino acid sequence of SEQ ID NO: 17.

In some embodiments, the diabetes mellitus type 1 antigen is zinc transporter 8 (ZNT8). In some embodiments, the zinc transporter 8 (ZNT8) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 18)
MEFLERTYLVNDKAAKMYAFTLESVELQQKPVNKDOCPRERPEELESGGM
YHCHSGSKPTEKGANEYAYAKWKLCSASAICFIFMIAEVVGGHIAGSLAV
VTDAAHLLIDLTSFLLSLFSLWLSSKPPSKRLTFGWHRAEILGALLSILC
IWVVTGVLVYLACERLLYPDYQIQATVMIIVSSCAVAANIVLIVVLHQRC
LGHNHKEVQANASVRAAFVHALGDLFQSISVLISALIIYFKPEYKIADPI
CTFIFSILVLASTITILKDFSILLMEGVPKSLNYSGVKELILAVDGVLSV
HSLHIWSLTMNQVILSAHVATAASRDSQVVRREIAKALSKSFTMHSLTIQ
MESPVDQDPDCLFCEDPCD.

In some embodiments, the zinc transporter 8 (ZNT8) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 18 and does not comprise the N-terminal methionine (M) residue. In some embodiments, the zinc transporter 8 (ZNT8) comprises the amino acid sequence of SEQ ID NO: 18.

In some embodiments, the diabetes mellitus type 1 antigen is receptor-type tyrosine-protein phosphatase-like N (IA2). In some embodiments, the receptor-type tyrosine-protein phosphatase-like N (IA2) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 19)
MRRPRRPGGLGGSGGLRLLLCLLLLSSRPGGCSAVSAHGCLFDRRLCSHL
EVCIQDGLFGQCQVGVGQARPLLQVTSPVLQRLQGVLRQLMSQGLSWHDD
LIQYVISQEMERIPRLRPPEPRPRDRSGLAPKRPGPAGELLLQDIPTGSA
PAAQHRLPQPPVGKGGAGASSSLSPLQAELLPPLLEHLLLPPQPPHPSLS
YEPALLQPYLFHQFGSRDGSRVSEGSPGMVSVGPLPKAEAPALFSRTASK
GIFGDHPGHSYGDLPGPSPAQLFQDSGLLYLAQELPAPSRARVPRLPEQG
SSSRAEDSPEGYEKEGLGDRGEKPASPAVQPDAALQRLAAVLAGYGVELR
QLTPEQLSTLLTLLQLLPKGAGRNPGGVVNVGADIKKTMEGPVEGRDTAE
LPARTSPMPGHPTASPTSSEVQQVPSPVSSEPPKAARPPVTPVLLEKKSP
LGQSQPTVAGQPSARPAAEEYGYIVTDQKPLSLAAGVKLLEILAEHVHMS
SGSFINISVVGPALTFRIRHNEQNLSLADVTQQAGLVKSELEAQTGLQIL
QTGVGQREEAAAVLPQTAHSTSPMRSVLLTLVALAGVAGLLVALAVALCV
RQHARQQDKERLAALGPEGAHGDTTFEYQDLCRQHMATKSLFNRAEGPPE
PSRVSSVSSQFSDAAQASPSSHSSTPSWCEEPAQANMDISTGHMILAYME
DHLRNRDRLAKEWQALCAYQAEPNTCATAQGEGNIKKNRHPDFLPYDHAR
IKLKVESSPSRSDYINASPIIEHDPRMPAYIATQGPLSHTIADFWQMVWE
SGCTVIVMLTPLVEDGVKQCDRYWPDEGASLYHVYEVNLVSEHIWCEDFL
VRSFYLKNVQTQETRTLTQFHFLSWPAEGTPASTRPLLDFRRKVNKCYRG
RSCPIIVHCSDGAGRTGTYILIDMVLNRMAKGVKEIDIAATLEHVRDQRP
GLVRSKDQFEFALTAVAEEVNAILKALPQ.

In some embodiments, the receptor-type tyrosine-protein phosphatase-like N (IA2) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 19 and does not comprise the N-terminal methionine (M) residue. In some embodiments, the receptor-type tyrosine-protein phosphatase-like N (IA2) comprises the amino acid sequence of SEQ ID NO: 19.

In some embodiments, the ulcerative colitis antigen is selected from the group consist of tropomyosin alpha-3 chain (TPM3), and granulocyte-macrophage colony-stimulating factor (GMCSF/CSF2).

In some embodiments, the ulcerative colitis antigen is tropomyosin alpha-3 chain (TPM3). In some embodiments, the tropomyosin alpha-3 chain (TPM3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 20)
MMEAIKKKMQMLKLDKENALDRAEQAEAEQKQAEERSKQLEDELAAMQKK
LKGTEDELDKYSEALKDAQEKLELAEKKAADAEAEVASLNRRIQLVEEEL
DRAQERLATALQKLEEAEKAADESERGMKVIENRALKDEEKMELQEIQLK
EAKHIAEEADRKYEEVARKLVIIEGDLERTEERAELAESKCSELEEELKN
VTNNLKSLEAQAEKYSQKEDKYEEEIKILTDKLKEAETRAEFAERSVAKL
EKTIDDLEDELYAQKLKYKAISEELDHALNDMTSI.

In some embodiments, the tropomyosin alpha-3 chain (TPM3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 20 and does not comprise the N-terminal methionine (M) residue. In some embodiments, the tropomyosin alpha-3 chain (TPM3) comprises the amino acid sequence of SEQ ID NO: 20.

In some embodiments, the ulcerative colitis antigen is granulocyte-macrophage colony-stimulating factor (GMCSF/CSF2). In some embodiments, the granulocyte-macrophage colony-stimulating factor (GMCSF/CSF2) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 21)
MWLQSLLLLGTVACSISAPARSPSPSTQPWEHVNAIQEARRLLNLSRDTA
AEMNETVEVISEMFDLQEPTCLQTRLELYKQGLRGSLTKLKGPLTMMASH
YKQHCPPTPETSCATQIITFESFKENLKDELLVIPFDCWEPVQE.

In some embodiments, the granulocyte-macrophage colony-stimulating factor (GMCSF/CSF2) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 21 and does not comprise the N-terminal methionine (M) residue. In some embodiments, the granulocyte-macrophage colony-stimulating factor (GMCSF/CSF2) comprises the amino acid sequence of SEQ ID NO: 21.

In some embodiments, the Crohn's disease antigen is selected from the group consist of tropomyosin alpha-3 chain (TPM3), and granulocyte-macrophage colony-stimulating factor (GMCSF/CSF2).

In some embodiments, the Crohn's disease antigen is tropomyosin alpha-3 chain (TPM3). In some embodiments, the tropomyosin alpha-3 chain (TPM3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 20. In some embodiments, the tropomyosin alpha-3 chain (TPM3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 20 and does not comprise the N-terminal methionine (M) residue. In some embodiments, the tropomyosin alpha-3 chain (TPM3) comprises the amino acid sequence of SEQ ID NO: 20.

In some embodiments, the Crohn's disease antigen is granulocyte-macrophage colony-stimulating factor (GMCSF/CSF2). In some embodiments, the granulocyte-macrophage colony-stimulating factor (GMCSF/CSF2) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 21. In some embodiments, the granulocyte-macrophage colony-stimulating factor (GMCSF/CSF2) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 21 and does not comprise the N-terminal methionine (M) residue. In some embodiments, the granulocyte-macrophage colony-stimulating factor (GMCSF/CSF2) comprises the amino acid sequence of SEQ ID NO: 21.

In some embodiments, the celiac disease antigen is selected from the group consisting of tropomyosin alpha-3 chain (TPM3), and granulocyte-macrophage colony-stimulating factor (GMCSF/CSF2).

In some embodiments, the celiac disease antigen is tropomyosin alpha-3 chain (TPM3). In some embodiments, the tropomyosin alpha-3 chain (TPM3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 20. In some embodiments, the tropomyosin alpha-3 chain (TPM3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 20 and does not comprise the N-terminal methionine (M) residue. In some embodiments, the tropomyosin alpha-3 chain (TPM3) comprises the amino acid sequence of SEQ ID NO: 20.

In some embodiments, the celiac disease antigen is granulocyte-macrophage colony-stimulating factor (GMCSF/CSF2). In some embodiments, the granulocyte-macrophage colony-stimulating factor (GMCSF/CSF2) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 21. In some embodiments, the granulocyte-macrophage colony-stimulating factor (GMCSF/CSF2) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 21 and does not comprise the N-terminal methionine (M) residue. In some embodiments, the granulocyte-macrophage colony-stimulating factor (GMCSF/CSF2) comprises the amino acid sequence of SEQ ID NO: 21.

In some embodiments, the systemic sclerosis antigen is selected from the group consisting of: RNA polymerase III complex subunit A (RNAP3), RNA polymerase III complex subunit B (RNAP3), RNA polymerase III complex subunit C (RNAP3), RNA polymerase III complex subunit D (RNAP3), RNA polymerase III complex subunit E (RNAP3), RNA polymerase III complex subunit F (RNAP3), RNA polymerase III complex subunit G (RNAP3), RNA polymerase III complex subunit H (RNAP3), RNA polymerase III complex subunit K (RNAP3), DNA topoisomerase 1 (TOPO1), type-1 angiotensin II receptor (AT1R),endothelin-1 receptor (ETAR), major centromere protein A (CENP), major centromere protein B (CENP), major centromere protein C (CENP), and tetraspanin-7 (TSPAN7).

In some embodiments, the systemic sclerosis antigen is RNA polymerase III complex subunit A (RNAP3). In some embodiments, the RNA polymerase III complex subunit A (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 22)
MVKEQFRETDVAKKISHICFGMKSPEEMRQQAHIQVVSKNLYSQDNQHAP
LLYGVLDHRMGTSEKDRPCETCGKNLADCLGHYGYIDLELPCFHVGYFRA
VIGILQMICKTCCHIMLSQEEKKQFLDYLKRPGLTYLQKRGLKKKISDKC
RKKNICHHCGAFNGTVKKCGLLKITHEKYKTNKKVVDPIVSNFLQSFETA
IEHNKEVEPLLGRAQENLNPLVVLNLFKRIPAEDVPLLLMNPEAGKPSDL
ILTRLLVPPLCIRPSVVSDLKSGTNEDDLIMKLTEIIFLNDVIKKHRISG
AKTQMIMEDWDFLQLQCALYINSELSGIPLNMAPKKWTRGFVQRLKGKQG
RFRGNLSGKRVDFSGRTVISPDPNLRIDEVAVPVHVAKILTFPEKVNKAN
INFLRKLVQNGPEVHPGANFIQQRHTQMKRFLKYGNREKMAQELKYGDIV
ERHLIDGDVVLFNRQPSLHKLSIMAHLARVKPHRTFRFNECVCTPYNADF
DGDEMNLHLPQTEEAKAEALVLMGTKANLVTPRNGEPLIAAIQDFLIGAY
LLTLKDIFFDRAKACQIIASILVGKDEKIKVRLPPPTILKPVTLWTGKQI
FSVILRPSDDNPVRANLRTKGKQYCGKGEDLCANDSYVTIQNSELMSGSM
DKGTLGSGSKNNIFYILLRDWGQNEAADAMSRLARLAPVYLSNRGFSIGI
GDVTPGQGLLKAKYELLNAGYKKCDEYIEALNTGKLQQQPGCTAEETLEA
LILKELSVIRDHAGSACLRELDKSNSPLTMALCGSKGSFINISQMIACVG
QQAISGSRVPDGFENRSLPHFEKHSKLPAAKGFVANSFYSGLTPTEFFFH
TMAGREGLVDTAVKTAETGYMQRRLVKSLEDLCSQYDLTVRSSTGDIIQF
IYGGDGLDPAAMEGKDEPLEFKRVLDNIKAVFPCPSEPALSKNELILTTE
SIMKKSEFLCCQDSFLQEIKKFIKGVSEKIKKTRDKYGINDNGTTEPRVL
YQLDRITPTQVEKFLETCRDKYMRAQMEPGSAVGALCAQSIGEPGTQMTL
KTFHFAGVASMNITLGVPRIKEIINASKAISTPIITAQLDKDDDADYARL
VKGRIEKTLLGEISEYIEEVFLPDDCFILVKLSLERIRLLRLEVNAETVR
YSICTSKLRVKPGDVAVHGEAVVCVTPRENSKSSMYYVLQFLKEDLPKVV
VQGIPEVSRAVIHIDEQSGKEKYKLLVEGDNLRAVMATHGVKGTRTTSNN
TYEVEKTLGIEAARTTIINEIQYTMVNHGMSIDRRHVMLLSDLMTYKGEV
LGITRFGLAKMKESVLMLASFEKTADHLFDAAYFGQKDSVCGVSECIIMG
IPMNIGTGLFKLLHKADRDPNPPKRPLIFDTNEFHIPLVT.

In some embodiments, the RNA polymerase III complex subunit A (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 22 and does not comprise the N-terminal methionine (M) residue. In some embodiments, the RNA polymerase III complex subunit A (RNAP3) comprises the amino acid sequence of SEQ ID NO: 22.

In some embodiments, the systemic sclerosis antigen is RNA polymerase III complex subunit B (RNAP3). In some embodiments, the RNA polymerase III complex subunit B (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 23)
MDVLAEEFGNLTPEQLAAPIPTVEEKWRLLPAFLKVKGLVKQHIDSFNYFINVEIKKIM
KANEKVTSDADPMWYLKYLNIYVGLPDVEESFNVTRPVSPHECRLRDMTYSAPITVDIE
YTRGSQRIIRNALPIGRMPIMLRSSNCVLIGKTPAEFAKLNECPLDPGGYFIVKGVEKV
ILIQEQLSKNRIIVEADRKGAVGASVISSTHEKKSRINMAVKQGRFYLRHNTLSEDIPI
VIIFKAMGVESDQEIVQMIGTEEHVMAAFGPSLEECQKAQIFTQMQALKYIGNKVRRQR
MWGGGPKKTKIEEARELLASTILTHVPVKEFNFRAKCIYTAVMVRRVILAQGDNKVDDR
DYYGNKRLELAGQLLSLLFEDLFKKENSEMKKIADQVIPKQRAAQFDVVKHMRQDQITN
GMVNAISTGNWSLKRFKMDRQGVTQVLSRLSYISALGMMTRISSQFEKTRKVSGPRSLQ
PSQWGMLCPSDTPEGEACGLVKNLALMTHITTDMEDGPIVKLASNLGVEDVNLLCGEEL
SYPNVFLVFLNGNILGVIRDHKKLVNTFRLMRRAGYINEFVSISTNLTDRCVYISSDGG
RLCRPYIIVKKQKPAVINKHMEELAQGYRNFEDFLHESLVEYLDVNEENDCNIALYEHT
INKDTTHLEIEPFTLLGVCAGLIPYPHHNQSPRNTYQCAMGKQAMGTIGYNQRNRIDTL
MYLLAYPQKPMVKTKTIELIEFEKLPAGQNATVAVMSYSGYDIEDALVLNKASLDRGFG
RCLVYKNAKCTLKRYTNQTFDKVMGPMLDAATRKPIWRHEILDADGICSPGEKVENKQV
LVNKSMPTVTQIPLEGSNVPQQPQYKDVPITYKGATDSYIEKVMISSNAEDAFLIKMLL
RQTRRPEIGDKFSSRHGQKGVCGLIVPQEDMPFCDSGICPDIIMNPHGFPSRMTVGKLI
ELLAGKAGVLDGRFHYGTAFGGSKVKDVCEDLVRHGYNYLGKDYVTSGITGEPLEAYIY
FGPVYYQKLKHMVLDKMHARARGPRAVLTRQPTEGRSRDGGLRLGEMERDCLIGYGASM
LLLERLMISSDAFEVDVCGQCGLLGYSGWCHYCKSSCHVSSLRIPYACKLLFQELQSMN
IIPRLKLSKYNE.

In some embodiments, the RNA polymerase III complex subunit B (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 23 and does not comprise the N-terminal methionine (M) residue. In some embodiments, the RNA polymerase III complex subunit B (RNAP3) comprises the amino acid sequence of SEQ ID NO: 23.

In some embodiments, the systemic sclerosis antigen is RNA polymerase III complex subunit C (RNAP3). In some embodiments, the RNA polymerase III complex subunit C (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 24)
MTQAEIKLCSLLLQEHFGEIVEKIGVHLIRTGSQPLRVIAHDTGTSLDQVKKALCVLVQ
HNLVSYQVHKRGVVEYEAQCSRVLRMLRYPRYIYTTKTLYSDTGELIVEELLLNGKLTM
SAVVKKVADRLTETMEDGKTMDYAEVSNTFVRLADTHFVQRCPSVPTTENSDPGPPPPA
PTLVINEKDMYLVPKLSLIGKGKRRRSSDEDAAGEPKAKRPKYTTDNKEPIPDDGIYWQ
ANLDRFHQHFRDQAIVSAVANRMDQTSSEIVRIMLRMSEITTSSSAPFTQPLSSNEIFR
SLPVGYNISKQVLDQYLTLLADDPLEFVGKSGDSGGGMYVINLHKALASLATATLESVV
QERFGSRCARIFRLVLQKKHIEQKQVEDFAMIPAKEAKDMLYKMLSENFMSLQEIPKTP
DHAPSRTFYLYTVNILSAARMLLHRCYKSIANLIERRQFETKENKRLLEKSQRVEAIIA
SMQATGAEEAQLQEIEEMITAPERQQLETLKRNVNKLDASEIQVDETIFLLESYIECTM
KRQ.

In some embodiments, the RNA polymerase III complex subunit C (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 24 and does not comprise the N-terminal methionine (M) residue. In some embodiments, the RNA polymerase III complex subunit C (RNAP3) comprises the amino acid sequence of SEQ ID NO: 24.

In some embodiments, the systemic sclerosis antigen is RNA polymerase III complex subunit D (RNAP3). In some embodiments, the RNA polymerase III complex subunit D (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 25)
MSEGNAAGEPSTPGGPRPLLTGARGLIGRRPAPPLTPGRLPSIRSRDLTLGGVKKKTFT
PNIISRKIKEEPKEEVTVKKEKRERDRDRQREGHGRGRGRPEVIQSHSIFEQGPAEMMK
KKGNWDKTVDVSDMGPSHIINIKKEKRETDEETKQILRMLEKDDFLDDPGLRNDTRNMP
VQLPLAHSGWLFKEENDEPDVKPWLAGPKEEDMEVDIPAVKVKEEPRDEEEEAKMKAPP
KAARKTPGLPKDVSVAELLRELSLTKEEELLFLQLPDTLPGQPPTQDIKPIKTEVQGED
GQVVLIKQEKDREAKLAENACTLADLTEGQVGKLLIRKSGRVQLLLGKVTLDVTMGTAC
SFLQELVSVGLGDSRTGEMTVLGHVKHKLVCSPDFESLLDHKHR.

In some embodiments, the RNA polymerase III complex subunit D (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 25 and does not comprise the N-terminal methionine (M) residue. In some embodiments, the RNA polymerase III complex subunit D (RNAP3) comprises the amino acid sequence of SEQ ID NO: 25.

In some embodiments, the systemic sclerosis antigen is RNA polymerase III complex subunit E (RNAP3). In some embodiments, the RNA polymerase III complex subunit E (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 26)
MANEEDDPVVQEIDVYLAKSLAEKLYLFQYPVRPASMTYDDIPHLSAKIKPKQQKVELE
MAIDTLNPNYCRSKGEQIALNVDGACADETSTYSSKLMDKQTFCSSQTTSNTSRYAAAL
YRQGELHLTPLHGILQLRPSFSYLDKADAKHREREAANEAGDSSQDEAEDDVKQITVRF
SRPESEQARQRRVQSYEFLQKKHAEEPWVHLHYYGLRDSRSEHERQYLLCPGSSGVENT
ELVKSPSEYLMMLMPPSQEEEKDKPVAPSNVLSMAQLRTLPLADQIKILMKNVKVMPFA
NLMSLLGPSIDSVAVLRGIQKVAMLVQGNWVVKSDILYPKDSSSPHSGVPAEVLCRGRD
FVMWKFTQSRWVVRKEVATVTKLCAEDVKDFLEHMAVVRINKGWEFILPYDGEFIKKHP
DVVQRQHMLWTGIQAKLEKVYNLVKETMPKKPDAQSGPAGLVCGDQRIQVAKTKAQQNH
ALLERELQRRKEQLRVPAVPPGVRIKEEPVSEEGEEDEEQEAEEEPMDTSPSGLHSKLA
NGLPLGRAAGTDSFNGHPPQGCASTPVARELKAFVEATFQRQFVLTLSELKRLFNLHLA
SLPPGHILFSGISDRMLQDTVLAAGCKQILVPFPPQTAASPDEQKVFALWESGDMSDQH
RQVLLEIFSKNYRVRRNMIQSRLTQECGEDLSKQEVDKVLKDCCVSYGGMWYLKGTVQ
S.

In some embodiments, the RNA polymerase III complex subunit E (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 26 and does not comprise the N-terminal methionine (M) residue. In some embodiments, the RNA polymerase III complex subunit E (RNAP3) comprises the amino acid sequence of SEQ ID NO: 26.

In some embodiments, the systemic sclerosis antigen is RNA polymerase III complex subunit F (RNAP3). In some embodiments, the RNA polymerase III complex subunit F (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 27)
MAEVKVKVQPPDADPVEIENRIIELCHQFPHGITDQVIQNEMPHIEAQQRAVAINRLLS
MGQLDLLRSNTGLLYRIKDSQNAGKMKGSDNQEKLVYQIIEDAGNKGIWSRDIRYKSNL
PLTEINKILKNLESKKLIKAVKSVAASKKKVYMLYNLQPDRSVTGGAWYSDQDFESEFV
EVLNQQCFKFLQSKAETARESKQNPMIQRNSSFASSHEVWKYICELGISKVELSMEDIE
TILNTLIYDGKVEMTIIAAKEGTVGSVDGHMKLYRAVNPIIPPTGLVRAPCGLCPVEDD
CHEGGEISPSNCIYMTEWLEF.

In some embodiments, the RNA polymerase III complex subunit F (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 27 and does not comprise the N-terminal methionine (M) residue. In some embodiments, the RNA polymerase III complex subunit F (RNAP3) comprises the amino acid sequence of SEQ ID NO: 27.

In some embodiments, the systemic sclerosis antigen is RNA polymerase III complex subunit G (RNAP3). In some embodiments, the RNA polymerase III complex subunit G (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 28)
MAGNKGRGRAAYTFNIEAVGFSRGEKLPDVVLKPPPLFPDTDYKPVPLKTGEDEDYMLA
LKQELRETVKRLPYFIEPPEEKQDDIERYSKRYMKVYKEEWVPDWRRLPREMMPRKKCK
KGDPKSKPSKAAAKATSLINSADVLKTIEELEKRGEGERSDEENEEKEGSKEKDKDDEE
DGEEDAEQEDYDEEEQEEENDYINSYFDNGDDFGVDSDDNMDEATY.

In some embodiments, the RNA polymerase III complex subunit G (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 28 and does not comprise the N-terminal methionine (M) residue. In some embodiments, the RNA polymerase III complex subunit G (RNAP3) comprises the amino acid sequence of SEQ ID NO: 28.

In some embodiments, the systemic sclerosis antigen is RNA polymerase III complex subunit H (RNAP3). In some embodiments, the RNA polymerase III complex subunit H (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 29)
MFVLVEMVDTVRIPPWQFERKLNDSIAEELNKKLANKVVYNVGLCICLFDITKLEDAYV
FPGDGASHTKVHFRCVVFHPFLDEILIGKIKGCSPEGVHVSLGFFDDILIPPESLQQPA
KFDEAEQVWVWEYETEEGAHDLYMDTGEEIRFRVVDESFVDTSPTGPSSADATTSSEEL
PKKEAPYTLVGSISEPGLGLLSWWTSN.

In some embodiments, the RNA polymerase III complex subunit H (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 29 and does not comprise the N-terminal methionine (M) residue. In some embodiments, the RNA polymerase III complex subunit H (RNAP3) comprises the amino acid sequence of SEQ ID NO: 29.

In some embodiments, the systemic sclerosis antigen is RNA polymerase III complex subunit K (RNAP3). In some embodiments, the RNA polymerase III complex subunit K (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 30)
MLLFCPGCGNGLIVEEGQRCHRFACNTCPYVHNITRKVTNRKYPKLKEV
DDVLGGAAAWENVDSTAESCPKCEHPRAYFMQLQTRSADEPMTTFYKCC
NAQCGHRWRD.

In some embodiments, the RNA polymerase III complex subunit K (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 30 and does not comprise the N-terminal methionine (M) residue. In some embodiments, the RNA polymerase III complex subunit K (RNAP3) comprises the amino acid sequence of SEQ ID NO: 30.

In some embodiments, the systemic sclerosis antigen is DNA topoisomerase 1 (TOPO1). In some embodiments, the DNA topoisomerase 1 (TOPO1) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 31)
MSGDHLHNDSQIEADFRLNDSHKHKDKHKDREHRHKEHKKEKDREKSKHSNSEHKDSEK
KHKEKEKTKHKDGSSEKHKDKHKDRDKEKRKEEKVRASGDAKIKKEKENGFSSPPQIKD
EPEDDGYFVPPKEDIKPLKRPRDEDDADYKPKKIKTEDTKKEKKRKLEEEEDGKLKKPK
NKDKDKKVPEPDNKKKKPKKEEEQKWKWWEEERYPEGIKWKFLEHKGPVFAPPYEPLPE
NVKFYYDGKVMKLSPKAEEVATFFAKMLDHEYTTKEIFRKNFFKDWRKEMTNEEKNIIT
NLSKCDFTQMSQYFKAQTEARKQMSKEEKLKIKEENEKLLKEYGFCIMDNHKERIANFK
IEPPGLFRGRGNHPKMGMLKRRIMPEDIIINCSKDAKVPSPPPGHKWKEVRHDNKVTWL
VSWTENIQGSIKYIMLNPSSRIKGEKDWQKYETARRLKKCVDKIRNQYREDWKSKEMKV
RQRAVALYFIDKLALRAGNEKEEGETADTVGCCSLRVEHINLHPELDGQEYVVEFDFLG
KDSIRYYNKVPVEKRVFKNLQLFMENKQPEDDLFDRLNTGILNKHLQDLMEGLTAKVER
TYNASITLQQQLKELTAPDENIPAKILSYNRANRAVAILCNHQRAPPKTFEKSMMNLQT
KIDAKKEQLADARRDLKSAKADAKVMKDAKTKKVVESKKKAVQRLEEQLMKLEVQATDR
EENKQIALGTSKLNYLDPRITVAWCKKWGVPIEKIYNKTQREKFAWAIDMADEDYEF.

In some embodiments, the DNA topoisomerase 1 (TOPO1) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 31 and does not comprise the N-terminal methionine (M) residue. In some embodiments, the DNA topoisomerase 1 (TOPO1) comprises the amino acid sequence of SEQ ID NO: 31.

In some embodiments, the systemic sclerosis antigen is type-1 angiotensin II receptor (AT1R). In some embodiments, the type-1 angiotensin II receptor (AT1R) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97% at least 98% at least 99% sequence identity to:

(SEQ ID NO: 32)
MILNSSTEDGIKRIQDDCPKAGRHNYIFVMIPTLYSIIFVVGIFGNSLVVIVIYFYMKL
KTVASVFLLNLALADLCFLLTLPLWAVYTAMEYRWPFGNYLCKIASASVSFNLYASVFL
LTCLSIDRYLAIVHPMKSRLRRTMLVAKVTCIIIWLLAGLASLPAIIHRNVFFIENTNI
TVCAFHYESQNSTLPIGLGLTKNILGFLFPFLIILTSYTLIWKALKKAYEIQKNKPRND
DIFKIIMAIVLFFFFSWIPHQIFTFLDVLIQLGIIRDCRIADIVDTAMPITICIAYENN
CLNPLFYGFLGKKFKRYFLQLLKYIPPKAKSHSNLSTKMSTLSYRPSDNVSSSTKKPAP
CFEVE.

In some embodiments, the type-1 angiotensin II receptor (AT1R) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 32 and does not comprise the N-terminal methionine (M) residue. In some embodiments, the type-1 angiotensin II receptor (AT1R) comprises the amino acid sequence of SEQ ID NO: 32.

In some embodiments, the systemic sclerosis antigen is endothelin-1 receptor (ETAR). In some embodiments, the endothelin-1 receptor (ETAR) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 33)
METLCLRASFWLALVGCVISDNPERYSTNLSNHVDDFTTFRGTELSFLVTTHQPTNLVL
PSNGSMHNYCPQQTKITSAFKYINTVISCTIFIVGMVGNATLLRIIYQNKCMRNGPNAL
IASLALGDLIYVVIDLPINVFKLLAGRWPFDHNDFGVFLCKLFPFLQKSSVGITVLNLC
ALSVDRYRAVASWSRVQGIGIPLVTAIEIVSIWILSFILAIPEAIGFVMVPFEYRGEQH
KTCMLNATSKFMEFYQDVKDWWLFGFYFCMPLVCTAIFYTLMTCEMLNRRNGSLRIALS
EHLKQRREVAKTVFCLVVIFALCWFPLHLSRILKKTVYNEMDKNRCELLSFLLLMDYIG
INLATMNSCINPIALYFVSKKFKNCFQSCLCCCCYQSKSLMTSVPMNGTSIQWKNHDQN
NHNTDRSSHKDSMN.

In some embodiments, the endothelin-1 receptor (ETAR) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 33 and does not comprise the N-terminal methionine (M) residue. In some embodiments, the endothelin-1 receptor (ETAR) comprises the amino acid sequence of SEQ ID NO: 33.

In some embodiments, the systemic sclerosis antigen is major centromere protein A (CENP). In some embodiments, the major centromere protein A (CENP) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 34)
MGPRRRSRKPEAPRRRSPSPTPTPGPSRRGPSLGASSHQHSRRRQGWLK
EIRKLQKSTHLLIRKLPFSRLAREICVKFTRGVDENWQAQALLALQEAA
EAFLVHLFEDAYLLTLHAGRVTLFPKDVQLARRIRGLEEGLG.

In some embodiments, the major centromere protein A (CENP) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 34 and does not comprise the N-terminal methionine (M) residue. In some embodiments, the major centromere protein A (CENP) comprises the amino acid sequence of SEQ ID NO: 34.

In some embodiments, the systemic sclerosis antigen is major centromere protein B (CENP). In some embodiments, the major centromere protein B (CENP) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 35)
MGPKRRQLTFREKSRIIQEVEENPDLRKGEIARRFNIPPSTLSTILKNKRAILASERKY
GVASTCRKINKLSPYDKLEGLLIAWFQQIRAAGLPVKGIILKEKALRIAEELGMDDFTA
SNGWLDRFRRRHGVVSCSGVARARARNAAPRTPAAPASPAAVPSEGSGGSTTGWRAREE
QPPSVAEGYASQDVFSATETSLWYDFLPDQAAGLCGGDGRPRQATQRLSVLLCANADGS
EKLPPLVAGKSAKPRAGQAGLPCDYTANSKGGVTTQALAKYLKALDIRMAAESRRVLLL
AGRLAAQSLDTSGLRHVQLAFFPPGTVHPLERGVVQQVKGHYRQAMLLKAMAALEGQDP
SGLQLGLTEALHFVAAAWQAVEPSDIAACFREAGFGGGPNATITTSLKSEGEEEEEEEE
EEEEEEGEGEEEEEEGEEEEEEGGEGEELGEEEEVEEEGDVDSDEEEEEDEESSSEGLE
AEDWAQGVVEAGGSFGAYGAQEEAQCPTLHFLEGGEDSDSDSEEEDDEEEDDEDEDDDD
DEEDGDEVPVPSFGEAMAYFAMVKRYLTSFPIDDRVQSHILHLEHDLVHVTRKNHARQA
GVRGLGHQS.

In some embodiments, the major centromere protein B (CENP) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 35 and does not comprise the N-terminal methionine (M) residue. In some embodiments, the major centromere protein B (CENP) comprises the amino acid sequence of SEQ ID NO: 35.

In some embodiments, the systemic sclerosis antigen is major centromere protein C (CENP). In some embodiments, the major centromere protein C (CENP) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 36)
MAASGLDHLKNGYRRRFCRPSRARDINTEQGQNVLEILQDCFEEKSLANDFSTNSTKSV
PNSTRKIKDTCIQSPSKECQKSHPKSVPVSSKKKEASLQFVVEPSEATNRSVQAHEVHQ
KILATDVSSKNTPDSKKISSRNINDHHSEADEEFYLSVGSPSVLLDAKTSVSQNVIPSS
AQKRETYTFENSVNMLPSSTEVSVKTKKRLNFDDKVMLKKIEIDNKVSDEEDKTSEGQE
RKPSGSSQNRIRDSEYEIQRQAKKSFSTLFLETVKRKSESSPIVRHAATAPPHSCPPDD
TKLIEDEFIIDESDQSFASRSWITIPRKAGSLKQRTISPAESTALLQGRKSREKHHNIL
PKTLANDKHSHKPHPVETSQPSDKTVLDTSYALIGETVNNYRSTKYEMYSKNAEKPSRS
KRTIKQKQRRKFMAKPAEEQLDVGQSKDENIHTSHITQDEFQRNSDRNMEEHEEMGNDC
VSKKQMPPVGSKKSSTRKDKEESKKKRFSSESKNKLVPEEVTSTVTKSRRISRRPSDWW
VVKSEESPVYSNSSVRNELPMHHNSSRKSTKKTNQSSKNIRKKTIPLKRQKTATKGNQR
VQKFLNAEGSGGIVGHDEISRCSLSEPLESDEADLAKKKNLDCSRSTRSSKNEDNIMTA
QNVPLKPQTSGYTCNIPTESNLDSGEHKTSVLEESGPSRLNNNYLMSGKNDVDDEEVHG
SSDDSKQSKVIPKNRIHHKLVLPSNTPNVRRTKRTRLKPLEYWRGERIDYQGRPSGGFV
ISGVLSPDTISSKRKAKENIGKVNKKSNKKRICLDNDERKINLMVNLGIPLGDPLQPTR
VKDPETREIILMDLVRPQDTYQFFVKHGELKVYKTLDTPFFSTGKLILGPQEEKGKQHV
GQDILVFYVNFGDLLCTLHETPYILSTGDSFYVPSGNYYNIKNLRNEESVLLFTQIKR.

In some embodiments, the major centromere protein C (CENP) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 36 and does not comprise the N-terminal methionine (M) residue. In some embodiments, the major centromere protein C (CENP) comprises the amino acid sequence of SEQ ID NO: 36.

In some embodiments, the systemic sclerosis antigen is tetraspanin-7 (TSPAN7). In some embodiments, the tetraspanin-7 (TSPAN7) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to:

(SEQ ID NO: 37)
MASRRMETKPVITCLKILLIIYSFVFWITGVILLAVGVWGKLTLGTYISLIAENSTNAP
YVLIGTGTTIVVFGLFGCFATCRGSPWMLKLYAMFLSLVFLAELVAGISGFVFRHEIKD
TFLRTYTDAMQTYNGNDERSRAVDHVQRSLSCCGVQNYTNWSTSPYFLEHGIPPSCCMN
ETDCNPQDLHNLTVAATKVNQKGCYDLVTSFMETNMGIIAGVAFGIAFSQLIGMLLACC
LSRFITANQYEMV.

In some embodiments, the tetraspamn-7 (TSPAN7) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 37 and does not comprise the N-terminal methionine (M) residue. In some embodiments, the tetraspanin-7 (TSPAN7) comprises the amino acid sequence of SEQ ID NO: 37.

In some embodiments, the graft versus host disease (GVHD) antigen is selected from the group consisting of: RNA polymerase III complex subunit A (RNAP3), RNA polymerase III complex subunit B (RNAP3), RNA polymerase III complex subunit C (RNAP3), RNA polymerase III complex subunit D (RNAP3), RNA polymerase III complex subunit E (RNAP3), RNA polymerase III complex subunit F (RNAP3), RNA polymerase III complex subunit G (RNAP3), RNA polymerase III complex subunit H (RNAP3), RNA polymerase III complex subunit K (RNAP3), DNA topoisomerase 1 (TOPO1), type-1 angiotensin II receptor (AT1R),endothelin-1 receptor (ETAR), major centromere protein A (CENP), major centromere protein B (CENP), major centromere protein C (CENP), and tetraspanin-7 (TSPAN7).

In some embodiments, the graft versus host disease (GVHD) antigen is RNA polymerase III complex subunit A (RNAP3). In some embodiments, the RNA polymerase III complex subunit A (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 22. In some embodiments, the RNA polymerase III complex subunit A (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 22 and does not comprise the N-terminal methionine (M) residue. In some embodiments, the RNA polymerase III complex subunit A (RNAP3) comprises the amino acid sequence of SEQ ID NO: 22.

In some embodiments, the graft versus host disease (GVHD) antigen is RNA polymerase III complex subunit B (RNAP3). In some embodiments, the RNA polymerase III complex subunit B (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 23. In some embodiments, the RNA polymerase III complex subunit B (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 23 and does not comprise the N-terminal methionine (M) residue. In some embodiments, the RNA polymerase III complex subunit B (RNAP3) comprises the amino acid sequence of SEQ ID NO: 23.

In some embodiments, the graft versus host disease (GVHD) antigen is RNA polymerase III complex subunit C (RNAP3). In some embodiments, the RNA polymerase III complex subunit C (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 24. In some embodiments, the RNA polymerase III complex subunit C (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 24 and does not comprise the N-terminal methionine (M) residue. In some embodiments, the RNA polymerase III complex subunit C (RNAP3) comprises the amino acid sequence of SEQ ID NO: 24.

In some embodiments, the graft versus host disease (GVHD) antigen is RNA polymerase III complex subunit D (RNAP3). In some embodiments, the RNA polymerase III complex subunit D (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 25. In some embodiments, the RNA polymerase III complex subunit D (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 25 and does not comprise the N-terminal methionine (M) residue. In some embodiments, the RNA polymerase III complex subunit D (RNAP3) comprises the amino acid sequence of SEQ ID NO: 25.

In some embodiments, the graft versus host disease (GVHD) antigen is RNA polymerase III complex subunit E (RNAP3). In some embodiments, the RNA polymerase III complex subunit E (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 26. In some embodiments, the RNA polymerase III complex subunit E (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 26 and does not comprise the N-terminal methionine (M) residue. In some embodiments, the RNA polymerase III complex subunit E (RNAP3) comprises the amino acid sequence of SEQ ID NO: 26.

In some embodiments, the graft versus host disease (GVHD) antigen is RNA polymerase III complex subunit F (RNAP3). In some embodiments, the RNA polymerase III complex subunit F (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 27. In some embodiments, the RNA polymerase III complex subunit F (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 27 and does not comprise the N-terminal methionine (M) residue. In some embodiments, the RNA polymerase III complex subunit F (RNAP3) comprises the amino acid sequence of SEQ ID NO: 27.

In some embodiments, the graft versus host disease (GVHD) antigen is RNA polymerase III complex subunit G (RNAP3). In some embodiments, the RNA polymerase III complex subunit G (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 28. In some embodiments, the RNA polymerase III complex subunit G (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 28 and does not comprise the N-terminal methionine (M) residue. In some embodiments, the RNA polymerase III complex subunit G (RNAP3) comprises the amino acid sequence of SEQ ID NO: 28.

In some embodiments, the graft versus host disease (GVHD) antigen is RNA polymerase III complex subunit H (RNAP3). In some embodiments, the RNA polymerase III complex subunit H (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 29. In some embodiments, the RNA polymerase III complex subunit H (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 29 and does not comprise the N-terminal methionine (M) residue. In some embodiments, the RNA polymerase III complex subunit H (RNAP3) comprises the amino acid sequence of SEQ ID NO: 29.

In some embodiments, the graft versus host disease (GVHD) antigen is RNA polymerase III complex subunit K (RNAP3). In some embodiments, the RNA polymerase III complex subunit K (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 30. In some embodiments, the RNA polymerase III complex subunit K (RNAP3) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 30 and does not comprise the N-terminal methionine (M) residue. In some embodiments, the RNA polymerase III complex subunit K (RNAP3) comprises the amino acid sequence of SEQ ID NO: 30.

In some embodiments, the graft versus host disease (GVHD) antigen is DNA topoisomerase 1 (TOPO1). In some embodiments, the DNA topoisomerase 1 (TOPO1) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 31. In some embodiments, the DNA topoisomerase 1 (TOPO1) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 31 and does not comprise the N-terminal methionine (M) residue. In some embodiments, the DNA topoisomerase 1 (TOPO1) comprises the amino acid sequence of SEQ ID NO: 31.

In some embodiments, the graft versus host disease (GVHD) antigen is type-1 angiotensin II receptor (ATIR). In some embodiments, the type-1 angiotensin II receptor (AT1R) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 32. In some embodiments, the type-1 angiotensin II receptor (AT1R) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 32 and does not comprise the N-terminal methionine (M) residue. In some embodiments, the type-1 angiotensin II receptor (AT1R) comprises the amino acid sequence of SEQ ID NO: 32.

In some embodiments, the graft versus host disease (GVHD) antigen is endothelin-1 receptor (ETAR). In some embodiments, the endothelin-1 receptor (ETAR) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 33. In some embodiments, the endothelin-1 receptor (ETAR) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 33 and does not comprise the N-terminal methionine (M) residue. In some embodiments, the endothelin-1 receptor (ETAR) comprises the amino acid sequence of SEQ ID NO: 33.

In some embodiments, the graft versus host disease (GVHD) antigen is major centromere protein A (CENP). In some embodiments, the major centromere protein A (CENP) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 34. In some embodiments, the major centromere protein A (CENP) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 34 and does not comprise the N-terminal methionine (M) residue. In some embodiments, the major centromere protein A (CENP) comprises the amino acid sequence of SEQ ID NO: 34.

In some embodiments, the graft versus host disease (GVHD) antigen is major centromere protein B (CENP). In some embodiments, the major centromere protein B (CENP) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 35. In some embodiments, the major centromere protein B (CENP) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 35 and does not comprise the N-terminal methionine (M) residue. In some embodiments, the major centromere protein B (CENP) comprises the amino acid sequence of SEQ ID NO: 35.

In some embodiments, the graft versus host disease (GVHD) antigen is major centromere protein C (CENP). In some embodiments, the major centromere protein C (CENP) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 36. In some embodiments, the major centromere protein C (CENP) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 36 and does not comprise the N-terminal methionine (M) residue. In some embodiments, the major centromere protein C (CENP) comprises the amino acid sequence of SEQ ID NO: 36.

In some embodiments, the graft versus host disease (GVHD) antigen is tetraspanin-7 (TSPAN7). In some embodiments, the tetraspanin-7 (TSPAN7) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 37. In some embodiments, the tetraspanin-7 (TSPAN7) comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 37 and does not comprise the N-terminal methionine (M) residue. In some embodiments, the tetraspanin-7 (TSPAN7) comprises the amino acid sequence of SEQ ID NO: 37.

In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any one of SEQ ID NO: 14, 15, 16, 17, 18, 19, 20, 21, 22, 23, 24, 25, 26, 27, 28, 29, 30, 31, 32, 33, 34, 35, 36, or 37. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 14. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 15. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 16. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 17. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 18. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 19. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 20. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 21. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 22. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 23. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 24. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 25. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 26. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 27. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 28. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 29. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 30. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 31. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 32. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 33. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 34. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 35. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 36. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 37.

In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any one of SEQ ID NO: 14, 15, 16, 17, 18, 19, 20, 21, 22, 23, 24, 25, 26, 27, 28, 29, 30, 31, 32, 33, 34, 35, 36, or 37 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 14 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 15 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 16 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 17 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 18 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 19 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 20 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 21 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 22 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 23 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 24 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 25 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 26 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 27 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 28 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 29 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 30 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 31 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 32 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 33 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 34 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 35 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 36 and does not comprise the N-terminal methionine (M) residue. In some embodiments, the antigen of interest comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 37 and does not comprise the N-terminal methionine (M) residue.

In some embodiments, the antigen of interest comprises an amino acid sequence of any one of SEQ ID NO: 14, 15, 16, 17, 18, 19, 20, 21, 22, 23, 24, 25, 26, 27, 28, 29, 30, 31, 32, 33, 34, 35, 36, or 37. In some embodiments, the antigen of interest comprises an amino acid sequence of SEQ ID NO: 14. In some embodiments, the antigen of interest comprises an amino acid sequence of SEQ ID NO: 15. In some embodiments, the antigen of interest comprises an amino acid sequence of SEQ ID NO: 16. In some embodiments, the antigen of interest comprises an amino acid sequence of SEQ ID NO: 17. In some embodiments, the antigen of interest comprises an amino acid sequence of SEQ ID NO: 18. In some embodiments, the antigen of interest comprises an amino acid sequence of SEQ ID NO: 19. In some embodiments, the antigen of interest comprises an amino acid sequence of SEQ ID NO: 20. In some embodiments, the antigen of interest comprises an amino acid sequence of SEQ ID NO: 21. In some embodiments, the antigen of interest comprises an amino acid sequence of SEQ ID NO: 22. In some embodiments, the antigen of interest comprises an amino acid sequence of SEQ ID NO: 23. In some embodiments, the antigen of interest comprises an amino acid sequence of SEQ ID NO: 24. In some embodiments, the antigen of interest comprises an amino acid sequence of SEQ ID NO: 25. In some embodiments, the antigen of interest comprises an amino acid sequence of SEQ ID NO: 26. In some embodiments, the antigen of interest comprises an amino acid sequence of SEQ ID NO: 27. In some embodiments, the antigen of interest comprises an amino acid sequence of SEQ ID NO: 28. In some embodiments, the antigen of interest comprises an amino acid sequence of SEQ ID NO: 29. In some embodiments, the antigen of interest comprises an amino acid sequence of SEQ ID NO: 30. In some embodiments, the antigen of interest comprises an amino acid sequence of SEQ ID NO: 31. In some embodiments, the antigen of interest comprises an amino acid sequence of SEQ ID NO: 32. In some embodiments, the antigen of interest comprises an amino acid sequence of SEQ ID NO: 33. In some embodiments, the antigen of interest comprises an amino acid sequence of SEQ ID NO: 34. In some embodiments, the antigen of interest comprises an amino acid sequence of SEQ ID NO: 35. In some embodiments, the antigen of interest comprises an amino acid sequence of SEQ ID NO: 36. In some embodiments, the antigen of interest comprises an amino acid sequence of SEQ ID NO: 37.

In some embodiments, the antigen of interest is not present on an immune cell, such as a lymphocyte. In some embodiments, the antigen of interest is not present on a T_{eff} cell. In some embodiments, the domain that binds to the antigen of interest does not bind to a protein present on an immune cells, such as a lymphocyte. In some embodiments, the domain that binds to the antigen of interest does not bind to a protein present on a T_{eff} cell. In some embodiments, the domain that binds to the antigen of interest does not bind to a receptor present on an immune cell, such as a lymphocyte, including, but not limited to a T_{eff} cell. In some embodiments, the antigen of interest is not an immune cell antigen. In some embodiments, “an immune cell antigen” refers to a protein present on the surface of an immune cell. In some embodiments, the “immune cell antigen” is a cell surface receptor present on the cell surface of the immune cell. In some embodiments, the cell surface receptor is on the surface of a T_{eff} cell. In some embodiments, the antigen of interest is not an immune cell antigen or the domain that binds to the antigen of interest does not bind to the immune cell antigen. In some embodiments, the immune cell antigen is CD47, CD3, CD8, CD4, TIGIT, CD45RA, CD45RO, CXCR3, CCR5, IL12Rβ2, CCR6, NK1.1, CCR10, CD127, CD25, CTLA-4, CXCR5, CD40L, ICOS, TCRγ/δ, IL23R, TCR Vα24, or TCR Vβ11. In some embodiments, the domain that binds to the antigen of interest does not bind to CD47, CD3, CD8, CD4, TIGIT, CD45RA, CD45RO, CXCR3, CCR5, IL12Rβ2, CCR6, NK1.1, CCR10, CD127, CD25, CTLA-4, CXCR5, CD40L, ICOS, TCRγ/δ, IL23R, TCR Vα24, or TCR Vβ11. In some embodiments, the domain that binds to the antigen of interest does not bind to CD3. In some embodiments, the domain that binds to the antigen of interest does not bind to TCRγ/δ. Thus, in some embodiments, the molecules provided herein comprise any one of the cell targeting domains provided herein, and an antigen of interest that is not an immune cell antigen. In some embodiments, the molecules provided herein comprise any one of the cell targeting domains provided herein, and an antigen of interest that is not an immune cell antigen, or the domain that binds to the antigen of interest does not bind to an immune cell antigen, such as, but not limited to, CD47, CD3, CD8, CD4, TIGIT, CD45RA, CD45RO, CXCR3, CCR5, IL12Rβ2, CCR6, NK1.1, CCR10, CD127, CD25, CTLA-4, CXCR5, CD40L, ICOS, TCRγ/δ, IL23R, TCR Vα24, or TCR Vβ11.

In some embodiments, the antigen does not activate a T cell that activates the immune system. In some embodiments, the molecule provided herein does not activate a Teff cell.

In some embodiments, provided is a molecule comprising:
a) a cell targeting domain that binds to cadherin-1 (CDH1), Trop2, CD74, EpCAM, syndecan 1 (SDC1), PlgR, CD23 antigen (FCER2), macrophage receptor MARCO, C-type lectin domain family 7 (CLEC7A), CD24, CD70, sialic acid-binding Ig-like lectin 10 (SIGLEC10), milk fat globule EGF and factor V/VIII domain containing (MFGE8), EGF like repeats and discoidin domains 3 (EDIL3), growth arrest-specific gene 6 (GAS6), T-cell membrane protein 4 (TIM4);
b) an autoimmune, alloimmune, allergic, inflammatory, or anti-drug immune response associated antigen of interest, such as those provided herein; and
c) optionally an Fc region, such as those provided herein,
wherein the cell targeting domain binds to a thymic cell, a splenic cell, a dendritic cell, or a B cell.

In some embodiments, provided is a molecule comprising:
a) a cell targeting domain that binds to cadherin-1 (CDH1);
b) an autoimmune, alloimmune, allergic, inflammatory, or anti-drug immune response associated antigen of interest, such as those provided herein; and
c) optionally an Fc region, such as those provided herein,
wherein the cell targeting domain binds to a thymic cell, a splenic cell, a dendritic cell, or a B cell.

In some embodiments, provided is a molecule comprising:
a) a cell targeting domain that binds to Trop2;
b) an autoimmune, alloimmune, allergic, inflammatory, or anti-drug immune response associated antigen of interest, such as those provided herein; and
c) optionally an Fc region, such as those provided herein,
wherein the cell targeting domain binds to a thymic cell, a splenic cell, a dendritic cell, or a B cell.

In some embodiments, provided is a molecule comprising:
a) a cell targeting domain that binds to CD74;
b) an autoimmune, alloimmune, allergic, inflammatory, or anti-drug immune response associated antigen of interest, such as those provided herein; and
c) optionally an Fc region, such as those provided herein, wherein the cell targeting domain binds to a thymic cell, a splenic cell, a dendritic cell, or a B cell.

In some embodiments, provided is a molecule comprising:
a) a cell targeting domain that binds to EpCAM;
b) an autoimmune, alloimmune, allergic, inflammatory, or anti-drug immune response associated antigen of interest, such as those provided herein; and
c) optionally an Fc region, such as those provided herein,
wherein the cell targeting domain binds to a thymic cell, a splenic cell, a dendritic cell, or a B cell.

In some embodiments, provided is a molecule comprising:
a) a cell targeting domain that binds to syndecan 1 (SDC1);
b) an autoimmune, alloimmune, allergic, inflammatory, or anti-drug immune response associated antigen of interest, such as those provided herein; and
c) optionally an Fc region, such as those provided herein,
wherein the cell targeting domain binds to a thymic cell, a splenic cell, a dendritic cell, or a B cell.

In some embodiments, provided is a molecule comprising:
a) a cell targeting domain that binds to PlgR;
b) an autoimmune, alloimmune, allergic, inflammatory, or anti-drug immune response associated antigen of interest, such as those provided herein; and
c) optionally an Fc region, such as those provided herein,
wherein the cell targeting domain binds to a thymic cell, a splenic cell, a dendritic cell, or a B cell.

In some embodiments, provided is a molecule comprising:
a) a cell targeting domain that binds to CD23 antigen (FCER2);
b) an autoimmune, alloimmune, allergic, inflammatory, or anti-drug immune response associated antigen of interest, such as those provided herein; and
c) optionally an Fc region, such as those provided herein,
wherein the cell targeting domain binds to a thymic cell, a splenic cell, a dendritic cell, or a B cell.

In some embodiments, provided is a molecule comprising:
a) a cell targeting domain that binds to macrophage receptor MARCO;
b) an autoimmune, alloimmune, allergic, inflammatory, or anti-drug immune response associated antigen of interest, such as those provided herein; and
c) optionally an Fc region, such as those provided herein,
wherein the cell targeting domain binds to a thymic cell, a splenic cell, a dendritic cell, or a B cell.

In some embodiments, provided is a molecule comprising:
a) a cell targeting domain that binds to C-type lectin domain family 7 (CLEC7A);
b) an autoimmune, alloimmune, allergic, inflammatory, or anti-drug immune response associated antigen of interest, such as those provided herein; and
c) optionally an Fc region, such as those provided herein,
wherein the cell targeting domain binds to a thymic cell, a splenic cell, a dendritic cell, or a B cell.

In some embodiments, provided is a molecule comprising:
a) a cell targeting domain that binds to CD63;
b) an autoimmune, alloimmune, allergic, inflammatory, or anti-drug immune response associated antigen of interest, such as those provided herein; and
c) optionally an Fc region, such as those provided herein,
wherein the cell targeting domain binds to a thymic cell, a splenic cell, a dendritic cell, or a B cell.

In some embodiments, provided is a molecule comprising:
a) a cell targeting domain that binds to CD24;
b) an autoimmune, alloimmune, allergic, inflammatory, or anti-drug immune response associated antigen of interest, such as those provided herein; and
c) optionally an Fc region, such as those provided herein,
wherein the cell targeting domain binds to a thymic cell, a splenic cell, a dendritic cell, or a B cell.

In some embodiments, provided is a molecule comprising:
a) a cell targeting domain that binds to CD70;
b) an autoimmune, alloimmune, allergic, inflammatory, or anti-drug immune response associated antigen of interest, such as those provided herein; and
c) optionally an Fc region, such as those provided herein,
wherein the cell targeting domain binds to a thymic cell, a splenic cell, a dendritic cell, or a B cell.

In some embodiments, provided is a molecule comprising:
a) a cell targeting domain sialic acid-binding Ig-like lectin 10 (SIGLEC10);
b) an autoimmune, alloimmune, allergic, inflammatory, or anti-drug immune response associated antigen of interest, such as those provided herein; and
c) optionally an Fc region, such as those provided herein,
wherein the cell targeting domain binds to a thymic cell, a splenic cell, a dendritic cell, or a B cell.

In some embodiments, provided is a molecule comprising:
a) a cell targeting domain milk fat globule EGF and factor V/VIII domain containing (MFGE8);
b) an autoimmune, alloimmune, allergic, inflammatory, or anti-drug immune response associated antigen of interest, such as those provided herein; and
c) optionally an Fc region, such as those provided herein,
wherein the cell targeting domain binds to a thymic cell, a splenic cell, a dendritic cell, or a B cell.

In some embodiments, provided is a molecule comprising:
a) a cell targeting domain growth arrest-specific gene 6 (GAS6);
b) an autoimmune, alloimmune, allergic, inflammatory, or anti-drug immune response associated antigen of interest, such as those provided herein; and
c) optionally an Fc region, such as those provided herein,
wherein the cell targeting domain binds to a thymic cell, a splenic cell, a dendritic cell, or a B cell.

In some embodiments, provided is a molecule comprising:
a) a cell targeting domain that binds to T-cell membrane protein 4 (TIM4);
b) an autoimmune, alloimmune, allergic, inflammatory, or anti-drug immune response associated antigen of interest, such as those provided herein; and
c) optionally an Fc region, such as those provided herein,
wherein the cell targeting domain binds to a thymic cell, a splenic cell, a dendritic cell, or a B cell.

Also provided are methods of using and making compounds.

In some embodiments, a polypeptide is provided that comprises a) a cell targeting domain; b) an Fc domain; and c) an antigen of interest. In some embodiments, a polypeptide is provided that comprises a) a cell targeting domain and b) an antigen of interest. In some embodiments, a polypeptide is provided that comprises a) a cell targeting domain and b) an Fc domain. In some embodiments, a polypeptide is provided that comprises a) an antigen of interest and b) an Fc domain. In some embodiments, a polypeptide is provided that comprises a cell targeting domain and an antigen of interest, i.e., without an Fc domain. In some embodiments, a polypeptide is provided that comprises a plurality of cell targeting domains and an Fc domain linked to each cell targeting domains. The Fe domains linked to each cell targeting domains can be the same or different. In some embodiments, a polypeptide is provided that comprises 1, or 2 cell targeting domains, each linked to a Fc domain. The Fc domains linked to each cell targeting domain can be the same or different. In some embodiments, the cell targeting domains are linked to the Fc domain to the N-terminus and/or the C-terminus of the Fc domain. In some embodiments, the Fc domain has a cell targeting domains linked to the N-terminus and the C-terminus of the Fc domain. In some embodiments, each cell targeting domain binds to the same thymic cell receptor. In some embodiments, each cell targeting domain binds to different thymic cell receptors.

In some embodiments, the polypeptide comprises from the N-terminus to the C-terminus: a) a cell targeting domain; b) an Fc domain; and c) an antigen of interest. In some embodiments, the polypeptide comprises from the N-terminus to the C-terminus a) an antigen of interest b) an Fc domain; and c) a cell targeting domain.

In some embodiments, the polypeptide comprises from the N-terminus to the C-terminus: a cell targeting domain and an antigen of interest. In some embodiments, the polypeptide comprises from the N-terminus to the C-terminus: an antigen of interest and a cell targeting domain.

In some embodiments, the polypeptide comprises from the N-terminus to the C-terminus: a cell targeting domain and a Fc domain. In some embodiments, the polypeptide comprises from the N-terminus to the C-terminus: an antigen of interest and an Fc domain.

In each of the embodiments, provided for herein, the domains can be linked to one another with a peptide linker, such as the non-limiting examples provided for herein, or without an intervening peptide linker.

In some embodiments, the polypeptide comprises a plurality of cell targeting domains that can bind to either the same thymic cell receptor or to two different thymic cell receptors. As provided for herein, in some embodiments, the polypeptide comprises two cell targeting domains that bind to the same or different thymic cell receptors.

In some embodiments, the Fc domain is linked to the cell targeting domain. In some embodiments, the Fc domain is linked to a C-terminus of the cell targeting domain. In some embodiments, when the cell targeting domain is an antibody, or the antigen-binding fragment thereof, the Fc domain is linked to the C-terminus of the heavy chain of the antibody, or the antigen-binding fragment thereof, that forms the cell targeting domain. In some embodiments, the N-terminus of the Fc domain is linked to the C-terminus of the cell targeting domain. In some embodiments, the Fc domain is directly linked, such as without a linker sequence, to the cell targeting domain. In some embodiments, the Fc domain is linked to the cell targeting domain through a linker, such as a peptide linker. In some embodiments, the linker is as provided for herein.

In some embodiments, the Fc domain is linked to the antigen of interest. In some embodiments, the Fc domain is linked to a C-terminus of the antigen of interest. In some embodiments, when the antigen of interest is an antibody, or the antigen-binding fragment thereof, a payload, or an antigen, and the Fc domain is linked to the C-terminus of the heavy chain of the antibody, or the antigen-binding fragment thereof, the payload, or the antigen that forms the antigen of interest. In some embodiments, the N-terminus of the Fc domain is linked to the C-terminus of the antigen of interest. In some embodiments, the Fc domain is directly linked, such as without a linker sequence, to the antigen of interest. In some embodiments, the Fc domain is linked to the antigen of interest through a linker, such as a peptide linker. In some embodiments, the linker is as provided for herein.

In some embodiments, the Fc domain is linked to the cell targeting domain and the antigen of interest. In some embodiments, the Fc domain is linked to a C-terminus of the cell targeting domain and the N-terminus of the antigen of interest. In some embodiments, when the cell targeting domain is an antibody, or the antigen-binding fragment thereof, the Fc domain is linked to the C-terminus of the heavy chain of the antibody, or the antigen-binding fragment thereof, that forms the cell targeting domain, and when the antigen of interest is an antibody, or the antigen-binding fragment thereof, a payload, or an antigen, the Fc domain is linked to the N-terminus of the heavy chain of the antibody, or the antigen-binding fragment thereof, the payload, or the antigen. In some embodiments, the N-terminus of the Fc domain is linked to the C-terminus of the cell targeting domain, and the C-terminus of the Fc domain is linked to the N-terminus of the antigen of interest. In some embodiments, the Fc domain is linked to the cell targeting domain and the antigen of interest. In some embodiments, the Fc domain is linked to a N-terminus of the cell targeting domain and the C-terminus of the antigen of interest. In some embodiments, when the cell targeting domain is an antibody, or the antigen-binding fragment thereof, the Fc domain is linked to the N-terminus of the heavy chain of the antibody, or the antigen-binding fragment thereof, that forms the cell targeting domain, and when the antigen of interest is an antibody, or the antigen-binding fragment thereof, a payload, or an antigen, the Fc domain is linked to the C-terminus of the heavy chain of the antibody, or the antigen-binding fragment thereof, the payload, or the antigen. In some embodiments, the C-terminus of the Fc domain is linked to the N-terminus of the cell targeting domain, and the N-terminus of the Fc domain is linked to the C-terminus of the antigen of interest.

In some embodiments, the Fc domain is directly linked, such as without a linker sequence, to the cell targeting domain and the antigen of interest. In some embodiments, the Fc domain is linked to the cell targeting domain and the antigen of interest through a linker, such as a peptide linker. In some embodiments, the linker is as provided for herein.

In some embodiments, the Fc domains can form a dimer. In some embodiments, dimerization interface centers around the human IgG1 CH2-CH3 domains, which dimerize via a contact interface spanning both CH2/CH2 and CH3/CH3. However, in order to achieve heterodimerization instead of homodimerization of each heavy chain, mutations are introduced in each CH3 domain. The heterodimerizing mutations include T366W mutation (Kabat) in one CH3 domain and T366S, L368A, and Y407V (Kabat) mutations in the other CH3 domain. The heterodimerizing interface may be further stabilized with de novo disulphide bonds via mutation of native residues to cysteine residues such as S354 and Y349 on opposite sides of the CH3/CH3 interface. The resulting bispecific antibodies shown have a total valence comprised of four binding units. With this approach, the overall molecule can be designed to have bispecificity at just one terminus and monospecificity at the other terminus (trispecificity overall) or bispecificity at either terminus with an overall molecular specificity of 2 or 4.

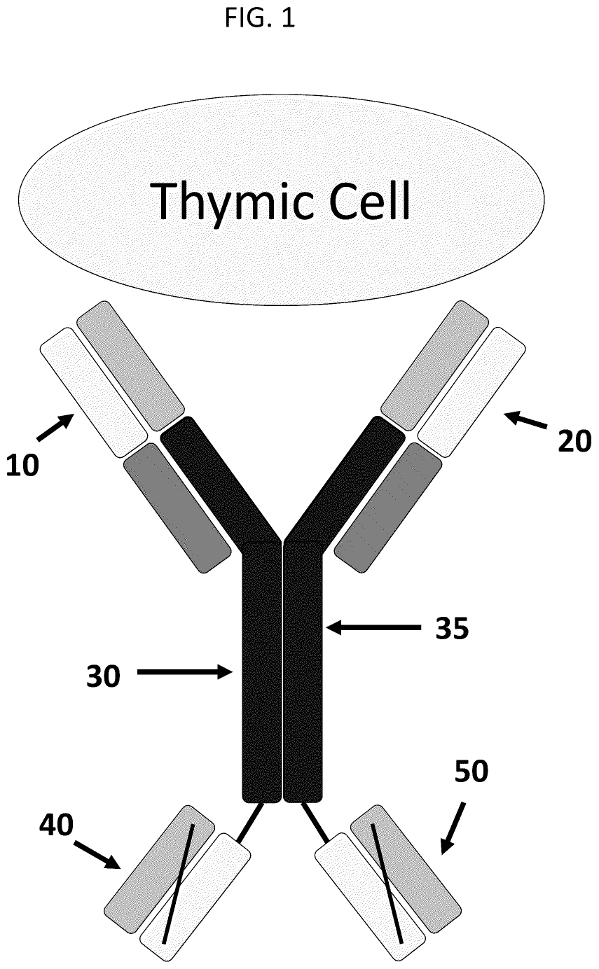

Another non-limiting example of a compound as provided for herein is illustrated in FIG. 1. Referencing FIG. 1, (10) illustrates a cell targeting domain as an antibody, or the antigen-binding fragment thereof, (e.g. Fab) that binds to a thymic cell receptor, such as those provided herein. (20) illustrates another cell targeting domain as an antibody, or the antigen-binding fragment thereof, that binds to a thymic cell receptor. The cell targeting domains of (10) and (20) can bind to the same thymic cell receptor or different thymic cell receptors.

Referencing FIG. 1, (30) and (35) illustrate Fc domains, such as those provided herein. Referencing FIG. 1, (40) and (50) illustrate antigens of interest, which are shown as a scFv antibody, or the antigen-binding fragment thereof. In some embodiments, (40) and (50) are the same, but they can have different structures, i.e. sequences. Additionally, (40) and (50) are illustrated as being scFv antibodies. However, (10), (20), (40) and (50) can be scFv, Fab, Fab′, F(ab′)2, and VHH antibodies. Furthermore, (40) and (50) can also be polypeptides that are not antibodies. In other non-limiting example of a compound as provided for herein, referencing FIG. 1, (40) can comprise a cell targeting domain as an antibody, or the antigen-binding fragment thereof, (e.g. Fab) that binds to a thymic cell receptor, such as those provided herein. (50) can comprise another cell targeting domain as an antibody, or the antigen-binding fragment thereof, that binds to a thymic cell receptor. The cell targeting domains of (40) and (50) can bind to the same thymic cell receptor or different thymic cell receptors. Referencing FIG. 1, (10) and (20) can comprise antigens of interest, which are shown as a scFv antibody, or the antigen-binding fragment thereof. In some embodiments, (10) and (20) are the same, but they can have different structures, i.e. sequences. However, (10), (20), (40) and (50) can be scFv, Fab, Fab′, F(ab′)2, and VHH antibodies.

Examples of such polypeptides include allergens and pollens, such as those provided herein.

In some embodiments, the Fc domain comprises mutations that render the Fc region “effectorless” and unable to bind Fc receptors. The mutations that render Fc regions effectorless are known in the art and any mutation or combination of mutations can be used. In some embodiments, the Fc domain comprises mutations that impair or inhibits recycling of the Fc. In some embodiments, the Fc domain comprises mutations that impair FcRn-mediated recycling of the Fc. In some embodiments, the Fc domain comprises a mutation, or a set of mutations, that impair binding of the Fc to an FcRn. In some embodiments, the Fc domain does not bind to an FcRn. In some embodiments, the Fc domain has reduced affinity to an FcRn. In some embodiments, the Fc domain comprising a mutation, or a set of mutations, that impairs binding of the Fc to an FcRn, exhibit better (i.e., more or enhanced) degradation as compared to an Fc domain not comprising the mutation, or set of mutations, that impairs its binding to the FcRn. In some embodiments, the Fc domain comprising a mutation, or a set of mutations, that impairs binding of the Fc to an FcRn, exhibit better lysosomal degradation as compared to an Fc domain not comprising the mutation, or set of mutations, that impairs its binding to the FcRn. In some embodiments, the Fc domain comprising a mutation, or a set of mutations, that impairs binding of the Fc to an FcRn, exhibit improved lysosomal degradation as compared to an Fc domain not comprising the mutation, or set of mutations, that impairs its binding to the FcRn. Without being bound to any particular theory, a Fc molecule that has improved lysosomal degradation will lead to the antigen being released from the Fc molecule through the lysosomal degradation pathway, and, therefore, improved antigen presentation on the surface of a cell, such as those cells provided herein. Therefore, in some embodiments, methods of providing antigen presentation on the surface of a cell are provided, wherein the antigen is linked or fused to a Fc that comprises a mutation that impairs the Fc's binding to the FcRn. In some embodiments, the Fc domain comprises a mutation, or a set of mutations, selected from H310A, and/or G435A, wherein the mutation, or set of mutations impairs binding of the Fc to the FcRn. In some embodiments, the Fc region is an effectorless Fc region, and the Fc region does not bind, or has reduced affinity, to FcRn.

In some embodiments, the mutations in the Fc region, which is according to the known numbering system, are selected from the group consisting of: L234A, L235A, L234F, L235E, P329G, P331S, N297A, N297G, N297Q, G236A, A330S, S239D, I332E, S267E, H268F, S324T, Y296W, T299A, V308P, H310A, R409K, Y435H, T307A, T309A, T309K, K322A, K326W, K334W, K326A, K334A, G237A, P238S, H268A, or any combination thereof. In some embodiments, the Fc comprises a mutation at L234 and/or L235 and/or G237. In some embodiments, the Fc comprises L234A and/or L235A mutations, which can be referred to as “LALA” mutations. In some embodiments, the Fc comprises L234A, L235A, and G237A mutations, which can be referred to as “LALAGA” or “AAA”. In some embodiments, the Fc comprises L234F, and L235E mutations, which can be referred to as “LAFE” mutations. In some embodiments, the Fc comprises L234A, L235A, and P329G mutations, which can be referred to as “LALAPG” mutations. In some embodiments, the Fc comprises L234A, L235A, P329G, and P331S mutations, which can be referred to as “LALAPGS” mutations. In some embodiments, the Fc comprises L234A, L235A, and P329S mutations, which can be referred to as “LALAPS” mutations. In some embodiments, the Fc comprises a N297A mutation. In some embodiments, the Fc mutations comprises a N297G mutation. In some embodiments, the Fc comprises a N297Q mutation. In some embodiments, the Fc comprises a P329G mutation. In some embodiments, the Fc comprises G236A, A330S, and P331S mutations, which can be referred to as “GASDALIE” mutations. In some embodiments, the Fc comprises S239D and I332E mutations, which can be referred to as “SIE” mutations. In some embodiments, the Fc comprises S267E, H268F, S324T, and 1332E mutations, which can be referred to as “SEHF_STIE” mutations. In some embodiments, the Fc comprises Y296W, T299A, and V308P mutations, which can be referred to as “YTEV” mutations. In some embodiments, the Fc comprises H310A, R409K, and Y435H mutations, which can be referred to as “HRY” mutations. In some embodiments, the Fc comprises T307A and T309A mutations, which can be referred to as “TATA” mutations. In some embodiments, the Fc comprises T307A and T309K mutations, which can be referred to as “TAKA” mutations. In some embodiments, the Fc comprises a K322A mutation. In some embodiments, the Fc comprises K326W and K334W mutations, which can be referred to as “WKWK” mutations. In some embodiments, the Fc comprises K326A and K334A mutations, which can be referred to as “AA” mutations. In some embodiments, the Fc comprises L234A, L235A, G237A, P238S, H268A, A330S, and P331S mutations.

In some embodiments, the Fc comprises a H310A mutation. In some embodiments, the Fc comprises a H435A mutation. In some embodiments, the Fc comprises H310A and H435A mutations. In some embodiments, the Fc mutations comprise L234A, L235A, and H310A. In some embodiments, the Fc mutations comprise L234F, L235E, and H310A. In some embodiments, the Fc mutations comprise L234A, L235A, P329G, and H310A. In some embodiments, the Fc mutations comprise L234A, L235A, P329G, P331S, and H310A. In some embodiments, the Fc mutations comprise L234A, L235A, P329S, and H310A. In some embodiments, the Fc mutations comprise N297A, and H310A. In some embodiments, the Fc mutations comprise N297G, and H310A. In some embodiments, the Fc mutations comprise N297Q, and H310A. In some embodiments, the Fc mutations comprise P329G, and H310A. In some embodiments, the Fc mutations comprise G236A, A330S, P331S, and H310A. In some embodiments, the Fc mutations comprise S239D, I332E, and H310A. In some embodiments, the Fc mutations comprise S267E, H268F, S324T, I332E, and H310A. In some embodiments, the Fc mutations comprise Y296W, T299A, V308P, and H310A. In some embodiments, the Fc mutations comprise H310A, R409K, Y435H, and H310A. In some embodiments, the Fc mutations comprise T307A, T309A, and H310A. In some embodiments, the Fc mutations comprise T307A, T309K, and H310A. In some embodiments, the Fc mutations comprise K322A, and H310A. In some embodiments, the Fc mutations comprise K326W, K334W, and H310A. In some embodiments, the Fc mutations comprise K326A, K334A, and H310A. In some embodiments, the Fc mutations comprise L234A, L235A, and H435A. In some embodiments, the Fc mutations comprise L234F, L235E, and H435A. In some embodiments, the Fc mutations comprise L234A, L235A, P329G, and H435A. In some embodiments, the Fc mutations comprise L234A, L235A, P329G, P331S, and H435A. In some embodiments, the Fc mutations comprise L234A, L235A, P329S, and H435A. In some embodiments, the Fc mutations comprise N297A, and H435A. In some embodiments, the Fc mutations comprise N297G, and H435A. In some embodiments, the Fc mutations comprise N297Q, and H435A. In some embodiments, the Fc mutations comprise P329G, and H435A. In some embodiments, the Fc mutations comprise G236A, A330S, P331S, and H435A. In some embodiments, the Fc mutations comprise S239D, I332E, and H435A. In some embodiments, the Fc mutations comprise S267E, H268F, S324T, I332E, and H435A. In some embodiments, the Fc mutations comprise Y296W, T299A, V308P, and H435A. In some embodiments, the Fc mutations comprise H435A, R409K, Y435H, and H435A. In some embodiments, the Fc mutations comprise T307A, T309A, and H435A. In some embodiments, the Fc mutations comprise T307A, T309K, and H435A. In some embodiments, the Fc mutations comprise K322A, and H435A. In some embodiments, the Fc mutations comprise K326W, K334W, and H435A. In some embodiments, the Fc mutations comprise K326A, K334A, and H435A. In some embodiments, the Fc mutations comprise L234A, L235A, H310, and H435A. In some embodiments, the Fc mutations comprise L234F, L235E, H310, and H435A. In some embodiments, the Fc mutations comprise L234A, L235A, P329G, H310, and H435A. In some embodiments, the Fc mutations comprise L234A, L235A, P329G, P331S, H310, and H435A. In some embodiments, the Fc mutations comprise L234A, L235A, P329S, H310, and H435A. In some embodiments, the Fc mutations comprise N297A, H310, and H435A. In some embodiments, the Fc mutations comprise N297G, H310, and H435A. In some embodiments, the Fc mutations comprise N297Q, H310, and H435A. In some embodiments, the Fc mutations comprise P329G, H310, and H435A. In some embodiments, the Fc mutations comprise G236A, A330S, P331S, H310, and H435A. In some embodiments, the Fc mutations comprise S239D, I332E, H310, and H435A. In some embodiments, the Fc mutations comprise S267E, H268F, S324T, I332E, H310, and H435A. In some embodiments, the Fc mutations comprise Y296W, T299A, V308P, H310, and H435A. In some embodiments, the Fc mutations comprise H435A, R409K, Y435H, H310, and H435A. In some embodiments, the Fc mutations comprise T307A, T309A, H310, and H435A. In some embodiments, the Fc mutations comprise T307A, T309K, H310, and H435A. In some embodiments, the Fc mutations comprise K322A, H310, and H435A. In some embodiments, the Fc mutations comprise K326W, K334W, H310, and H435A. In some embodiments, the Fc mutations comprise K326A, K334A, H310, and H435A.

The mutations and positions of the Fc region, which can also be referred to as the Fc domain, are according to Kabat numbering.

As used herein, a Fc region/domain comprising a mutation at a specific position is as compared to the wild-type Fe according to the numbering system (Kabat numbering) as referenced herein.

Examples of peptide linkers that can be used are known in the art and non-limiting examples are provide for herein.

In some embodiments, the polypeptide does not comprise a Fc domain. Thus, in some embodiments, a polypeptide or a molecule is provided that comprises one or more cell targeting domains linked to an antigen of interest. In some embodiments, the C-terminus of the cell targeting domain is linked to the N-terminus of antigen of interest. In some embodiments, the N-terminus of the cell targeting domain is linked to the C-terminus of the antigen of interest. In some embodiments, the different domains are linked together through a peptide linker. Non-limiting examples of such linkers are provided for herein.

As provided for herein the domains can be linked to together with a linker domain or region. Any linker region described herein can be used as a linker. Linkers can be for example, glycine/serine linkers. In some embodiments, the linker can comprise one or more repeats of GGGGS (SEQ ID NO: 1). In some embodiments, the linker comprises 1, 2, 3, 4, or 5 repeats. In some embodiments, the linker comprises GGGGSGGGGS (SEQ ID NO: 2). In some embodiments, the linker comprises GGGGSGGGGSGGGGS (SEQ ID NO: 3). In some embodiments, the linker comprises: GGGGS (SEQ ID NO: 1), (GGGGS)₃ (SEQ ID NO: 3), (GGGGS)ₙ(n=1, 2, 3, 4) (SEQ ID NO: 1-4), (Gly)₈ (SEQ ID NO: 5), (Gly)₆ (SEQ ID NO: 6), (EAAAK)₃ (SEQ ID NO: 7), (EAAK)ₙ (n=1-3) (SEQ ID NO: 8-10), A(EAAAK)₄ALEA(EAAAK)₄A (SEQ ID NO: 11), or AEAAAKEAAAKA (SEQ ID NO: 12). These linkers can be used in any of the compounds or compositions provided herein. These peptide linkers are non-limiting examples and other peptide linkers can also be used.

In some embodiments, two (or more) linkers associate, either covalently or non-covalently, e.g., to form a hetero or homo-dimeric therapeutic compound. In some embodiments, the linker can comprise an Fc region and two Fc regions associate with one another. In some embodiments of a therapeutic compound comprising two linker regions, the linker regions can self-associate, e.g., as two identical Fc regions. In some embodiments of a therapeutic compound comprising two linker regions, the linker regions are not capable of, or not capable of substantial, self-association, e.g., the two Fc regions can be members of a knob and hole pair. In some embodiments, the polypeptide comprises a first polypeptide and a second polypeptide. In some embodiments, the first polypeptide comprises a knob mutation, and the second polypeptide comprises a hole mutation. In some embodiments, the first polypeptide comprises a hole mutation, and the second polypeptide comprises a knob mutation. In some embodiments, the knob mutation is such as those provided herein.

In some embodiments, a polypeptide can associate with another polypeptide. In some embodiments, the polypeptide associated with another polypeptide forms a dimer molecule.

In some embodiments, the dimer is a homodimer molecule. In some embodiments, the dimer is a heterodimer molecule.

As used herein, the term “non-covalently conjugated” can mean that a polypeptide is tethered to another polypeptide through a linker. In some embodiments, the linker is a peptide linker. Non-limiting examples of peptide linkers that can be used are known in the art and are provided for herein.

As discussed herein the different domains, molecules, or polypeptide can be linked together with a linker domain or region. Any linker region described herein can be used as a linker. Linkers can be for example, glycine/serine linkers. In some embodiments, the linker can comprise one or more repeats of GGGGS (SEQ ID NO: 1). In some embodiments, the linker comprises 1, 2, 3, 4, or 5 repeats. In some embodiments, the linker comprises GGGGSGGGGS (SEQ ID NO: 2). In some embodiments, the linker comprises GGGGSGGGGSGGGGS (SEQ ID NO: 3). In some embodiments, the linker comprises: GGGGS (SEQ ID NO: 1), (GGGGS)₃ (SEQ ID NO: 3), (GGGGS)ₙ (n=1, 2, 3, 4) (SEQ ID NO: 1-4), (Gly)₈ (SEQ ID NO: 5), (Gly)₆ (SEQ ID NO: 6), (EAAAK)₃ (SEQ ID NO: 7), (EAAK)ₙ (n=1-3) (SEQ ID NO: 8-10), A(EAAAK)₄ALEA(EAAAK)₄A (SEQ ID NO: 11), or AEAAAKEAAAKA (SEQ ID NO: 12). These linkers can be used in any of the compounds or compositions provided herein.

In some embodiments, the cell targeting domain is conjugated to the N-terminus of Fc region, and the antigen of interest, or a domain that binds to the antigen of interest, is conjugated to a C-terminus of the Fc polypeptide domain. In some embodiments, the cell targeting domain is conjugated to the C-terminus of Fc region, and the antigen of interest, or a domain that binds to the antigen of interest, is conjugated to the N-terminus of the Fc polypeptide domain. In some embodiments, the cell targeting domain and the antigen of interest, or the domain that binds to the antigen of interest, are directly conjugated.

In some embodiments, the molecule is a dimer molecule. In some embodiments, the dimer molecule comprises a first molecule and a second molecule. In some embodiments, the first molecule is any molecule provided herein. In some embodiments, the second molecule is any molecule provided herein.

Non-limiting exemplary configurations of therapeutic compounds comprise the following (e.g., in N to C terminal order):
R-Linker_1-R2
R3-Linker_2-R4,
   wherein,
R1, and R3, each independently comprises a cell targeting domain; and
R2, and R4, each independently comprises an antigen of interest, e.g., an antigen, a payload, an allergen, a pollen, or a fragment thereof; or is absent, provided that either R2 or R4 are present.

Linker_1 and Linker_2 comprise moieties that can associate with one another, e.g., Linker_1 and Linker_2, each comprises an Fc moiety provided that an antigen of interest and a cell targeting moiety are present.

Another non-limiting exemplary configuration of therapeutic compounds comprise the following (e.g., in N to C terminal order):
R2-Linker_1-R1
R4-Linker_2-R3,
   wherein,
R1, and R3, each independently comprises a cell targeting domain; and
R2, and R4, each independently comprises an antigen of interest, e.g., an antigen, a payload, an allergen, a pollen, or a fragment thereof, such as those provided herein; or is absent, provided that either R2 or R4 are present

Linker_1 and Linker_2 comprise moieties that can associate with one another, e.g., Linker_1 and Linker_2, each comprises an Fc moiety provided that an antigen of interest and a cell targeting moiety are present.

In some embodiments, molecules are providing having a formula from N-terminus to C-terminus,
R1-Linker_1-R2; and
R3-Linker_2-R4, wherein R1, R3, R3, or R4 each independently comprise a cell targeting domain that binds to a thymic cell, or is absent; R1, R2, R3, or R4 each independently comprise an antigen of interest, or is absent, provided that R1, R2, R3, or R4 each independently comprise a cell targeting domain that binds to a thymic cell, an antigen of interest, or is absent; and Linker_1 and Linker_2 each independently comprise an Fc region, a protein linker, a chemical linker, or is absent.

Another non-limiting exemplary configuration of therapeutic compounds comprises the following (e.g., in N to C terminal order):
R1-R2
R3-R4,
   wherein,
R1, and R3, each independently comprises a cell targeting domain; and
R2, and R4, each independently comprises an antigen of interest, e.g., an antigen, a payload, an allergen, a pollen, or a fragment thereof; or is absent, provided that either R2 or R4 are present.

Another non-limiting exemplary configuration of therapeutic compounds comprises the following (e.g., in N to C terminal order):
R2-R1
R4-R3,
   wherein,
R1, and R3, each independently comprises a cell targeting domain; and
R2, and R4, each independently comprises an antigen of interest, e.g., an antigen, a payload, an allergen, a pollen, or a fragment thereof, such as those provided herein; or is absent, provided that either R2 or R4 are present.

In some embodiments:
R1 comprises an antigen of interest, e.g., an antigen, a payload, an allergen, a pollen, or a fragment thereof, such as those provided herein, or is absent;
R2 comprises a cell targeting domain that binds to a receptor on the surface of a thymic cell;
R3 comprises an antigen of interest, e.g., an antigen, a payload, an allergen, a pollen, or a fragment thereof, such as those provided herein;
R4 comprises a cell targeting domain that binds to a receptor on the surface of a thymic cell; and
Linker_1 and Linker_2 comprise moieties that can associate with one another, e.g., Linker_1 and Linker_2, each comprises an Fc moiety provided that an antigen of interest and a cell targeting moiety are present.

In some embodiments:
R1 is absent;
R2 comprises a cell targeting domain that binds to a receptor on the surface of a thymic cell;
R3 comprises an antigen of interest, e.g., an antigen, a payload, an allergen, a pollen, or a fragment thereof, such as those provided herein;
R4 comprises a cell targeting domain that binds to a receptor on the surface of a thymic cell; and
Linker_1 and Linker_2 comprise moieties that can associate with one another, e.g., Linker_1 and Linker_2, each comprises an Fc moiety provided that an antigen of interest and a cell targeting moiety are present.

In some embodiments:
R1 comprises an antigen of interest, e.g., an antigen, a payload, an allergen, a pollen, or a fragment thereof, such as those provided herein;
R2 comprises a cell targeting domain that binds to a receptor on the surface of a thymic cell;
R3 is absent;
R4 comprises a cell targeting domain that binds to a receptor on the surface of a thymic cell; and
Linker_1 and Linker_2 comprise moieties that can associate with one another, e.g., Linker_1 and Linker_2, each comprises an Fc moiety provided that an antigen of interest and a cell targeting moiety are present.

In some embodiments:
R1 is absent;
R2 comprises a cell targeting domain that binds to a receptor on the surface of a thymic cell;
R3 comprises an antigen of interest, e.g., an antigen, a payload, an allergen, a pollen, or a fragment thereof, such as those provided herein; and
R4 comprises a cell targeting domain that binds to a receptor on the surface of a thymic cell.

In some embodiments:
R1 comprises an antigen of interest, e.g., an antigen, a payload, an allergen, a pollen, or a fragment thereof, such as those provided herein;
R2 comprises a cell targeting domain that binds to a receptor on the surface of a thymic cell;
R3 is absent; and
R4 comprises a cell targeting domain that binds to a receptor on the surface of a thymic cell.

In some embodiments, the polypeptides or molecules disclosed herein are comprised of four polypeptide chains comprising the following:

nt-VH1-CH1-CH2-CH3-Linker A-scFv[VL2-Linker B-VH2]-ct Chain 1:

nt-VH1-CH1-CH2-CH3-Linker A-scFv[VL2-Linker B-VH2]-ct Chain 2:

nt-VL1-CL-ct Chain 3:

nt-VL1-CL-ct Chain 4:

In some embodiments, the polypeptides or molecules disclosed are comprised of four polypeptide chains comprising the following:

nt-VH1-CH1-CH2-CH3-Linker A-scFv[VH2-Linker B-VL2]-ct Chain 1:

nt-VH1-CH1-CH2-CH3-Linker A-scFv[VH2-Linker B-VL2]-et Chain 2:

nt-VL1-CL-ct Chain 3:

nt-VL1-CL-ct. Chain 4:

In some embodiments, the polypeptides or molecules disclosed are comprised of four polypeptide chains that do not contain the Fc region, which can be illustrated as having the following formula:

nt-VH1-CH1-Linker A-scFv[VH2-Linker B-VL2]-ct Chain 1:

nt-VH1-CH1-Linker A-scFv[VH2-Linker B-VL2]-ct Chain 2:

nt-VL1-CL-ct Chain 3:

nt-VL1-CL-ct. Chain 4:

In some embodiments, the polypeptides or molecules disclosed are comprised of four polypeptide chains that do not contain the Fc region, which can be illustrated as having the following formula:

nt-VH1-Linker A-scFv[VH2-Linker B-VL2]-ct Chain 1:

nt-VH1-Linker A-scFv[VH2-Linker B-VL2]-ct Chain 2:

nt-VL1-CL-ct Chain 3:

nt-VL1-CL-ct. Chain 4:

In some embodiments, the polypeptides or molecules disclosed are comprised of four polypeptide chains that do not contain the Fc region, which can be illustrated as having the following formula:

nt-VH1-CH1-Linker A-scFv[VL2-Linker B-VH2]-ct Chain 1:

nt-VH1-CH1-Linker A-scFv[VL2-Linker B-VH2]-ct Chain 2:

nt-VL1-CL-ct Chain 3:

nt-VL1-CL-ct. Chain 4:

In some embodiments, the polypeptides or molecules disclosed are comprised of four polypeptide chains that do not contain the Fc region, which can be illustrated as having the following formula:

nt-VH1-Linker A-scFv[VL2-Linker B-VH2]-ct Chain 1:

nt-VH1-Linker A-scFv[VL2-Linker B-VH2]-ct Chain 2:

nt-VL1-CL-ct Chain 3:

nt-VL1-CL-ct. Chain 4:

In some embodiments, chains 1 and 2 are identical to each other, and chains 3 and 4 are identical to each other. In some embodiments, chains 3 and 4 are identical and chains 1 and 2 are different from one another or are different from one another at the N or C terminus or both. In some embodiments, each of the chains have different sequences. In some embodiments, wherein chain 1 forms a homodimer with chain 2; and chain 3 and 4 associate with chain 1 and chain 2. That is, when each light chain associates with each heavy chain, VL1 associates with VH1 and CL associates with CH1 to form two functional Fab units. Without being bound to any particular theory, each scFv unit is intrinsically functional since VL2 and VH2 are covalently linked in tandem with a linker as provided herein (e.g. GGGGSG (SEQ ID NO: 13), GGGGS (SEQ ID NO: 1), GGGGSGGGGSGGGGSGGGGS (SEQ ID NO: 4), GGGGSGGGGSGGGGS (SEQ ID NO: 3) or GGGGSGGGGS (SEQ ID NO: 2)). The sequences of Linker A and Linker B, which are independent of one another can be the same or different and as otherwise described throughout the present application. Thus, in some embodiments, Linker A comprises GGGGS (SEQ ID NO: 1), GGGGSGGGGS (SEQ ID NO: 2), GGGGSGGGGSGGGGS (SEQ ID NO: 3), or GGGGSGGGGSGGGGSGGGGS (SEQ ID NO: 4). In some embodiments, Linker B comprises GGGGS (SEQ ID NO: 1), GGGGSGGGGS (SEQ ID NO: 2), GGGGSGGGGSGGGGS (SEQ ID NO: 3), or GGGGSGGGGSGGGGSGGGGS (SEQ ID NO: 4). In some embodiments, Linker A comprises 1, 2, 3, 4, or 5 GGGGS (SEQ ID NO: 1) repeats. In some embodiments, Linker B comprises 1, 2, 3, 4, or 5 GGGGS (SEQ ID NO: 1) repeats. For the avoidance of doubt, the sequences of Linker A and Linker B, which are used throughout this application, are independent of one another. Therefore, in some embodiments, Linker A and Linker B can be the same or different. In some embodiments, Linker A comprises GGGGS (SEQ ID NO: 1), GGGGSGGGGS (SEQ ID NO: 2), GGGGSGGGGSGGGGS (SEQ ID NO: 3), or GGGGSGGGGSGGGGSGGGGS (SEQ ID NO: 4). In some embodiments, Linker B comprises GGGGS (SEQ ID NO: 1), GGGGSGGGGS (SEQ ID NO: 2), GGGGSGGGGSGGGGS (SEQ ID NO: 3), or GGGGSGGGGSGGGGSGGGGS (SEQ ID NO: 4). In some embodiments, the Linker A or Linker B comprises: GGGGS (SEQ ID NO: 1), (GGGGS)₃ (SEQ ID NO: 3), (GGGGS)ₙ (n=1, 2, 3, 4) (SEQ ID NO: 1-4), (Gly)₈ (SEQ ID NO: 5), (Gly)₆ (SEQ ID NO: 6), (EAAAK)₃ (SEQ ID NO: 7), (EAAK)ₙ (n=1-3) (SEQ ID NO: 8-10), A(EAAAK)₄ALEA(EAAAK)₄A (SEQ ID NO: 11), or AEAAAKEAAAKA (SEQ ID NO: 12).

The scFv may also be arranged in the NT-VH2-VL2-CT or NT-VL2-VH2-CT orientation. NT or nt stands for N-terminus and CT or ct stands for C-terminus of the protein. In some embodiments, the CH1, CH2, and CH3 are the domains from the IgG Fc region, and CL stands for Constant Light chain, which can be either kappa or lambda family light chains. The other definitions stand for the way they are normally used in the art. In some embodiments, the CH2 portions when present on the strands are different from one another. In some embodiments, the CH2 portions are the same.

In some embodiments, the compound comprises a light chain and a heavy chain. In some embodiments, the light and heavy chain begin at the N-terminus with the VH domain of a cell targeting domain followed by the CH1 domain of a human IgG1, which is fused to a Fc region (e.g. CH2-CH3) of human IgG1. In some embodiments, at the C-terminus of the Fc region is fused to a linker as provided herein, such as but not limited to, GGGGS (SEQ ID NO; 1), GGGGSGGGGS (SEQ ID NO: 2) or GGGGSGGGGSGGGGS (SEQ ID NO: 3). The linker can then be fused to FcγRII binding effector domain. The polypeptides can dimerize because through the heavy chain dimerization, which results in a therapeutic compound having two antigens of interest.

In some embodiments, the linker includes a click chemistry handle. In particular embodiments, the linker is formed by the reaction between a click-chemistry reaction pair. The term “click-chemistry reaction pair” as used herein, means a pair of reactive groups that participates in a modular reaction with high yield and a high thermodynamic gain, thus producing a click-chemistry linker. In some embodiments, one of the reactive groups is attached to the N-terminus, and the other reactive group is attached to the C-terminus of the polypeptide. In some embodiments, one reactive group (e.g., a strained alkyne) is attached to the N- or C-terminus of one polypeptide and the other reactive group (e.g., an azide) is attached to the N- or C-terminus of another polypeptide. Exemplary reactions and click-chemistry pairs include a Huisgen 1,3-dipolar cycloaddition reaction between an alkynyl group and an azido group to form a triazole-containing linker; a Diels-Alder reaction between a diene having a 4π electron system (e.g., an optionally substituted 1,3-unsaturated compound, such as optionally substituted 1,3-butadiene, 1-methoxy-3-trimethylsilyloxy-1,3-butadiene, cyclopentadiene, cyclohexadiene, or furan) and a dienophile or heterodienophile having a 2π electron system (e.g., an optionally substituted alkenyl group or an optionally substituted alkynyl group); a ring opening reaction with a nucleophile and a strained heterocyclyl electrophile; and a reductive amination reaction with an aldehyde group and an amino group (Kolb et al., Angew. Chem. Int. Ed., 40:2004-2021 (2001); Van der Eycken et al., QSAR Comb. Sci., 26:1115-1326 (2007)).

In some embodiments, the 3′-terminus of a polypeptide is linked to the 5′-terminus of a polypeptide by means of a triazole-containing linker formed by the reaction between an alkynyl group and an azido group click-chemistry pair. In some embodiments, the azido group may be attached to the 3′-terminus of the polypeptide and the alkynyl group may be attached to the 5′-terminus of the polypeptide. Alternatively, the azido group may be attached to the 5′-terminus of polypeptide and the alkynyl group may be attached to the 3′-terminus of the polypeptide. In some embodiments, the reaction between an azido group and the alkynyl group is uncatalyzed, and in other embodiments the reaction is catalyzed by a copper(I) catalyst (e.g., copper(I) iodide), a copper(II) catalyst in the presence of a reducing agent (e.g., copper(II) sulfate or copper(II) acetate with sodium ascorbate), or a ruthenium-containing catalyst (e.g., Cp*RuCl(PPh3)2 or Cp*RuCl(COD)).

Exemplary linkers include linkers containing monofluorocyclooctyne (MFCO), difluorocyclooctyne (DFCO), cyclooctyne (OCT), dibenzocyclooctyne (DBCO), biarylazacyclooctyne (BARAC), difluorobenzocyclooctyne (DIFBO), and bicyclo[6.1.0]nonyne (BCN).

The linkers may be conjugated through reacting click chemistry handle pairs. The term “click chemistry handle,” as used herein, refers to a reactant, or a reactive group, that can partake in a click chemistry reaction. For example, a strained alkyne, e.g., a cyclooctyne, is a click chemistry handle, since it can partake in a strain-promoted cycloaddition. In general, click chemistry reactions require at least two molecules comprising click chemistry handles that can react with each other. For example, an azide is a partner click chemistry handle to a cyclooctyne or any other alkyne. Additional examples of partner click chemistry handle pairs include a diene and a dienophile, an azide and a terminal alkyne, an azide and a strained alkyne, an azide and an activated alkyne, an azide and an electron-deficient alkyne, an azide and an aryne, a tetrazine and an alkene, a tetrazole and an alkene, a dithioester and a diene, an anthracene and a maleimide, a thiol and an alkene, a thiol and an enone, a thiol and a maleimide, a thiol and para-fluoro, and an amine and para-fluoro. Other suitable click chemistry handles are known to those of skill in the art.

Additional click chemistry handles suitable for use in the methods described herein are well known to those of skill in the art, and such click chemistry handles include, but are not limited to, the click chemistry reaction partners, groups, and handles described in H. C. Kolb, M. G. Finn, K. B. Sharpless, Angew. Chem. 2001, 113, 2056-2075; Angew. Chem. Int. Ed. 2001, 40, 2004-2021; C. J. Hawker, K. L. Wooley, Science 2005, 309, 1200-1205; D. Fournier, R. Hoogenboom, U. S. Schubert, Chem. Soc. Rev. 2007, 36, 1369-1380; W. H. Binder, R. Sachsenhofer, Macromol. Rapid Commun. 2007, 28, 15-54; H. C. Kolb, K. B. Sharpless, Drug Discovery Today 2003, 8, 1128-1137; V. D. Bock, H. Hiemstra, J. H. van Maarseveen, Eur. J. Org. Chem. 2006, 51-68; V. O. Rodionov, V. V. Fokin, M. G. Finn, Angew. Chem. 2005, 117, 2250-2255; Angew. Chem. Int. Ed. 2005, 44, 2210-2215; P. L. Golas, N. V. Tsarevsky, B. S. Sumerlin, K. Matyjaszewski, Macromolecules 2006, 39, 6451-6457; C. N. Urbani, C. A. Bell, M. R. Whittaker, M. J. Monteiro, Macromolecules 2008, 41, 1057-1060; S. Chassaing, A. S. S. Sido, A. Alix, M. Kumarraja, P. Pale, J. Sommer, Chem. Eur. J. 2008, 14, 6713-6721; B. C. Boren, S. Narayan, L. K. Rasmussen, L. Zhang, H. Zhao, Z. Lin, G. Jia, V. V. Fokin, J. Am. Chem. Soc. 2008, 130, 8923-8930; B. Saba, S. Sharma, D. Sawant, B. Kundu, Synlett 2007, 1591-1594; J. F. Lutz, Angew. Chem. 2008, 120, 2212-2214; Angew. Chem. Int. Ed. 2008, 47, 2182-2184; Q. Wang, T. R. Chan, R. Hilgraf, V. V. Fokin, K. B. Sharpless, M. G. Finn, J. Am. Chem. Soc. 2003, 125, 3192-3193; J. Gierlich, G. A. Burley, P. M. E. Gramlich, D. M. Hammond, T. Carell, Org. Lett. 2006, 8, 3639-3642; J. M. Baskin, J. A. Prescher, S. T. Laughlin, N. J. Agard, P. V. Chang, I. A. Miller, A. Lo, J. A. Codelli, C. R. Bertozzi, Proc. Natl. Acad. Sci. USA 2007, 104, 16793-16797; S. T. Laughlin, J. M. Baskin, S. L. Amacher, C. R. Bertozzi, Science 2008, 320, 664-667; J. A. Johnson, J. M. Baskin, C. R. Bertozzi, J. F. Koberstein, N. J. Turro, Chem. Commun. 2008, 3064-3066; J. A. Codelli, J. M. Baskin, N. J. Agard, C. R. Bertozzi, J. Am. Chem. Soc. 2008, 130, 11486-11493; E. M. Sletten, C. R. Bertozzi, Org. Lett. 2008, 10, 3097-3099; f) J. M. Baskin, C. R. Bertozzi, QSAR Comb. Sci. 2007, 26, 1211-1219; G. Wittig, A. Krebs, Chem. Ber. Recl. 1961, 94, 3260-3275; A. T. Blomquist, L. H. Liu, J. Am. Chem. Soc. 1953, 75, 2153-2154; D. H. Ess, G. O. Jones, K. N. Houk, Org. Lett. 2008, 10, 1633-1636; W. D. Sharpless, P. Wu, T. V. Hansen, J. G. Lindberg, J. Chem. Educ. 2005, 82, 1833-1836; Y. Zou, J. Yin, Bioorg. Med. Chem. Lett. 2008, 18, 5664-5667; X. Ning, J. Guo, M. A. Wolfert, G. J. Boons, Angew. Chem. 2008, 120, 2285-2287; Angew. Chem. Int. Ed. 2008, 47, 2253-2255; S. Sawoo, P. Dutta, A. Chakraborty, R. Mukhopadhyay, O. Bouloussa, A. Sarkar, Chem. Commun. 2008, 5957-5959; Z. Li, T. S. Seo, J. Ju, Tetrahedron Lett. 2004, 45, 3143-3146; S. S. van Berkel, A. J. Dirkes, M. F. Debets, F. L. van Delft, J. J. L. Cornelissen, R. J. M. Nolte, F. P. J. Rutjes, Chem Bio Chem 2007, 8, 1504-1508; S. S. van Berkel, A. J. Dirks, S. A. Meeuwissen, D. L. L. Pingen, O. C. Boerman, P. Laverman, F. L. van Delft, J. J. L. Cornelissen, F. P. J. Rutjes, Chem Bio-Chem 2008, 9, 1805-1815; F. Shi, J. P. Waldo, Y. Chen, R. C. Larock, Org. Lett. 2008, 10, 2409-2412; L. Campbell-Verduyn, P. H. Elsinga, L. Mirfeizi, R. A. Dierckx, B. L. Feringa, Org. Biomol. Chem. 2008, 6, 3461-3463; The Chemistry of the Thiol Group (Ed.: S. Patai), Wiley, New York, 1974; A. F. Jacobine, In Radiation Curing in Polymer Science and Technology III (Eds.: J. D. Fouassier, J. F. Rabek), Elsevier, London, 1993, Chap. 7, pp. 219-268; C. E. Hoyle, T. Y. Lee, T. Roper, J. Polym. Sci. Part A 2008, 42, 5301-5338; L. M. Campos, K. L. Killops, R. Sakai, J. M. J. Paulusse, D. Damiron, E. Drockenmuller, B. W. Messmore, C. J. Hawker, Macromolecules 2008, 41, 7063-7070; R. L. A. David, J. A. Kornfield, Macromolecules 2008, 41, 1151-1161; C. Nilsson, N. Simpson, M. Malkoch, M. Johansson, E. Malmstrom, J. Polym. Sci. Part A 2008, 46, 1339-1348; A. Dondoni, Angew. Chem. 2008, 120, 9133-9135; Angew. Chem. Int. Ed. 2008, 47, 8995-8997; J. F. Lutz, H. Schlaad, Polymer 2008, 49, 817-824; A. Gress, A. Voelkel, H. Schlaad, Macromolecules 2007, 40, 7928-7933; N. ten Brummelhuis, C. Diehl, H. Schlaad, Macromolecules 2008, 41, 9946-9947; K. L. Killops, L. M. Campos, C. J. Hawker, J. Am. Chem. Soc. 2008, 130, 5062-5064; J. W. Chan, B. Yu, C. E. Hoyle, A. B. Lowe, Chem. Commun. 2008, 4959-4961; G. Moad, E. Rizzardo, S. H. Thang, Acc. Chem. Res. 2008, 41, 1133-1142; C. Barner-Kowollik, M. Buback, B. Charleux, M. L. Coote, M. Drache, T. Fukuda, A. Goto, B. Klumperman, A. B. Lowe, J. B. McLeary, G. Moad, M. J. Monterio, R. D. Sanderson, M. P. Tonge, P. Vana, J. Polym. Sci. Part A 2006, 44, 5809-5831; R. J. Pounder, M. J. Stanford, P. Brooks, S. P. Richards, A. P. Dove, Chem. Commun. 2008, 5158-5160; b) M. J. Stanford, A. P. Dove, Macromolecules 2009, 42, 141-147; M. Li, P. De, S. R. Gondi, B. S. Sumerlin, J. Polym. Sci. Part A 2008, 46, 5093-5100; Z. J. Witczak, D. Lorchak, N. Nguyen, Carbohydr. Res. 2007, 342, 1929-1933; D. Samaroo, M. Vinodu, X. Chen, C. M. Drain, J. Comb. Chem. 2007, 9, 998-1011; X. Chen, D. A. Foster, C. M. Drain, Biochemistry 2004, 43, 10918-10929; D. Samaroo, C. E. Soll, L. J. Todaro, C. M. Drain, Org. Lett. 2006, 8, 4985-4988; P. Battioni, O. Brigaud, H. Desvaux, D. Mansuy, T. G. Traylor, Tetrahedron Lett. 1991, 32, 2893-2896; C. Ott, R. Hoogenboom, U. S. Schubert, Chem. Commun. 2008, 3516-3518; V. Ladmiral, G. Mantovani, G. J. Clarkson, S. Cauet, J. L. Irwin, D. M. Haddleton, J. Am. Chem. Soc. 2006, 128, 4823-4830; S. G. Spain, M. I. Gibson, N. R. Cameron, J. Polym. Sci. Part A 2007, 45, 2059-2072; C. R. Becer, K. Babiuch, K. Pilz, S. Hornig, T. Heinze, M. Gottschaldt, U. S. Schubert, Macromolecules 2009, 42, 2387-2394; Otto Paul Hermann Diels and Kurt Alder first documented the reaction in 1928. They received the Nobel Prize in Chemistry in 1950 for their work on the eponymous reaction; H. L. Holmes, R. M. Husband, C. C. Lee, P. Kawulka, J. Am. Chem. Soc. 1948, 70, 141-142; M. Lautens, W. Klute, W. Tam, Chem. Rev. 1996, 96, 49-92; K. C. Nicolaou, S. A. Snyder, T. Montagnon, G. Vassilikogiannakis, Angew. Chem. 2002, 114, 1742-1773; Angew. Chem. Int. Ed. 2002, 41, 1668-1698; E. J. Corey, Angew. Chem. 2002, 114, 1724-1741; Angew. Chem. Int. Ed. 2002, 41, 1650-1667; H. Durmaz, A. Dag, O. Altintas, T. Erdogan, G. Hizal, U. Tunca, Macromolecules 2007, 40, 191-198; H. Durmaz, A. Dag, A. Hizal, G. Hizal, U. Tunca, J. Polym. Sci. Part A 2008, 46, 7091-7100; A. Dag, H. Durmaz, E. Demir, G. Hizal, U. Tunca, J. Polym. Sci. Part A 2008, 46, 6969-6977; B. Gacal, H. Akat, D. K. Balta, N. Arsu, Y. Yagci, Macromolecules 2008, 41, 2401-2405; A. Dag, H. Durmaz, U. Tunca, G. Hizal, J. Polym. Sci. Part A 2009, 47, 178-187; M. L. Blackman, M. Royzen, J. M. Fox, J. Am. Chem. Soc. 2008, 130, 13518-13519; N. K. Devaraj, R. Weissleder, S. A. Hilderbrand, Bioconjugate Chem. 2008, 19, 2297-2299; W. Song, Y. Wang, J. Qu, Q. Lin, J. Am. Chem. Soc. 2008, 130, 9654-9655; W. Song, Y. Wang, J. Qu, M. M. Madden, Q. Lin, Angew. Chem. 2008, 120, 2874-2877; Angew. Chem. Int. Ed. 2008, 47, 2832-2835; A. Dag, H. Durmaz, G. Hizal, U. Tunca, J. Polym. Sci. Part A 2008, 46, 302-313; A. J. Inglis, S. Sinnwell, T. P. Davis, C. Barner-Kowollik, M. H. Stenzel, Macromolecules 2008, 41, 4120-4126; S. Sinnwell, A. J. Inglis, T. P. Davis, M. H. Stenzel, C. Barner-Kowollik, Chem. Commun. 2008, 2052-2054; A. J. Inglis, S. Sinwell, M. H. Stenzel, C. Bamer-Kowollik, Angew. Chem. 2009, 121, 2447-2450; Angew. Chem. Int. Ed. 2009, 48, 2411-2414. All references cited above are incorporated herein by reference in their entirety.

The polypeptides, molecules, or antibodies provided for herein may also be conjugated to a chemical moiety. The chemical moiety may be, inter alia, a polymer, a radionuclide or a cytotoxic factor. In some embodiments, this can be referred to as an antibody, or the antigen-binding fragment thereof, drug conjugate. In some embodiments, the chemical moiety is a polymer which increases the half-life of the antibody, or the antigen-binding fragment thereof, molecule in the body of a subject. Suitable polymers include, but are not limited to, polyethylene glycol (PEG) (e.g., PEG with a molecular weight of 1 kDA, 2 kDa, 5 kDa, 10 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)). Examples of chemical moieties include, but are not limited to, anti-mitotics, such as calicheamicins (e.g. ozogamicin), monomethyl auristatin E, mertansine, and the like. Other examples include, but are not limited to, biologically active anti-microtubule agents, alkylating agents and DNA minor groove binding agents. Other examples of are provided herein and below. The chemical moiety can be linked to the antibody, or the antigen-binding fragment thereof, through a linking group (maleimide), a cleavable linker, such as a cathepsin cleavable linkers (valine-citrulline), and in some embodiments, one or more spacers (e.g. para-aminobenzylcarbamate). Without being bound to any particular theory, after the cell targeting antibody, or the antigen-binding fragment thereof, conjugate binds to a thymic cell receptor it can be internalized and the antigen of interest presenting the antigen, allergen, or pollen can be released inside they thymic cell, processed intracellularly for MHC I and/or MHC II presentation, and presented on the surface of the thymic cell as MHC and peptide. “MHC and peptide”, as used herein, is meant to refer to the MHC/peptide complex as it is presented on the surface of a thymic cell. Cytoplasmic antigens are processed into peptides by cytoplasmic proteases and the proteasome. As used herein, the term “MHC and peptide”includes such naturally occurring complexes, and in addition includes peptides that differ from native antigen-derived peptides but which are nonetheless able to form a complex with class I or II that is effective to maintain functional cells.

The polypeptides, molecules, or antibodies provided herein may also be conjugated with labels such as ⁹⁹Tc, ⁹⁰Y, ¹¹¹In ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷CU, ²¹⁷Ci, ²¹At, ²¹²Pb, ⁴⁷Sc, ¹⁰⁹Pd, ²³⁴Th, and ⁴⁰K, ¹⁵⁷Gd, ⁵⁵Mn, ⁵²Tr and ⁵⁶Fe.

The polypeptides, molecules, or antibodies provided herein may also be conjugated with fluorescent or chemiluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The polypeptides, molecules, or antibodies provided herein may also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, Momordica charantia inhibitor, curcin, crotin, Saponaria officinalis inhibitor, mitogellin, restrictocin, phenomycin, and enomycin.

Any method known in the art for conjugating the antibody, or the antigen-binding fragment thereof, molecules of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies are conventional and very well known in the art.

Antibody, or the antigen-binding fragment thereof, molecule, as that term is used herein, refers to a polypeptide, e.g., an immunoglobulin chain or fragment thereof, comprising at least one functional immunoglobulin variable domain sequence. An antibody, or the antigen-binding fragment thereof, molecule encompasses antibodies (e.g., full-length antibodies) and antibody, or the antigen-binding fragment thereof, fragments. In some embodiments, an antibody, or the antigen-binding fragment thereof, molecule comprises an antigen binding or functional fragment of a full-length antibody, or the antigen-binding fragment thereof, or a full-length immunoglobulin chain. For example, a full-length antibody, or the antigen-binding fragment thereof, is an immunoglobulin (Ig) molecule (e.g., an IgG antibody, or the antigen-binding fragment thereof,) that is naturally occurring or formed by normal immunoglobulin gene fragment recombinatorial processes). In embodiments, an antibody, or the antigen-binding fragment thereof, molecule refers to an immunologically active, antigen-binding portion of an immunoglobulin molecule, such as an antibody, or the antigen-binding fragment thereof, fragment. An antibody, or the antigen-binding fragment thereof, fragment, e.g., functional fragment, comprises a portion of an antibody, or the antigen-binding fragment thereof, e.g., Fab, Fab′, F(ab′)2, F(ab)2, variable fragment (Fv), domain antibody, or the antigen-binding fragment thereof, (dAb), or single chain variable fragment (scFv). A functional antibody, or the antigen-binding fragment thereof, fragment binds to the same antigen as that recognized by the intact (e.g., full-length) antibody, or the antigen-binding fragment thereof. The terms “antibody, or the antigen-binding fragment thereof, fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody, or the antigen-binding fragment thereof, fragment does not include portions of antibodies without antigen binding activity, such as Fc fragments or single amino acid residues. Exemplary antibody, or the antigen-binding fragment thereof, molecules include full-length antibodies and antibody, or the antigen-binding fragment thereof, fragments, e.g., dAb (domain antibody, or the antigen-binding fragment thereof,), single chain, Fab, Fab′, and F(ab′)2 fragments, and single chain variable fragments (scFvs).

The term “antibody, or the antigen-binding fragment thereof, molecule” also encompasses whole or antigen binding fragments of domain, or single domain, antibodies, which can also be referred to as “sdAb” or “VHH.” Domain antibodies comprise either V_{H} or V_{L} that can act as stand-alone, antibody, or the antigen-binding fragment thereof, fragments. Additionally, domain antibodies include heavy-chain-only antibodies (HCAbs). Domain antibodies also include a CH2 domain of an IgG as the base scaffold into which CDR loops are grafted. It can also be generally defined as a polypeptide or protein comprising an amino acid sequence that is comprised of four framework regions interrupted by three complementarity determining regions. This is represented as FR1-CDR1-FR2-CDR2-FR3-CDR3-FR4. sdAbs can be produced in camelids such as llamas, but can also be synthetically generated using techniques that are well known in the art. The numbering of the amino acid residues of a sdAb or polypeptide is according to the general numbering for VH domains given by Kabat et al. (“Sequence of proteins of immunological interest,” US Public Health Services, NIH Bethesda, MD, Publication No. 91, which is hereby incorporated by reference). According to this numbering, FR1 of a sdAb comprises the amino acid residues at positions 1-30, CDR1 of a sdAb comprises the amino acid residues at positions 31-36, FR2 of a sdAb comprises the amino acids at positions 36-49, CDR2 of a sdAb comprises the amino acid residues at positions 50-65, FR3 of a sdAb comprises the amino acid residues at positions 66-94, CDR3 of a sdAb comprises the amino acid residues at positions 95-102, and FR4 of a sdAb comprises the amino acid residues at positions 103-113. Domain antibodies are also described in WO2004041862 and WO2016065323, each of which is hereby incorporated by reference. The domain antibodies can be a targeting moiety as described herein.

Antibody, or the antigen-binding fragment thereof, molecules can be monospecific (e.g., monovalent or bivalent), bispecific (e.g., bivalent, trivalent, tetravalent, pentavalent, or hexavalent), trispecific (e.g., trivalent, tetravalent, pentavalent, hexavalent), or with higher orders of specificity (e.g, tetraspecific) and/or higher orders of valency beyond hexavalency. An antibody, or the antigen-binding fragment thereof, molecule can comprise a functional fragment of a light chain variable region and a functional fragment of a heavy chain variable region, or heavy and light chains may be fused together into a single polypeptide.

Effector, as that term is used herein, refers to an entity, e.g., a cell or molecule, e.g., a soluble or cell surface molecule, which mediates an immune response. In some embodiments, the effector is an antibody, or the antigen-binding fragment thereof. In some embodiments, the effectors binding domains as provided for herein, refers to a polypeptide (e.g.) that has sufficient binding specificity that it can bind the effector with sufficient specificity that it can serve as an effector binding/modulating molecule. In some embodiments, it binds to effector with at least 10, 20, 30, 40, 50, 60, 70, 80, 90, or 95% of the affinity of the naturally occurring counter-ligand. In some embodiments, it has at least 60, 70, 80, 90, 95, 99, or 100% sequence identity, or substantial sequence identity, with a naturally occurring counter-ligand for the effector.

Elevated risk, as used herein, refers to the risk of a disorder in a subject, wherein the subject has one or more of a medical history of the disorder or a symptom of the disorder, a biomarker associated with the disorder or a symptom of the disorder, or a family history of the disorder or a symptom of the disorder.

The domains provided herein can have similarity to those as provided for herein or those that are incorporated by reference. Sequence identity, percentage identity, and related terms, as those terms are used herein, refer to the relatedness of two sequences, e.g., two nucleic acid sequences or two amino acid or polypeptide sequences. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of nucleotide sequence, such as those encoding for the domains, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers to polypeptides that have a substantially identical amino acid sequence to the naturally-occurring sequence, or are encoded by a substantially identical nucleotide sequence, and are capable of having one or more activities of the naturally-occurring sequence. For example, a Fc variant can have the sequence of a Fc domain but comprise a mutation that affects its binding to the Fc receptor.

Calculations of homology or sequence identity between sequences (the terms are used interchangeably herein) can be performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In some embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiments, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to for example any nucleic acid sequence provided herein. BLAST protein searches can be performed with the XBLAST program, score=50, wordlength=3 to obtain amino acid sequences homologous to protein molecules provided herein. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov. As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6X sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the molecules and compounds of the present embodiments may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The molecules provided for herein can be used to treat autoimmune, alloimmune, allergic, or inflammatory diseases. In some embodiments, the compounds provided for herein can also be used to treat, and/or reduce an anti-drug immune response, wherein the anti-drug immune response is a response to a biologic or a viral vector. Thus, in some embodiments, embodiments are provided for methods of treating an autoimmune, alloimmune, allergic, or inflammatory disease or disorder in a subject. In some embodiments, the methods comprise administering to the subject a compound as provided for herein. In some embodiments, the subject has or is at risk of having an autoimmune, alloimmune, allergic, or inflammatory disorder. In some embodiments, the autoimmune, alloimmune, allergic, or inflammatory disorder is asthma, severe asthma. neutrophilic asthma, paucigranulocytic asthma, eosinophilic asthma, osteoarthritis, solid organ transplant, atopic dermatitis, eosinophilic esophagitis, dermatitis herpitoformis, chronic rhinosinusitis, chronic spontaneous urticaria, food allergy, anti-biologic drug immune response, anti-viral immune response, anti-AAV immune response, anti-adenoviral immune response, anti-retroviral immune response, anti-lentiviral immune response, and -viral-like particle (VLP), alzheimer's disease, amyotrophic lateral sclerosis (ALS), Parkinson's disease, dementia with Lewy bodies, Duchenne muscular dystrophy, cutaneous lupus, immune response, myocarditis, postmyocardial infarction syndrome, postpericardiotomy syndrome, subacute bacterial endocarditis, anti-glomerular basement membrane nephritis, interstitial cystitis, lupus nephritis, membranous glomerulonephropathy, chronic kidney disease (“CKD”), autoimmune hepatitis, primary biliary cirrhosis, primary sclerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease (LAD), morphea, pemphigus, pemphigus vulgaris, hidradenitis suppuritiva, pityriasis lichenoides et varioliformis acuta, Mucha-Habermann disease, psoriasis, guttate psoriasis, erythrodermic psoriasis, plaque psoriasis, pustular psoriasis, systemic sclerosis, skin manifestation associated with systemic sclerosis, vitiligo, Addison's disease, autoimmune polyendocrine syndrome (APS) type 1, autoimmune polyendocrine syndrome (APS) type 2, autoimmune polyendocrine syndrome (APS) type 3, autoimmune pancreatitis (AIP), diabetes mellitus type 1, autoimmune thyroiditis, Ord's thyroiditis, Graves' disease, non radiographic axial spondyloarthropathy, autoimmune oophoritis, endometriosis, autoimmune orchitis, Sjogren's syndrome, dermatitis herpatiformis, autoimmune enteropathy, coeliac disease, Crohn's disease, immune related adverse events (irAEs) secondary to immune checkpoint inhibitors, microscopic colitis, ulcerative colitis, autoimmune thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, rheumatoid arthritis, sarcoidosis, Schnitzler syndrome, systemic lupus erythematosus (SLE), undifferentiated connective tissue disease (UCTD), dermatomyositis, fibromyalgia, inclusion body myositis, myositis, myasthenia gravis, neuromyotonia, paraneoplastic cerebellar degeneration, polymyositis, acute disseminated encephalomyelitis (ADEM), acute motor axonal neuropathy, anti-N-methyl-D-aspartate (anti-NMDA) receptor encephalitis, balo concentric sclerosis, Bickerstaffs encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, oshtoran syndrome, pediatric autoimmune neuropsychiatric disorder associated with streptococcus (PANDAS), progressive inflammatory neuropathy, restless leg syndrome, stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Mdniere's disease, Behcet's disease, eosinophilic granulomatosis with polyangiitis (EGPA), giant cell arteritis, granulmatosis with polyangiitis (GPA), IgA vasculitis (IgAV), Kawasaki's disease, leukocytoclastic vasculitis, lupus vasculitis, rheumatoid vasculitis, microscopic polyangiitis (MPA), polyarteritis nodosa (PAN), polymyalgia rheumaticia, vasculitis, primary immune deficiency, Gaucher disease, Pompe disease, Fabry disease, Schindler disease, Hurler syndrome (MPS1), Tay-Sachs disease, Nieman-Pick disease, Farber lipogranulomatosis, Krabbe disease, Morquio syndrome (MPS4), mucopolysaccharidosis type VI (MPS6), Hunter syndrome (MPS2), Sanfilippo syndrome (MPS3), Sly sundrome (MPS7), hyaluronidase deficiency (MPS9), mucolipidosis type 1 (ML1), I-cell disease, mucolipidosis II (ML2), mucolipidosis III gamma (ML3C), glycogen storage disease Ia, glycogen storage disease Ib, Wilson disease, phenylketonuria, Stargardt disease type I, myocilin-associated glaucoma, hemophilia A, hemophilia B, hereditary or acquired thrombotic thrombocytopathic purpura (TTP), von Willebrand disease, thrombocytopenia platelet autoantigen, heparin-induced thrombocytopenia, tolerance induction for AAV gene therapy, tolerance induction for ex vivo gene editing, tolerance induction for in vivo gene editing, acquired hemophilia, antiphospholipid syndrome, aplastic anemia, autoimmune lymphoproliferative syndrome, autoimmune neutropenia, autoimmune thrombocytopenic purpura, cold agglutinin disease, essential mixed cryoglobulinemia, Evans syndrome, IgG4-related diseases, pure red cell aplasia, warm autoimmune hemolytic anemia, autoimmune gastritis, celiac disease, pernicious anemia, anti-sperm antibodies, autoimmune polyendocrine syndrome type 1, autoimmune polyendocrine syndrome type 2, autoimmune polyendocrine syndrome type 3, autoimmune myocarditis, cryptogenic organizing pneumonia, idiopathic pulmonary fibrosis, IgA nephropathy, post-myocardial infarction syndrome, primary biliary cholangitis, primary idiopathic dilated cardiomyopathy, anti-N-methyl-D-aspartate receptor encephalitis, autoimmune encephalitis, encephalopathy associated with autoimmune thyroid disease, episcleritis, Graves' ophthalmopathy, myelin oligodendrocyte glycoprotein disease, narcolepsy with cataplexy, cutaneous lupus erythematosus, adult-onset Still's disease, ankylosing spondylitis, juvenile arthritis, juvenile dermatomyositis, anti-neutrophil cytoplasmic antibody-associated vasculitis, polymyalgia rheumatic, purpura rheumatic, Takayasu arteritis, graft versus host disease (GVHD), transplant, xenotransplantation, and the like.

In some embodiments, the transplant is an organ or a cell transplant. In some embodiments, the transplant is an allogeneic organ or an allogeneic cell transplant. In some embodiments, the allogeneic organ transplant, is a heart transplant, a lung transplant, a liver transplant, a pancreas transplant, a cornea transplant, a trachea transplant, a kidney transplant, a skin transplant, or a vascular tissue transplant. In some embodiments, the allogeneic cell transplant, is a stem cell transplant or a bone marrow transplant. In some embodiments, the cell transplant is an allogenic stem cell transplant, a non-myeloablative transplant, a syngeneic stem cell transplant, or a haploidentical transplant.

In some embodiments, the allogeneic organ or the allogeneic cell transplant is from an allogeneic donor, wherein the MHC of said allogeneic donor is not matched to the MHC of said recipient.

The term “MHC-matched” as used herein refers to the condition in which the donor has the same human leukocyte antigens (HLA) as the recipient. Accordingly, an allogeneic donor that is non-matched to the MHC of the recipient has different HLA as the recipient. In some embodiments, the allogeneic donor organ has different HLA than the recipient. In some embodiments, the allogeneic donor cell has different HLA than the recipient. In some embodiments, the allogeneic donor organ has related, but not identical, HLA than the recipient. In some embodiments, the allogeneic donor cell has related, but not identical, HLA than the recipient.

In some embodiments, the polypeptides, molecules, and pharmaceutical compositions provided for herein are used in a method of modulating a cell, or a plurality of cells, such as a thymic cell, the method comprising contacting the cell with the polypeptide, the molecule, or the pharmaceutical composition thereof. In some embodiments, cell is a thymic cell, such as medullary thymic epithelial cell (mTEC), mimetic mTEC, mTEC progenitor cell, mimetic mTEC progenitor cell, junction thymic epithelial cell (jTEC), thymic progenitor epithelial cell (TPEC), proliferating TPEC, thymic epithelial cell (TEC), proliferating TEC, cortical thymic epithelial cell (cTEC), proliferating cTEC, or proliferating mTEC.

In some embodiments, the polypeptides, molecules, and pharmaceutical compositions provided for herein are used in a method of transforming a cell, or plurality of cells, such as a thymic cell, the method comprising contacting the cell with the polypeptide, the molecule, or the pharmaceutical composition thereof. In some embodiments, cell is a thymic cell, such as medullary thymic epithelial cell (mTEC), mimetic mTEC, mTEC progenitor cell, mimetic mTEC progenitor cell, junction thymic epithelial cell (jTEC), thymic progenitor epithelial cell (TPEC), proliferating TPEC, thymic epithelial cell (TEC), proliferating TEC, cortical thymic epithelial cell (cTEC), proliferating cTEC, or proliferating mTEC. In some embodiments, contacting the cell with the polypeptide, the molecule, or the pharmaceutical composition thereof, transforms the cell into an antigen-presenting cell.

In some embodiments, the polypeptides, molecules, and pharmaceutical compositions provided for herein are used in a method of inducing immune tolerance, such as T cell tolerance to a molecule, such as an antigen, auto-antigen, self-antigen, tolerance inducing antigen, or an effector peptide, the method comprising contacting the cell with the polypeptide, the molecule, or the pharmaceutical composition thereof. In some embodiments, the T cell is such as those provided in Table 1. In some embodiments, the T cell is a T cell is a CD4+CD8+ T cell, a CD4+ T cell, or a CD8+ T cell. In some embodiments, the CD4+CD8+ T cell, the CD4+ T cell, or the CD8+ T cell is specific against the molecule, such as an antigen, auto-antigen, self-antigen, tolerance inducing antigen, or an effector peptide, such as those provided herein.

In some embodiments, the polypeptides, molecules, and pharmaceutical compositions provided for herein are used in a method of inducing negative selection of an immune cell, such as a T cell that targets a molecule, such as an antigen, auto-antigen, self-antigen, tolerance inducing antigen, or an effector peptide, the method comprising contacting the cell with the polypeptide, the molecule, or the pharmaceutical composition thereof. In some embodiments, the T cell is such as those provided in Table 1. In some embodiments, the T cell is a T cell is a CD4+CD8+ T cell, a CD4+ T cell, or a CD8+ T cell. In some embodiments, the CD4+CD8+ T cell, the CD4+ T cell, or the CD8+ T cell is specific against the molecule, such as an antigen, auto-antigen, self-antigen, tolerance inducing antigen, or an effector peptide, such as those provided herein.

In some embodiments, the polypeptides, molecules, and pharmaceutical compositions provided for herein are used in a method of inducing positive selection of an immune cell, such as positive selection of a Treg cell, the method comprising contacting the cell with the polypeptide, the molecule, or the pharmaceutical composition thereof. In some embodiments, the T cell is such as those provided in Table 1. In some embodiments, the Treg cell is a CD4+CD25+ T cell, or a Foxp3+CD4+CD25+ Treg. In some embodiments, the CD4+CD25+ Treg cell, or a Foxp3+CD4+CD25+ Treg cell is specific against the molecule, such as an antigen, auto-antigen, self-antigen, tolerance inducing antigen, or an effector peptide, such as those provided herein.

In some embodiments, the polypeptides, molecules, and pharmaceutical compositions provided for herein are used in a method of improving transplant therapy in a subject, the method comprising administering the polypeptide, the molecule, or the pharmaceutical composition thereof, wherein the antigen of interest comprises an HLA or tissue antigen, or a fragment thereof. In some embodiments, the transplant is an allogeneic organ or an allogeneic cell transplant. In some embodiments, the HLA or tissue antigen, or a fragment thereof, is of the same class as the allogeneic donor organ or the allogeneic donor cell transplant. In some embodiments, the HLA or tissue antigen, or a fragment thereof, is selected from HLA-A, HLA-B, HLA-C, HLA-E, HLA-L, HLA-J, HLA-K, HLA-H, HLA-G, HLA-DM, HLA-DO, HLA-DP, HLA-DQ, and HLA-DR.

In some embodiments, the polypeptides, molecules, and pharmaceutical compositions provided for herein are used in a method of reducing transplant rejection in a subject, the method comprising administering the polypeptide, the molecule, or the pharmaceutical composition thereof, wherein the antigen of interest comprises an HLA or tissue antigen, or a fragment thereof. In some embodiments, the transplant is an allogeneic organ or an allogeneic cell transplant. In some embodiments, the HLA a or tissue antigen, or a fragment thereof, is of the same class as the allogeneic donor organ or the allogeneic donor cell transplant. In some embodiments, the HLA or tissue antigen, or a fragment thereof, is selected from HLA-A, HLA-B, HLA-C, HLA-E, HLA-L, HLA-J, HLA-K, HLA-H, HLA-G, HLA-DM, HLA-DO, HLA-DP, HLA-DQ, and HLA-DR.

In some embodiments, the polypeptides, molecules, and pharmaceutical compositions provided for herein are used in a method of inducing tolerance to a transgene. In some embodiments, the polypeptides, molecules, and pharmaceutical compositions provided for herein are used in a method of inducing tolerance to a transgene while using an anti-capsid directed approach that involves generating a localized immune-suppressive environment in the target tissue, allowing for successful gene therapy without triggering an immune response against the viral vector or the therapeutic transgene. Without wishing to be bound to a particular theory, by leveraging the bystander effect, regulatory T cells (Tregs) specific for the viral capsid antigen can be generated and localized to the target tissue. The “bystander effect,” as used herein, refers to a phenomenon in which immune cells, particularly regulatory T cells (Tregs), can be utilized to suppress immune responses against both the viral vector and the therapeutic transgene, ensuring sustained transgene expression and improving treatment efficacy. These Tregs can suppress immune responses not only against the capsid but also against the transgene product, through the release of immunosuppressive cytokines and other mechanisms. This approach aims to prevent immune-mediated elimination of transduced cells and ensure sustained transgene expression, ultimately improving the safety and efficacy of gene therapy treatments.

In some embodiments, a method of inducing tolerance to a viral capsid protein in a subject is provided. In some embodiments, the method comprises administering any one, or any combination of the polypeptide, the molecule, or the pharmaceutical composition provided herein. In some embodiments, the method further comprises inducing tolerance to a transgene. In some embodiments, inducing tolerance to the transgene enables persistence of the transgene. In some embodiments, inducing tolerance achieves mitigation of immunological side effects of the therapy.

In some embodiments, a method of inducing antigen-specific Treg expansion in a subject is provided. In some embodiments, the method comprises administering any one, or any combination of the polypeptide, the molecule, or the pharmaceutical composition provided herein. In some embodiments, the method comprises systemically administering any one, or any combination of the polypeptide, the molecule, or the pharmaceutical composition provided herein. In some embodiments, the Treg is a thymic Treg, or a splenic Treg. In some embodiments, the Treg is a CD4+ FoxP3+ Treg. In some embodiments, the Treg is a CD4+ FoxP3+CD73− Treg. In some embodiments, the CD4+ FoxP3+CD73− Treg is a Treg that developed in the thymus. In some embodiments, the method does not significantly induce antigen-specific Teff cells. In some embodiments, systemic administration of the polypeptide, the molecule, or the pharmaceutical composition provided herein achieves targeted induction of thymic T cells.

In some embodiments, a method of inducing immune tolerance against an antigen of interest, such as T cell tolerance to the antigen of interest in a subject is provided. In some embodiments, the method comprises administering any one, or any combination of the polypeptide, the molecule, or the pharmaceutical composition provided herein. In some embodiments, the method comprises systemically administering any one, or any combination of the polypeptide, the molecule, or the pharmaceutical composition provided herein. In some embodiments, the T cell is a CD4+CD8+ T cell, a CD4+ T cell, or a CD8+ T cell. In some embodiments, the T cell is a thymic Treg, or a splenic Treg. In some embodiments, the T cell is a CD4+ FoxP3+ Treg. In some embodiments, the Treg is a CD4+ FoxP3+CD73− Treg. In some embodiments, the CD4+ FoxP3+CD73− Treg is a Treg that developed in the thymus. In some embodiments, systemic administration of the polypeptide, the molecule, or the pharmaceutical composition provided herein achieves targeted induction of thymic T cells.

In some embodiments, a method of systemically administering any one, or any combination of the polypeptide, the molecule, or the pharmaceutical composition is provided herein. In some embodiments, systemically administering any one, or any combination of the polypeptide, the molecule, or the pharmaceutical composition provided herein leads to targeted effect on thymic T cells.

In some embodiments the subject is a healthy subject. In some embodiments, the subject is in need of treatment. In some embodiments, the subject is subject receiving a viral therapy. In some embodiments, the subject is subject receiving an antibody therapy.

In some embodiments, the viral capsid protein is an AAV capsid protein, such as those disclosed herein.

In some embodiments, the method further comprises re-dosing the subject with any one, or any combination of the polypeptide, the molecule, or the pharmaceutical composition provided herein.

The contacting can occur, for example, by administration of the polypeptides provided for herein to a subject.

### Pharmaceutical Compositions and Kits

In some embodiments, the present embodiments provide compositions, e.g., pharmaceutically acceptable compositions, which include a therapeutic compound described herein, formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intrathymic, intraperitoneal, intrathecal, intracerebroventricular, intramuscular, subcutaneous, parenteral, oral, rectal, local, ophthalmic, topical, spinal or epidermal administration (e.g. by injection or infusion). As used herein, the term “carrier” means a diluent, adjuvant, or excipient with which a compound is administered. In some embodiments, pharmaceutical carriers can also be liquids, such as water and oils, including those of petroleum, animal, vegetable, or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used. The carriers can be used in pharmaceutical compositions comprising the therapeutic compounds provided for herein.

The compositions and compounds of the embodiments provided for herein may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical compositions are in the form of injectable or infusible solutions. In some embodiments, the mode of administration is parenteral (e.g., intravenous, subcutaneous, intraperitoneal, intramuscular). In some embodiments, the therapeutic molecule is administered by intravenous infusion or injection. In some embodiments, the therapeutic molecule is administered by intramuscular or subcutaneous injection. In some embodiments, the therapeutic molecule is administered locally, e.g., by injection, or topical application, to a target site.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, intrathymic, epidural and intrasternal injection and infusion.

In some embodiments, the molecule, the polypeptide, or the pharmaceutical composition provided herein may be administered systemically. In some embodiments, the molecule, the polypeptide, or the pharmaceutical composition provided herein administered systemically may be administered without limitation, via intravenous, intramuscular, intraarterial, intrathecal, intracapsular, intraperitoneal, subcutaneous, and epidural injection and infusion.

The compositions typically should be sterile and stable under the conditions of manufacture and storage. The composition can be formulated as a solution, microemulsion, dispersion, liposome, or other ordered structure suitable to high therapeutic molecule concentration. Sterile injectable solutions can be prepared by incorporating the active compound (i.e., therapeutic molecule) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, a therapeutic compound can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specifications for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a therapeutic compound is 0.1-30 mg/kg, more preferably 1-25 mg/kg. Dosages and therapeutic regimens of the therapeutic compound can be determined by a skilled artisan. In some embodiments, the therapeutic compound is administered by injection (e.g., intravenously, or intrathymically) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In some embodiments, the therapeutic compound is administered at a dose from about 10 to 20 mg/kg every other week. The therapeutic compound can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In some embodiments, the infusion rate of about 110 to 130 mg/m² achieves a level of about 3 mg/kg. In some embodiments, the therapeutic compound can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or, about 10 mg/m². In some embodiments, the therapeutic compound is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions may include a “therapeutically effective amount” or a “prophylactically effective amount” of a therapeutic molecule. A “therapeutically effective amount” refers to an amount effective, at dosages and for periods of time necessary, to achieve the desired therapeutic result. A therapeutically effective amount of a therapeutic molecule may vary according to factors such as the disease state, age, sex, and weight of the individual, and the ability of the therapeutic compound to elicit a desired response in the individual. A therapeutically effective amount is also one in which any toxic or detrimental effects of a therapeutic molecule t is outweighed by the therapeutically beneficial effects. A “therapeutically effective dosage” preferably inhibits a measurable parameter, e.g., immune attack at least about 20%, more preferably by at least about 40%, even more preferably by at least about 60%, and still more preferably by at least about 80% relative to untreated subjects. The ability of a compound to inhibit a measurable parameter, e.g., immune attack, can be evaluated in an animal model system predictive of efficacy in transplant rejection or autoimmune disorders. Alternatively, this property of a composition can be evaluated by examining the ability of the compound to inhibit, such inhibition in vitro by assays known to the skilled practitioner. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the embodiments is a kit comprising a therapeutic compound described herein. The kit can include one or more other elements including: instructions for use; other reagents, e.g., a label, a therapeutic agent, or an agent useful for chelating, or otherwise coupling, a therapeutic molecule to a label or other therapeutic agent, or a radioprotective composition; devices or other materials for preparing the therapeutic molecule for administration; pharmaceutically acceptable carriers; and devices or other materials for administration to a subject.

### ENUMERATED EMBODIMENTS

1. A molecule for inducing antigen-specific thymic Tregs against an antigen of interest by systemic administration, wherein the molecule comprises a thymic cell targeting domain linked to the antigen of interest or a domain that binds to the antigen of interest.
2. A molecule comprising a cell targeting domain linked to an antigen of interest or a domain that binds to the antigen of interest, wherein the cell targeting domain binds to a thymic cell.
3. A molecule comprising a cell targeting domain linked to an antigen of interest or a domain that binds to the antigen of interest, wherein the cell targeting domain binds to a thymic cell, a splenic cell, a dendritic cell, or a B cell.
4. A molecule for inducing tolerance against an antigen of interest, wherein the molecule comprises a cell targeting domain linked to the antigen of interest or a domain that binds to the antigen of interest, wherein the targeting domain binds to a thymic cell, a splenic cell, a dendritic cell, or a B cell.
5. A molecule for inducing antigen-specific Tregs against an antigen of interest, wherein the molecule comprises a cell targeting domain linked to the antigen of interest or a domain that binds to the antigen of interest, wherein the targeting domain binds to a thymic cell, a splenic cell, a dendritic cell, or a B cell.
6. A molecule for inducing antigen-specific Tregs against an antigen of interest, wherein the molecule comprises a cell targeting domain linked to the antigen of interest or a domain that binds to the antigen of interest, wherein the targeting domain binds to a thymic cell, a splenic cell, a dendritic cell, or a B cell, wherein the molecule does not significantly induce antigen-specific T_{eff} cells.
7. The molecule of any one of embodiments 1-6, wherein the cell targeting domain binds to Trop2, macrophage receptor MARCO, cadherin-1 (CDH1), Trop2, CD74, EpCAM, syndecan 1 (SDC1), PlgR, CD23 antigen (FCER2), CD166 antigen, OX-2 membrane glycoprotein, T-lymphocyte activation antigen CD86, RANK, CCL19, CCL21, LTβR, MHCII, CD40, CD80, CD86, lymphocyte antigen 6D, L1CAM, LIFR, integrin alpha-6, integrin beta-4, podoplanin, CCR7, CD24, CaSR, CD63, nAChRalpha1, CD70, acetylcholine receptor subunit delta, claudin-1, CD80, claudin-4, CD81, claudin-6, CD9, immunoglobulin superfamily member 1, large neutral amino acids transporter small subunit 2, cadherin-2, somatostatin receptor type 2, C-type lectin domain family 7 (CLEC7A), TRIC-A, HER3, IL-20RB, lymphocyte antigen 6D, DEC-205, LY6/PLAUR domain containing 3, oncostatin-M-specific receptor subunit beta, syndecan 1, syndecan-4, HAI-2, sialic acid-binding Ig-like lectin 10 (SIGLEC10), milk fat globule EGF and factor V/VIII domain containing (MFGE8), growth arrest-specific gene 6 (GAS6), or T-cell membrane protein 4 (TIM4).
8. The molecule of any one of embodiments 1-6, wherein the thymic cell is a medullary thymic epithelial cell (mTEC), a mimetic mTEC, an AIRE+ mTEC, an AIRE− mTEC, a mTEC progenitor cell, a mimetic mTEC progenitor cell, a junction thymic epithelial cell (jTEC), a thymic progenitor epithelial cell (TPEC), a proliferating TPEC, a thymic epithelial cell (TEC), a proliferating TEC, a cortical thymic epithelial cell (cTEC), a proliferating cTEC, or a proliferating mTEC.
9. The molecule of embodiment 8, wherein the mimetic mTEC is a basal (skin/lung) mTEC, an enterocyte/hepatocyte mTEC, a ciliated mTEC, an ionocyte mTEC, a keratinocyte mTEC, a microfold mTEC, a muscle mTEC, a neuroendocrine mTEC, a parathyroid mTEC, a secretory mTEC, a thyroid mTEC, or a tuft mTEC.
10. The molecule of embodiment 8, wherein the cell targeting domain binds to Trop2, macrophage receptor MARCO, cadherin-1 (CDH1), Trop2, CD74, EpCAM, syndecan 1 (SDC1), PlgR, CD23 antigen (FCER2), CD166 antigen, OX-2 membrane glycoprotein, T-lymphocyte activation antigen CD86, RANK, CCL19, CCL21, LTβR, MHCII, CD40, CD80, CD86, lymphocyte antigen 6D, L1CAM, LIFR, integrin alpha-6, integrin beta-4, podoplanin, CCR7, CD24, CaSR, CD63, nAChRalpha1, CD70, acetylcholine receptor subunit delta, claudin-1, CD80, claudin-4, CD81, claudin-6, CD9, immunoglobulin superfamily member 1, large neutral amino acids transporter small subunit 2, cadherin-2, somatostatin receptor type 2, C-type lectin domain family 7 (CLEC7A), TRIC-A, HER3, IL-20RB, lymphocyte antigen 6D, DEC-205, LY6/PLAUR domain containing 3, oncostatin-M-specific receptor subunit beta, syndecan 1, syndecan-4, HAI-2, sialic acid-binding Ig-like lectin 10 (SIGLEC10), milk fat globule EGF and factor V/VIII domain containing (MFGE8), growth arrest-specific gene 6 (GAS6), or T-cell membrane protein 4 (TIM4).
11. The molecule of embodiment 9, wherein the cell targeting domain binds to Trop2, macrophage receptor MARCO, cadherin-1 (CDH1), CD74, EpCAM, syndecan 1 (SDC1), PlgR, CD23 antigen (FCER2), CD166 antigen, OX-2 membrane glycoprotein, T-lymphocyte activation antigen CD86, RANK, CCL19, CCL21, LTβR, MHCII, CD40, CD80, CD86, lymphocyte antigen 6D, L1CAM, LIFR, integrin alpha-6, integrin beta-4, podoplanin, CCR7, CD24, CaSR, CD63, nAChRalpha1, CD70, acetylcholine receptor subunit delta, claudin-1, CD80, claudin-4, CD81, claudin-6, CD9, immunoglobulin superfamily member 1, large neutral amino acids transporter small subunit 2, cadherin-2, somatostatin receptor type 2, C-type lectin domain family 7 (CLEC7A), TRIC-A, HER3, IL-20RB, lymphocyte antigen 6D, DEC-205, LY6/PLAUR domain containing 3, oncostatin-M-specific receptor subunit beta, syndecan 1, syndecan-4, HAI-2, sialic acid-binding Ig-like lectin 10 (SIGLEC10), milk fat globule EGF and factor V/VIII domain containing (MFGE8), growth arrest-specific gene 6 (GAS6), or T-cell membrane protein 4 (TIM4).
12. The molecule of any one of embodiments 1-11, wherein the cell targeting domain is a polypeptide.
13. The molecule of embodiment 12, wherein the polypeptide is an antibody, or an antigen-binding fragment thereof.
14. The molecule of any one of embodiments 1-13, wherein the cell targeting domain binds to a cell membrane surface protein or glycoprotein on the surface of the thymic cell, splenic cell, dendritic cell, or B cell.
15. The molecule of embodiment 14, wherein the cell membrane surface protein is cadherin-1 (CDH1), Trop2, CD74, EpCAM, syndecan 1 (SDC1), PlgR, CD23 antigen (FCER2), macrophage receptor MARCO, C-type lectin domain family 7 (CLEC7A), CD24, CD70, sialic acid-binding Ig-like lectin 10 (SIGLEC10), milk fat globule EGF and factor V/VIII domain containing (MFGE8), EGF like repeats and discoidin domains 3 (EDIL3), growth arrest-specific gene 6 (GAS6), or T-cell membrane protein 4 (TIM4).
16. The molecule of any one of embodiments 1-15, wherein the cell targeting domain is an antibody that is: an anti-cadherin-1 (CDH1) antibody, or antigen-binding fragment thereof; an anti-Trop2 antibody, or antigen-binding fragment thereof; an anti-CD74 antibody, or antigen-binding fragment thereof; an anti-EpCAM antibody, or antigen-binding fragment thereof; an anti-syndecan 1 antibody, or antigen-binding fragment thereof; an anti-PlgR antibody, or antigen-binding fragment thereof; an anti-CD23 antigen (FCER2) antibody, or antigen-binding fragment thereof; an anti-macrophage receptor MARCO antibody, or antigen-binding fragment thereof; an anti-sialic acid-binding Ig-like lectin 10 (SIGLEC10) antibody, or antigen-binding fragment thereof; an anti-milk fat globule EGF and factor V/VIII domain containing (MFGE8) antibody, or antigen-binding fragment thereof; an anti-growth arrest-specific gene 6 (GAS6) antibody, or antigen-binding fragment thereof; an anti-T-cell membrane protein 4 (TIM4) antibody, or antigen-binding fragment thereof; an anti-CD166 antigen antibody, or antigen-binding fragment thereof; an anti-OX-2 membrane glycoprotein antibody, or antigen-binding fragment thereof; an anti-T-lymphocyte activation antigen CD86 antibody, or antigen-binding fragment thereof; an anti-RANK antibody, or antigen-binding fragment thereof; an anti-CCL19 antibody, or antigen-binding fragment thereof; an anti-CCL21 antibody, or antigen-binding fragment thereof; an anti-LTβR antibody, or antigen-binding fragment thereof; an anti-MHCII antibody, or antigen-binding fragment thereof; an anti-CD40 antibody, or antigen-binding fragment thereof; an anti-CD80 antibody, or antigen-binding fragment thereof; an anti-CD86 antibody, or antigen-binding fragment thereof; an anti-lymphocyte antigen 6D antibody, or antigen-binding fragment thereof; an anti-L1CAM antibody, or antigen-binding fragment thereof; an anti-LIFR antibody, or antigen-binding fragment thereof; an anti-integrin alpha-6 antibody, or antigen-binding fragment thereof; an anti-integrin beta-4 antibody, or antigen-binding fragment thereof; an anti-podoplanin antibody, or antigen-binding fragment thereof; an anti-CCR7 antibody, or antigen-binding fragment thereof; an anti-CD24 antibody, or antigen-binding fragment thereof; an anti-CaSR antibody, or antigen-binding fragment thereof; an anti-CD63 antibody, or antigen-binding fragment thereof; an anti-nAChRalpha1 antibody, or antigen-binding fragment thereof; an anti-CD70 antibody, or antigen-binding fragment thereof; an anti-acetylcholine receptor subunit delta antibody, or antigen-binding fragment thereof; an anti-claudin-1 antibody, or antigen-binding fragment thereof; an anti-CD80 antibody, or antigen-binding fragment thereof; an anti-claudin-4 antibody, or antigen-binding fragment thereof; an anti-CD81 antibody, or antigen-binding fragment thereof; an anti-claudin-6 antibody, or antigen-binding fragment thereof; an anti-CD9 antibody, or antigen-binding fragment thereof; an anti-immunoglobulin superfamily member 1 antibody, or antigen-binding fragment thereof; an anti-cadherin-1 antibody, or antigen-binding fragment thereof; an anti-large neutral amino acids transporter small subunit 2 antibody, or antigen-binding fragment thereof; an anti-cadherin-2 antibody, or antigen-binding fragment thereof; an anti-somatostatin receptor type 2 antibody, or antigen-binding fragment thereof; an anti-dectin-1 antibody, or antigen-binding fragment thereof; an anti-TRIC-A antibody, or antigen-binding fragment thereof; an anti-HER3 antibody, or antigen-binding fragment thereof; an anti-IL-20RB antibody, or antigen-binding fragment thereof; an anti-lymphocyte antigen 6D antibody, or antigen-binding fragment thereof; an anti-DEC-205 antibody, or antigen-binding fragment thereof; an anti-LY6/PLAUR domain containing 3 antibody, or antigen-binding fragment thereof; an anti-oncostatin-M-specific receptor subunit beta antibody, or antigen-binding fragment thereof; an anti-syndecan-4 antibody, or antigen-binding fragment thereof; an anti-HAI-2 antibody, or antigen-binding fragment thereof.
17. The molecule of any one of embodiments 1-16, wherein cell targeting domain is a ligand that is from CD6, cell surface glycoprotein CD200 receptor 1, P-selectin, sialic acid-binding Ig-like lectin 10, metalloproteinase inhibitor 1, lysosome-associated membrane glycoprotein 1, lysosome-associated membrane glycoprotein 2, integrin beta-1, CD27 antigen, macrophage migration inhibitory factor, CD44 antigen, cytotoxic T-lymphocyte protein 4, programmed cell death 1 ligand 1, T-cell-specific surface glycoprotein CD28, T-lymphocyte activation antigen CD86, complement receptor type 2, prostaglandin F2 receptor negative regulator, immunoglobulin superfamily member 8, integrin beta-1, B-lymphocyte antigen CD19, CD44 antigen, T-lymphocyte activation antigen CD80, T-cell-specific surface glycoprotein CD28, cytotoxic T-lymphocyte protein 4, membrane cofactor protein, proheparin-binding EGF-like growth factor, immunoglobulin superfamily member 8, prostaglandin F2 receptor negative regulator, integrin alpha-3, integrin alpha-6, integrin beta-1, epidermal growth factor receptor, integrin alpha-E, killer cell lectin-like receptor subfamily G member 1, hepatocyte growth factor receptor, cadherin-2, fibroblast growth factor receptor 1, low-density lipoprotein receptor-related protein 5, integrin beta-4, epithelial cell adhesion molecule, cadherin-1, pro-neuregulin-1, pro-neuregulin-2, complement receptor type 2, integrin alpha-X, integrin beta-2, neurogenic locus notch homolog protein 2, interleukin-19, interleukin-20, interleukin-24, receptor-type tyrosine-protein phosphatase C, anterior gradient protein 2 homolog, secretoglobin family 3A member 2, cardiotrophin-1, leukemia inhibitory factor, oncostatin-M, kappa-casein, fibroblast growth factor 2, extracellular glycoprotein lacritin, fibroblast growth factor 2, fibroblast growth factor 2, hepatocyte growth factor, hepatocyte growth factor activator, trypsin-3, prostasin, suppressor of tumorigenicity 14 protein, or transmembrane protease serine 13, or a liganding binding partner fragment thereof of any of the foregoing.
18. The molecule of any one of embodiments 1-17, wherein the antigen of interest is an antibody, or antigen-binding fragment thereof; an antigen, or a fragment thereof; an auto-antigen, or a fragment thereof; a self-antigen, or a fragment thereof; a tolerance inducing antigen, or a fragment thereof; or a ligand, or a fragment thereof.
19. The molecule of any one of embodiments 1-18, wherein the antigen of interest is an antigen that induces an autoimmune, alloimmune, allergic, inflammatory, or anti-drug immune response.
20. The molecule of embodiment 19, wherein the autoimmune, alloimmune, allergic, inflammatory, or anti-drug immune response is a disorder or disease selected from the group consisting of: diabetes mellitus type 1, ulcerative colitis, Crohn's disease, celiac disease, systemic sclerosis, graft versus host disease (GVHD), rheumatoid arthritis, anti-AAV immune response, asthma, severe asthma. neutrophilic asthma, paucigranulocytic asthma, eosinophilic asthma, osteoarthritis, solid organ transplant, atopic dermatitis, eosinophilic esophagitis, dermatitis herpitoformis, chronic rhinosinusitis, chronic spontaneous urticaria, food allergy, anti-biologic drug immune response, anti-viral immune response, anti-adenoviral immune response, anti-retroviral immune response, anti-lentiviral immune response, and -VLP immune response, Alzheimer's disease, amyotrophic lateral sclerosis (ALS), Parkinson's disease, dementia with Lewy bodies, Duchenne muscular dystrophy, cutaneous lupus myocarditis, postmyocardial infarction syndrome, postpericardiotomy syndrome, subacute bacterial endocarditis, anti-glomerular basement membrane nephritis, interstitial cystitis, lupus nephritis, membranous glomerulonephropathy, chronic kidney disease (“CKD”), autoimmune hepatitis, primary biliary cirrhosis, primary sclerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease (LAD), morphea, pemphigus, pemphigus vulgaris, hidradenitis suppuritiva, pityriasis lichenoides et varioliformis acuta, Mucha-Habermann disease, psoriasis, guttate psoriasis, erythrodermic psoriasis, plaque psoriasis, pustular psoriasis, skin manifestation associated with systemic sclerosis, vitiligo, Addison's disease, autoimmune polyendocrine syndrome (APS) type 1, autoimmune polyendocrine syndrome (APS) type 2, autoimmune polyendocrine syndrome (APS) type 3, autoimmune pancreatitis (AIP), autoimmune thyroiditis, Ord's thyroiditis, Graves' disease, non radiographic axial spondyloarthropathy, autoimmune oophoritis, endometriosis, autoimmune orchitis, Sjogren's syndrome, dermatitis herpatiformis, autoimmune enteropathy, coeliac disease, immune related adverse events (irAEs) secondary to immune checkpoint inhibitors, microscopic colitis, autoimmune thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, sarcoidosis, Schnitzler syndrome, systemic lupus erythematosus (SLE), undifferentiated connective tissue disease (UCTD), dermatomyositis, fibromyalgia, inclusion body myositis, myositis, myasthenia gravis, neuromyotonia, paraneoplastic cerebellar degeneration, polymyositis, acute disseminated encephalomyelitis (ADEM), acute motor axonal neuropathy, anti-N-methyl-D-aspartate (anti-NMDA) receptor encephalitis, balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, oshtoran syndrome, pediatric autoimmune neuropsychiatric disorder associated with streptococcus (PANDAS), progressive inflammatory neuropathy, restless leg syndrome, stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Mdniere's disease, Behcet's disease, eosinophilic granulomatosis with polyangiitis (EGPA), giant cell arteritis, granulmatosis with polyangiitis (GPA), IgA vasculitis (IgAV), Kawasaki's disease, leukocytoclastic vasculitis, lupus vasculitis, rheumatoid vasculitis, microscopic polyangiitis (MPA), polyarteritis nodosa (PAN), polymyalgia rheumaticia, vasculitis, primary immune deficiency, Gaucher disease, Pompe disease, Fabry disease, Schindler disease, Hurler syndrome (MPS1), Tay-Sachs disease, Nieman-Pick disease, Farber lipogranulomatosis, Krabbe disease, Morquio syndrome (MPS4), mucopolysaccharidosis type VI (MPS6), Hunter syndrome (MPS2), Sanfilippo syndrome (MPS3), Sly sundrome (MPS7), hyaluronidase deficiency (MPS9), mucolipidosis type 1 (ML1), I-cell disease, mucolipidosis II (ML2), mucolipidosis III gamma (ML3C), glycogen storage disease Ia, glycogen storage disease Ib, Wilson disease, phenylketonuria, Stargardt disease type I, myocilin-associated glaucoma, hemophilia A, hemophilia B, hereditary or acquired thrombotic thrombocytopathic purpura (TTP), von Willebrand disease, thrombocytopenia platelet autoantigen, heparin-induced thrombocytopenia, tolerance induction for AAV gene therapy, tolerance induction for ex vivo gene editing, tolerance induction for in vivo gene editing, acquired hemophilia, antiphospholipid syndrome, aplastic anemia, autoimmune lymphoproliferative syndrome, autoimmune neutropenia, autoimmune thrombocytopenic purpura, cold agglutinin disease, essential mixed cryoglobulinemia, Evans syndrome, IgG4-related diseases, pure red cell aplasia, warm autoimmune hemolytic anemia, autoimmune gastritis, pernicious anemia, anti-sperm antibodies, autoimmune polyendocrine syndrome type 1, autoimmune polyendocrine syndrome type 2, autoimmune polyendocrine syndrome type 3, autoimmune myocarditis, cryptogenic organizing pneumonia, idiopathic pulmonary fibrosis, IgA nephropathy, post-myocardial infarction syndrome, primary biliary cholangitis, primary idiopathic dilated cardiomyopathy, anti-N-methyl-D-aspartate receptor encephalitis, autoimmune encephalitis, encephalopathy associated with autoimmune thyroid disease, episcleritis, Graves' ophthalmopathy, myelin oligodendrocyte glycoprotein disease, narcolepsy with cataplexy, cutaneous lupus erythematosus, adult-onset Still's disease, ankylosing spondylitis, juvenile arthritis, juvenile dermatomyositis, anti-neutrophil cytoplasmic antibody-associated vasculitis, polymyalgia rheumatic, purpura rheumatic, Takayasu arteritis, transplant, and xenotransplantation.

21. The molecule of any one of embodiments 1-20, wherein the antigen of interest is selected from the group consisting of: glutamate decarboxylase 1 (GAD), glutamate decarboxylase 2 (GAD65), proinsulin, islet-specific glucose-6-phosphatase catalytic subunit-related protein (IGRP), zinc transporter 8 (Znt8), receptor-type tyrosine-protein phosphatase-like N (IA2), tropomyosin alpha-3 chain (TPM3), granulocyte-macrophage colony-stimulating factor (GMCSF/CSF2), RNA polymerase III complex subunit A (RNAP3), RNA polymerase III complex subunit B (RNAP3), RNA polymerase III complex subunit C (RNAP3), RNA polymerase III complex subunit D (RNAP3), RNA polymerase III complex subunit E (RNAP3), RNA polymerase III complex subunit F (RNAP3), RNA polymerase III complex subunit G (RNAP3), RNA polymerase III complex subunit H (RNAP3), RNA polymerase III complex subunit K (RNAP3), DNA topoisomerase 1 (TOPO1), type-1 angiotensin II receptor (AT1R), endothelin-1 receptor (ETAR), major centromere protein A (CENP), major centromere protein B (CENP), major centromere protein C (CENP), tetraspanin-7 (TSPAN7), 21-hydroxylase (21OH), 17alpha-hydroxylase (17OH), Th/To, ANA, dsDNA, HMGCR, myosin, SSA/Ro, SSB/La, U1RNP, U3RNP, PM-Sci, myelin basic protein (MBP), IL17/IL22, ASMA, actin, amylase α2, GM-CSF, cyclic citrullinated proteins (CCP), snRNP, Ro52, Ro60, La, Jo-1, SRP, IFIH1, CENPA, insulin, RNA-P, U1-70K, Sm-D3, MDA-5, PL7, PL-12, centromere proteins, PM/SCL, RNA polymerase 3 complex, tetraspanin-7, peptidylprolyl isomerase like 2, Mlh1, proteinase-3, immunoglobulin G, immunoglobulin M, myeloperoxidase, acetylcholine receptor (AchR), MUSK, LRP4, HSP90. HSPA5, kallikrein 13, ADAMTS13, IFN-7, IFN-α/ω, IL6, IL12/IL23, desmogleins (e.g., desmoglein-3, desmoglein-1), aquaporin-4, myelin oligodendrocyte glycoprotein (MOG), GRIN1, GRIA1, GRIA2, leucine rich glioma inactivated 1 protein (LGI1), neurexin family protein contactin associated protein 2 (CNTNAP2), collagen (e.g., collagen IV-alpha 3 chain (COL4A3), COL17), BP180, phospholipase A2 receptor (PLA2R), thrombospondin type-1 domain-containing 7A (THSD7A), thyroid hormone stimulating receptor (THSR), NMDAR, FGF23, acid beta-glycosidase, acid alpha-glycosidase, alpha-galactosidase A, alpha-N-acetylgalactosaminidase, alpha-L-iduronohydrolase, beta-hexosaminidase A, sphingomyelin phosphodiesterase 1, N-acetylsphingosine amidohydrolase 1, galactocerebrosidase, N-acetylgalactosamine-sulfate sulfatase, arylsulfatase B, iduronate 2-sulfatase, heparan-alpha-glucosaminide-N-acetyltransferase, beta-glucuronidase, hyaluronidase, sialidase 1, N-acetylglucosamine-1-phosphotransferase alpha/beta, N-acetylglucosamine-1-phosphotransferase gamma, glucose-6-phosphatase catalytic subunit, glucose-6-phosphate transporter, ATP7B, Phenylalanine hydroxylase, ATP-binding cassette subfamily A member 4, myocilin, coagulation factor VIII, coagulation factor IX, von Willebrand factor, glycoprotein Ib alpha, glycoprotein Ib beta, glycoprotein V, glycoprotein VI, glycoprotein IX, glycoprotein IIb, glycoprotein IIIa, platelet factor 4, AAV capsid protein (such as AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.eB, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9, AAV9 K449R (or K449R AAV9), AAV9.16, AAV9.24, AAV9.45, AAbiodisV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74 (also referred to as AAVrh74), AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, 7m8, Spark100, AAVMYO and variants thereof), SpCas9, SaCas9, FnCas12a, LbCas12a, AcCas12a, Cas13b, APOBEC1, E. coli TadA, Moloney Murine Leukemia Virus Reverse Transcriptase (M-MLV RT), HLA-A, HLA-B, HLA-C, HLA-E, HLA-L, HLA-J, HLA-K, HLA-H, HLA-G, HLA-DM, HLA-DO, HLA-DP, HLA-DQ, and HLA-DR.

22. The molecule of any one of 1-21, wherein the antigen of interest has an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence selected from any one of SEQ ID NO: 14, 15, 16, 17, 18, 19, 20, 21, 22, 23, 24, 25, 26, 27, 28, 29, 30, 31, 32, 33, 34, 35, 36, or 37.
23. The molecule of any one of embodiments 1-22, wherein the domain that binds to the antigen of interest is an antibody, or antigen-binding fragment thereof; in the format of a scFv, Fab, Fab′, F(ab′)2, and VHH antibody, or antigen-binding fragment thereof.
24. The molecule of embodiment 23, wherein the antibody, or antigen-binding fragment thereof, binds to the antigen, or the fragment thereof; the auto-antigen, or the fragment thereof; the self-antigen, or the fragment thereof; the tolerance inducing antigen, or the fragment thereof, is selected from glutamate decarboxylase 1 (GAD), glutamate decarboxylase 2 (GAD65), proinsulin, islet-specific glucose-6-phosphatase catalytic subunit-related protein (IGRP), zinc transporter 8 (Znt8), receptor-type tyrosine-protein phosphatase-like N (IA2), tropomyosin alpha-3 chain (TPM3), granulocyte-macrophage colony-stimulating factor (GMCSF/CSF2), RNA polymerase III complex subunit A (RNAP3), RNA polymerase III complex subunit B (RNAP3), RNA polymerase III complex subunit C (RNAP3), RNA polymerase III complex subunit D (RNAP3), RNA polymerase III complex subunit E (RNAP3), RNA polymerase III complex subunit F (RNAP3), RNA polymerase III complex subunit G (RNAP3), RNA polymerase III complex subunit H (RNAP3), RNA polymerase III complex subunit K (RNAP3), DNA topoisomerase 1 (TOPO1), type-1 angiotensin II receptor (ATIR), endothelin-1 receptor (ETAR), major centromere protein A (CENP), major centromere protein B (CENP), major centromere protein C (CENP), tetraspanin-7 (TSPAN7), 21-hydroxylase (21OH), 17alpha-hydroxylase (17OH), Th/To, ANA, dsDNA, HMGCR, myosin, SSA/Ro, SSB/La, U1RNP, U3RNP, PM-Sci, myelin basic protein (MBP), IL17/IL22, ASMA, actin, amylase α2, GM-CSF, cyclic citrullinated proteins (CCP), snRNP, Ro52, Ro60, La, Jo-1, SRP, IFIH1, CENPA, insulin, RNA-P, U1-70K, Sm-D3, MDA-5, PL7, PL-12, centromere proteins, PM/SCL, RNA polymerase 3 complex, tetraspanin-7, peptidylprolyl isomerase like 2, Mlh1, proteinase-3, immunoglobulin G, immunoglobulin M, myeloperoxidase, acetylcholine receptor (AchR), MUSK, LRP4, HSP90. HSPA5, kallikrein 13, ADAMTS13, IFN-7, IFN-α/ω, IL6, IL12/IL23, desmogleins (e.g., desmoglein-3, desmoglein-1), aquaporin-4, myelin oligodendrocyte glycoprotein (MOG), GRIN1, GRIA1, GRIA2, leucine rich glioma inactivated 1 protein (LGI1), neurexin family protein contactin associated protein 2 (CNTNAP2), collagen (e.g., collagen IV-alpha 3 chain (COL4A3), COL17), BP180, phospholipase A2 receptor (PLA2R), thrombospondin type-1 domain-containing 7A (THSD7A), thyroid hormone stimulating receptor (THSR), NMDAR, FGF23, acid beta-glycosidase, acid alpha-glycosidase, alpha-galactosidase A, alpha-N-acetylgalactosaminidase, alpha-L-iduronohydrolase, beta-hexosaminidase A, sphingomyelin phosphodiesterase 1, N-acetylsphingosine amidohydrolase 1, galactocerebrosidase, N-acetylgalactosamine-sulfate sulfatase, arylsulfatase B, iduronate 2-sulfatase, heparan-alpha-glucosaminide-N-acetyltransferase, beta-glucuronidase, hyaluronidase, sialidase 1, N-acetylglucosamine-1-phosphotransferase alpha/beta, N-acetylglucosamine-1-phosphotransferase gamma, glucose-6-phosphatase catalytic subunit, glucose-6-phosphate transporter, ATP7B, Phenylalanine hydroxylase, ATP-binding cassette subfamily A member 4, myocilin, coagulation factor VIII, coagulation factor IX, von Willebrand factor, glycoprotein Ib alpha, glycoprotein Ib beta, glycoprotein V, glycoprotein VI, glycoprotein IX, glycoprotein IIb, glycoprotein IIIa, platelet factor 4, AAV capsid protein (such as AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.eB, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9, AAV9 K449R (or K449R AAV9), AAV9.16, AAV9.24, AAV9.45, AAbiodisV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74 (also referred to as AAVrh74), AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, 7m8, Spark100, AAVMYO and variants thereof), SpCas9, SaCas9, FnCas12a, LbCas12a, AcCas12a, Cas13b, APOBEC1, E. coli TadA, Moloney Murine Leukemia Virus Reverse Transcriptase (M-MLV RT), HLA-A, HLA-B, HLA-C, HLA-E, HLA-L, HLA-J, HLA-K, HLA-H, HLA-G, HLA-DM, HLA-DO, HLA-DP, HLA-DQ, and HLA-DR.
25. The molecule of any one of embodiments 21-24, wherein the antigen of interest has an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence selected from any one of SEQ ID NO: 14, 15, 16, 17, 18, 19, 20, 21, 22, 23, 24, 25, 26, 27, 28, 29, 30, 31, 32, 33, 34, 35, 36, or 37.
26. The molecule of any one of embodiments 1-17, wherein the antigen of interest is an effector peptide or molecule.
27. The molecule of embodiment 26, wherein the effector peptide or molecule is capable of eliciting an autoimmune response in a subject.
28. The molecule of embodiments 26 or 27, wherein the effector peptide or molecule is an allergen.
29. The molecule of any one of embodiments 1-28, wherein the molecule further comprises an Fc polypeptide domain.
30. The molecule of embodiment 29, wherein the Fc polypeptide comprises an IgG1, IgG2, IgG3, IgG4 Fc polypeptide domain comprising any one, or any combination of L234A, L235A, L234F, L235E, P329G, P331S, N297A, N297G, N297Q, G236A, A330S, S239D, I332E, S267E, H268F, S324T, Y296W, T299A, V308P, H310A, R409K, Y435H, T307A, T309A, T309K, K322A, K326W, K334W, K326A, K334A, G237A, P238S, and H268A mutations, wherein the positions are according to EU numbering.
31. The molecule of embodiments 29 or 30, wherein the Fc polypeptide domain is effectorless.
32. The molecule of any one of embodiments 29-31, wherein the Fc polypeptide domain does not bind, or has reduced affinity, to FcRn.
33. The molecule of any one of embodiments 29-32, wherein the Fc polypeptide domain is effectorless, and wherein the Fc polypeptide domain does not bind, or has reduced affinity, to FcRn.
34. The molecule of any one of embodiments 29-33, wherein the Fc polypeptide domain enhances presentation of the antigen of interest on the surface of a cell.
35. The molecule of embodiment 34, wherein the cell is a thymic cell.
36. The molecule of any one of embodiments 1-28, wherein the molecule does not comprise an Fc polypeptide domain.
37. The molecule of any one of embodiments 1-36, wherein the cell targeting domain is conjugated to the N-terminus of a Fc polypeptide domain, and the antigen of interest, or a domain that binds to the antigen of interest, is conjugated to a C-terminus of the Fc polypeptide domain.
38. The molecule of any one of embodiments 1-36, wherein the cell targeting domain is conjugated to the C-terminus of a Fc polypeptide domain, and the antigen of interest, or a domain that binds to the antigen of interest, is conjugated to the N-terminus of the Fc polypeptide domain.
39. The molecule of any one of embodiments 1-38, wherein the cell targeting domain and the antigen of interest, or the domain that binds to the antigen of interest, are linked by a linker.
40. The molecule of embodiment 39, wherein the linker is a peptide linker.
41. The molecule of any one of embodiments 1-40, wherein the cell targeting domain and the antigen of interest, or the domain that binds to the antigen of interest, are directly conjugated.
42. A molecule comprising:
a) a cell targeting domain that binds to cadherin-1 (CDH1), Trop2, CD74, EpCAM, syndecan 1 (SDC1), PlgR, CD23 antigen (FCER2), macrophage receptor MARCO, C-type lectin domain family 7 (CLEC7A), CD24, CD70, sialic acid-binding Ig-like lectin 10 (SIGLEC10), milk fat globule EGF and factor V/VIII domain containing (MFGE8), EGF like repeats and discoidin domains 3 (EDIL3), growth arrest-specific gene 6 (GAS6), T-cell membrane protein 4 (TIM4);
b) an autoimmune, alloimmune, allergic, inflammatory, or anti-drug immune response associated antigen of interest; and
c) optionally an Fc region,
wherein the cell targeting domain binds to a thymic cell, a splenic cell, a dendritic cell, or a B cell.
   43. The molecule of embodiment 42, wherein the thymic cell is a medullary thymic epithelial cell (mTEC), a mimetic mTEC, an AIRE+ mTEC, an AIRE− mTEC, a mTEC progenitor cell, a mimetic mTEC progenitor cell, a junction thymic epithelial cell (jTEC), a thymic progenitor epithelial cell (TPEC), a proliferating TPEC, a thymic epithelial cell (TEC), a proliferating TEC, a cortical thymic epithelial cell (cTEC), a proliferating cTEC, or a proliferating mTEC.
   44. The molecule of embodiment 43, wherein the mimetic mTEC is a basal (skin/lung) mTEC, an enterocyte/hepatocyte mTEC, a ciliated mTEC, an ionocyte mTEC, a keratinocyte mTEC, a microfold mTEC, a muscle mTEC, a neuroendocrine mTEC, a parathyroid mTEC, a secretory mTEC, a thyroid mTEC, or a tuft mTEC.
   45. The molecule of any one of embodiments 42-44, wherein the cell targeting domain is a polypeptide.
   46. The molecule of any one of embodiments 42-45, wherein the cell targeting domain binds to a cell membrane surface protein or glycoprotein on the surface of the thymic cell, splenic cell, dendritic cell, or B cell.
   47. The molecule of any one of embodiments 42-46, wherein the cell targeting domain is an antibody that is: an anti-cadherin-1 (CDH1) antibody, or antigen-binding fragment thereof; an anti-Trop2 antibody, or antigen-binding fragment thereof; an anti-CD74 antibody, or antigen-binding fragment thereof; an anti-EpCAM antibody, or antigen-binding fragment thereof; an anti-syndecan 1 antibody, or antigen-binding fragment thereof; an anti-PlgR antibody, or antigen-binding fragment thereof; an anti-CD23 antigen (FCER2) antibody, or antigen-binding fragment thereof; an anti-macrophage receptor MARCO antibody, or antigen-binding fragment thereof; an anti-sialic acid-binding Ig-like lectin 10 (SIGLEC10) antibody, or antigen-binding fragment thereof; an anti-milk fat globule EGF and factor V/VIII domain containing (MFGE8) antibody, or antigen-binding fragment thereof; an anti-growth arrest-specific gene 6 (GAS6) antibody, or antigen-binding fragment thereof; an anti-T-cell membrane protein 4 (TIM4) antibody, or antigen-binding fragment thereof; an anti-CD166 antigen antibody, or antigen-binding fragment thereof; an anti-OX-2 membrane glycoprotein antibody, or antigen-binding fragment thereof; an anti-T-lymphocyte activation antigen CD86 antibody, or antigen-binding fragment thereof; an anti-RANK antibody, or antigen-binding fragment thereof; an anti-CCL19 antibody, or antigen-binding fragment thereof; an anti-CCL21 antibody, or antigen-binding fragment thereof; an anti-LTβR antibody, or antigen-binding fragment thereof; an anti-MHCII antibody, or antigen-binding fragment thereof; an anti-CD40 antibody, or antigen-binding fragment thereof; an anti-CD80 antibody, or antigen-binding fragment thereof; an anti-CD86 antibody, or antigen-binding fragment thereof; an anti-lymphocyte antigen 6D antibody, or antigen-binding fragment thereof; an anti-L1CAM antibody, or antigen-binding fragment thereof; an anti-LIFR antibody, or antigen-binding fragment thereof; an anti-integrin alpha-6 antibody, or antigen-binding fragment thereof; an anti-integrin beta-4 antibody, or antigen-binding fragment thereof; an anti-podoplanin antibody, or antigen-binding fragment thereof; an anti-CCR7 antibody, or antigen-binding fragment thereof; an anti-CD24 antibody, or antigen-binding fragment thereof; an anti-CaSR antibody, or antigen-binding fragment thereof; an anti-CD63 antibody, or antigen-binding fragment thereof; an anti-nAChRalpha1 antibody, or antigen-binding fragment thereof; an anti-CD70 antibody, or antigen-binding fragment thereof; an anti-acetylcholine receptor subunit delta antibody, or antigen-binding fragment thereof; an anti-claudin-1 antibody, or antigen-binding fragment thereof; an anti-CD80 antibody, or antigen-binding fragment thereof; an anti-claudin-4 antibody, or antigen-binding fragment thereof; an anti-CD81 antibody, or antigen-binding fragment thereof; an anti-claudin-6 antibody, or antigen-binding fragment thereof; an anti-CD9 antibody, or antigen-binding fragment thereof; an anti-immunoglobulin superfamily member 1 antibody, or antigen-binding fragment thereof; an anti-cadherin-1 antibody, or antigen-binding fragment thereof; an anti-large neutral amino acids transporter small subunit 2 antibody, or antigen-binding fragment thereof; an anti-cadherin-2 antibody, or antigen-binding fragment thereof; an anti-somatostatin receptor type 2 antibody, or antigen-binding fragment thereof; an anti-dectin-1 antibody, or antigen-binding fragment thereof; an anti-TRIC-A antibody, or antigen-binding fragment thereof; an anti-HER3 antibody, or antigen-binding fragment thereof; an anti-IL-20RB antibody, or antigen-binding fragment thereof; an anti-lymphocyte antigen 6D antibody, or antigen-binding fragment thereof; an anti-DEC-205 antibody, or antigen-binding fragment thereof; an anti-LY6/PLAUR domain containing 3 antibody, or antigen-binding fragment thereof; an anti-oncostatin-M-specific receptor subunit beta antibody, or antigen-binding fragment thereof; an anti-syndecan-4 antibody, or antigen-binding fragment thereof; an anti-HAI-2 antibody, or antigen-binding fragment thereof; or any combination thereof.
   48. The molecule of any one of embodiments 42-46, wherein the cell targeting domain is a ligand, or fragment thereof, is selected from CD6, cell surface glycoprotein CD200 receptor 1, P-selectin, sialic acid-binding Ig-like lectin 10, metalloproteinase inhibitor 1, lysosome-associated membrane glycoprotein 1, lysosome-associated membrane glycoprotein 2, integrin beta-1, CD27 antigen, macrophage migration inhibitory factor, CD44 antigen, cytotoxic T-lymphocyte protein 4, programmed cell death 1 ligand 1, T-cell-specific surface glycoprotein CD28, T-lymphocyte activation antigen CD86, complement receptor type 2, prostaglandin F2 receptor negative regulator, immunoglobulin superfamily member 8, integrin beta-1, B-lymphocyte antigen CD19, CD44 antigen, T-lymphocyte activation antigen CD80, T-cell-specific surface glycoprotein CD28, cytotoxic T-lymphocyte protein 4, membrane cofactor protein, proheparin-binding EGF-like growth factor, immunoglobulin superfamily member 8, prostaglandin F2 receptor negative regulator, integrin alpha-3, integrin alpha-6, integrin beta-1, epidermal growth factor receptor, integrin alpha-E, killer cell lectin-like receptor subfamily G member 1, hepatocyte growth factor receptor, cadherin-2, fibroblast growth factor receptor 1, low-density lipoprotein receptor-related protein 5, integrin beta-4, epithelial cell adhesion molecule, cadherin-1, pro-neuregulin-1, pro-neuregulin-2, complement receptor type 2, integrin alpha-X, integrin beta-2, neurogenic locus notch homolog protein 2, interleukin-19, interleukin-20, interleukin-24, receptor-type tyrosine-protein phosphatase C, anterior gradient protein 2 homolog, secretoglobin family 3A member 2, cardiotrophin-1, leukemia inhibitory factor, oncostatin-M, kappa-casein, fibroblast growth factor 2, extracellular glycoprotein lacritin, fibroblast growth factor 2, fibroblast growth factor 2, hepatocyte growth factor, hepatocyte growth factor activator, trypsin-3, prostasin, suppressor of tumorigenicity 14 protein, transmembrane protease serine 13, and any combination thereof.
   49. The molecule of any one of embodiments 42-48, wherein the antigen of interest is an antibody, or antigen-binding fragment thereof; an antigen, or a fragment thereof; an auto-antigen, or a fragment thereof; a self-antigen, or a fragment thereof; a tolerance inducing antigen, or a fragment thereof; or a ligand, or a fragment thereof.
   50. The molecule of embodiment 49, wherein the autoimmune, alloimmune, allergic, inflammatory, or anti-drug immune response is elicited by a disorder or disease selected from the group consisting of: diabetes mellitus type 1, ulcerative colitis, Crohn's disease, celiac disease, systemic sclerosis, graft versus host disease (GVHD), rheumatoid arthritis, anti-AAV immune response, asthma, severe asthma. neutrophilic asthma, paucigranulocytic asthma, eosinophilic asthma, osteoarthritis, solid organ transplant, atopic dermatitis, eosinophilic esophagitis, dermatitis herpitoformis, chronic rhinosinusitis, chronic spontaneous urticaria, food allergy, anti-biologic drug immune response, anti-viral immune response, anti-adenoviral immune response, anti-retroviral immune response, anti-lentiviral immune response, and -VLP immune response, Alzheimer's disease, amyotrophic lateral sclerosis (ALS), Parkinson's disease, dementia with Lewy bodies, Duchenne muscular dystrophy, cutaneous lupus myocarditis, postmyocardial infarction syndrome, postpericardiotomy syndrome, subacute bacterial endocarditis, anti-glomerular basement membrane nephritis, interstitial cystitis, lupus nephritis, membranous glomerulonephropathy, chronic kidney disease (“CKD”), autoimmune hepatitis, primary biliary cirrhosis, primary sclerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease (LAD), morphea, pemphigus, pemphigus vulgaris, hidradenitis suppuritiva, pityriasis lichenoides et varioliformis acuta, Mucha-Habermann disease, psoriasis, guttate psoriasis, erythrodermic psoriasis, plaque psoriasis, pustular psoriasis, skin manifestation associated with systemic sclerosis, vitiligo, Addison's disease, autoimmune polyendocrine syndrome (APS) type 1, autoimmune polyendocrine syndrome (APS) type 2, autoimmune polyendocrine syndrome (APS) type 3, autoimmune pancreatitis (AIP), autoimmune thyroiditis, Ord's thyroiditis, Graves' disease, non radiographic axial spondyloarthropathy, autoimmune oophoritis, endometriosis, autoimmune orchitis, Sjogren's syndrome, dermatitis herpatiformis, autoimmune enteropathy, coeliac disease, immune related adverse events (irAEs) secondary to immune checkpoint inhibitors, microscopic colitis, autoimmune thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, sarcoidosis, Schnitzler syndrome, systemic lupus erythematosus (SLE), undifferentiated connective tissue disease (UCTD), dermatomyositis, fibromyalgia, inclusion body myositis, myositis, myasthenia gravis, neuromyotonia, paraneoplastic cerebellar degeneration, polymyositis, acute disseminated encephalomyelitis (ADEM), acute motor axonal neuropathy, anti-N-methyl-D-aspartate (anti-NMDA) receptor encephalitis, balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, oshtoran syndrome, pediatric autoimmune neuropsychiatric disorder associated with streptococcus (PANDAS), progressive inflammatory neuropathy, restless leg syndrome, stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Mdniere's disease, Behcet's disease, eosinophilic granulomatosis with polyangiitis (EGPA), giant cell arteritis, granulmatosis with polyangiitis (GPA), IgA vasculitis (IgAV), Kawasaki's disease, leukocytoclastic vasculitis, lupus vasculitis, rheumatoid vasculitis, microscopic polyangiitis (MPA), polyarteritis nodosa (PAN), polymyalgia rheumaticia, vasculitis, primary immune deficiency, Gaucher disease, Pompe disease, Fabry disease, Schindler disease, Hurler syndrome (MPS1), Tay-Sachs disease, Nieman-Pick disease, Farber lipogranulomatosis, Krabbe disease, Morquio syndrome (MPS4), mucopolysaccharidosis type VI (MPS6), Hunter syndrome (MPS2), Sanfilippo syndrome (MPS3), Sly sundrome (MPS7), hyaluronidase deficiency (MPS9), mucolipidosis type 1 (ML1), I-cell disease, mucolipidosis II (ML2), mucolipidosis III gamma (ML3C), glycogen storage disease Ia, glycogen storage disease Ib, Wilson disease, phenylketonuria, Stargardt disease type I, myocilin-associated glaucoma, hemophilia A, hemophilia B, hereditary or acquired thrombotic thrombocytopathic purpura (TTP), von Willebrand disease, thrombocytopenia platelet autoantigen, heparin-induced thrombocytopenia, tolerance induction for AAV gene therapy, tolerance induction for ex vivo gene editing, tolerance induction for in vivo gene editing, acquired hemophilia, antiphospholipid syndrome, aplastic anemia, autoimmune lymphoproliferative syndrome, autoimmune neutropenia, autoimmune thrombocytopenic purpura, cold agglutinin disease, essential mixed cryoglobulinemia, Evans syndrome, IgG4-related diseases, pure red cell aplasia, warm autoimmune hemolytic anemia, autoimmune gastritis, pernicious anemia, anti-sperm antibodies, autoimmune polyendocrine syndrome type 1, autoimmune polyendocrine syndrome type 2, autoimmune polyendocrine syndrome type 3, autoimmune myocarditis, cryptogenic organizing pneumonia, idiopathic pulmonary fibrosis, IgA nephropathy, post-myocardial infarction syndrome, primary biliary cholangitis, primary idiopathic dilated cardiomyopathy, anti-N-methyl-D-aspartate receptor encephalitis, autoimmune encephalitis, encephalopathy associated with autoimmune thyroid disease, episcleritis, Graves' ophthalmopathy, myelin oligodendrocyte glycoprotein disease, narcolepsy with cataplexy, cutaneous lupus erythematosus, adult-onset Still's disease, ankylosing spondylitis, juvenile arthritis, juvenile dermatomyositis, anti-neutrophil cytoplasmic antibody-associated vasculitis, polymyalgia rheumatic, purpura rheumatic, Takayasu arteritis, transplant, and xenotransplantation.
   51. The molecule of embodiment 50, wherein the antigen of interest is selected from the group consisting of: glutamate decarboxylase 1 (GAD), glutamate decarboxylase 2 (GAD65), proinsulin, islet-specific glucose-6-phosphatase catalytic subunit-related protein (IGRP), zinc transporter 8 (Znt8), receptor-type tyrosine-protein phosphatase-like N (IA2), tropomyosin alpha-3 chain (TPM3), granulocyte-macrophage colony-stimulating factor (GMCSF/CSF2), RNA polymerase III complex subunit A (RNAP3), RNA polymerase III complex subunit B (RNAP3), RNA polymerase III complex subunit C (RNAP3), RNA polymerase III complex subunit D (RNAP3), RNA polymerase III complex subunit E (RNAP3), RNA polymerase III complex subunit F (RNAP3), RNA polymerase III complex subunit G (RNAP3), RNA polymerase III complex subunit H (RNAP3), RNA polymerase III complex subunit K (RNAP3), DNA topoisomerase 1 (TOPO1), type-1 angiotensin II receptor (AT1R), endothelin-1 receptor (ETAR), major centromere protein A (CENP), major centromere protein B (CENP), major centromere protein C (CENP), tetraspanin-7 (TSPAN7), 21-hydroxylase (21OH), 17alpha-hydroxylase (17OH), Th/To, ANA, dsDNA, HMGCR, myosin, SSA/Ro, SSB/La, U1RNP, U3RNP, PM-Sci, myelin basic protein (MBP), IL17/IL22, ASMA, actin, amylase α2, GM-CSF, cyclic citrullinated proteins (CCP), snRNP, Ro52, Ro60, La, Jo-1, SRP, IFIH1, CENPA, insulin, RNA-P, U1-70K, Sm-D3, MDA-5, PL7, PL-12, centromere proteins, PM/SCL, RNA polymerase 3 complex, tetraspanin-7, peptidylprolyl isomerase like 2, Mlh1, proteinase-3, immunoglobulin G, immunoglobulin M, myeloperoxidase, acetylcholine receptor (AchR), MUSK, LRP4, HSP90. HSPA5, kallikrein 13, ADAMTS13, IFN-7, IFN-α/ω, IL6, IL12/IL23, desmogleins (e.g., desmoglein-3, desmoglein-1), aquaporin-4, myelin oligodendrocyte glycoprotein (MOG), GRIN1, GRIA1, GRIA2, leucine rich glioma inactivated 1 protein (LGI1), neurexin family protein contactin associated protein 2 (CNTNAP2), collagen (e.g., collagen IV-alpha 3 chain (COL4A3), COL17), BP180, phospholipase A2 receptor (PLA2R), thrombospondin type-1 domain-containing 7A (THSD7A), thyroid hormone stimulating receptor (THSR), NMDAR, FGF23, acid beta-glycosidase, acid alpha-glycosidase, alpha-galactosidase A, alpha-N-acetylgalactosaminidase, alpha-L-iduronohydrolase, beta-hexosaminidase A, sphingomyelin phosphodiesterase 1, N-acetylsphingosine amidohydrolase 1, galactocerebrosidase, N-acetylgalactosamine-sulfate sulfatase, arylsulfatase B, iduronate 2-sulfatase, heparan-alpha-glucosaminide-N-acetyltransferase, beta-glucuronidase, hyaluronidase, sialidase 1, N-acetylglucosamine-1-phosphotransferase alpha/beta, N-acetylglucosamine-1-phosphotransferase gamma, glucose-6-phosphatase catalytic subunit, glucose-6-phosphate transporter, ATP7B, Phenylalanine hydroxylase, ATP-binding cassette subfamily A member 4, myocilin, coagulation factor VIII, coagulation factor IX, von Willebrand factor, glycoprotein Ib alpha, glycoprotein Ib beta, glycoprotein V, glycoprotein VI, glycoprotein IX, glycoprotein IIb, glycoprotein IIIa, platelet factor 4, AAV capsid protein (such as AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.eB, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9, AAV9 K449R (or K449R AAV9), AAV9.16, AAV9.24, AAV9.45, AAbiodisV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74 (also referred to as AAVrh74), AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, 7m8, Spark100, AAVMYO and variants thereof), SpCas9, SaCas9, FnCas12a, LbCas12a, AcCas12a, Cas13b, APOBEC1, E. coli TadA, Moloney Murine Leukemia Virus Reverse Transcriptase (M-MLV RT), HLA-A, HLA-B, HLA-C, HLA-E, HLA-L, HLA-J, HLA-K, HLA-H, HLA-G, HLA-DM, HLA-DO, HLA-DP, HLA-DQ, and HLA-DR.
   52. The molecule of any one of embodiments 49-51, wherein the antigen of interest has an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence selected from any one of SEQ ID NO: 14, 15, 16, 17, 18, 19, 20, 21, 22, 23, 24, 25, 26, 27, 28, 29, 30, 31, 32, 33, 34, 35, 36, or 37.
   53. The molecule of any one of embodiments 42-52, wherein the antigen of interest is an antibody, or antigen-binding fragment thereof; in the format of a scFv, Fab, Fab′, F(ab′)2, and VHH antibody, or antigen-binding fragment thereof.
   54. The molecule of embodiment 53, wherein the antibody, or antigen-binding fragment thereof, binds to the antigen, or the fragment thereof; the auto-antigen, or the fragment thereof; the self-antigen, or the fragment thereof; the tolerance inducing antigen, or the fragment thereof, is selected from glutamate decarboxylase 1 (GAD), glutamate decarboxylase 2 (GAD65), proinsulin, islet-specific glucose-6-phosphatase catalytic subunit-related protein (IGRP), zinc transporter 8 (Znt8), receptor-type tyrosine-protein phosphatase-like N (IA2), tropomyosin alpha-3 chain (TPM3), granulocyte-macrophage colony-stimulating factor (GMCSF/CSF2), RNA polymerase III complex subunit A (RNAP3), RNA polymerase III complex subunit B (RNAP3), RNA polymerase III complex subunit C (RNAP3), RNA polymerase III complex subunit D (RNAP3), RNA polymerase III complex subunit E (RNAP3), RNA polymerase III complex subunit F (RNAP3), RNA polymerase III complex subunit G (RNAP3), RNA polymerase III complex subunit H (RNAP3), RNA polymerase III complex subunit K (RNAP3), DNA topoisomerase 1 (TOPO1), type-1 angiotensin II receptor (AT1R), endothelin-1 receptor (ETAR), major centromere protein A (CENP), major centromere protein B (CENP), major centromere protein C (CENP), tetraspanin-7 (TSPAN7), 21-hydroxylase (21OH), 17alpha-hydroxylase (17OH), Th/To, ANA, dsDNA, HMGCR, myosin, SSA/Ro, SSB/La, U1RNP, U3RNP, PM-Sci, myelin basic protein (MBP), IL17/IL22, ASMA, actin, amylase α2, GM-CSF, cyclic citrullinated proteins (CCP), snRNP, Ro52, Ro60, La, Jo-1, SRP, IFIH1, CENPA, insulin, RNA-P, U1-70K, Sm-D3, MDA-5, PL7, PL-12, centromere proteins, PM/SCL, RNA polymerase 3 complex, tetraspanin-7, peptidylprolyl isomerase like 2, Mlh1, proteinase-3, immunoglobulin G, immunoglobulin M, myeloperoxidase, acetylcholine receptor (AchR), MUSK, LRP4, HSP90. HSPA5, kallikrein 13, ADAMTS13, IFN-7, IFN-α/ω, IL6, IL12/IL23, desmogleins (e.g., desmoglein-3, desmoglein-1), aquaporin-4, myelin oligodendrocyte glycoprotein (MOG), GRIN1, GRIA1, GRIA2, leucine rich glioma inactivated 1 protein (LGI1), neurexin family protein contactin associated protein 2 (CNTNAP2), collagen (e.g., collagen IV-alpha 3 chain (COL4A3), COL17), BP180, phospholipase A2 receptor (PLA2R), thrombospondin type-1 domain-containing 7A (THSD7A), thyroid hormone stimulating receptor (THSR), NMDAR, FGF23, acid beta-glycosidase, acid alpha-glycosidase, alpha-galactosidase A, alpha-N-acetylgalactosaminidase, alpha-L-iduronohydrolase, beta-hexosaminidase A, sphingomyelin phosphodiesterase 1, N-acetylsphingosine amidohydrolase 1, galactocerebrosidase, N-acetylgalactosamine-sulfate sulfatase, arylsulfatase B, iduronate 2-sulfatase, heparan-alpha-glucosaminide-N-acetyltransferase, beta-glucuronidase, hyaluronidase, sialidase 1, N-acetylglucosamine-1-phosphotransferase alpha/beta, N-acetylglucosamine-1-phosphotransferase gamma, glucose-6-phosphatase catalytic subunit, glucose-6-phosphate transporter, ATP7B, Phenylalanine hydroxylase, ATP-binding cassette subfamily A member 4, myocilin, coagulation factor VIII, coagulation factor IX, von Willebrand factor, glycoprotein Ib alpha, glycoprotein Ib beta, glycoprotein V, glycoprotein VI, glycoprotein IX, glycoprotein IIb, glycoprotein IIIa, platelet factor 4, AAV capsid protein (such as AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.eB, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9, AAV9 K449R (or K449R AAV9), AAV9.16, AAV9.24, AAV9.45, AAbiodisV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74 (also referred to as AAVrh74), AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, 7m8, Spark100, AAVMYO and variants thereof), SpCas9, SaCas9, FnCas12a, LbCas12a, AcCas12a, Cas13b, APOBEC1, E. coli TadA, Moloney Murine Leukemia Virus Reverse Transcriptase (M-MLV RT), HLA-A, HLA-B, HLA-C, HLA-E, HLA-L, HLA-J, HLA-K, HLA-H, HLA-G, HLA-DM, HLA-DO, HLA-DP, HLA-DQ, and HLA-DR.
   55. The molecule of any one of embodiments 53-54, wherein the antigen of interest has an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence selected from any one of SEQ ID NO: 14, 15, 16, 17, 18, 19, 20, 21, 22, 23, 24, 25, 26, 27, 28, 29, 30, 31, 32, 33, 34, 35, 36, or 37.
   56. The molecule of embodiment 42, wherein the antigen of interest is an effector peptide or molecule.
   57. The molecule of embodiment 56, wherein the effector peptide or molecule is capable of eliciting an autoimmune response in a subject.
   58. The molecule of any one of embodiments 56-57, wherein the effector peptide or molecule is an allergen.
   59. The molecule of any one of embodiments 42-58, wherein the molecule further comprises an Fc polypeptide domain.
   60. The molecule of embodiment 59, wherein the Fc polypeptide comprises any one, or any combination of L234A, L235A, L234F, L235E, P329G, P331S, N297A, N297G, N297Q, G236A, A330S, S239D, I332E, S267E, H268F, S324T, Y296W, T299A, V308P, H310A, R409K, Y435H, T307A, T309A, T309K, K322A, K326W, K334W, K326A, K334A, G237A, P238S, and H268A mutations.
   61. The molecule of any one of embodiments 59-60, wherein the Fc polypeptide is an effectorless Fc region.
   62. The molecule of any one of embodiments 59-61, wherein the Fc polypeptide does not bind, or has reduced affinity, to FcRn.
   63. The molecule of any one of embodiments 59-62, wherein the Fc polypeptide is an effectorless Fc region, and wherein the Fc region does not bind, or has reduced affinity, to FcRn.
   64. The molecule of any one of embodiments 59-63, wherein the Fc polypeptide enhances antigen presentation on the surface of a cell.
   65. The molecule of any one of embodiments 59-64, wherein the cell is a thymic cell.
   66. The molecule of any one of embodiments 42-65, wherein the molecule does not comprise an Fc region.
   67. The molecule of any one of embodiments 42-66, wherein the cell targeting domain is conjugated to the N-terminus of Fc region, and the antigen of interest, or a domain that binds to the antigen of interest, is conjugated to a C-terminus of the Fc polypeptide domain.
   68. The molecule of any one of embodiments 42-66, wherein the cell targeting domain is conjugated to the C-terminus of Fc region, and the antigen of interest, or a domain that binds to the antigen of interest, is conjugated to the N-terminus of the Fc polypeptide domain.
   69. The molecule of any one of embodiments 42-68, wherein the cell targeting domain and the antigen of interest, or the domain that binds to the antigen of interest, are linked by a linker.
   70. The molecule of embodiment 69, wherein the linker is a peptide linker.
   71. The molecule of any one of embodiments 42-70, wherein the cell targeting domain and the antigen of interest, or the domain that binds to the antigen of interest, are directly conjugated.
   72. A dimer molecule comprising a first molecule and a second molecule, wherein: the first molecule is any one molecule of embodiments 1-71; and the second molecule is any one molecule of embodiments 1-71.
   73. The dimer molecule of embodiment 72, wherein the first polypeptide and the second polypeptide are the same polypeptides, or different polypeptides.
   74. A pharmaceutical composition comprising the molecule of any one of embodiments 1-71, or the dimer molecule of any one of embodiments 72-73.
   75. A method of modulating a cell, or a plurality of cells, the method comprising contacting the cell with the molecule of any one of embodiments 1-71, the dimer molecule of any one of embodiments 72-73, or the pharmaceutical composition of embodiment 74.
   76. The method of embodiment 75, wherein the method further comprises the cell, or plurality of cells, internalizing the molecule of any one of embodiments 1-71, or the dimer molecule of any one of embodiments 72-73, to produce a modulated cell, or a plurality of modulated cells comprising an effector domain.
   77. The method of embodiment 75, wherein the modulated cell, or plurality of modulated cells, comprises any one of the antigens of interest of any one of embodiments 1-72 on the membrane surface of the modulated cell.
   78. The method of any one of embodiments 75-77, wherein the cell is a thymic cell.
   79. The method of embodiment 78, wherein the thymic cell is a medullary thymic epithelial cell (mTEC), a mimetic mTEC, an AIRE+ mTEC, an AIRE− mTEC, a mTEC progenitor cell, a mimetic mTEC progenitor cell, a junction thymic epithelial cell (jTEC), a thymic progenitor epithelial cell (TPEC), a proliferating TPEC, a thymic epithelial cell (TEC), a proliferating TEC, a cortical thymic epithelial cell (cTEC), a proliferating cTEC, or a proliferating mTEC.
   80. A method of modulating a thymic cell, or a plurality of thymic cells, the method comprising contacting the thymic cell, or the plurality of thymic cells, with the molecule of any one of embodiments 1-71, the dimer molecule of any one of embodiments 72-73, or the pharmaceutical composition of embodiment 74.
   81. The method of embodiment 80, wherein the method further comprises the thymic cell, or the plurality of thymic cells, internalizing the molecule of any one of embodiments 1-71, or the dimer molecule of any one of embodiments 72-73, to produce a modulated thymic cell, or a plurality of modulated thymic cells, comprising the antigen of interest.
   82. The method of embodiment 81, wherein the modulated thymic cell, or plurality of modulated thymic cells, comprises any one of the antigens of interest of any one of embodiments 1-71 on the membrane surface of the modulated thymic cell.
   83. The method of any one of embodiments 80-82, wherein the thymic cell is a medullary thymic epithelial cell (mTEC), a mimetic mTEC, an AIRE+ mTEC, an AIRE− mTEC, a mTEC progenitor cell, a mimetic mTEC progenitor cell, a junction thymic epithelial cell (jTEC), a thymic progenitor epithelial cell (TPEC), a proliferating TPEC, a thymic epithelial cell (TEC), a proliferating TEC, a cortical thymic epithelial cell (cTEC), a proliferating cTEC, or a proliferating mTEC.
   84. A method of inducing antigen-specific Treg expansion in a subject, the method comprising administering to the subject the molecule of any one of embodiments 1-71, the dimer molecule of any one of embodiments 72-73, or the pharmaceutical composition of embodiment 74.
   85. The method of embodiment 84, wherein the Treg is a thymic Treg, or a splenic Treg.
   86. The method of embodiment 85, wherein the Treg is a CD4+ FoxP3+ Treg.
   87. The method of embodiment 86, wherein the Treg is a CD4+ FoxP3+CD73− Treg.
   88. The method of any one of embodiments 84-87, wherein the molecule of any one of embodiments 1-71, the dimer molecule of any one of embodiments 72-73, or the pharmaceutical composition of embodiment 74 is administered to the subject systematically.
   89. The method of any one of embodiments 84-88, wherein the method does not significantly induce antigen-specific T_{eff} cells.
   90. A method of inducing immune tolerance against an antigen of interest, such as T cell tolerance to the antigen of interest in a subject, the method comprising administering to the subject the molecule of any one of embodiments 1-71, or the dimer molecule of any one of embodiments 72-73, or the pharmaceutical composition of embodiment 74.
   91. The method of embodiment 90, wherein the T cell is specific against the antigen, auto-antigen, self-antigen, tolerance inducing antigen, effector peptide, or a fragment thereof, of any one of embodiments 1-71.
   92. The method of any one of embodiments 90-91, wherein the T cell is a CD4+CD8+ T cell, a CD4+ T cell, or a CD8+ T cell.
   93. The method of embodiment 92, wherein the T cell is a CD4+ FoxP3+ Treg.
   94. The method of embodiment 93, wherein the Treg is a CD4+ FoxP3+CD73− Treg.
   95. The method of any one of embodiments 90-94, wherein the molecule of any one of embodiments 1-71, or the dimer molecule of any one of embodiments 72-73, or the pharmaceutical composition of embodiment 74 is administered to the subject systematically.
   96. A method of treating or reducing an autoimmune, alloimmune, allergic, inflammatory, or anti-drug immune response in a subject, the method comprising administering the molecule of any one of embodiments 1-71, or the dimer molecule of any one of embodiments 72-73, or the pharmaceutical composition of embodiment 74, to the subject.
   97. The method of embodiment 96, wherein the autoimmune, alloimmune, allergic, inflammatory, or anti-drug immune response is elicited by a disorder, such as asthma, severe asthma. neutrophilic asthma, paucigranulocytic asthma, eosinophilic asthma, osteoarthritis, solid organ transplant, atopic dermatitis, eosinophilic esophagitis, dermatitis herpitoformis, chronic rhinosinusitis, chronic spontaneous urticaria, food allergy, anti-biologic drug immune response, anti-viral immune response, anti-AAV immune response, anti-adenoviral immune response, anti-retroviral immune response, anti-lentiviral immune response, and -VLP immune response, Alzheimer's disease, amyotrophic lateral sclerosis (ALS), Parkinson's disease, dementia with Lewy bodies, Duchenne muscular dystrophy, cutaneous lupus myocarditis, postmyocardial infarction syndrome, postpericardiotomy syndrome, subacute bacterial endocarditis, anti-glomerular basement membrane nephritis, interstitial cystitis, lupus nephritis, membranous glomerulonephropathy, chronic kidney disease (“CKD”), autoimmune hepatitis, primary biliary cirrhosis, primary sclerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease (LAD), morphea, pemphigus, pemphigus vulgaris, hidradenitis suppuritiva, pityriasis lichenoides et varioliformis acuta, Mucha-Habermann disease, psoriasis, guttate psoriasis, erythrodermic psoriasis, plaque psoriasis, pustular psoriasis, systemic sclerosis, skin manifestation associated with systemic sclerosis, vitiligo, Addison's disease, autoimmune polyendocrine syndrome (APS) type 1, autoimmune polyendocrine syndrome (APS) type 2, autoimmune polyendocrine syndrome (APS) type 3, autoimmune pancreatitis (AIP), diabetes mellitus type 1, autoimmune thyroiditis, Ord's thyroiditis, Graves' disease, non radiographic axial spondyloarthropathy, autoimmune oophoritis, endometriosis, autoimmune orchitis, Sjogren's syndrome, dermatitis herpatiformis, autoimmune enteropathy, coeliac disease, Crohn's disease, immune related adverse events (irAEs) secondary to immune checkpoint inhibitors, microscopic colitis, ulcerative colitis, autoimmune thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, rheumatoid arthritis, sarcoidosis, Schnitzler syndrome, systemic lupus erythematosus (SLE), undifferentiated connective tissue disease (UCTD), dermatomyositis, fibromyalgia, inclusion body myositis, myositis, myasthenia gravis, neuromyotonia, paraneoplastic cerebellar degeneration, polymyositis, acute disseminated encephalomyelitis (ADEM), acute motor axonal neuropathy, anti-N-methyl-D-aspartate (anti-NMDA) receptor encephalitis, balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, oshtoran syndrome, pediatric autoimmune neuropsychiatric disorder associated with streptococcus (PANDAS), progressive inflammatory neuropathy, restless leg syndrome, stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Mdniere's disease, Behcet's disease, eosinophilic granulomatosis with polyangiitis (EGPA), giant cell arteritis, granulmatosis with polyangiitis (GPA), IgA vasculitis (IgAV), Kawasaki's disease, leukocytoclastic vasculitis, lupus vasculitis, rheumatoid vasculitis, microscopic polyangiitis (MPA), polyarteritis nodosa (PAN), polymyalgia rheumaticia, vasculitis, primary immune deficiency, Gaucher disease, Pompe disease, Fabry disease, Schindler disease, Hurler syndrome (MPS1), Tay-Sachs disease, Nieman-Pick disease, Farber lipogranulomatosis, Krabbe disease, Morquio syndrome (MPS4), mucopolysaccharidosis type VI (MPS6), Hunter syndrome (MPS2), Sanfilippo syndrome (MPS3), Sly sundrome (MPS7), hyaluronidase deficiency (MPS9), mucolipidosis type 1 (ML1), I-cell disease, mucolipidosis II (ML2), mucolipidosis III gamma (ML3C), glycogen storage disease Ia, glycogen storage disease Ib, Wilson disease, phenylketonuria, Stargardt disease type I, myocilin-associated glaucoma, hemophilia A, hemophilia B, hereditary or acquired thrombotic thrombocytopathic purpura (TTP), von Willebrand disease, thrombocytopenia platelet autoantigen, heparin-induced thrombocytopenia, tolerance induction for AAV gene therapy, tolerance induction for ex vivo gene editing, tolerance induction for in vivo gene editing, acquired hemophilia, antiphospholipid syndrome, aplastic anemia, autoimmune lymphoproliferative syndrome, autoimmune neutropenia, autoimmune thrombocytopenic purpura, cold agglutinin disease, essential mixed cryoglobulinemia, Evans syndrome, IgG4-related diseases, pure red cell aplasia, warm autoimmune hemolytic anemia, autoimmune gastritis, celiac disease, pernicious anemia, anti-sperm antibodies, autoimmune polyendocrine syndrome type 1, autoimmune polyendocrine syndrome type 2, autoimmune polyendocrine syndrome type 3, autoimmune myocarditis, cryptogenic organizing pneumonia, idiopathic pulmonary fibrosis, IgA nephropathy, post-myocardial infarction syndrome, primary biliary cholangitis, primary idiopathic dilated cardiomyopathy, anti-N-methyl-D-aspartate receptor encephalitis, autoimmune encephalitis, encephalopathy associated with autoimmune thyroid disease, episcleritis, Graves' ophthalmopathy, myelin oligodendrocyte glycoprotein disease, narcolepsy with cataplexy, cutaneous lupus erythematosus, adult-onset Still's disease, ankylosing spondylitis, juvenile arthritis, juvenile dermatomyositis, anti-neutrophil cytoplasmic antibody-associated vasculitis, polymyalgia rheumatic, purpura rheumatic, Takayasu arteritis, graft versus host disease (GVHD), transplant, and xenotransplantation.
   98. The method of any one of embodiments 96-97, wherein the molecule of any one of embodiments 1-71, or the dimer molecule of any one of embodiments 72-73, or the pharmaceutical composition of embodiment 74 is administered to the subject systematically.
   99. A method of improving transplant therapy in a subject in need of a transplant, the method comprising administering the molecule of any one of embodiments 1-71, or the dimer molecule of any one of embodiments 72-73, or the pharmaceutical composition of embodiment 74 to the subject, wherein the effector domain comprises an HLA or tissue antigen, or a fragment thereof.
   100. The method of embodiment 99, wherein the subject is in need of an allogeneic donor organ transplant or an allogeneic donor cell transplant.
   101. The method of any one of embodiments 99-100, wherein the HLA or tissue antigen, or a fragment thereof, is of the same class as the allogeneic donor organ or the allogeneic donor cell transplant and is the not the same as an HLA or tissue antigen of the subject.
   102. The method of embodiment 101, wherein the HLA auto-antigen, self-antigen, or a fragment thereof, is selected from HLA-A, HLA-B, HLA-C, HLA-E, HLA-L, HLA-J, HLA-K, HLA-H, HLA-G, HLA-DM, HLA-DO, HLA-DP, HLA-DQ, and HLA-DR.
   103. The method of any one of embodiments 100-102, wherein the molecule of any one of embodiments 1-71, or the dimer molecule of any one of embodiments 72-73, or the pharmaceutical composition of embodiment 74, is administered prior to, concurrently with, or following transplantation.
   104. The method of any one of embodiments 100-103, wherein the molecule of any one of embodiments 1-71, or the dimer molecule of any one of embodiments 72-73, or the pharmaceutical composition of embodiment 74 is administered to the subject systematically.
   105. A method of reducing transplant rejection or graft versus host disease (GVHD) in a subject in need of a transplant, the method comprising administering the molecule of any one of embodiments 1-71, or the dimer molecule of any one of embodiments 72-73, or the pharmaceutical composition of embodiment 74 to the subject, wherein the effector domain comprises an HLA or tissue antigen, or a fragment thereof.
   106. The method of embodiment 105, wherein the transplant is an allogeneic organ transplant or an allogeneic cell transplant.
   107. The method of any one of embodiments 105-106, wherein the HLA or tissue antigen, or a fragment thereof, is of the same class as the allogeneic donor organ or the allogeneic donor cell transplant and not present in the subject.
   108. The method of embodiment 107, wherein the HLA or tissue antigen, or a fragment thereof, is selected from HLA-A, HLA-B, HLA-C, HLA-E, HLA-L, HLA-J, HLA-K, HLA-H, HLA-G, HLA-DM, HLA-DO, HLA-DP, HLA-DQ, and HLA-DR.
   109. The method of any one of embodiments 105-108, wherein the molecule of any one of embodiments 1-71, or the dimer molecule of any one of embodiments 72-73, or the pharmaceutical composition of embodiment 74, is administered to the subject prior to, concurrently with, or following transplantation.
   110. The method of any one of embodiments 105-109, wherein the molecule of any one of embodiments 1-71, or the dimer molecule of any one of embodiments 72-73, or the pharmaceutical composition of embodiment 74 is administered to the subject systematically.
   111. A method of inducing tolerance to a viral capsid protein in a subject, the method comprising administering the molecule of any one of embodiments 1-71, or the dimer molecule of any one of embodiments 72-73, or the pharmaceutical composition of embodiment 74 to the subject.
   112. The method of embodiment 111, wherein the subject is a healthy subject, or a subject receiving a viral therapy.
   113. The method of embodiment 111, wherein the viral capsid protein is an AAV capsid protein, such as those disclosed herein.
   114. The method of any one of embodiments 111-113, wherein the method further comprises inducing tolerance to a transgene.
   115. The method of embodiment 114, wherein inducing tolerance to the transgene enables persistence of the transgene.
   116. The method of any one of embodiments 111-115, wherein inducing tolerance achieves mitigation of immunological side effects of the therapy.
   117. The method of any one of embodiments 111-116, wherein the method further comprises re-dosing the subject with the molecule of any one of embodiments 1-71, or the dimer molecule of any one of embodiments 72-73, or the pharmaceutical composition of embodiment 74.
   118. The method of any one of embodiments 111-117, wherein the molecule of any one of embodiments 1-71, or the dimer molecule of any one of embodiments 72-73, or the pharmaceutical composition of embodiment 74 is administered to the subject systematically.

The following examples are illustrative, but not limiting, of the compounds, compositions and methods described herein. Other suitable modifications and adaptations known to those skilled in the art are within the scope of the following embodiments.

### EXAMPLES

Example 1: EpCAM and Insulin dual targeted polypeptide Fc is used to treat autoimmune disorder. A polypeptide comprising an antibody, or the antigen-binding fragment thereof, that binds to EpCAM thymic cell receptor, and an antigen of interest that binds to insulin B chain, which are linked with a Fc region is administered to a subject with an autoimmune disorder and the subject is treated for the autoimmune disorder. The Fc region may also be effectorless, and could have the LALAGA mutations (AAA) as provided for herein.

Example 2: EpCAM and myelin oligodendrocyte glycoprotein (MOG) dual targeted polypeptide Fe is used to treat autoimmune disorder. A polypeptide comprising an antibody, or the antigen-binding fragment thereof, that binds to EpCAM thymic cell receptor, and an antigen of interest that binds to MOG, which are linked with a Fc region is administered to a subject with an autoimmune disorder and the subject is treated for the autoimmune disorder. The Fc region may also be effectorless, and could have the LALAGA mutations (AAA) as provided for herein.

Example 3: EpCAM and myelin basic protein (MBP) dual targeted polypeptide Fc is used to treat autoimmune disorder. A polypeptide comprising an antibody, or the antigen-binding fragment thereof, that binds to EpCAM thymic cell receptor, and an antigen of interest that binds to MBP, which are linked with a Fc region is administered to a subject with an autoimmune disorder and the subject is treated for the autoimmune disorder. The Fc region may also be effectorless, and could have the LALAGA mutations (AAA) as provided for herein.

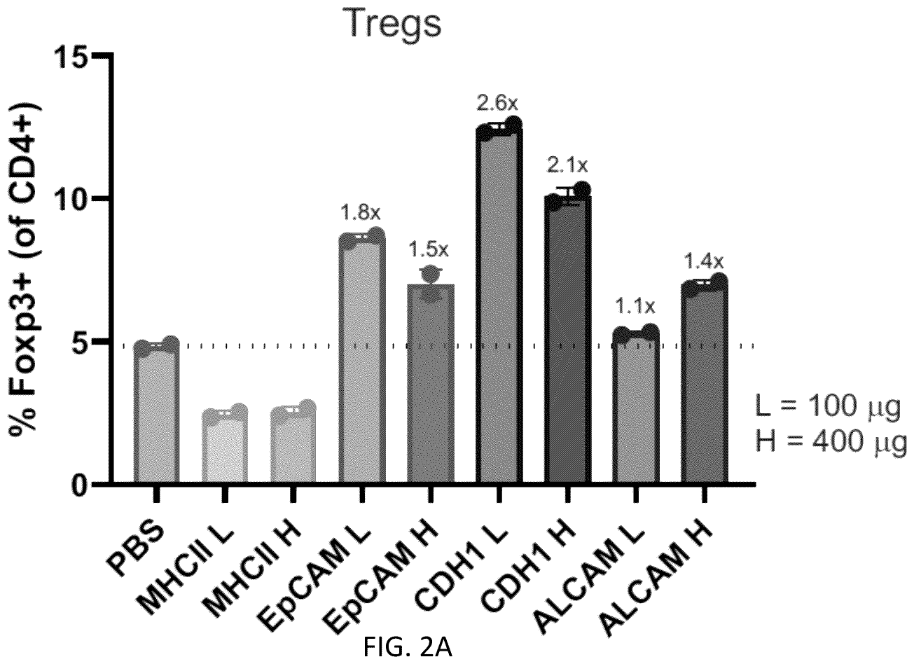

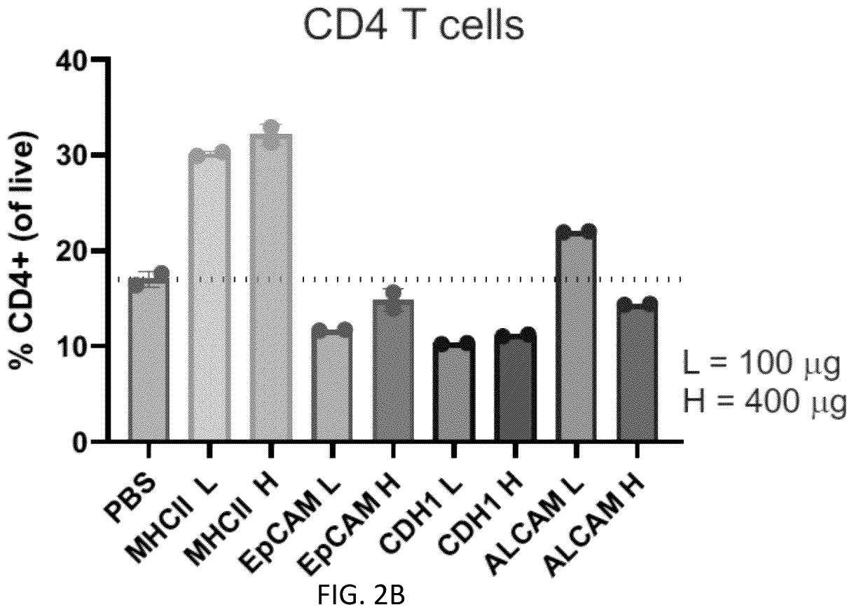

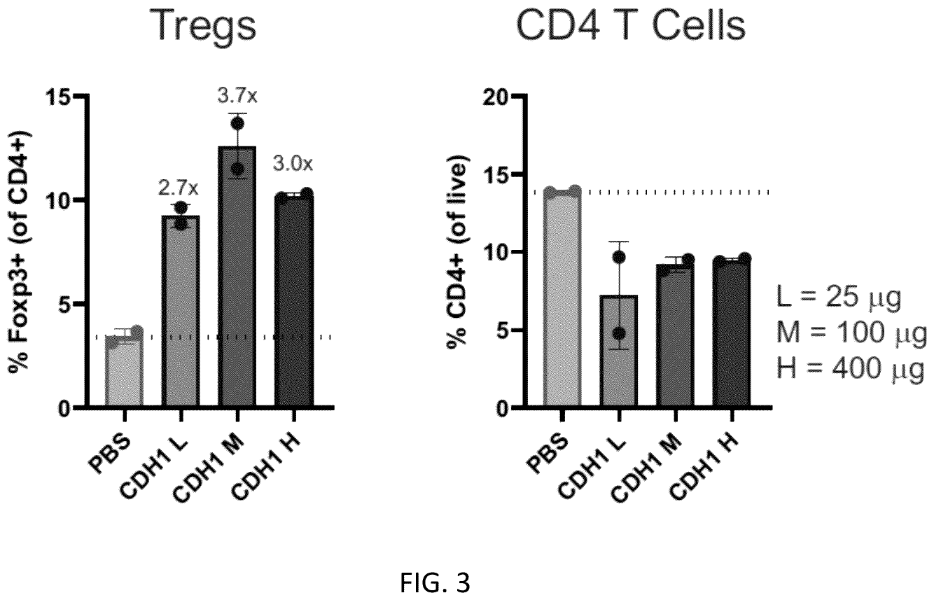

Example 4: Test articles comprising the OVA antigen and a targeting domain selected from MHCII, EpCAM (G8.8 antibody clone, BioLegend cat. no. 118211), CDH1 (DECMA-1 antibody clone, ThermoFisher cat. no. 14-3249-82), or ALCAM, were administered intravenously into 6-8 week old OT-II female mice at doses of 100 ug (L), and 400 ug (H) of OVA equivalents (the amount/mass (in ug) of OVA antigen appended to the antibody). Spleens were harvested at 10 days, and splenocytes were stained with a Treg antibody panel. As shown in FIGS. 2A and 3, EpCAM and CDH1 targeted OVA molecules drove the greatest increase in proportion of FoxP3+ Tregs. A decrease in proportion of total CD4 T cells was also observed (FIG. 2B).

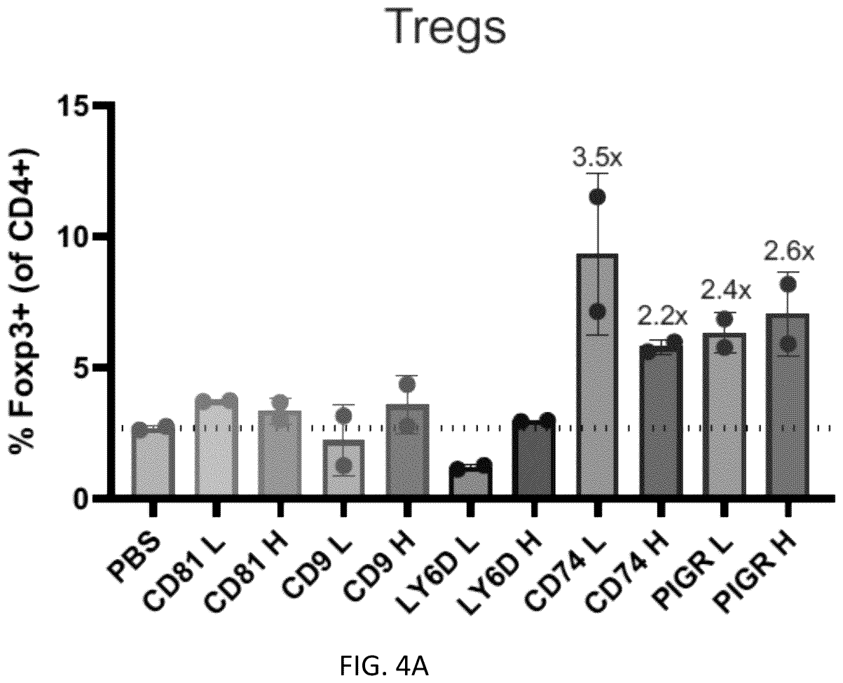

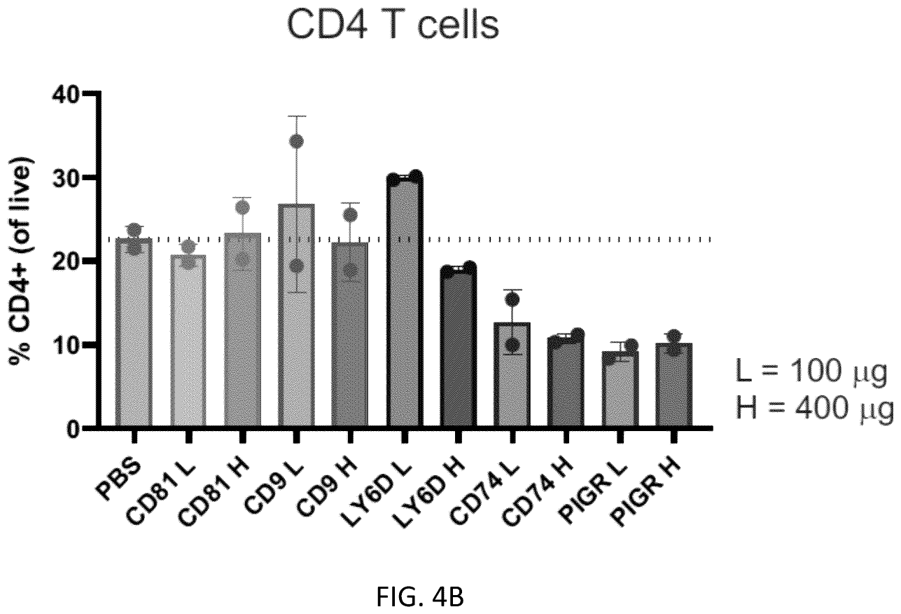

Example 5: Test articles comprising the OVA antigen and a targeting domain selected from CD81, CD9, LY6D, CD74 (ln1/CD74 antibody clone, BioLegend cat. no. 151002), or P1GR (an anti-PlGR antibody, or an antigen-binding fragment thereof, as disclosed in U.S. Publication No. 20220112276, which is hereby incorporated by reference in its entirety), were administered intravenously into 6-8 week old OT-II female mice at doses of 100 ug, and 400 ug of OVA equivalents. Spleens were harvested at 10 days, and splenocytes were stained with a Treg antibody panel. As shown in FIG. 4A, CD74 and P1GR targeted OVA molecules drove the greatest increase in proportion of FoxP3+ Tregs. A decrease in proportion of total CD4 T cells was also observed (FIG. 4B).

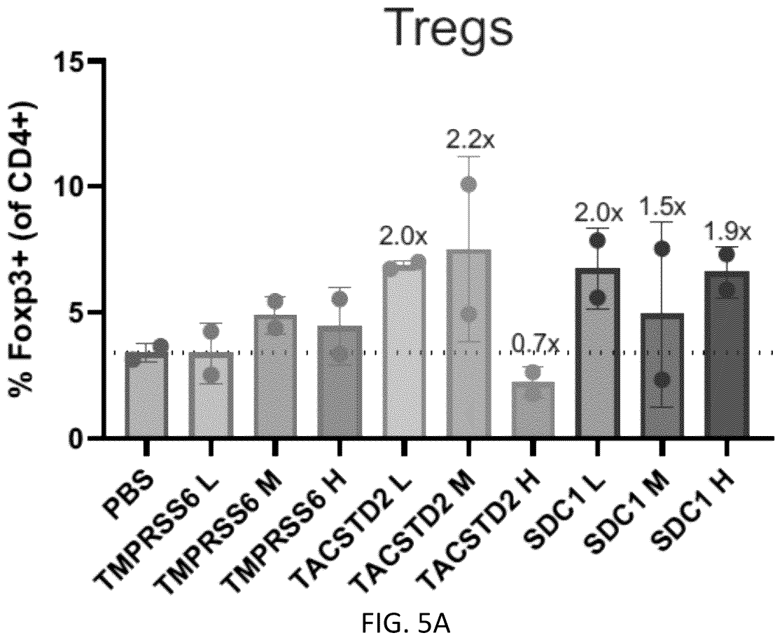

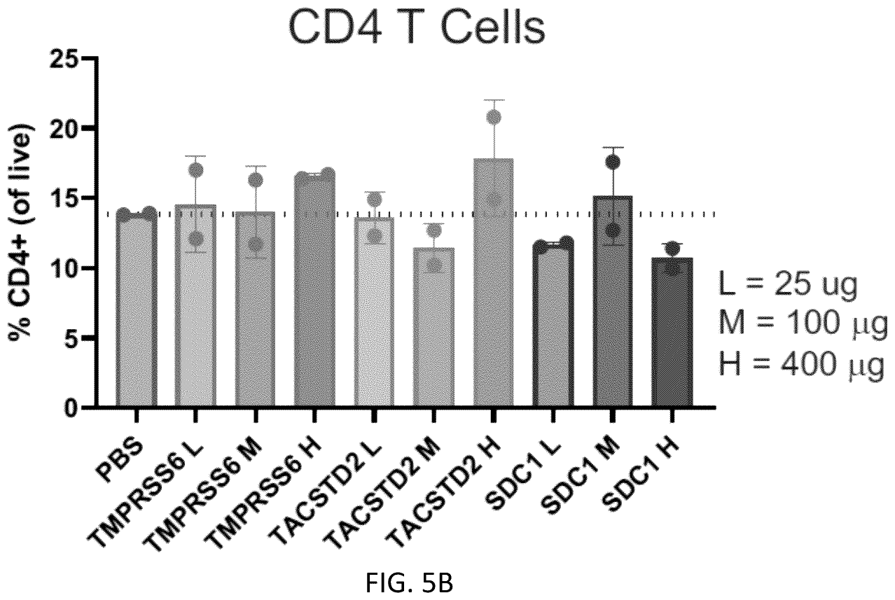

Example 6: Test articles comprising the OVA antigen and a targeting domain selected from TMPRSS6, TACSTD2, or SCD1 were administered intravenously into 6-8 week old OT-II female mice at doses of 25 ug, 100 ug, and 400 ug of OVA equivalents. Spleens were harvested at 10 days, and splenocytes were stained with a Treg antibody panel. As shown in FIG. 5A, TACSTD2 and SCD1 targeted OVA molecules drove the greatest increase in proportion of FoxP3+ Tregs.

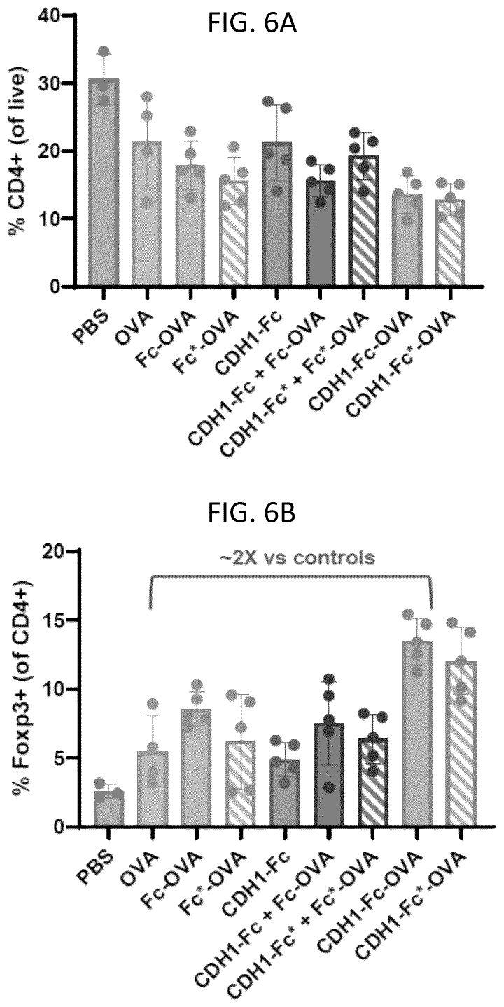

Example 7: Test articles comprising the OVA antigen, a CDH1 targeting domain (DECMA-1 antibody clone, ThermoFisher cat. no. 14-3249-82), and a wild-type Fc polypeptide or an Fc polypeptide comprising the “LALAPG” (L234A, L235A, P329G) mutations rendering the Fc polypeptide effectorless were administered intravenously into 6-8 week old OT-II female mice at a dose of 100 ug of OVA equivalent. Spleens were harvested at 10 days, and splenocytes were stained with a Treg antibody panel. In spleen, CDH1 targeted OVA increases proportion of FoxP3+ cells by at least ˜2X vs full range of controls (PBS, OVA, Fc-OVA, and CDH1-Fc OVA) (FIG. 6B). Effectorless Fc performed similarly to WT (FIG. 6A).

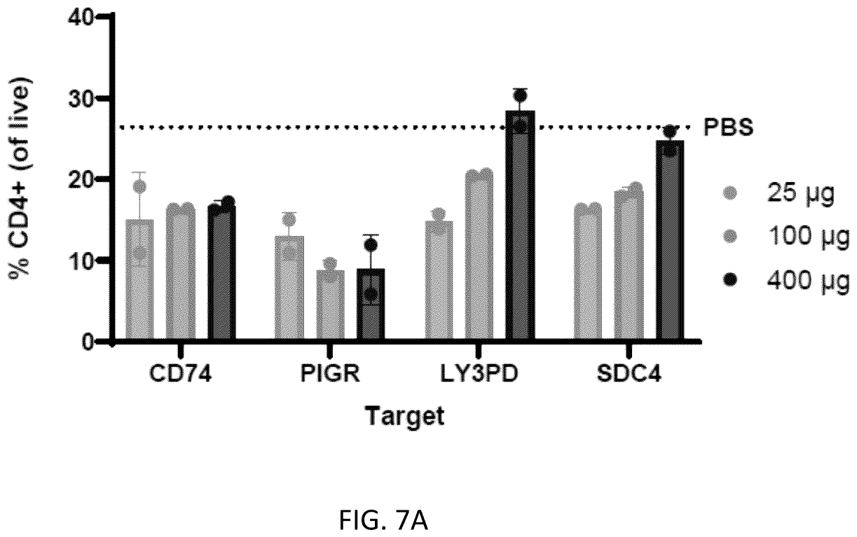

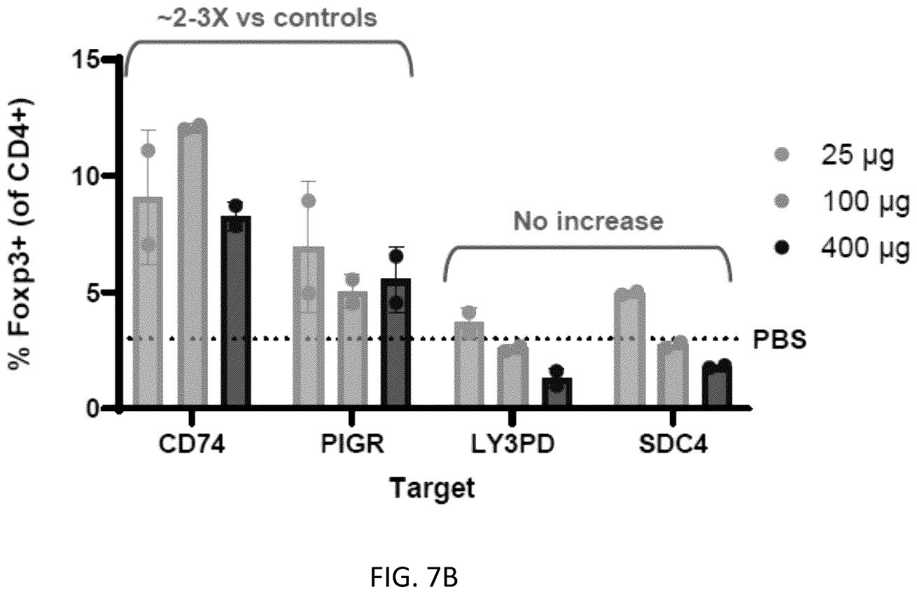

Example 8: Test articles comprising the OVA antigen and a targeting domain selected from CD74 (ln1/CD74 antibody clone, BioLegend cat. no. 151002), P1GR, LY3PD, or SDC4 were administered intravenously into 6-8 week old OT-II female mice at doses of 25 ug, 100 ug, and 400 ug of OVA equivalents. Spleens were harvested at 10 days, and splenocytes were stained with a Treg antibody panel. CD74 and P1GR targeted OVA molecules drove the increase in proportion of FoxP3+ Tregs, while LY3PD and SDC4 targeted OVA molecules did not produce a significant increase in proportion of FoxP3+ Tregs (FIG. 7B).

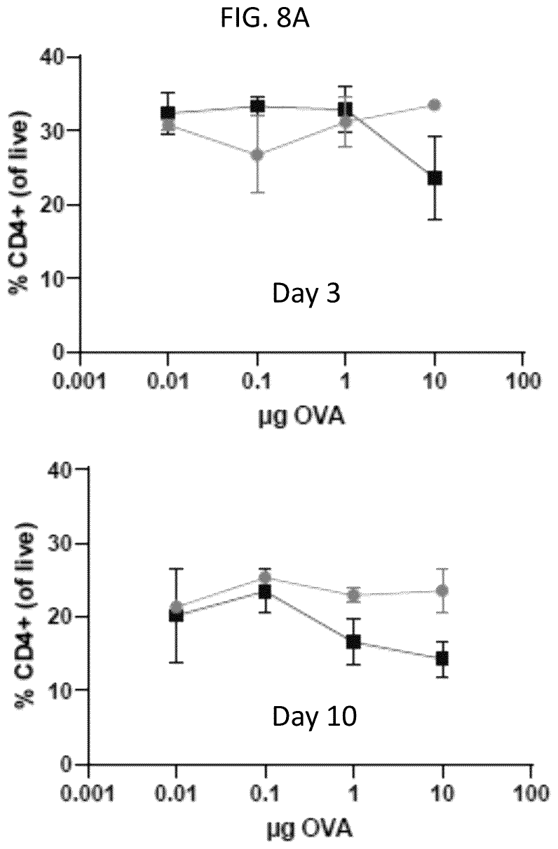

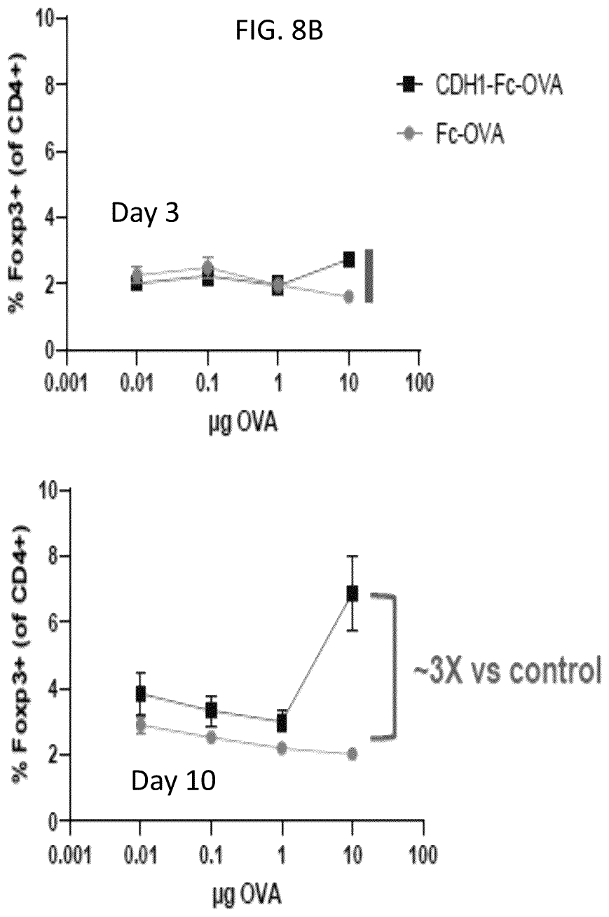

Example 9: A molecule comprising the OVA antigen and an effectorless Fc polypeptide, or a molecule comprising the OVA antigen, a CDH1 targeting domain (DECMA-1 antibody clone, ThermoFisher cat. no. 14-3249-82), and an effectorless Fc polypeptide were administered intrathecally into 6-8 week old OT-II female mice at doses of 0.01 ug, 0.1 ug, 1,ug, or 10 ug of OVA equivalents. Spleens were harvested at 3 and 10 days, and splenocytes were stained with a Treg antibody panel. Intrathylmic administration of CDH1 targeted OVA resulted in a 3X increase in proportion of FoxP3+ Tregs by day 10 in a dose-dependent manner (FIG. 8B).

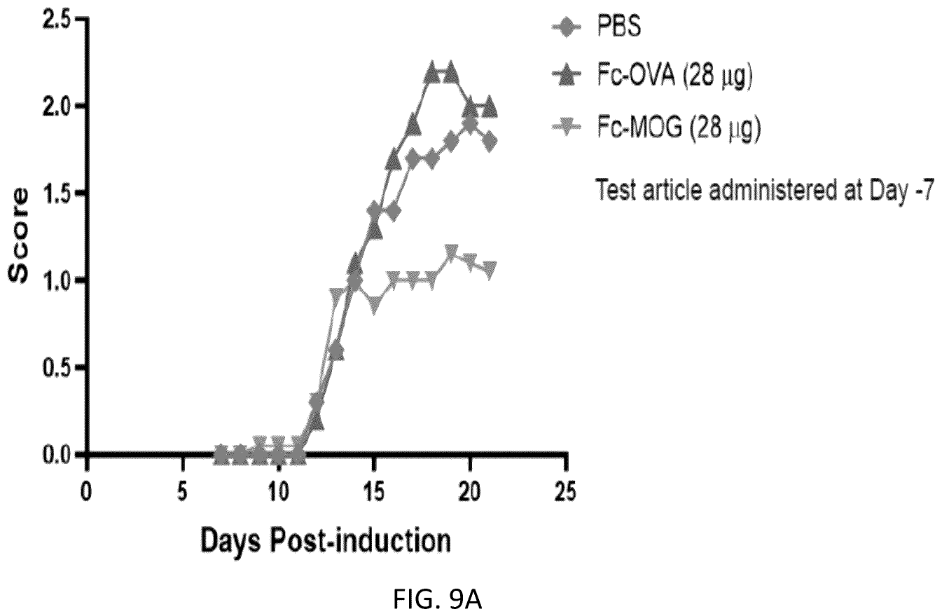

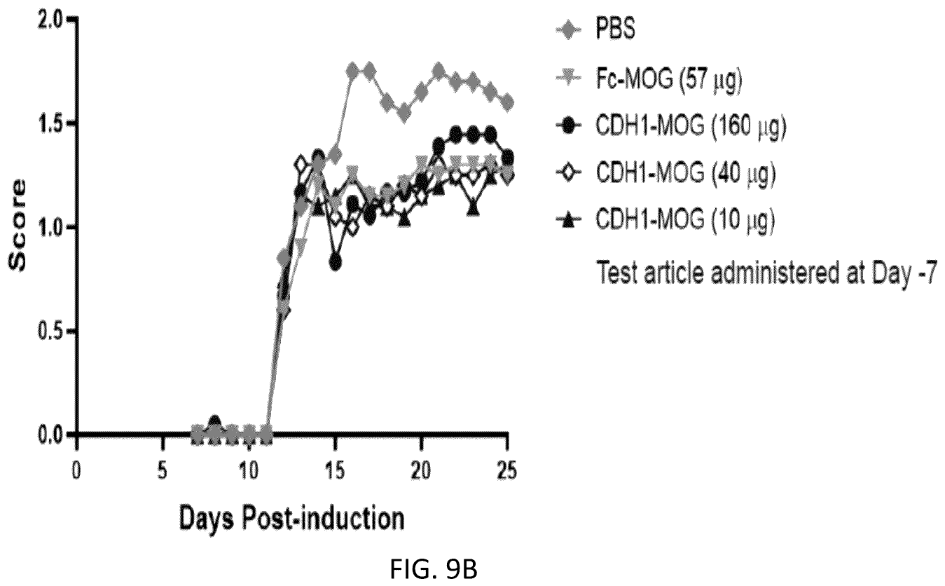

Example 10: Test articles comprising the OVA antigen, or the MOG antigen, and a wild-type Fc polypeptide, were administered intrathymcally at a dose of 28 ug into 4 week old C57Bl/6 female mice. Next, experimental autoimmune encephalomyelitis (EAE) was induced in said mice by administering MOG35-55 and 85 ng PTX at 1 week following administration of test articles. Administration of Fc-MOG showed a reduction in EAE score, as shown in FIG. 9A.

Example 11: Test articles comprising the MOG antigen and a wild-type Fc polypeptide, or the MOG antigen and the CDH1 cell targeting domain (DECMA-1 antibody clone, ThermoFisher cat. no. 14-3249-82) were administered intravenously into EAE mice established in Experiment 10 at day 7 at doses of 57 ug (Fc-MOG), 10 ug (CDH1-MOG), 40 ug (CDH1-MOG), and 160 ug (CDH1-MOG). Administration of Fc-MOG and CDH1-MOG showed a reduction in EAE score, as shown in FIG. 9B.

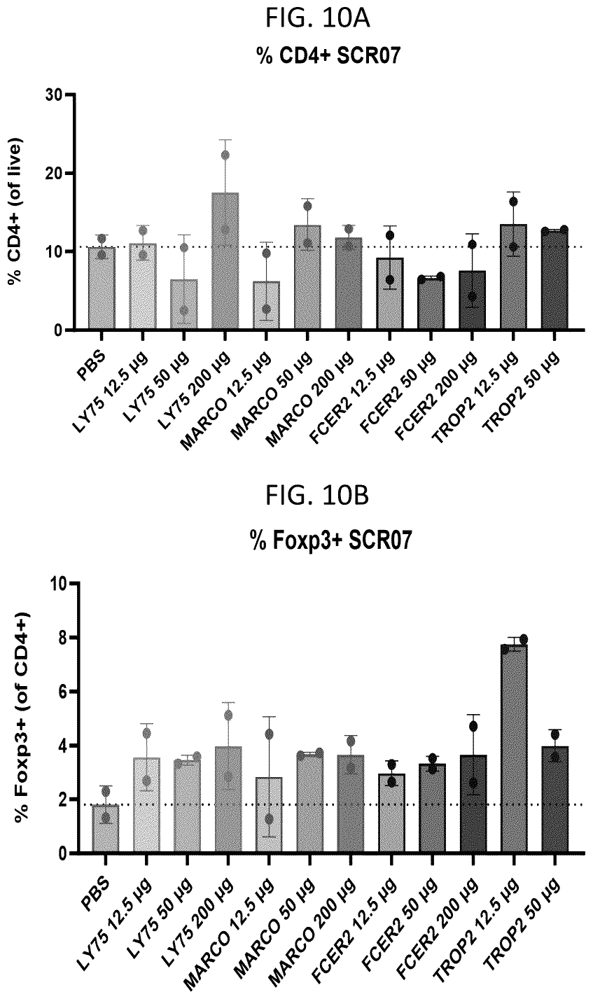

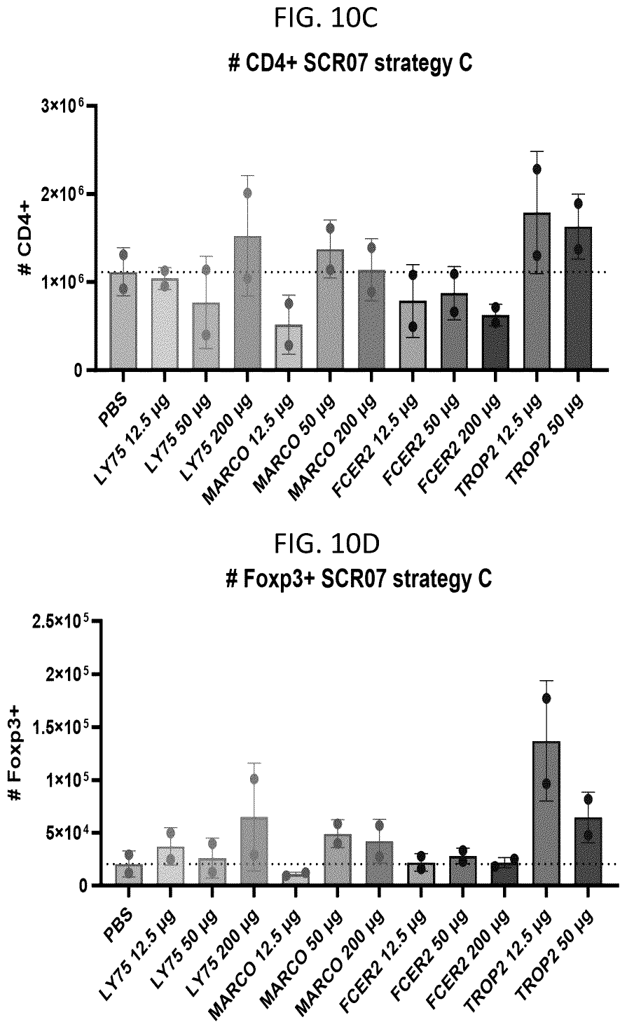

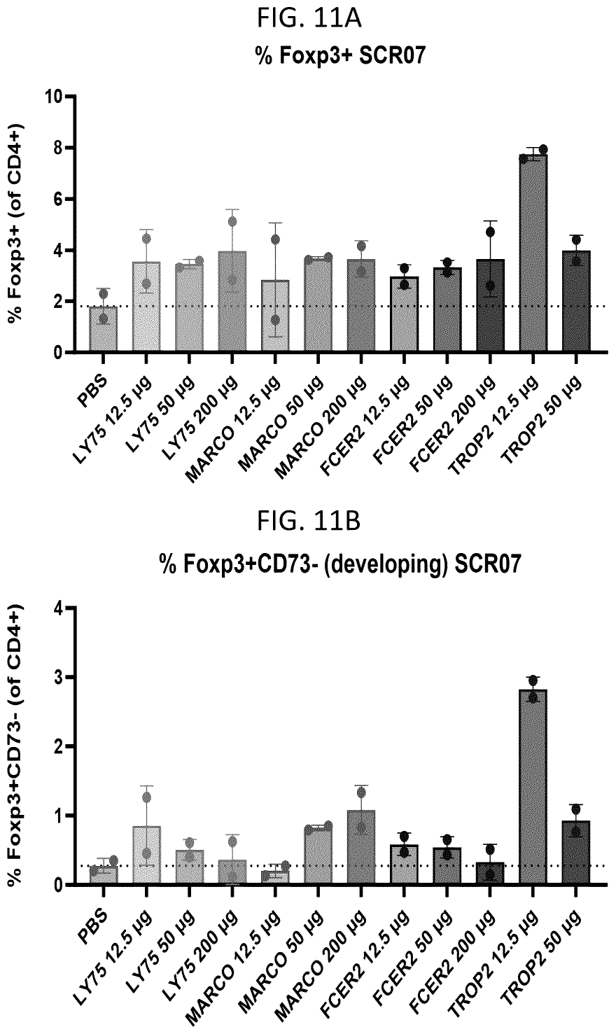

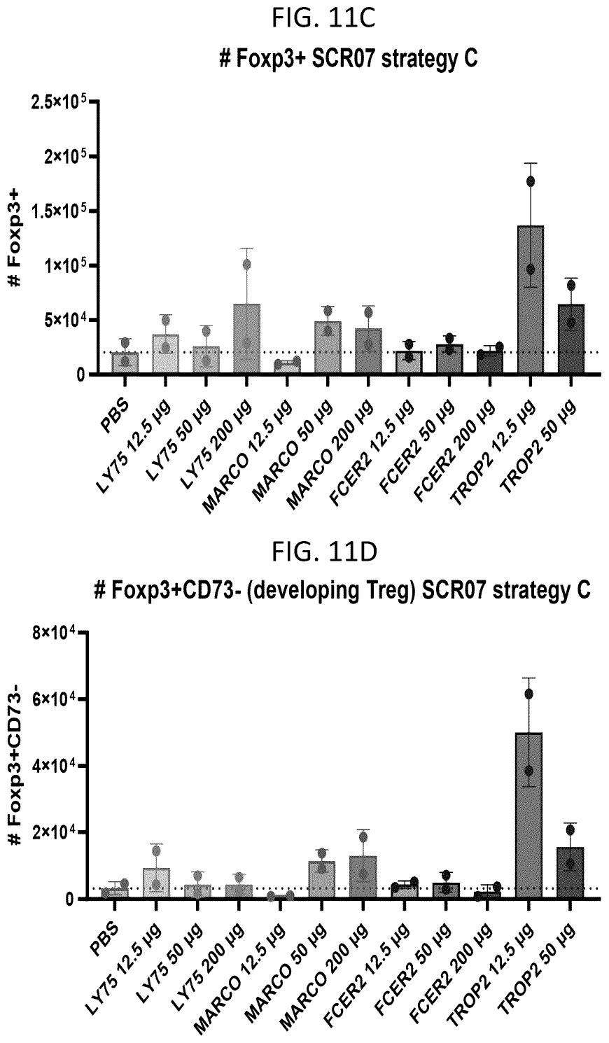

Example 12: Test articles comprising the OVA antigen, a wild-type Fc polypeptide, and a targeting domain selected from LY75 (HD83 antibody clone, BioLegend cat. no. 359202), MARCO (an anti-MARCO antibody, or an antigen-binding fragment thereof, as disclosed in U.S. Publication No. 20220153832, which is hereby incorporated by reference in its entirety), FCER2 (B3B4 antibody clone, ThermoFisher cat. no. 14-0232-81), or TROP2 (Sp293 antibody clone, Abcam cat. no. ab310311) were administered intravenously into 6-8 week old OT-II female mice at doses of 12.5 ug, 50 ug, and 200 ug of OVA equivalents as shown in FIGS. 10A-10D. Thymi were harvested at 10 days, and thymocytes were stained with a Treg antibody panel. TROP2 targeted OVA molecules drove the increase in proportion of FoxP3+ Tregs (4x as compared to control), and the increase in absolute number of FoxP3+ Tregs (7x as compared to control) (FIGS. 10B and 10D). TROP2 and MARCO targeted OVA molecules also drove expansion (proportion and absolute numbers) of thymically-induced FoxP3+CD73− Treg cells, which are Treg cells that developed in the thymus, as opposed to FoxP3+CD73+ Treg cells, which are recirculating Treg cells (FIGS. 11B and 11D). This data indicates that systemically-administered test articles are capable of targeting thymic cells.

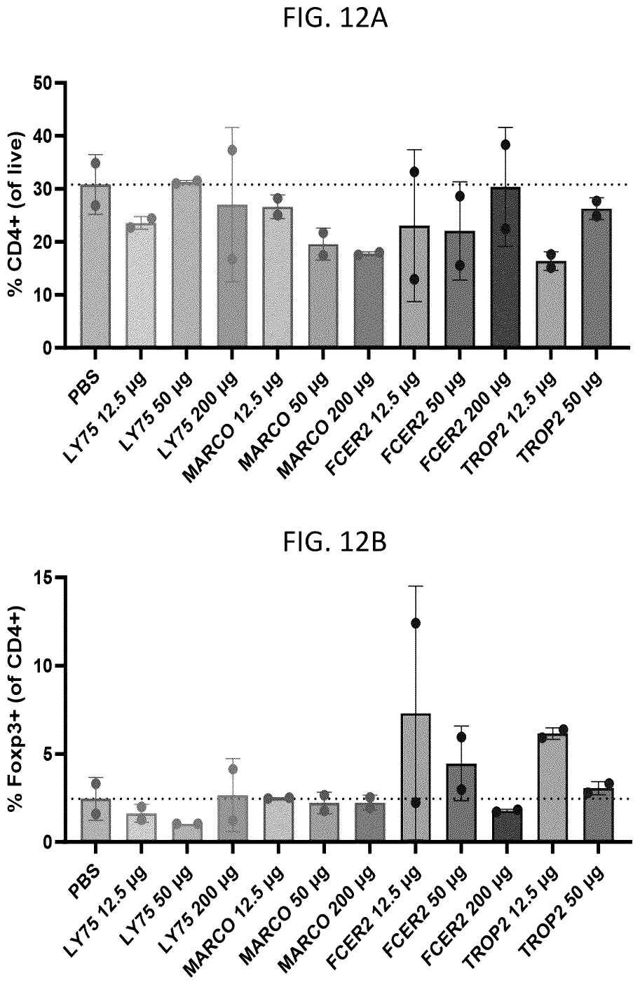

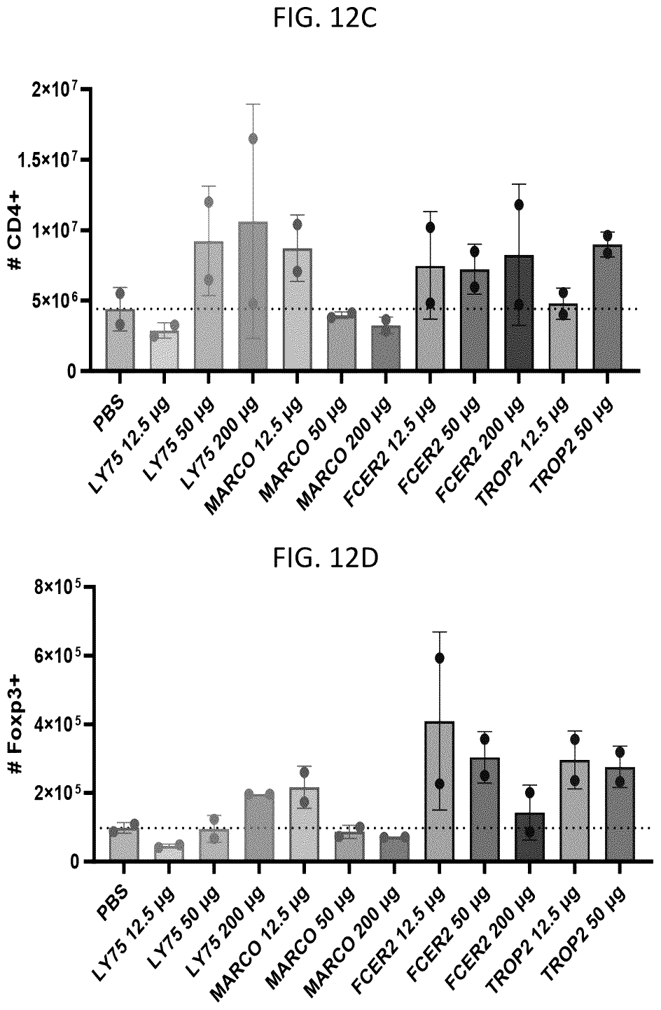

In the same experiment, spleens were harvested at 10 days, and splenocytes were stained with a Treg antibody panel. TROP2, FCER2, and MARCO targeted OVA molecules drove the increase in proportion of FoxP3+ Tregs, and the increase in absolute number of FoxP3+ Tregs, as compared to control (FIGS. 12B and 12D).

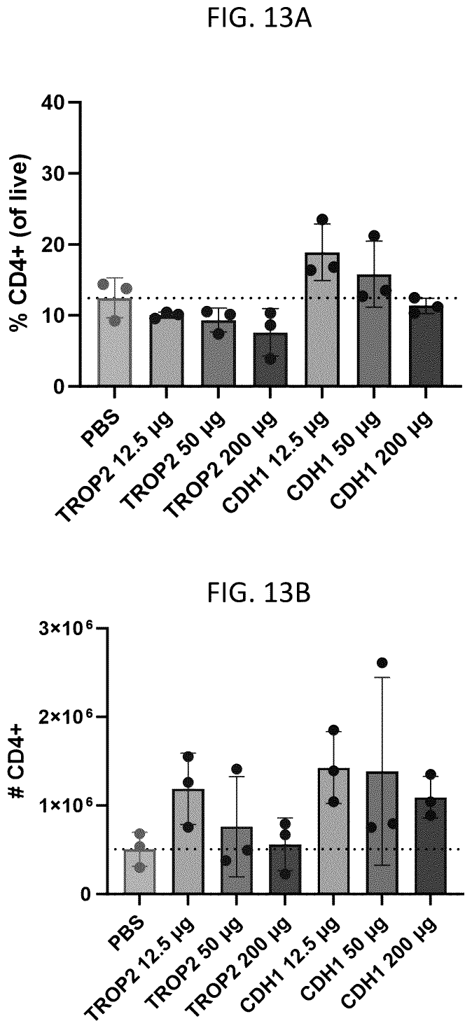

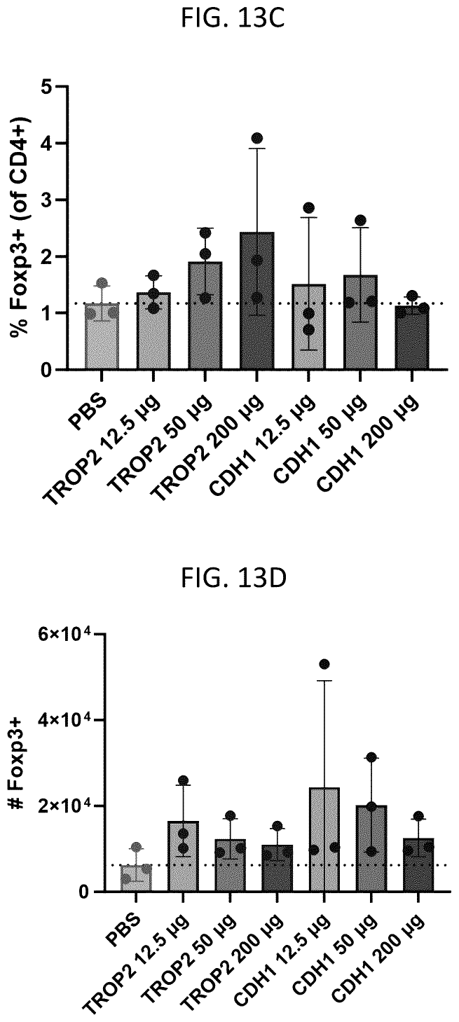

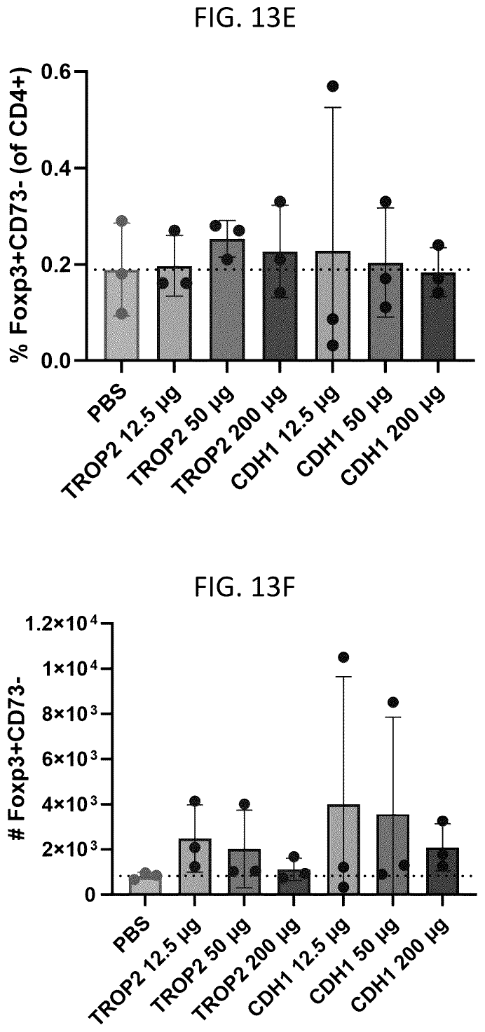

Example 13: Test articles comprising the OVA antigen, an effectorless Fc polypeptide comprising the “LALAPG” mutations as disclosed herein, and a targeting domain selected from TROP2 (Sp293 antibody clone, Abcam cat. no. ab310311), or CDH1 (DECMA-1 antibody clone, ThermoFisher cat. no. 14-3249-82) were administered intravenously into 6-8 week old OT-II female mice at doses of 12.5 ug, 50 ug, and 200 ug of OVA equivalents as shown in FIGS. 13A-13F. Thymi were harvested at 10 days, and thymocytes were stained with a Treg antibody panel. TROP2 and CDH1 targeted OVA molecules drove the increase in proportion of FoxP3+ Tregs, and the increase in absolute number of FoxP3+ Tregs, as compared to control (FIG. 13C-13D).

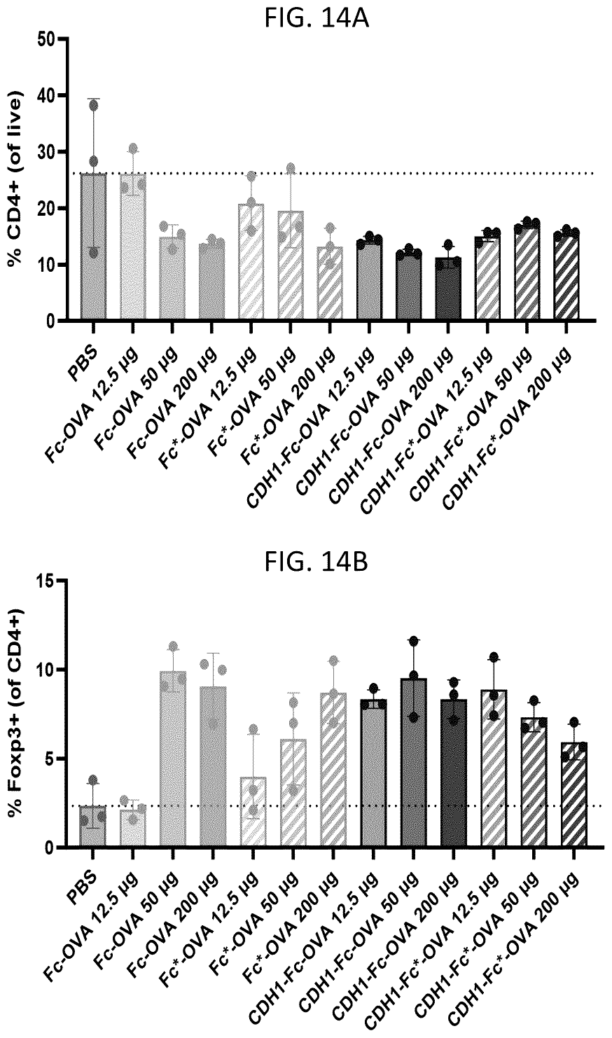

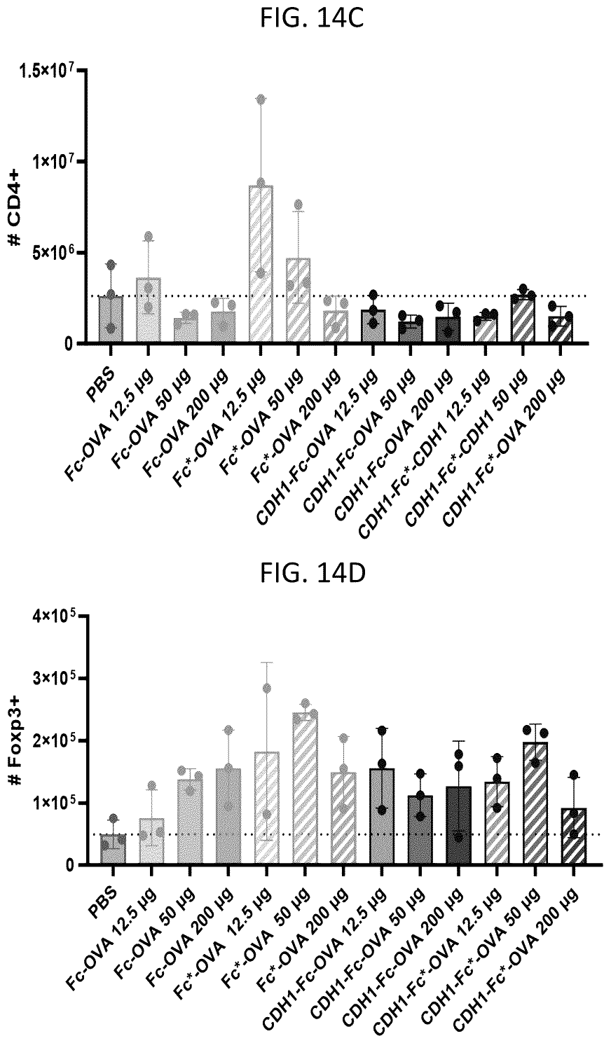

Example 14: Test articles comprising the OVA antigen, a wild-type Fc polypeptide or an effectorless Fc polypeptide comprising the “LALAPG” mutations as disclosed herein (shown in FIGS. 14A-14D using “*”), and the CDH1 (DECMA-1 antibody clone, ThermoFisher cat. no. 14-3249-82) targeting domain were administered intravenously into 6-8 week old OT-II female mice at doses of 12.5 ug, 50 ug, and 200 ug of OVA equivalents as shown in FIGS. 14A-14D. Spleens were harvested at 10 days, and splenocytes were stained with a Treg antibody panel. CDH1 targeted OVA molecules comprising either the wild-type Fc polypeptide or the effectorless Fc polypeptide both drove the increase in proportion of FoxP3+ Tregs, the increase in absolute number of FoxP3+ Tregs, the increase in the proportion of FoxP3+CD73− Tregs, and the increase in absolute number of FoxP3+CD73− Tregs, as compared to control (FIGS. 14B and 14D).

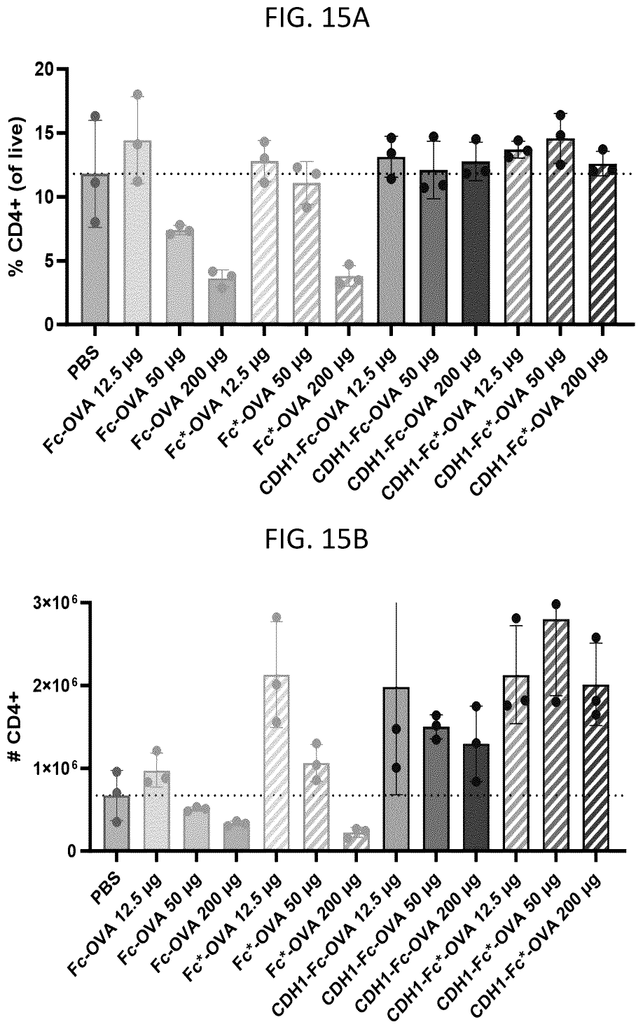

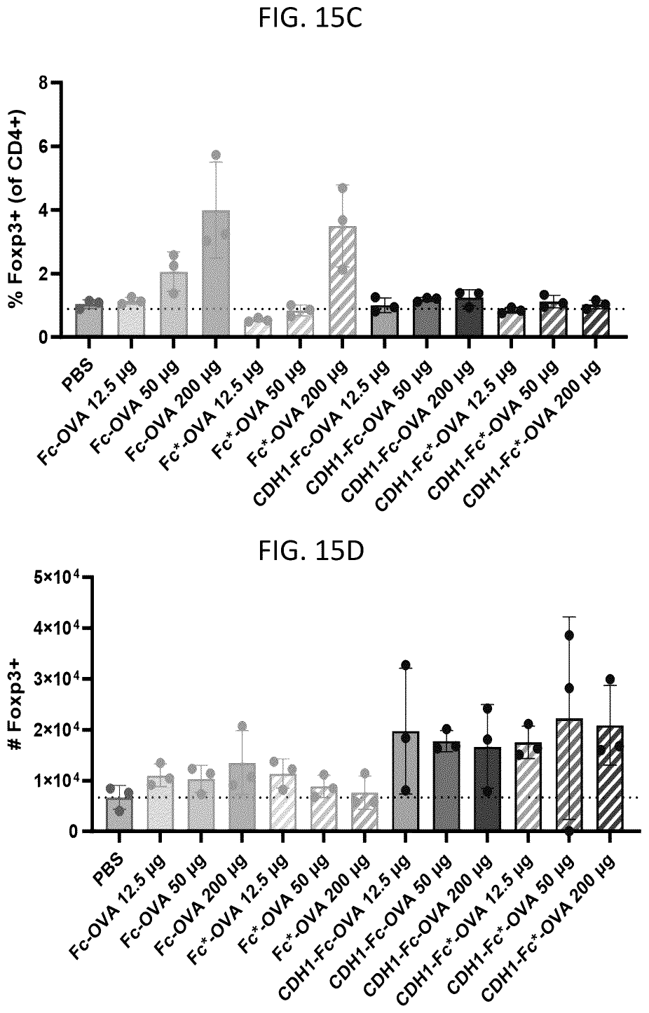

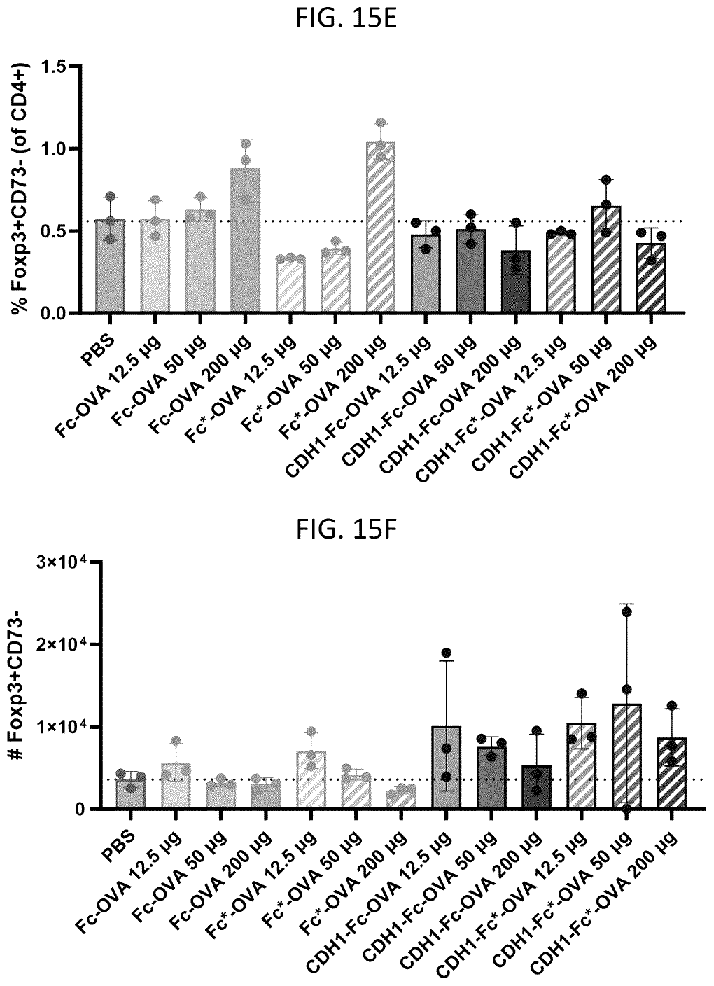

In the same experiment, thymi were harvested at 10 days, and thymocytes were stained with a Treg antibody panel. CDH1 targeted OVA molecules comprising either the wild-type Fc polypeptide or the effectorless Fc polypeptide both drove the increase in proportion of FoxP3+ Tregs, the increase in absolute number of FoxP3+ Tregs, the increase in the proportion of FoxP3+CD73− Tregs, and the increase in absolute number of FoxP3+CD73− Tregs, as compared to control (FIG. 15C-15F). However, the test articles also increased the absolute number of Teff cells, as shown in FIG. 15B.

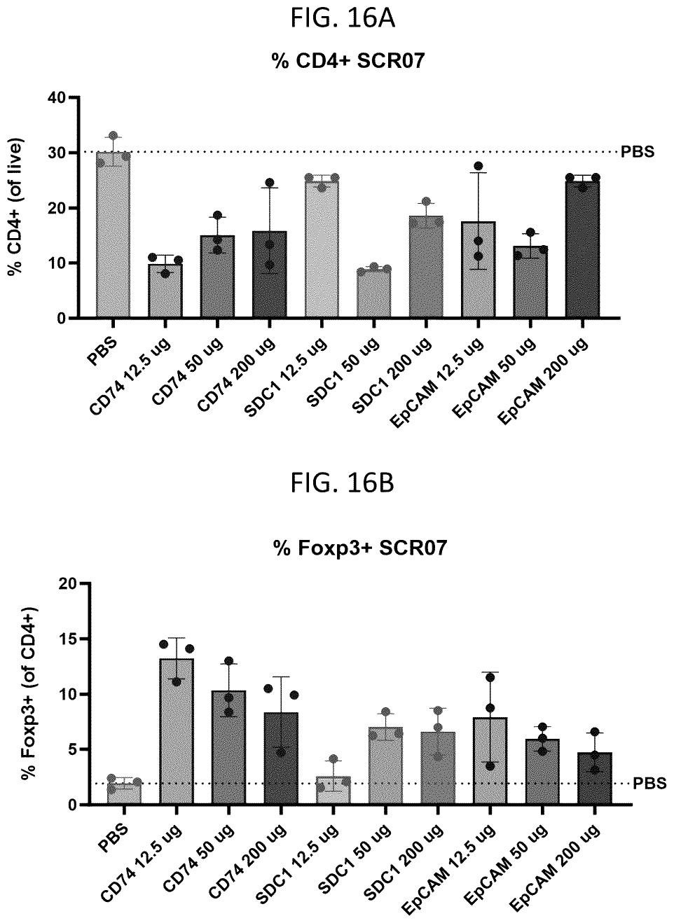

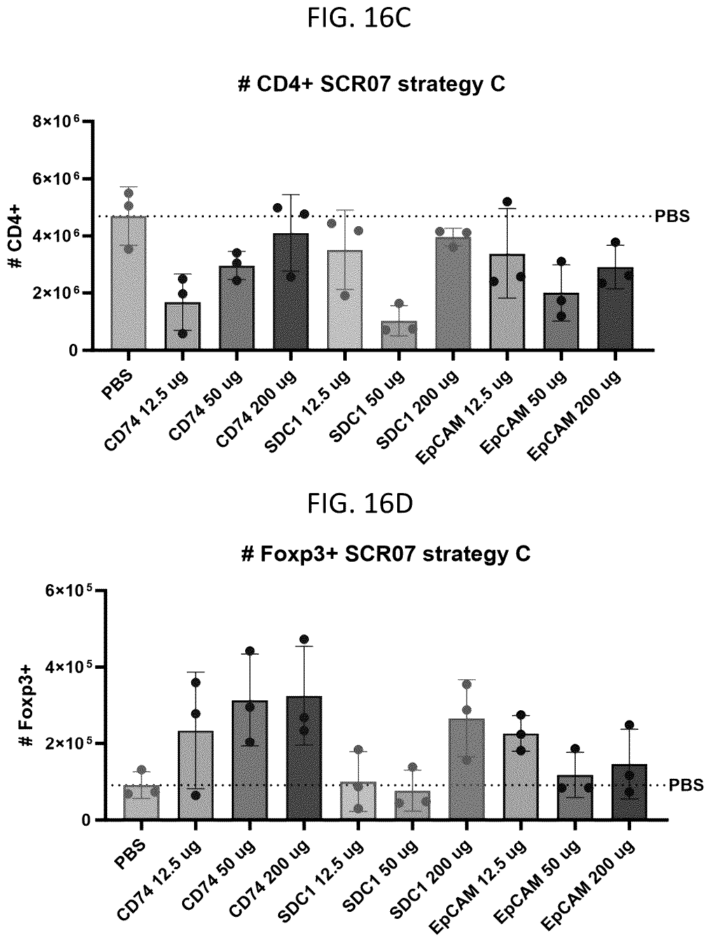

Example 15: Test articles comprising the OVA antigen, an effectorless Fc polypeptide comprising the “LALAPG” mutations as disclosed herein (shown in FIGS. 16A-16D using “*”), and a targeting domain selected from CD74 (ln1/CD74 antibody clone, BioLegend cat. no. 151002), SDC1, or EpCAM (G8.8 antibody clone, BioLegend cat. no. 118211) were administered intravenously into 6-8 week old OT-II female mice at doses of 12.5 ug, 50 ug, and 200 ug of OVA equivalents as shown in FIGS. 16A-16D. Spleens were harvested at 10 days, and splenocytes were stained with a Treg antibody panel. All test articles lead to a dose-dependent increase in proportion of FoxP3+ Tregs, and a dose-dependent increase in absolute number of FoxP3+ Tregs, as compared to control (FIGS. 16B and 16D). The test articles also caused a dose-dependent reduction in population of CD4+ T cells, as shown in FIG. 16A.

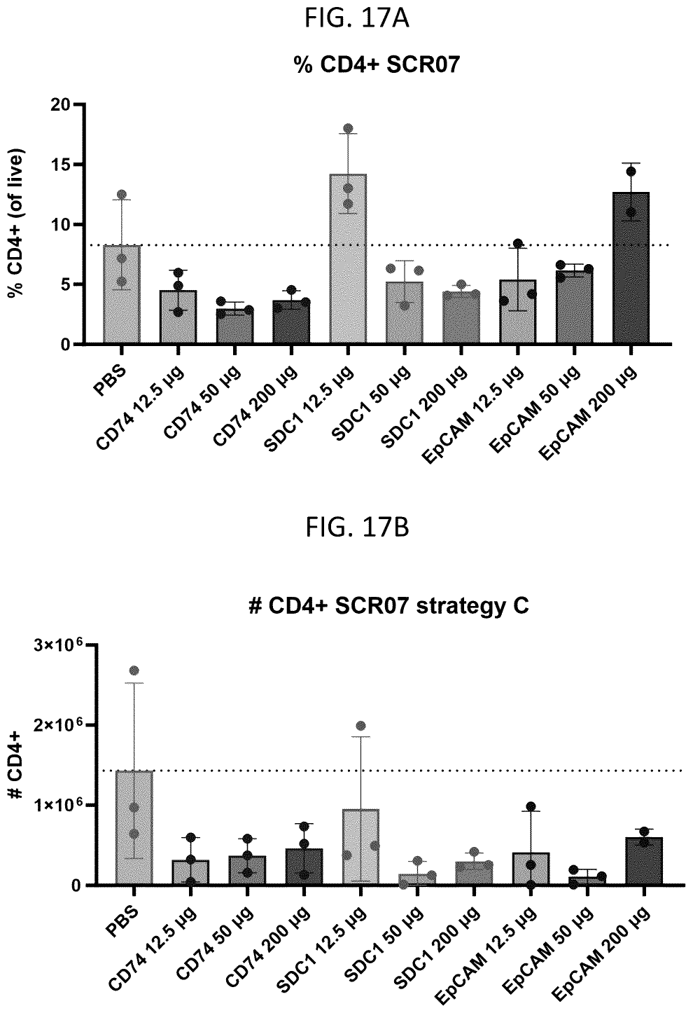

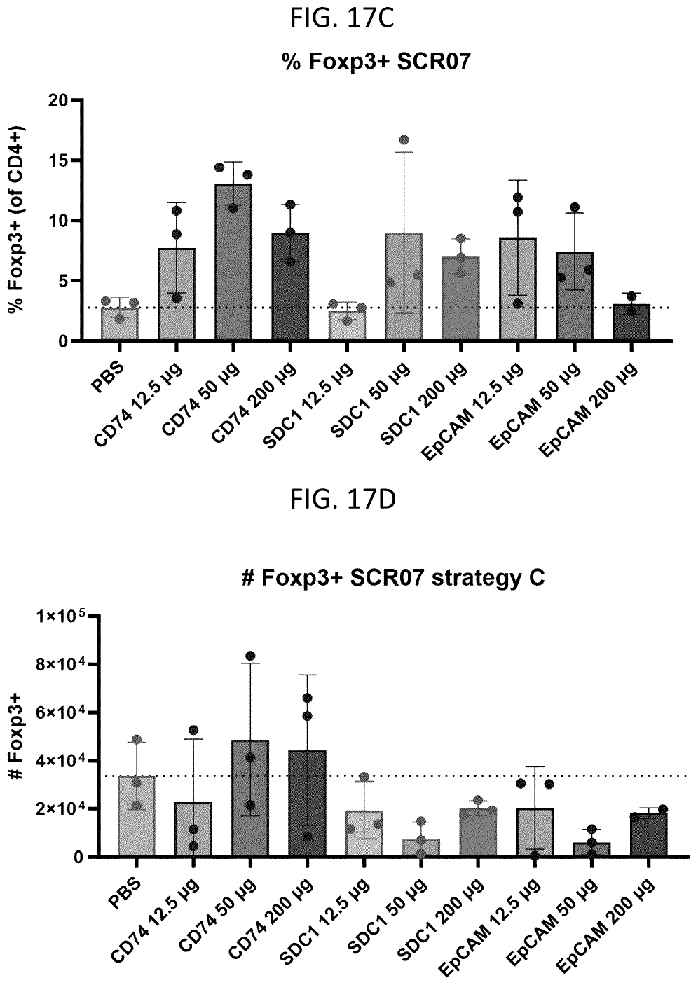

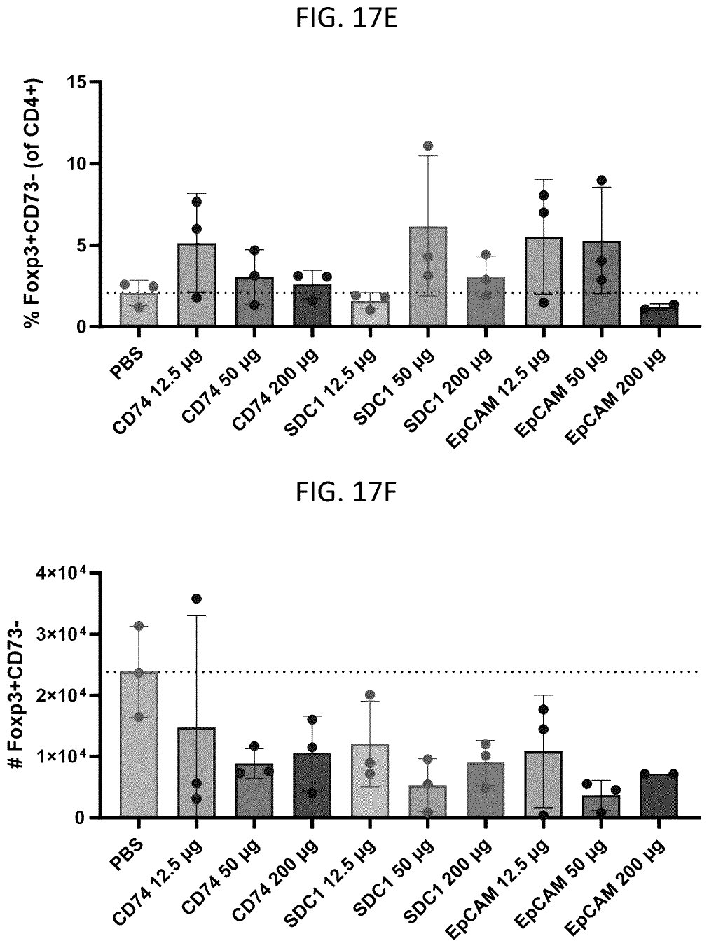

In the same experiment, thymi were harvested at 10 days, and thymocytes were stained with a Treg antibody panel. All test articles lead to a dose-dependent increase in proportion of FoxP3+ Tregs, and a dose-dependent increase in the proportion of FoxP3+CD73− Tregs, as compared to control (FIGS. 17C and 17E). CD74 targeted OVA molecules drove a dose-dependent increase in the absolute number of FoxP3+ Tregs, as compared to control (FIG. 17D). The test articles lead to a dose-dependent reduction in population of FoxP3+CD73− Tregs, as shown in FIG. 17F. SDC1 and EpCAM targeted OVA molecules also lead to an increase in proportion of CD4+ T cells (FIG. 17A).

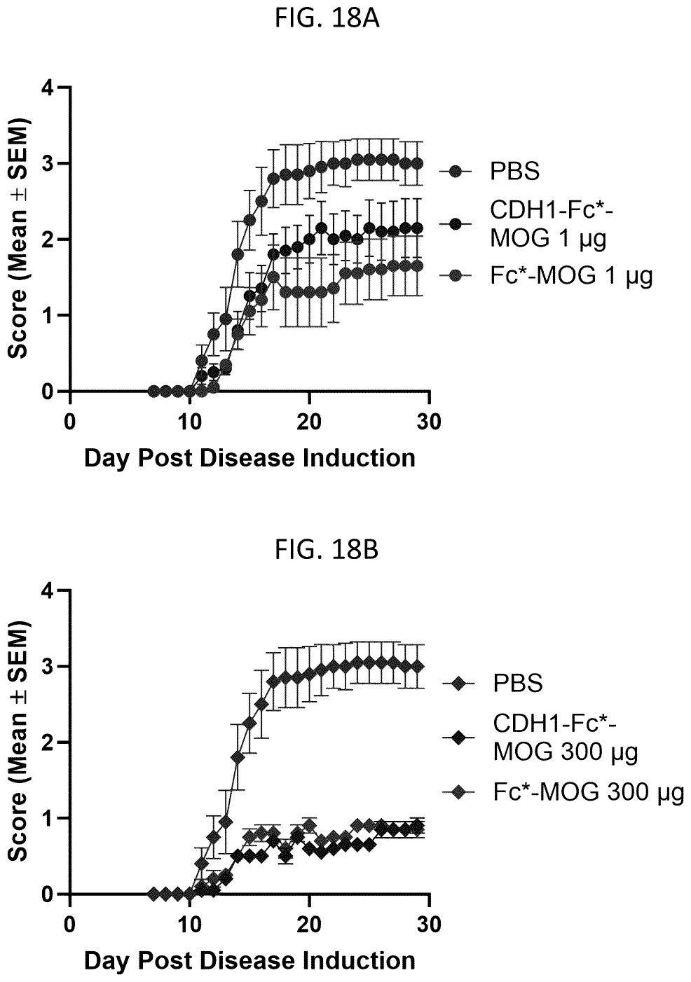

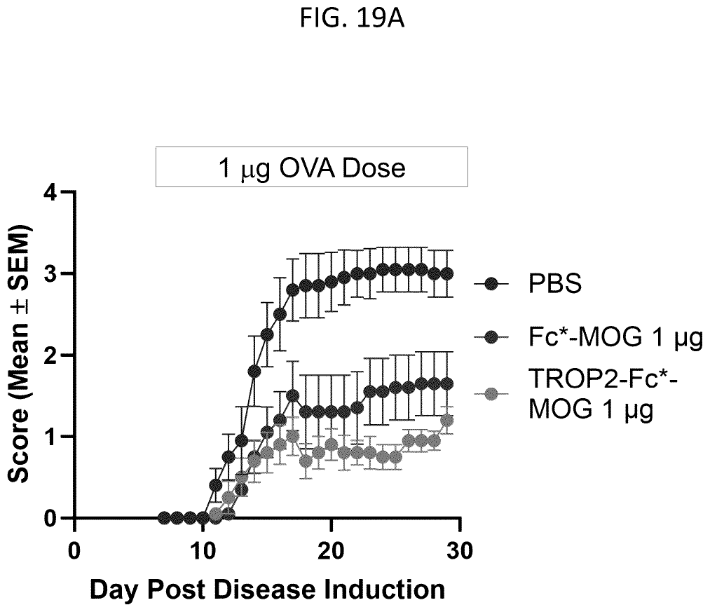

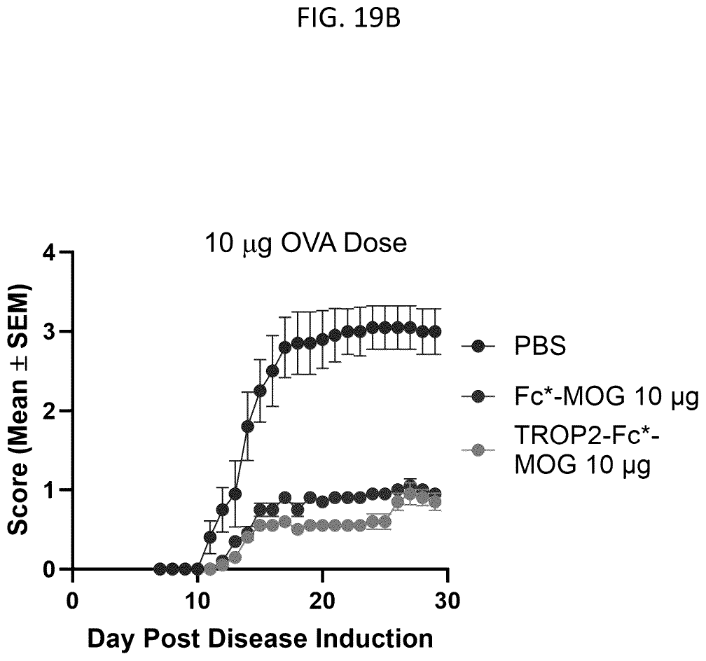

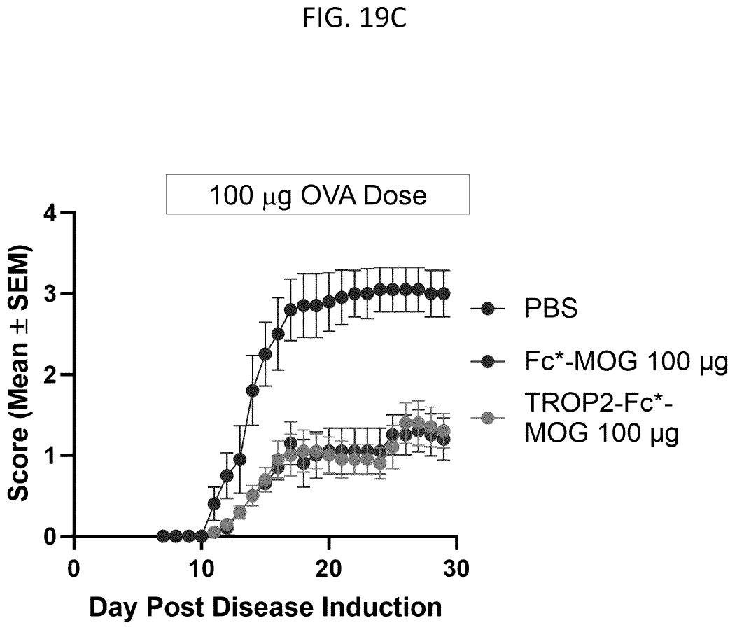

Example 16: Test articles comprising the MOG antigen and the effectorless (“LALAPG” variant) Fc polypeptide; the MOG antigen, the effectorless Fc polypeptide, and the CDH1 cell targeting domain (DECMA-1 antibody clone, ThermoFisher cat. no. 14-3249-82); or the MOG antigen, the effectorless Fc polypeptide, and the TROP2 cell targeting domain (Sp293 antibody clone, Abcam cat. no. ab310311) were administered intravenously at doses specified in the table below into 7 week old C57Bl/6 female mice. Next, EAE was induced in said mice by administering MOG35-55 and 85 ng PTX at 1 week following administration of test articles. CDH1 targeted MOG molecules with the effectorless Fc polypeptide reduced the EAE score in a dose-dependent manner, as shown in FIGS. 18A and 18B. TROP2 targeted MOG molecules with the effectorless Fc polypeptide showed even better reduction the EAE score in a dose-dependent manner than non-targeted MOG, as shown in FIGS. 19A-19C.

| | | |
|---|---|---|
| Test Article | Dose | n |

| | | |
|---|---|---|
| PBS | N/A | 10 |

| | | | |
|---|---|---|---|
| Fc*-MOG | 1 | μg | 10 |
| Fc*-MOG | 10 | μg | 10 |
| Fc*-MOG | 100 | μg | 10 |
| Fc*-MOG | 300 | μg | 10 |
| CDH1-Fc*-MOG | 1 | μg | 10 |
| CDH1-Fc*-MOG | 300 | μg | 10 |
| TROP2-Fc*-MOG | 1 | μg | 10 |
| TROP2-Fc*-MOG | 10 | μg | 10 |
| TROP2-Fc*-MOG | 100 | μg | 10 |

The disclosures of each and every patent, patent application, and publication cited herein are hereby incorporated herein by reference in their entirety. While various embodiments have been disclosed with reference to specific aspects, it is apparent that other aspects and variations of these embodiments may be devised by others skilled in the art without departing from the true spirit and scope of the embodiments. The appended claims are intended to be construed to include all such aspects and equivalent variations.

1. A molecule for inducing antigen-specific thymic Tregs against an antigen of interest by systemic administration, wherein the molecule comprises a thymic cell targeting domain linked to the antigen of interest or a domain that binds to the antigen of interest.

2. A molecule comprising a cell targeting domain linked to an antigen of interest or a domain that binds to the antigen of interest, wherein the cell targeting domain binds to a thymic cell.

3. (canceled)

4. A molecule for inducing tolerance against an antigen of interest, wherein the molecule comprises a cell targeting domain linked to the antigen of interest or a domain that binds to the antigen of interest, wherein the targeting domain binds to a thymic cell, a splenic cell, a dendritic cell, or a B cell.

5-6. (canceled)

7. The molecule of claim 1, wherein the cell targeting domain binds to Trop2, macrophage receptor MARCO, cadherin-1 (CDH1), Trop2, CD74, EpCAM, syndecan 1 (SDC1), PlgR, CD23 antigen (FCER2), CD166 antigen, OX-2 membrane glycoprotein, T-lymphocyte activation antigen CD86, RANK, CCL19, CCL21, LTβR, MHCII, CD40, CD80, CD86, lymphocyte antigen 6D, L1CAM, LIFR, integrin alpha-6, integrin beta-4, podoplanin, CCR7, CD24, CaSR, CD63, nAChRalpha1, CD70, acetylcholine receptor subunit delta, claudin-1, CD80, claudin-4, CD81, claudin-6, CD9, immunoglobulin superfamily member 1, large neutral amino acids transporter small subunit 2, cadherin-2, somatostatin receptor type 2, C-type lectin domain family 7 (CLEC7A), TRIC-A, HER3, IL-20RB, lymphocyte antigen 6D, DEC-205, LY6/PLAUR domain containing 3, oncostatin-M-specific receptor subunit beta, syndecan 1, syndecan-4, HAI-2, sialic acid-binding Ig-like lectin 10 (SIGLEC10), milk fat globule EGF and factor V/VIII domain containing (MFGE8), growth arrest-specific gene 6 (GAS6), or T-cell membrane protein 4 (TIM4).

8. The molecule of claim 1, wherein the thymic cell is a medullary thymic epithelial cell (mTEC), a mimetic mTEC, an AIRE+ mTEC, an AIRE-mTEC, a mTEC progenitor cell, a mimetic mTEC progenitor cell, a junction thymic epithelial cell (jTEC), a thymic progenitor epithelial cell (TPEC), a proliferating TPEC, a thymic epithelial cell (TEC), a proliferating TEC, a cortical thymic epithelial cell (cTEC), a proliferating cTEC, or a proliferating mTEC.

9. The molecule of claim 8, wherein the mimetic mTEC is a basal (skin/lung) mTEC, an enterocyte/hepatocyte mTEC, a ciliated mTEC, an ionocyte mTEC, a keratinocyte mTEC, a microfold mTEC, a muscle mTEC, a neuroendocrine mTEC, a parathyroid mTEC, a secretory mTEC, a thyroid mTEC, or a tuft mTEC.

10-11. (canceled)

12. The molecule of claim 1, wherein the cell targeting domain is a polypeptide, such as an antibody, or an antigen-binding fragment thereof.

13. (canceled)

14. The molecule of claim 1, wherein the cell targeting domain binds to a cell membrane surface protein or glycoprotein on the surface of the thymic cell, splenic cell, dendritic cell, or B cell.

15. The molecule of claim 14, wherein the cell membrane surface protein is cadherin-1 (CDH1), Trop2, CD74, EpCAM, syndecan 1 (SDC1), PlgR, CD23 antigen (FCER2), macrophage receptor MARCO, C-type lectin domain family 7 (CLEC7A), CD24, CD70, sialic acid-binding Ig-like lectin 10 (SIGLEC10), milk fat globule EGF and factor V/VIII domain containing (MFGE8), EGF like repeats and discoidin domains 3 (EDIL3), growth arrest-specific gene 6 (GAS6), or T-cell membrane protein 4 (TIM4).

16. The molecule of claim 1, wherein the cell targeting domain is an antibody that is: an anti-cadherin-1 (CDH1) antibody, or antigen-binding fragment thereof; an anti-Trop2 antibody, or antigen-binding fragment thereof; an anti-CD74 antibody, or antigen-binding fragment thereof; an anti-EpCAM antibody, or antigen-binding fragment thereof; an anti-syndecan 1 antibody, or antigen-binding fragment thereof; an anti-PlgR antibody, or antigen-binding fragment thereof; an anti-CD23 antigen (FCER2) antibody, or antigen-binding fragment thereof; an anti-macrophage receptor MARCO antibody, or antigen-binding fragment thereof; an anti-sialic acid-binding Ig-like lectin 10 (SIGLEC10) antibody, or antigen-binding fragment thereof; an anti-milk fat globule EGF and factor V/VIII domain containing (MFGE8) antibody, or antigen-binding fragment thereof; an anti-growth arrest-specific gene 6 (GAS6) antibody, or antigen-binding fragment thereof; an anti-T-cell membrane protein 4 (TIM4) antibody, or antigen-binding fragment thereof; an anti-CD166 antigen antibody, or antigen-binding fragment thereof; an anti-OX-2 membrane glycoprotein antibody, or antigen-binding fragment thereof; an anti-T-lymphocyte activation antigen CD86 antibody, or antigen-binding fragment thereof; an anti-RANK antibody, or antigen-binding fragment thereof; an anti-CCL19 antibody, or antigen-binding fragment thereof; an anti-CCL21 antibody, or antigen-binding fragment thereof; an anti-LTβR antibody, or antigen-binding fragment thereof; an anti-MHCII antibody, or antigen-binding fragment thereof; an anti-CD40 antibody, or antigen-binding fragment thereof; an anti-CD80 antibody, or antigen-binding fragment thereof; an anti-CD86 antibody, or antigen-binding fragment thereof; an anti-lymphocyte antigen 6D antibody, or antigen-binding fragment thereof; an anti-LiCAM antibody, or antigen-binding fragment thereof; an anti-LIFR antibody, or antigen-binding fragment thereof; an anti-integrin alpha-6 antibody, or antigen-binding fragment thereof; an anti-integrin beta-4 antibody, or antigen-binding fragment thereof; an anti-podoplanin antibody, or antigen-binding fragment thereof; an anti-CCR7 antibody, or antigen-binding fragment thereof; an anti-CD24 antibody, or antigen-binding fragment thereof; an anti-CaSR antibody, or antigen-binding fragment thereof; an anti-CD63 antibody, or antigen-binding fragment thereof; an anti-nAChRalpha1 antibody, or antigen-binding fragment thereof; an anti-CD70 antibody, or antigen-binding fragment thereof; an anti-acetylcholine receptor subunit delta antibody, or antigen-binding fragment thereof; an anti-claudin-1 antibody, or antigen-binding fragment thereof; an anti-CD80 antibody, or antigen-binding fragment thereof; an anti-claudin-4 antibody, or antigen-binding fragment thereof; an anti-CD81 antibody, or antigen-binding fragment thereof; an anti-claudin-6 antibody, or antigen-binding fragment thereof; an anti-CD9 antibody, or antigen-binding fragment thereof; an anti-immunoglobulin superfamily member 1 antibody, or antigen-binding fragment thereof; an anti-cadherin-1 antibody, or antigen-binding fragment thereof; an anti-large neutral amino acids transporter small subunit 2 antibody, or antigen-binding fragment thereof; an anti-cadherin-2 antibody, or antigen-binding fragment thereof; an anti-somatostatin receptor type 2 antibody, or antigen-binding fragment thereof; an anti-dectin-1 antibody, or antigen-binding fragment thereof; an anti-TRIC-A antibody, or antigen-binding fragment thereof; an anti-HER3 antibody, or antigen-binding fragment thereof; an anti-IL-20RB antibody, or antigen-binding fragment thereof; an anti-lymphocyte antigen 6D antibody, or antigen-binding fragment thereof; an anti-DEC-205 antibody, or antigen-binding fragment thereof; an anti-LY6/PLAUR domain containing 3 antibody, or antigen-binding fragment thereof; an anti-oncostatin-M-specific receptor subunit beta antibody, or antigen-binding fragment thereof; an anti-syndecan-4 antibody, or antigen-binding fragment thereof; an anti-HAI-2 antibody, or antigen-binding fragment thereof.

17. The molecule of claim 1, wherein cell targeting domain is a ligand that is from CD6, cell surface glycoprotein CD200 receptor 1, P-selectin, sialic acid-binding Ig-like lectin 10, metalloproteinase inhibitor 1, lysosome-associated membrane glycoprotein 1, lysosome-associated membrane glycoprotein 2, integrin beta-1, CD27 antigen, macrophage migration inhibitory factor, CD44 antigen, cytotoxic T-lymphocyte protein 4, programmed cell death 1 ligand 1, T-cell-specific surface glycoprotein CD28, T-lymphocyte activation antigen CD86, complement receptor type 2, prostaglandin F2 receptor negative regulator, immunoglobulin superfamily member 8, integrin beta-1, B-lymphocyte antigen CD19, CD44 antigen, T-lymphocyte activation antigen CD80, T-cell-specific surface glycoprotein CD28, cytotoxic T-lymphocyte protein 4, membrane cofactor protein, proheparin-binding EGF-like growth factor, immunoglobulin superfamily member 8, prostaglandin F2 receptor negative regulator, integrin alpha-3, integrin alpha-6, integrin beta-1, epidermal growth factor receptor, integrin alpha-E, killer cell lectin-like receptor subfamily G member 1, hepatocyte growth factor receptor, cadherin-2, fibroblast growth factor receptor 1, low-density lipoprotein receptor-related protein 5, integrin beta-4, epithelial cell adhesion molecule, cadherin-1, pro-neuregulin-1, pro-neuregulin-2, complement receptor type 2, integrin alpha-X, integrin beta-2, neurogenic locus notch homolog protein 2, interleukin-19, interleukin-20, interleukin-24, receptor-type tyrosine-protein phosphatase C, anterior gradient protein 2 homolog, secretoglobin family 3A member 2, cardiotrophin-1, leukemia inhibitory factor, oncostatin-M, kappa-casein, fibroblast growth factor 2, extracellular glycoprotein lacritin, fibroblast growth factor 2, fibroblast growth factor 2, hepatocyte growth factor, hepatocyte growth factor activator, trypsin-3, prostasin, suppressor of tumorigenicity 14 protein, or transmembrane protease serine 13, or a liganding binding partner fragment thereof of any of the foregoing.

18. The molecule of claim 1, wherein the antigen of interest is an antibody, or antigen-binding fragment thereof; an antigen, or a fragment thereof; an auto-antigen, or a fragment thereof; a self-antigen, or a fragment thereof; a tolerance inducing antigen, or a fragment thereof; a ligand, or a fragment thereof; or an effector peptide or molecule, such as an allergen; and wherein the antigen of interest is an antigen that induces an autoimmune, alloimmune, allergic, inflammatory, or anti-drug immune response.

19. (canceled)

20. The molecule of claim 18, wherein the autoimmune, alloimmune, allergic, inflammatory, or anti-drug immune response is a disorder or disease selected from the group consisting of: diabetes mellitus type 1, ulcerative colitis, Crohn's disease, celiac disease, systemic sclerosis, graft versus host disease (GVHD), rheumatoid arthritis, anti-AAV immune response, asthma, severe asthma, neutrophilic asthma, paucigranulocytic asthma, eosinophilic asthma, osteoarthritis, solid organ transplant, atopic dermatitis, eosinophilic esophagitis, dermatitis herpitoformis, chronic rhinosinusitis, chronic spontaneous urticaria, food allergy, anti-biologic drug immune response, anti-viral immune response, anti-adenoviral immune response, anti-retroviral immune response, anti-lentiviral immune response, and -VLP immune response, Alzheimer's disease, amyotrophic lateral sclerosis (ALS), Parkinson's disease, dementia with Lewy bodies, Duchenne muscular dystrophy, cutaneous lupus myocarditis, postmyocardial infarction syndrome, postpericardiotomy syndrome, subacute bacterial endocarditis, anti-glomerular basement membrane nephritis, interstitial cystitis, lupus nephritis, membranous glomerulonephropathy, chronic kidney disease (“CKD”), autoimmune hepatitis, primary biliary cirrhosis, primary sclerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease (LAD), morphea, pemphigus, pemphigus vulgaris, hidradenitis suppuritiva, pityriasis lichenoides et varioliformis acuta, Mucha-Habermann disease, psoriasis, guttate psoriasis, erythrodermic psoriasis, plaque psoriasis, pustular psoriasis, skin manifestation associated with systemic sclerosis, vitiligo, Addison's disease, autoimmune polyendocrine syndrome (APS) type 1, autoimmune polyendocrine syndrome (APS) type 2, autoimmune polyendocrine syndrome (APS) type 3, autoimmune pancreatitis (AIP), autoimmune thyroiditis, Ord's thyroiditis, Graves' disease, non radiographic axial spondyloarthropathy, autoimmune oophoritis, endometriosis, autoimmune orchitis, Sjogren's syndrome, dermatitis herpatiformis, autoimmune enteropathy, coeliac disease, immune related adverse events (irAEs) secondary to immune checkpoint inhibitors, microscopic colitis, autoimmune thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, sarcoidosis, Schnitzler syndrome, systemic lupus erythematosus (SLE), undifferentiated connective tissue disease (UCTD), dermatomyositis, fibromyalgia, inclusion body myositis, myositis, myasthenia gravis, neuromyotonia, paraneoplastic cerebellar degeneration, polymyositis, acute disseminated encephalomyelitis (ADEM), acute motor axonal neuropathy, anti-N-methyl-D-aspartate (anti-NMDA) receptor encephalitis, balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, oshtoran syndrome, pediatric autoimmune neuropsychiatric disorder associated with streptococcus (PANDAS), progressive inflammatory neuropathy, restless leg syndrome, stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Ménière's disease, Behcet's disease, eosinophilic granulomatosis with polyangiitis (EGPA), giant cell arteritis, granulmatosis with polyangiitis (GPA), IgA vasculitis (IgAV), Kawasaki's disease, leukocytoclastic vasculitis, lupus vasculitis, rheumatoid vasculitis, microscopic polyangiitis (MPA), polyarteritis nodosa (PAN), polymyalgia rheumaticia, vasculitis, primary immune deficiency, Gaucher disease, Pompe disease, Fabry disease, Schindler disease, Hurler syndrome (MPS1), Tay-Sachs disease, Nieman-Pick disease, Farber lipogranulomatosis, Krabbe disease, Morquio syndrome (MPS4), mucopolysaccharidosis type VI (MPS6), Hunter syndrome (MPS2), Sanfilippo syndrome (MPS3), Sly sundrome (MPS7), hyaluronidase deficiency (MPS9), mucolipidosis type 1 (ML1), I-cell disease, mucolipidosis II (ML2), mucolipidosis III gamma (ML3C), glycogen storage disease Ia, glycogen storage disease Tb, Wilson disease, phenylketonuria, Stargardt disease type I, myocilin-associated glaucoma, hemophilia A, hemophilia B, hereditary or acquired thrombotic thrombocytopathic purpura (TTP), von Willebrand disease, thrombocytopenia platelet autoantigen, heparin-induced thrombocytopenia, tolerance induction for AAV gene therapy, tolerance induction for ex vivo gene editing, tolerance induction for in vivo gene editing, acquired hemophilia, antiphospholipid syndrome, aplastic anemia, autoimmune lymphoproliferative syndrome, autoimmune neutropenia, autoimmune thrombocytopenic purpura, cold agglutinin disease, essential mixed cryoglobulinemia, Evans syndrome, IgG4-related diseases, pure red cell aplasia, warm autoimmune hemolytic anemia, autoimmune gastritis, pernicious anemia, anti-sperm antibodies, autoimmune polyendocrine syndrome type 1, autoimmune polyendocrine syndrome type 2, autoimmune polyendocrine syndrome type 3, autoimmune myocarditis, cryptogenic organizing pneumonia, idiopathic pulmonary fibrosis, IgA nephropathy, post-myocardial infarction syndrome, primary biliary cholangitis, primary idiopathic dilated cardiomyopathy, anti-N-methyl-D-aspartate receptor encephalitis, autoimmune encephalitis, encephalopathy associated with autoimmune thyroid disease, episcleritis, Graves' ophthalmopathy, myelin oligodendrocyte glycoprotein disease, narcolepsy with cataplexy, cutaneous lupus erythematosus, adult-onset Still's disease, ankylosing spondylitis, juvenile arthritis, juvenile dermatomyositis, anti-neutrophil cytoplasmic antibody-associated vasculitis, polymyalgia rheumatic, purpura rheumatic, Takayasu arteritis, transplant, and xenotransplantation.

21. The molecule of claim 18, wherein the antigen of interest is selected from the group consisting of: glutamate decarboxylase 1 (GAD), glutamate decarboxylase 2 (GAD65), proinsulin, islet-specific glucose-6-phosphatase catalytic subunit-related protein (IGRP), zinc transporter 8 (Znt8), receptor-type tyrosine-protein phosphatase-like N (IA2), tropomyosin alpha-3 chain (TPM3), granulocyte-macrophage colony-stimulating factor (GMCSF/CSF2), RNA polymerase III complex subunit A (RNAP3), RNA polymerase III complex subunit B (RNAP3), RNA polymerase III complex subunit C (RNAP3), RNA polymerase III complex subunit D (RNAP3), RNA polymerase III complex subunit E (RNAP3), RNA polymerase III complex subunit F (RNAP3), RNA polymerase III complex subunit G (RNAP3), RNA polymerase III complex subunit H (RNAP3), RNA polymerase III complex subunit K (RNAP3), DNA topoisomerase 1 (TOPO1), type-1 angiotensin II receptor (AT1R), endothelin-1 receptor (ETAR), major centromere protein A (CENP), major centromere protein B (CENP), major centromere protein C (CENP), tetraspanin-7 (TSPAN7), 21-hydroxylase (21OH), 17alpha-hydroxylase (17OH), Th/To, ANA, dsDNA, HMGCR, myosin, SSA/Ro, SSB/La, U1RNP, U3RNP, PM-Sci, myelin basic protein (MBP), IL17/IL22, ASMA, actin, amylase α2, GM-CSF, cyclic citrullinated proteins (CCP), snRNP, Ro52, Ro60, La, Jo-1, SRP, IFIH1, CENPA, insulin, RNA-P, U1-70K, Sm-D3, MDA-5, PL7, PL-12, centromere proteins, PM/SCL, RNA polymerase 3 complex, tetraspanin-7, peptidylprolyl isomerase like 2, Mlh1, proteinase-3, immunoglobulin G, immunoglobulin M, myeloperoxidase, acetylcholine receptor (AchR), MUSK, LRP4, HSP90, HSPA5, kallikrein 13, ADAMTS13, IFN-γ, IFN-α/ω, IL6, IL12/IL23, desmogleins (e.g., desmoglein-3, desmoglein-1), aquaporin-4, myelin oligodendrocyte glycoprotein (MOG), GRIN1, GRIA1, GRIA2, leucine rich glioma inactivated 1 protein (LGI1), neurexin family protein contactin associated protein 2 (CNTNAP2), collagen (e.g., collagen IV-alpha 3 chain (COL4A3), COL17), BP180, phospholipase A2 receptor (PLA2R), thrombospondin type-1 domain-containing 7A (THSD7A), thyroid hormone stimulating receptor (THSR), NMDAR, FGF23, acid beta-glycosidase, acid alpha-glycosidase, alpha-galactosidase A, alpha-N-acetylgalactosaminidase, alpha-L-iduronohydrolase, beta-hexosaminidase A, sphingomyelin phosphodiesterase 1, N-acetylsphingosine amidohydrolase 1, galactocerebrosidase, N-acetylgalactosamine-sulfate sulfatase, arylsulfatase B, iduronate 2-sulfatase, heparan-alpha-glucosaminide-N-acetyltransferase, beta-glucuronidase, hyaluronidase, sialidase 1, N-acetylglucosamine-1-phosphotransferase alpha/beta, N-acetylglucosamine-1-phosphotransferase gamma, glucose-6-phosphatase catalytic subunit, glucose-6-phosphate transporter, ATP7B, Phenylalanine hydroxylase, ATP-binding cassette subfamily A member 4, myocilin, coagulation factor VIII, coagulation factor IX, von Willebrand factor, glycoprotein Ib alpha, glycoprotein Ib beta, glycoprotein V, glycoprotein VI, glycoprotein IX, glycoprotein IIb, glycoprotein IIIa, platelet factor 4, AAV capsid protein (such as AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.eB, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9, AAV9 K449R (or K449R AAV9), AAV9.16, AAV9.24, AAV9.45, AAbiodisV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV 114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74 (also referred to as AAVrh74), AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, 7m8, Spark100, AAVMYO and variants thereof), SpCas9, SaCas9, FnCas12a, LbCas12a, AcCas12a, Cas13b, APOBEC1, E. coli TadA, Moloney Murine Leukemia Virus Reverse Transcriptase (M-MLV RT), HLA-A, HLA-B, HLA-C, HLA-E, HLA-L, HLA-J, HLA-K, HLA-H, HLA-G, HLA-DM, HLA-DO, HLA-DP, HLA-DQ, and HLA-DR.

22. The molecule of claim 1, wherein the antigen of interest has an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence selected from any one of SEQ ID NO: 14, 15, 16, 17, 18, 19, 20, 21, 22, 23, 24, 25, 26, 27, 28, 29, 30, 31, 32, 33, 34, 35, 36, or 37.

23-28. (canceled)

29. The molecule of claim 1, wherein the molecule further comprises an Fc polypeptide domain, wherein:
the cell targeting domain is conjugated to the N-terminus of an Fc polypeptide domain, and the antigen of interest, or a domain that binds to the antigen of interest, is conjugated to a C-terminus of the Fc polypeptide domain; or
the cell targeting domain is conjugated to the C-terminus of a Fc polypeptide domain, and the antigen of interest, or a domain that binds to the antigen of interest, is conjugated to the N-terminus of the Fc polypeptide domain, and
wherein the cell targeting domain and the antigen of interest, or the domain that binds to the antigen of interest, are linked by a linker, such as a peptide linker.

30. The molecule of claim 29, wherein the Fc polypeptide comprises an IgG1, IgG2, IgG3, IgG4 Fc polypeptide domain comprising any one, or any combination of L234A, L235A, L234F, L235E, P329G, P331S, N297A, N297G, N297Q, G236A, A330S, S239D, I332E, S267E, H268F, S324T, Y296W, T299A, V308P, H310A, R409K, Y435H, T307A, T309A, T309K, K322A, K326W, K334W, K326A, K334A, G237A, P238S, and H268A mutations, wherein the positions are according to EU numbering.

31-89. (canceled)

90. A method of inducing immune tolerance against an antigen of interest, such as T cell tolerance to the antigen of interest in a subject, the method comprising administering to the subject the molecule of claim 2, or the pharmaceutical composition comprising the same.

91-118. (canceled)

119. The molecule of claim 2, wherein the cell targeting domain binds to cadherin-1 (CDH1), Trop2, CD74, EpCAM, syndecan 1 (SDC1), PlgR, CD23 antigen (FCER2), macrophage receptor MARCO, C-type lectin domain family 7 (CLEC7A), CD24, CD70, sialic acid-binding Ig-like lectin 10 (SIGLEC10), milk fat globule EGF and factor V/VIII domain containing (MFGE8), EGF like repeats and discoidin domains 3 (EDIL3), growth arrest-specific gene 6 (GAS6), or T-cell membrane protein 4 (TIM4).

120. The molecule of claim 4, wherein the cell targeting domain binds to cadherin-1 (CDH1), Trop2, CD74, EpCAM, syndecan 1 (SDC1), PlgR, CD23 antigen (FCER2), macrophage receptor MARCO, C-type lectin domain family 7 (CLEC7A), CD24, CD70, sialic acid-binding Ig-like lectin 10 (SIGLEC10), milk fat globule EGF and factor V/VIII domain containing (MFGE8), EGF like repeats and discoidin domains 3 (EDIL3), growth arrest-specific gene 6 (GAS6), or T-cell membrane protein 4 (TIM4).
